SEQUENCE LISTING (192 sequences)

SEQ ID NO: 1 - protein, 336 aa, Agrotis segetum
MAQGVQTTTILREEEPSLTFVVPQEPRKYQIVYPNLITFGYWHIAGLYGLYLCFTSAKWQTILFSFMLVVLAELGITAGAHRLWAHKTYKAKLPLQIILMILNSIAFQNSAIDWVRDHRLHHKYSDTDADPHNATRGFFYSHVGWLLVRKHPEVKRRGKELDMSDIYNNPVLRFQKKYAIPFIGAMCFGLPTFIPVYFWGETWSNAWHITMLRYILNLNITFLVNSAAHIWGYKPYDIKILPAQNIAVSIVTGGEVSITTTTFFPWDYRAAELGNNYLNLTTKFIDFFAWIGWAYDLKTVSSDVIKSKAERTGDGTNLWGLEDKGEEDFLKIWKDN

SEQ ID NO: 2 - protein, 326 aa, Amyelois transitella
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 3 - protein, 351 aa, Amyelois transitella
MPPQGQDRESWVLYETDDKTQDGGTHVVPPSAEKRVWKIVWRNVIAFALLHIGGVYGAYLFLFKAMWLTDLFAVFLYLCSGLGVTAGAHRLWAHKSYKARLPLRLLLTVFNTIAFQDAVIDWARDHRLHHKYSETDADPHNATRGFFFSHIGWLLVRKHPQIKEKGPTIDLNDLRADPVLHFQKKYYLYLMPLACFVMPTLVPTLWGESLWNAYFVCAIFRYIYVLNVTWLVNSAAHAWGSKPYDKNINPVETKPVSLVVLGEGFHNYHHTFPWDYKTAELGDYSLNFSKLFIDAMAKIGWAYDLKTVSPDVIEKRVKRTGDGSHHVWGWDDKDVPVEEKEEATIFNPSKD

SEQ ID NO: 4 - protein, 352 aa, Amyelois transitella
MAPNATDANGVLFETDAATPDLALPHAPVQQADNYPKKYVWRNIILFAYLHIAALYGGYLFLFHAKWQTDIFAYILYVMSGLGITAGAHRLWAHKSYKAKWPLRLILVIFNTLAFQDSAIDWSRDHRMHHKYSETDADPHNATRGFFFSHIGWLLVRKHPELKRKGKGLDLSDLYADPILRFQKKYYLILMPLTCFVLPTVIPVYYWGETWTNAFFVAALFRYAFILNVTWLVNSAAHKWGDKPYDRNIKPSENISVSMFALGEGFHNYHHTFPWDYKTAELGNNMLNFTTNFINFFAKIGWAYDLKTVSDEIVRSRAKRTGDGSHHLWGWGDKDHSREEMAAAIRIHPKDD

SEQ ID NO: 5 - protein, 349 aa, Chauliognathus lugubris
MAPNSNDATGVLQETDDDVSSNQVLQQITKSEKSKLIIVWSNVMYFVILHVGALYGLWLLLTSAQIWTCLWVFAMYEFGEICITAGVHRLWSHRSYKAKWPLRLFHTMGQTLAFQDAVVDWARDHRVHHKYSETDADPHNAKRGFFFSHMGWLMCRKSKQVKEKGKEPDISDLYADPILRYQKKYYMLFMPLMCFAFPTVVPLYFWNESLKTAFFVNIFRYIFSLHATWLVNSAAHLYGEKPYNKHINPAENLAVSLIVNGERWHNYHHTFPWDYKAGEFGRYGTNLTTVFINAMAKIGLAYDLKFVPEDVVKRRVHKTGDGSHAVWGWGDKDQTVEEISKTIVAYNQS

SEQ ID NO: 6 - protein, 371 aa, Chilo supprealis
MDFESELQNCSNKSQKRNIMAPNASDVNGVLFEDDAATPDMALSNTPVQKADNYPKKLVWRNIIAFAYLHIAAVYGGYLFLFAAKWQTDIFAYILYVMSGLGITAGAHRLWAHKSYKAKWPLRLILVFFNTLAFQDSAIDWARDHRMHHKYSETDADPHNATRGFFFSHIGWLLVRKHPELKQKGKGLDLSDLYADPILRFQKKYYLILMPIACFILPTVIPLYYWGENWVTAFFVAALFRYAFILNVTWLVNSAAHKWGDKPYDKNIKPSENISVSIFALGEGFHNYHHTFPWDYKTAELGNNRLNFTTNFINFFAKIGWAYDMKTVSDEIVQSRVKRTGDGSHHLWGWGDKDHCKEEVAAAIRINPKDD

SEQ ID NO: 7 - protein, 334 aa, Choristoneura parallela
MAPNVEDMESDMPESEKWEKLVAPQAAPRKYEIIYTNLLTFGYGHIAGLYGLYLCFTSAKWQTVILAIILNEMAILGITAGAHRLWSHRSYKAAVPLQIILMIFNSLAFQNSAINWVRDHRMHHKYSDTDGDPHNASRGFFYSHVGWLLVKKHPEVKKRGKMIDMSDIYSNPVLRFQKKYAIPFIGMICFVLPTIIPMYFWGETLSNAWHITMLRYVFSLNSIFLVNSAAHLYGYRPYDKNILPAENKIALIACLGDSFHNYHHVFPWDYRASELGNIGMNWTAQFIDFFAWIGWAYDLKTASDENINSRMKRTGDGTDISGQKYSCESSEVLQ

SEQ ID NO: 8 - protein, 335 aa, Choristoneura rosaceana
MAPNVEDMESDLPESEEKLEKLVAPQAAPRKYQIIYTNLLTFGYWHIAGLYGLYLCFTSAKWQTIILALILNEMAILGITAGAHRLWAHRSYKATVPLQIILIIFNSLSFQNSAIHWIRDHRMHHKYSDTDGDPHNASRGFFYSHVGWLLVKKHPEVKKRAKTIDMSDIYSNPILRFQKKYAIPFIGMICFVLPTIIPMYFWGETLSNAWHITMLRYVFSLNSIFLVNSAAHLYGYRPYDKNILPAENKMTFIACLGENFHNYHHVFPWDYRASELGNIGMNWTAKFIDFFAWIGWAYDLKTASDENIKSRMKRTGDGTDVSGQKYSCESSEVLQ

SEQ ID NO: 9 - protein, 348 aa, Cydia pomonella
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKN

SEQ ID NO: 10 - protein, 349 aa, Cydia pomonella
MPPQGQPPAWVLDESDAVTEDKDVATPAPEAEKRKLQIVWRNVTLFVFLHIGALYGGYLFFTKAMWTTRIFTVLLYIMSGLGITAGAHRLWAHKSYKARLPLRLLLTLFNTIAFQDSVLDWARDHRMHHKYSETDADPHNATRGFFFSHVGWLLVRKHPQIKAKGHTIDMSDLLADPVLRFQKKYYLTLMPLCCFILPSYIPTLWGESLWNAYFVCAIFRYCYVLNVTWLVNSAAHKWGDRPYDKNINPVETKPVSLVVFGEGFHNYHHTFPWDYKTAELGGYSLNLSKLFIDTMSKIGWAYDLKSVSPDIVEKRVKRTGDGSHHVWGWDDAPSEQKVAATIVNPDKTE

SEQ ID NO: 11 - protein, 334 aa, Cydia pomonella
MAPYSEEYEILKENTKPVSPQAAPREYTVVYSVVLIFVYWHIGALYGLYLGFTSAKWATIIFNYLIYVSGGFAITAGSHRLWSHRAFKAKLPLQILLMLLQTMSCQKSVLNWVRDHRLHHMYCDTDADPYNSTRGIFYSHIGWLMVKKHPEVIRKGRTIDMSDLENNPVLKFQKKFYPILVTLMAFILPALIPVIFWQESLNIAHHVSLVHLVVGSHMTFAINSIAHAFGSKPCDKTISPTQSISLSLVTFGEGYHNYHHVFPFDYRVAELGNNYLNLTTNFIDFFAWIGWAYDLKYASPDMVAKRAKRTGDGTDLWGRAIEHADIQAKRVHPS

SEQ ID NO: 12 - protein, 352 aa, Dendrolimus punctatus
MAPKEADVNGVLFESDATTPDMALPTTPVQQADNYPKKLVWRNILLFAYLHLAALYGGYLFLFSAKWQTDIFAYILYIMSGLGITAGAHRLWAHKSYKAKWPLRLILVLFNTLAFQDSAIDWARDHRMHHKYSETDADPHNATRGFFFSHIGWLLVRKHPELKKKGKGLDISDLYADPILRFQKKYYLLLMPLGCFILPTVIPVYLWNETWSNAFLVAALFRYAVILNVTWLVNSAAHKWGDKPYDKSIKPSENLSVALFALGEGFHNYHHTFPWDYKTAELGNNRLNFTTTFINFFAKIGWAYDMKTVSDEIIQNRVKRTGDGSHHLWGWGDKDHSKEEINAAIRINPKDD

SEQ ID NO: 13 - protein, 416 aa, Drosophila grimshawi
MFSEYYVYRRKLLGISSVQIARQIDRLIKPSTLKMPPNAISSPPDALPTCPEDQVLSGSQSGAGGASKQTGVLFEADADTNDGGLTMDITQFKNAEKRKLKWVWRNIVLFAYVHVAALYGGYLLVTQAKWATIVFSMFLYSAGMIGITGGAHRLWAHRSYKAKWPLRVILVAFNSIAFQDAAYHWARDHRVHHKFSETDADPHNATRGFFFSHVGWLLCKKHPDVVAKGKSLDLSDLRADRVLMFQKKHYFVLMPLACFILPTCIPMLCWNESLLCSVLVPTIFRWCLQLNMTWLVNSAAHKFGGRPYDKNINPSQSPYVSLFTLGEGWHNYHHVFPWDYKTAEWGNYSLNMTTAFIDLFAKIGWAYDLKSVVPETVERRVRRTGDGSHELWGWGDKDLTPEDGQSVLHVLDKKSN

SEQ ID NO: 14 - protein, 361 aa, Drosophila melanogaster
MAPYSRIYHQDKSSRETGVLFEDDAQTVDSDLTTDRFQLKRAEKRRLPLVWRNIILFALVHLAALYGLHSIFTRAKLATTLFAAGLYIIGMLGVTAGAHRLWAHRTYKAKWPLRLLLVIFNTIAFQDAVYHWARDHRVHHKYSETDADPHNATRGFFFSHVGWLLCKKHPDIKEKGRGLDLSDLRADPILMFQRKHYYILMPLACFVLPTVIPMVYWNETLASSWFVATMFRWCFQLNMTWLVNSAAHKFGNRPYDKTMNPTQNAFVSAFTFGEGWHNYHHAFPWDYKTAEWGCYSLNITTAFIDLFAKIGWAYDLKTVAPDVIQRRVLRTGDGSHELWGWGDKDLTAEDARNVLLVDKSR

SEQ ID NO: 15 - protein, 381 aa, Drosophila virilis
MPPNAITSPSNVLATCPEVEQLPSGECASTASSKQTGVLFEGDADTADCALDVDVKKLKKAEKRKLKLVWRNIMLFGYLHLAAVYGGYLMLTQAKWATVVFSFFLYTAGMIGITGGAHRLWAHRSYKAKWPLRVILVTFNTIAFQDAAFHWARDHRVHHKFSETDADPHNATRGFFFSHVGWLLCKKHPDVVAKGKSLDVTDLRADRILMFQLKHYFVLMPLACFILPTIIPMICWNESLLCSWFVATMFRWCFQLNMTWLVNSAAHKFGGRPYDKNINPSQSPYVSAFTFGEGWHNYHHVFPWDYKTAEWGRYSLNMTTAFIDFFAKIGWAYELKSVAPETIERRVRRTGDGTHELWGWGDKDLTAEDAQHVLFVDKKAA

SEQ ID NO: 16 - protein, 332 aa, Epiphyas postvittana
MAPNVEEIETDLTETEEKWEKLVAPQAAPRKHEILYTNLLIFGYGHLAGLYGLYLCFTSARLQTIILAFILHAMAILGITAGAHRLWTHRSYKATMPLQIILIIFNSLSFQNSAINWVRDHRSHHKYCDTDADPHNAARGLFYSHIGWLLVKKHPEVKKRGKMTDMSDVYRNPVLRFQKKYAVPFIGTICFVLPTIIPMYFWGESLNNAWHITLLRYIFSMHTIFLVNSVAHLWGNRPYDKNILPADNRTLSIATLGEASHNYHHTFPWDYRSTELGYLPTNFTTNFIDFFAWIGWAYDLKTTSGEIINSRIQRTGDGTHSRSKKNISTQDE

SEQ ID NO: 17 - protein, 346 aa, Grapholita molesta
MPPESKNVPIQQNFRKPLEFLPRKYDVVYENVFLHIAGHISAAYGLYLCFTVAKWQTIALAFVWYHLGKIGIICGAHRLWSHRCYKAKMPLHIILMICNCIGFENTAINWVRNHRMHHKHSDTDGDPHNSNRGAFFSHIGWLCVRKHPETRNCKVDMSDIYSNPVLVFQKRYKYPLVGFLCYGLPTFIPMYFWGETLVTAWHVNILRYFLSMNAVFLVNSLAHLYGNKPYDISICPRQSPFVSLLTIGEGFHNYHHTFPWDYRAAELGNNYLNVGKWVIDFFAMIGWAYDLKTVPDETIKRRMKRTGDGTNCWGWGDKDMTREDRDIAKIIYPESISKEERDIIAM

SEQ ID NO: 18 - protein, 341 aa, Grapholita molesta
MTPDAREETLLEDDGYVRLVAPQASSGKQEASYLNIAMYSFFHTAGAYGLYLAATEAKWATIFLAFILHEAAILGVTAGAHRLWAHRTYKAKLPLQIFLMLLHSFACRYSAFHWARDHRLHHKYSDTDGDPHNASRGLFFSHIGWLMFKKHPEAKKRMGKIDVSDLKANKVLMFQKKYSIPFIGTICFILPTLIPMYFWDETFSSAFFVTVLRVIVSLHVTFLVNSFAHAFGNRPYDRYIKPSQSIPISLATLGEGYHNYHHAFPWDYKASEFGNTIGNFTTMVINFFAWLGWAYDLKSVGADMIAKKKEKTGDGTNLWGWGDKDMSKEHKEIANVTYKEK

SEQ ID NO: 19 - protein, 338 aa, Helicoverpa zea
MAQSYQSTTVLSEEKELTLQHLVPQASPRKYQIVYPNLITFGYWHIAGLYGLYLCFTSAKWATILFSYILFVLAEIGITAGAHRLWAHKTYKAKLPLEILLMVFNSIAFQNSAIDWVRDHRLHHKYSDTDADPHNASRGFFYSHVGWLLVRKHPEVKKRGKELNMSDIYNNPVLRFQKKYAIPFIGAVCFALPTMIPVYFWGETWSNAWHITMLRYIMNLNVTFLVNSAAHIWGNKPYDAKILPAQNVAVSVATGGEGFHNYHHVFPWDYRAAELGNNSLNLTTKFIDLFAAIGWAYDLKTVSEDMIKQRIKRTGDGTDLWGHEQNCDEVWDVKDKSS

SEQ ID NO: 20 - protein, 337 aa, Lobesia botrana
MVPRAASEETDLKEATQLEPRKYEIVYTNVIYFTYWHIAGLYGLYLCFTSAKWETIVFAWAWYVLGELGVIAGAHRLWAHRTYKAKMPLQIILMLFNCIGFQNTATDWVRDHRVHHKHSDTDADPHNSQRGFFFSHVGWLLTRKHRLVKEKGEAVDMTDIYSNPVLRFQKKYSLPLIGTLCFALPTLLPAYCWGEAVGTAWNINLLRYCLNLNGTFLTNSAAHKFGSKPYDKTIPPTQNLLVSFMTLGEGFHNYHHVFSWDYRAAELGNTYLNMTTIFIDFFALIGWAYDLKTVPEDVIKKRMARTGDGTNLWGWGDKDMTKEDVVDTEIRFHAKKE

SEQ ID NO: 21 - protein, 352 aa, Lobesia botrana
MPPNVTDVNGVLFESDAATPDLALATPPVQKADNTPRVYVWRNIILFGYLHIAALYGGYLFLVSAKWQTDIFAYLLYVASGLGITAGAHRLWAHKSYKAKWPLRVILTIFNTIAFQDSAIDWARDHRMHHKYSETDADPHNATRGFFFSHIGWLLVRKHPELKRKGKGLDLSDLYADPILRFQKKYYLILMPLACFIMPTLIPVYFWGETWTNAFFVAALFRYAFILNITWLVNSAAHKWGDKPYDKTIKPSENLSVSLFAFGEGFHNYHHTFPWDYKTAELGNHRLNFTTKFINFFAKLGWAYDMKTVSDEIVQKRVQRTGDGSHHLWGWGDKDQSKDEVDAAIRINPKDD

SEQ ID NO: 22 - protein, 341 aa, Manducta sexta
MAPNFGNEVSSPIVAEESYEKLIPPQAAPRKYKYLYANMIYFTYWHIAGLYGIYLAITTAKWATIILAYLLFVAGEIGITAGAHRLWAHKSYKAKLPLQILLMLFNSTAFQNSVITWVKDHRMHHKYSDTDADPHNATRGFFYSHVGWLMVKRHPEAIKRGKSLDMSDIYNNPVLKFQKKYAIPLITTVAFVLPTIIPMYFWDESFNVAWHMTMLRYIINLNTIFLVNSVAHMWGYKPYDKNIAPTQNYIATFATLGEGFHNYHHAFPWDYRASELGNNYLNLTTKFIDFFAWIGWAYDLKTVPEDLLQKRMERTGDGTNLWGRGDKNMKKDYVKSTDVHE

SEQ ID NO: 23 - protein, 329 aa, Ostrinia nubilalis
MVPYATTADGHPEKDECFEDNEIKSNSLPKLEILYFNVMTFTFLHLSALYGLYLGFTSVKWATIGLGIIFYFFAEIGITAGAHRLWSHRSYKAKLPLEILLMVFNSMAFQNTALSWARDHRVHHKCPDTNGDPHNANRGFFYSHVGWLMTKKSDEVIKQGKLCDVADLYSNPVLRFQKKYAVPFIGTLCFVLPTLIPMYFWGETLNNAWHFNMFRYVINLNATFCVNSVVHKWGYKPYDKNICPTQNVLLNLAVLGEAFHNYHHVFPWDYRAAELGNQKMNPTTLFIDFFAWIGWAYDLKTASKEMIKSRSERTGDGTDLWGHSADKLK

SEQ ID NO: 24 - protein, 339 aa, Pectinophora gossypiella
MAPNTKTSESLLKTPEDLKSSEDTKQKLVHLNLLTFGYGHLAGLYGLYLCFTSAKWPTIFLNYVLLIISELGVTAGAHRLWAHKTYKAKLPLQILLMIFNSVAFQNTATDWVRDHRMHHKYSDTDADPHNSNRGFFYSHVGWLLMRKHPEVKRLGKGIEMRDIYSNPVLRFQKRYALPVIGAACFILPTVIPVYCWGETLSNSWHVTIFRYVVNLNVTFLVNSAAHIWGNKPYDKTLRPAQNIPVALLTLGEGFHNYHHVFPWDYRTAELGNNKLNVTTFFINFFAWIGWAYDLKTAPDDIIRKRVERTGDGTNMWGWGDKDMSKDDRESVKILGEKFL

SEQ ID NO: 25 - protein, 368 aa, Pelargonium hortorum
MGVLLNICSSPFPVVASAASTSISKVNHIRKVGVTGVMAPQKIEIFKSMEEWGKHNILPLAKPVEKSWQPTDFLPDPSSEGFMEEYNAFKERTRELPDEYFVVLAGDMITEEALPTYQTLVNRPDEVADETGHSESPWAVWSRAWTAEENRHGDLLNKYLYLSGKLDMRQVEKTIQYLIALGQDIGTEKNPYHLFIYTSFQERATFISHANTAKLAQQHGDKQLAQICGTIAADEKRHETAYTRIVDKLFELDPDETMSCLAHMMKRKITMPAHLMRDGRDPHLFQHFSVVASRTGVYTVMDYINILEHFVEKWNIEKITAGLSDKGREAQDYVCKLGERLRKVEERAHQRVVQADPIPFSWIFDRKV

SEQ ID NO: 26 - protein, 348 aa, Plutella xylostella
MCPKSPEQASVVEEREEKSAEFEKLIAPQAGPRKFNIVYFNLLTFGYWHLAGAYGLYLCFTSAKFATVLFAILTYTAAEIGITAGAHRLWSHKAYKAKLPLQIILMTFNTLAFQNSAIEWVRDHRLHHKYSDTDADPHNATRGFFYSHVGWLLVRKHSEVKKRGKTIDMSDMYSNPVLAFQKRYIVPWVILVTFLLPTIIPVYLWNESLWTSWHVTMLRYVANLNATFLVNSAAHLWGYKPYDKNIMPAQNISVSLATFGEGFHNYHHVFPWDYKAAELGNNRYNLTTKFIDFFAWIGWAYDLKSVSEELVVNRMRRTGDGSNLWGWGDKDMTEEDRNGALLTTKSVV

SEQ ID NO: 27 - protein, 396 aa, Ricinus communis
MALKLNPFLSQTQKLPSFALPPMASTRSPKFYMASTLKSGSKEVENLKKPFMPPREVHVQVTHSMPPQKIEIFKSLDNWAEENILVHLKPVEKCWQPQDFLPDPASDGFDEQVRELRERAKEIPDDYFVVLVGDMITEEALPTYQTALNRGDGVRDETGASPTSWAIWTRAWTAEENRHGDLLNKYLYLSGRVDMRQIEKTIQYLIGSGMDVRVENSPYLLFIYTSFQERATFISHGNTARQAKEHGDIKLAQICGTIAADEKRHETAYTKIVEKLFEIDPDGTVLAFADMMRKKISMPAHLMYDGRDDNLFDHFSAVAQRLGVYTAKDYADILEFLVGRWKVDKLTGLSAEGQKAQDYVCRLPPRIRRLEERAQGRAKEAPTMPFSWIFDRQVKL

SEQ ID NO: 28 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSLAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 29 - protein, 338 aa, Spodoptera exigua
MAQTIQTTTILEQKEEKTVTLLVPQAGKRKFEFVYQNLITFAYWHIAGLYGLYLCFTSAKWATILFSFILFVIAEIGITAGAHRLWSHKSYKVKLPLEILLMVMNSIAFQNTVIDWVRDHRLHHKYSDTDADPHNASRGFFYSHIGWLFVRKHPEVKKRGKELDMSDIYNNPVLRFQRKYAVPFIGAVCFGLPTLIPVYCWGESWTNAWHITMLRYIMNLNATFLVNSAAHIYGKRPYDKKILPAQNIGVSIATFGEGFHNYHHVFPWDYRAAELGNNGLNLTTKFIDFFAWIGWAYDLKTVSKEMIKQRSKRTGDGTNLWGLEDKDTPENLKNIKGE

SEQ ID NO: 30 - protein, 338 aa, Spodoptera littoralis
MAQCVQTTTILEQKEEKTVTLLVPQAGKRKFEIVYFNIITFAYWHIAGLYGLYLCFTSTKWATVLFSFFLFVVAEVGVTAGSHRLWSHKTYKAKLPLQILLMVMNSLAFQNTVIDWVRDHRLHHKYSDTDADPHNASRGFFYSHVGWLLVRKHPDVKKRGKEIDISDIYNNPVLRFQKKYAIPFIGAVCFVLPTLIPVYGWGETWTNAWHVAMLRYIMNLNVTFLVNSAAHIYGKRPYDKKILPSQNIAVSIATFGEGFHNYHHVFPWDYRAAELGNNSLNFPTKFIDFFAWIGWAYDLKTVSKEMIKQRSKRTGDGTNLWGLEDVDTPEDLKNTKGE

SEQ ID NO: 31 - protein, 338 aa, Spodoptera littoralis; identical to SEQ ID NO: 30

SEQ ID NO: 32 - protein, 338 aa, Spodoptera litura
MAQTIQTTTILEQKEEKTVTLLVPQAGKRKFEIVYFNLVSFAYWHIAGLYGLYLCFTSAKWATILFSFFLFVVAEVGVTAGAHRLWSHKTYKAKLPLQILLMVMNSLAFQNTAIDWVRDHRLHHKYSDTDADPHNASRGFFYSHIGWLFVRKHPDVKKRGKEIDISDIYNNPVLRFQKKYAIPFIGAVCFALPTLIPVYGWGETWTNAWHVAMLRYIMNLNVTFLVNSAAHIYGKRPYDKKILPSQNIAVSIATFGEGFHNYHHVFPWDYRAAELGNNCLNFTTKFIDFFAWIGWAYDLKTVSKEMIKQRSKRTGDGTNLWGLEDVDTPEDLKNTKGE

SEQ ID NO: 33 - protein, 353 aa, Spodoptera litura
MAPNISEDVNGVLFESDAATPDLALARPPVQKADNKPKQLVWRNIILFAYLHLAALYGGYLFLFSAKWQTDIFAYILYVISGLGITAGAHRLWAHKSYKAKWPLKVILIIFNTVAFQDAAMDWARDHRMHHKYSETDADPHNATRGFFFSHIGWLLVRKHPDLKEKGKGLDMSDLLADPVLRFQKKYYLLLMPLACFVMPTVIPVYLWGETWTNAFFVAAMFRYAFILNVTWLVNSAAHKWGDKPYDKSIKPSENMSVAMFALGEGFHNYHHTFPWDYKTAEFGNNKLNFTTAFINFFAKIGWAYDMKTVSEDIVKNRVKRTGDGSHHLWGWGDENQPKEEIEAAIRINPKDD

SEQ ID NO: 34 - protein, 347 aa, Thaumetopoea pityocampa
MAPNTRENETIYDEVEHKLEKLVPPQAGPWNYKIVYLNLLTFSYWLIAGAYGLYLCFTSAKWATIIFEFILFFFAEMGITAGAHRLWTHKSYKAKLPLEIFLMVLNSVAFQNTATDWVRDHRLHHKYSDTDADPHNAARGLFFSHVGWLLVRKHDEVKKRGKFTDMSDIYNNPVLKFQKKYAIPFIGAVCFILPTVIPMYFWGESLNNAWHICILRYAMNLNVTFSVNSLAHIWGNKPYDKDIKPAQNFGVTLATFGEGFHNYHHVFPWDYRTSELGDNKFNFTTKFINFFERIGLAYDLKTVSDDVIAQRAKRTGDGTHLWDCADKNNNDVVQTKAQIDTLCTKHE

SEQ ID NO: 35 - protein, 302 aa, Tribolium castaneum
MFLRTITSKFYSDQIVWRNVFLLLILHIISLQGWYFVLTTTNWPTLIYGFIFGALTGQGIKLGAHRLWAHRCYKAKLPLRIFLCFLQTVTLQNPLYEWVRDHQVHHKYTDTNADPLNATRGFFFSHMGWLLVRKHPNVIAKGKTLDLSDLEEDPVVMFQKKYYKIIAPVLTLAIPALIPWYFFGEDLYLSWVTTCVLPYFITLHSTWAVNSVAHIWGTKPYNKNILPTENIAVAIVAYGEGWHNYHHVFPWDYKAAELGNYRPNLSTAFIDFMAKIAWAYDLKSVSPEMLRKRKMRTGDCDY

SEQ ID NO: 36 - protein, 389 aa, Tribolium castaneum
MAPNLLGNSTLFLAETNSAEPIQIISKPGLQDVLPQVKPQISSRSSVSQYRWQIVWRNVLIFIYLHIAGIYGLYYAIAQAQWKTLLWGYLVILASGIGVTAGAHRLWAHRTYKAKLPLRIYLAFCQTVALQNDIYEWVRDHRVHHKFTDTDADPHNSNRGFFFSHMGWLLVKKHKDVFVKGKTVDMSDVEADPVVRFQRKYYIILTPILTFVFPAIVPWYFWNETPTVCFYSVAIFRYILTLHGTWLVNSAAHIWGYRPYDKNINATENKSVSILAFGEGWHNYHHVFPWDYKAAELGNYRMNFTTAFLDLMSKIGQAYDLKTVSVDMINKRRKRTGDGTGLVDEELLENEDKHHHHHDDSIWGWGDKDMKQDDMDMVQVHNPSREKFD

SEQ ID NO: 37 - protein, 349 aa, Trichoplusia ni
MAVMAQTVQETATVLEEEARTVTLVAPKTTPRKYKYIYTNFLTFSYAHLAALYGLYLCFTSAKWETLLFSFVLFHMSNIGITAGAHRLWTHKTFKAKLPLEIVLMIFNSLAFQNTAITWAREHRLHHKYSDTDADPHNASRGFFYSHVGWLLVKKHPDVLKYGKTIDMSDVYNNPVLKFQKKYAVPLIGTVCFALPTLIPVYCWGESWNNAWHIALFRYIFNLNVTFLVNSAAHIWGNKPYDKSILPAQNLLVSFLASGEGFHNYHHVFPWDYRTAELGNNFLNLTTLFIDFCAWFGWAYDLKSVSEDIIKQRAKRTGDGSSGVIWGWDDKDMDRDIKSKANIFYAKKE

SEQ ID NO: 38 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 39 - DNA, 1011 bp, Agrotis segetum
atggctcaaggtgttcaaactaccaccattttgagagaagaagaaccatctttgaccttcgttgttccacaagaacctagaaagtaccaaatcgtttacccaaacttgatcactttcggttactggcatattgctggtttgtacggtttgtatttgtgtttcacttctgctaagtggcaaactatcttgttctccttcatgttggttgttttggccgaattgggtattactgctggtgctcatagattgtgggctcataagacttacaaagctaagttgccattgcaaatcatcttgatgatcttgaactccattgccttccaaaactctgctattgattgggttagagatcacagattgcatcacaagtactctgatactgatgctgatccacataatgctactagaggttttttctactctcacgtcggttggttattggttagaaaacatccagaagtcaagagaagaggtaaagaattggatatgtccgacatctacaacaacccagttttgagattccaaaagaagtacgccattccattcattggtgctatgtgttttggtttgccaactttcatcccagtttacttttggggtgaaacttggtctaatgcttggcatattaccatgttgagatatatcttgaatttgaacatcactttcttggtcaactccgctgctcatatttggggttacaaaccatacgacattaagattttgccagcccaaaacattgccgtttctatagttactggtggtgaagtttctattaccaccactacattttttccatgggattacagagctgccgaattaggtaacaattacttgaacttgaccaccaagttcatcgatttctttgcttggattggttgggcctacgatttgaaaactgtttcctctgatgtcatcaagtccaaggctgaaagaactggtgatggtactaatttgtggggtttggaagataagggtgaagaagatttcttgaagatctggaaggacaactaa

SEQ ID NO: 40 - DNA, 981 bp, Amyelois transitella
atggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 41 - DNA, 1059 bp, Amyelois transitella
atgccgcctcagggtcaagatcgcgagtcctgggttctgtacgagaccgacgacaagacgcaagatggtggcactcacgtggtgcctccctccgccgagaaaagagtgtggaaaatcgtctggaggaatgtcatcgcatttgctctgctccacatcggaggcgtttacggcgcttatttgttcctcttcaaagctatgtggctgaccgatttgtttgcggtatttctgtacctgtgctccggcctgggcgtcactgcgggcgcgcaccggctgtgggcacacaagtcgtacaaggcgcggctgcctctccgcctgttgcttactgtcttcaacaccatagcttttcaggatgccgtgatcgactgggctcgcgaccaccgcctgcaccataagtactcggagacggacgccgatccccacaacgcaacaagggggttcttcttctcccacattggctggttgctcgtcaggaaacacccacaaatcaaggagaaagggcccaccatcgacctgaacgacttgagggccgatcccgtactccacttccagaagaaatactatttataccttatgcccttggcatgtttcgtcatgcccacattagtacctacactctggggtgaatctctatggaacgcgtatttcgtatgcgctatcttcagatacatatatgttctaaacgtcacttggctcgtaaactcagctgcgcacgcttggggaagcaaaccgtacgacaaaaacatcaaccctgtggaaaccaagcctgtatccttggtggtccttggagaaggcttccacaattaccaccacactttcccgtgggattacaagactgccgaattaggagattactctttaaacttctccaaattattcatcgatgctatggcaaaaataggttgggcttatgacctgaaaacggtctctcctgatgtgatcgagaagcgtgtgaagaggacgggagacggcagtcaccacgtctggggctgggacgacaaagacgtcccggttgaagaaaaagaagaagccaccatctttaacccttcgaaagatgaataa

SEQ ID NO: 42 - DNA, 1059 bp, Amyelois transitella
atggctccaaatgccacagatgctaatggggttctgtttgagacggatgcagctaccccagacctggccctacctcacgcccctgtacagcaagcggacaattaccctaaaaaatatgtgtggagaaatataatattatttgcctatctccacattgctgcactctatggtggctacttgttcttgttccatgctaaatggcagacagatatatttgcttacattctgtatgtgatgtcaggcttgggaataacggcaggagcccatcgtctgtgggcccacaagtcctacaaagctaaatggccactcagattgatacttgtcatctttaacactctggcattccaggactctgcaatcgactggtctcgcgaccaccgaatgcaccacaaatactcggaaactgatgctgatccccacaacgcgacccgcggtttcttcttctcccacattggctggctgctggttaggaagcaccctgaacttaagaggaaaggcaaaggtctggaccttagtgacttatatgctgaccccattttgagattccaaaagaagtactacttgatcctgatgcccctcacttgcttcgtgttacctacggtcatccccgtgtattactggggcgagacttggaccaacgccttcttcgtggccgctcttttccggtatgccttcatccttaacgtcacctggctggtcaattctgctgctcacaagtggggcgataagccttacgatagaaacatcaagccttcggaaaatatttccgtctccatgttcgcgctcggagaaggcttccacaactaccaccatactttcccctgggactacaagaccgcggaacttggcaacaacatgctcaatttcacgactaacttcatcaatttctttgctaaaatcggttgggcgtacgatctgaaaactgtttccgatgaaattgttaggagccgcgctaagagaacaggagatggttctcaccatctatggggctggggtgataaggatcactctcgtgaggaaatggccgccgctataaggatacacccgaaagatgactag

SEQ ID NO: 43 - DNA, 1050 bp, Chauliognathus lugubris
atggctcccaactctaacgacgccaccggcgtgctgcaagagactgacgacgacgtgtcctctaaccaggtcctgcagcagatcaccaagtctgagaagtctaagctgatcatcgtgtggtctaacgtgatgtacttcgtgatcctgcacgtgggcgccctgtacggcctgtggctgctgctgacctctgctcagatctggacctgcctgtgggtgttcgccatgtacgagttcggcgagatctgcatcaccgctggcgtgcaccgactgtggtcccaccgatcttacaaggccaagtggcccctgcgactgttccacaccatgggacagaccctggccttccaggacgccgtggtggactgggcccgagatcaccgagtgcaccacaagtactctgaaaccgacgctgaccctcacaacgccaagcgaggcttcttcttctctcacatgggctggctgatgtgccgaaagtccaagcaggtcaaggaaaagggcaaggaacccgacatctctgacctgtacgctgatcccatcctgcgataccaaaagaagtactacatgctgttcatgcccctgatgtgcttcgctttccccaccgtggtgcccctgtacttctggaacgagtctctcaagaccgccttcttcgtgaacatcttccggtacatcttctccctgcacgccacctggctggtgaactctgccgctcacctctacggcgagaagccctacaacaagcacattaaccccgccgagaacctggccgtgtctctgatcgtgaacggcgaacgatggcacaactaccaccacacattcccttgggactacaaggccggcgagttcggacgatacggcaccaacctgaccaccgtgttcatcaacgccatggccaagatcggcctggcctacgacctgaagttcgtgcccgaggacgtggtgaagcgacgagtgcacaagaccggcgacggctctcacgccgtgtggggctggggcgacaaggaccagaccgtggaagagatttctaagaccatcgtggcctacaaccagtcttaa

SEQ ID NO: 44 - DNA, 1116 bp, Chilo supprealis
atggacttcgagtctgagctgcagaactgctctaacaagtctcaaaagcgaaacatcatggctcccaacgcctctgacgtgaacggcgtgctgttcgaggacgacgccgctactcccgacatggccctgtctaacacccctgtgcagaaggccgacaactaccccaagaagctggtctggcgaaacattatcgccttcgcctacctgcacattgccgccgtgtacggcggatacctgttcctgttcgccgccaagtggcagaccgacattttcgcctacatcctgtacgtgatgtctggcctgggcatcaccgctggcgcccaccgactgtgggctcacaagtcttacaaggctaagtggcccctgcgactgatcctggtgttcttcaacaccctggccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagctgaagcagaagggcaagggcctcgacctgtctgacctgtacgctgatcccatcctgcgattccagaagaagtactacctgattctgatgcccattgcctgcttcattctgcccaccgtgattcctctgtactactggggcgagaactgggtgaccgccttcttcgtggccgctctgttccgatacgcctttatcctgaacgtgacctggctggtgaactctgctgcccacaagtggggcgacaagccctacgacaagaacatcaagccctctgagaacatctctgtgtctatcttcgccctcggcgagggattccacaactaccaccacacattcccttgggactacaagaccgccgagctgggcaacaaccgactgaacttcactaccaacttcatcaactttttcgccaagatcggctgggcctacgacatgaagaccgtgtctgacgagatcgtgcagtctcgagtgaagcgaaccggcgacggctctcaccacctgtggggctggggagacaaggaccactgcaaggaagaggtggccgctgccatccgaatcaaccccaaggacgactag

SEQ ID NO: 45 - DNA, 1005 bp, Choristoneura parallela
atggctcccaacgtcgaggacatggaatctgacatgcccgagtctgagaagtgggagaagctggtggctccccaggctgctccccgaaagtacgagatcatctacaccaacctgctgaccttcggctacggccacattgccggcctgtacggactgtacctgtgcttcacctctgccaagtggcagaccgtgatcctggccatcatcctgaacgagatggccattctgggcatcaccgctggcgcccaccgactgtggtcccaccgatcttacaaggccgctgtgcccctgcagatcattctgatgatcttcaactctctggccttccagaactctgccatcaactgggtgcgagatcaccgaatgcaccacaagtactctgacaccgacggcgaccctcacaacgcctctcgaggcttcttctactctcacgtcggctggctgctggtgaagaagcaccccgaggtcaaaaagcgaggcaagatgatcgacatgagcgacatctactctaaccccgtgctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatcgccctgatcgcctgcctgggcgactctttccacaactaccaccacgtgttcccttgggactaccgagcctctgagctgggcaacatcggaatgaactggaccgctcagttcatcgactttttcgcctggatcggctgggcctacgacctcaagaccgcctctgacgagaacatcaactctcgaatgaagcgaaccggcgacggcaccgacatctctggacagaagtactcttgcgagtcctctgaggtgctgcagtaa

SEQ ID NO: 46 - DNA, 1008 bp, Choristoneura rosaceana
atggctcccaacgtcgaggacatggaatctgacctgcctgagtctgaggaaaagctcgagaagctggtggctccccaggctgctccccgaaagtaccagatcatctacaccaacctgctgaccttcggctactggcacattgccggcctgtacggactgtacctgtgcttcacctctgccaagtggcagaccatcattctggccctgatcctgaacgagatggccattctgggcatcaccgctggcgcccaccgactgtgggctcaccgatcttacaaggccaccgtgcctctgcagatcatcctgatcatcttcaactccctgtctttccagaactctgccatccactggatccgagatcaccgaatgcaccacaagtactctgacaccgacggcgaccctcacaacgcctctcgaggcttcttctactctcacgtcggctggctgctggtgaagaagcaccccgaggtcaagaagcgagccaagaccatcgacatgtctgacatctactctaaccccatcctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatgaccttcattgcctgcctgggcgagaacttccacaactaccaccacgtgttcccttgggactaccgagcctctgagctgggcaacatcggaatgaactggaccgccaagttcatcgactttttcgcctggatcggctgggcctacgacctcaagaccgcctctgacgagaacatcaagtctcgaatgaagcgaaccggcgacggcaccgacgtgtctggacagaagtactcttgcgagtcctctgaggtgctgcagtaa

SEQ ID NO: 47 - DNA, 1047 bp, Cydia pomonella
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagaactaa

SEQ ID NO: 48 - DNA, 1050 bp, Cydia pomonella
atgccccctcagggacagcctcccgcctgggtcctggacgagtccgatgcagtgaccgaggacaaggacgtggccacccccgctcccgaagccgagaagcgaaagctgcagatcgtttggagaaacgtgaccctgttcgtgtttctgcacatcggagctctgtacggaggatacctgttctttaccaaggccatgtggactacccgaattttcactgtgctgctgtacattatgtctgggctgggtatcaccgccggcgcccatcgactctgggctcacaagtcttacaaggcccgactgcccctgcgactgctgctgaccctcttcaacaccatcgcctttcaggactccgttctggattgggcccgagatcaccgaatgcaccataagtactctgaaaccgacgcagatccccacaatgctacccgaggcttcttcttctctcacgtgggctggctgctggtgcgaaagcacccccagatcaaggccaagggacatactatcgacatgtctgacctgctggccgatcccgtgctgcgattccagaagaagtactacctgacactgatgcccttgtgctgcttcatcctgccctcttacattcccaccctctggggagagtctctgtggaacgcttactttgtgtgcgccatcttccgatactgttacgttctgaacgtgacttggctggtgaactccgctgcccacaaatggggtgaccgaccttacgacaagaacatcaaccctgtggagactaagcctgtgtctctggtggttttcggagagggattccacaactaccaccacaccttcccctgggattacaagaccgccgagctgggcggatactctctgaacctttccaaactgttcatcgatactatgtccaagattggatgggcctacgacctgaagtccgtttcccctgacatcgtggagaagcgagtgaagcgcaccggcgacggatctcaccacgtgtggggatgggacgatgctccctctgagcaaaaggtggctgccaccatcgtgaaccccgataagaccgagtaa

SEQ ID NO: 49 - DNA, 1005 bp, Cydia pomonella
atggccccctactctgaggagtacgagatcctgaaggagaatactaagcccgtatctccccaggccgcccccagagagtacaccgttgtgtactctgtggtgcttatctttgtttactggcacatcggagccctgtacggactgtacctgggcttcacctccgccaagtgggccaccatcatctttaactacctgatctacgtgtctggcggcttcgccattactgctggatcccatcgactgtggtctcaccgagccttcaaggctaagctccccctgcagatcctgctcatgcttctgcagaccatgtcttgtcagaagtctgtgctgaactgggtgcgagatcaccgactgcaccacatgtactgtgataccgatgccgacccttacaactctactcgaggaatcttctactctcacatcggctggctgatggtgaagaagcatcctgaggtgatccgaaagggccgaaccatcgacatgtccgatctggagaacaaccctgtgctgaagttccagaagaagttctaccccatcctcgtgaccctgatggcctttatcctgcctgccctgatccccgttattttctggcaggagtctctgaacatcgctcaccacgtttctcttgtgcacctggtcgtgggctcccacatgacctttgccattaactctattgcccacgccttcggatctaagccttgcgacaagaccatctctcccactcagtccatttccctgtctctggtgaccttcggcgaaggctaccataactaccaccacgtgttcccctttgattaccgagtggccgagctgggcaacaactacctgaacctgaccaccaacttcatcgacttcttcgcctggattggctgggcctacgacctgaagtacgcctctcccgatatggttgctaagcgagccaagcgaaccggcgacggaactgacctgtggggacgagctattgagcacgccgatattcaggctaagcgggtgcacccctcttaa

SEQ ID NO: 50 - DNA, 1059 bp, Dendrolimus punctatus
atggctcccaaggaagccgacgtcaacggcgtgctgttcgagtctgacgccaccactcctgacatggctctgcccactactcccgtgcagcaggccgacaactaccccaagaagctggtctggcgaaacatcctgctgttcgcctacctgcacctggccgctctgtacggcggctacctgttcctgttctctgccaagtggcagaccgacatcttcgcctacatcctgtacatcatgtctggcctgggcatcaccgctggcgcccaccgactgtgggctcacaagtcttacaaggctaagtggcccctgcgactgatcctggtgctgttcaacaccctggccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagcttaagaagaagggcaagggcctcgacatctctgacctgtacgctgatcccatcctgcgattccagaagaagtactacctgctgctgatgcccctgggctgcttcatcctgcctaccgtgattcccgtgtacctgtggaacgagacttggtctaacgccttcctggtggctgccctgttccgatacgccgtgatcctgaacgtgacctggctggtgaactctgctgcccacaagtggggcgacaagccctacgacaagtctatcaagccctctgagaacctgtctgtggccctgttcgccctcggcgagggattccacaactaccaccacacattcccttgggactacaagaccgccgagctgggcaacaaccgactgaacttcactactaccttcatcaactttttcgccaagatcggctgggcctacgacatgaagaccgtgtctgacgagatcatccagaaccgagtgaagcgaaccggcgacggctctcaccacctgtggggctggggagacaaggaccactctaaggaagagatcaacgccgccatccgaatcaaccccaaggacgactaa

SEQ ID NO: 51 - DNA, 1149 bp, Drosophila grimshawi
atgcctccaaacgctatctcttctcctcctgacgctctgcccacctgtcctgaggaccaggtcctgtctggctctcagtctggcgctggcggagcctctaagcagaccggcgtgctgttcgaggccgacgccgacaccaacgacggcggcctgaccatggacatcacccagttcaagaacgccgagaagcgaaagctgaagtgggtgtggcgaaacatcgtcctgttcgcctacgtccacgtggccgctctgtacggcggctacctgctggtgacccaggccaagtgggccactatcgtgttctctatgttcctgtactctgccggcatgatcggcatcaccggcggagcccaccgactgtgggctcaccgatcttacaaggctaagtggcccctgcgagtgatcctggtggccttcaactctatcgccttccaggacgccgcctaccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacctgtctgacctgcgagccgaccgagtgctgatgttccaaaagaagcactacttcgttctgatgcccctggcctgcttcattctgcctacctgcattcccatgctgtgctggaacgagtctctgctgtgctctgtgctggtgcccaccatcttccgatggtgcctgcagctgaacatgacctggctggtgaactctgccgctcacaagttcggcggacgaccctacgacaagaacatcaacccttctcagtctccctacgtgtctctgttcaccctcggcgaaggctggcacaactaccaccacgtgttcccttgggactacaagaccgccgagtggggcaactactctctcaacatgaccaccgccttcattgacctgttcgctaagatcggctgggcctacgacctgaagtctgtggtgcccgagactgtcgagcgacgagtgcgacgaaccggcgacggctctcacgagctgtggggctggggcgacaaggacctgacacctgaggacggccagtctgtgctgcacgtgctggacaagaagtctaactag

SEQ ID NO: 52 - DNA, 1086 bp, Drosophila melanogaster
atggctccctactctcgaatctaccaccaggacaagtcgtcccgagagactggcgtgctgttcgaggacgacgcccagaccgtggactctgacctgaccaccgaccgattccagctgaagcgagccgagaagcgacgactgcccctggtgtggcgaaacatcatcctgttcgccctggtgcacctggccgctctgtacggcctgcactctatcttcacccgagccaagctggccaccactctgttcgctgccggcctgtacatcatcggcatgctgggcgtgaccgctggcgcccaccgactgtgggctcaccgaacctacaaggccaagtggcccctgcgactgctgctggtgatcttcaacaccattgccttccaggacgccgtgtaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggccgaggcctggacctgtctgacctgcgagctgaccccatcctgatgttccagcgaaagcactactacattctgatgcccctggcctgcttcgtgctgcccaccgtgattcccatggtgtactggaacgagactctggcctcttcctggttcgtggccaccatgttccgatggtgcttccagctcaacatgacctggctggtgaactctgccgctcacaagttcggcaaccgaccttacgacaagactatgaaccccactcagaacgccttcgtgtctgccttcaccttcggcgaaggctggcacaactaccaccacgcattcccttgggactacaagaccgccgagtggggctgctactctctgaacatcaccaccgccttcatcgacctgttcgctaagatcggctgggcctacgacctcaagaccgtggctcccgacgtgatccagcgacgagtgctgcgaaccggcgacggctctcacgagctgtggggctggggcgacaaggacctgaccgctgaggacgcccgaaacgtcctgctggtggacaagtctcgataa

SEQ ID NO: 53 - DNA, 1146 bp, Drosophila virilis
atgcctccaaacgccatcacttctccctctaacgtgctggccacctgtcctgaggtcgagcagctgccctctggcgagtgcgcctctaccgcctcttctaagcagaccggcgtgctgttcgagggcgacgccgacaccgccgactgcgccctggacgtggacgtgaagaagctgaagaaggccgagaagcgaaagctgaagctggtgtggcgaaacatcatgctgttcggctacctgcacctggccgccgtgtacggcggatacctgatgctgacccaggccaagtgggctaccgtggtgttctctttcttcctgtacaccgccggcatgatcggcatcaccggcggagcccaccgactgtgggctcaccgatcttacaaggctaagtggcccctgcgagtgatcctggtgaccttcaacaccattgccttccaggacgccgccttccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacgtgaccgacctgcgagccgaccgaatcctgatgttccagctgaagcactacttcgtgctgatgcccctggcctgcttcattctgcccaccatcattcccatgatctgctggaacgagtctctgctgtgctcctggttcgtggccaccatgttccgatggtgcttccagctcaacatgacctggctggtgaactctgccgctcacaagttcggcggacgaccctacgacaagaacatcaacccctctcagtctccctacgtgtctgccttcaccttcggcgaaggctggcacaactaccaccacgtgttcccttgggactacaagaccgccgagtggggccgatactctctgaacatgactaccgccttcatcgactttttcgccaagatcggctgggcctacgagctgaagtctgtggctcccgagactatcgagcgacgagtgcgacgaaccggcgacggcacccacgagctgtggggctggggcgacaaggacctgaccgctgaggacgcccagcacgtcctgttcgtggacaagaaggctgcctag

SEQ ID NO: 54 - DNA, 999 bp, Epiphyas postvittana
atggcccccaacgtggaggagatcgagactgacctcaccgagactgaggagaaatgggagaagctggtggctccccaggccgcccctagaaagcacgagatcctgtacaccaacctgctgattttcggctacggacacctggccggactgtatggactgtacctgtgtttcacctctgcccgacttcagaccatcatcctggccttcattctgcacgccatggccatcctgggaattactgccggcgctcaccgactgtggactcaccgttcttacaaggccaccatgcccctgcagattatcctgattatcttcaactctctgtcctttcaaaactccgccatcaactgggtccgagatcaccgatctcaccacaagtactgtgacaccgacgcagatcctcataacgccgctcgaggcctgttctacagccatatcggatggctgctggtgaagaagcaccccgaggtcaagaagcgaggaaagatgaccgacatgtctgatgtgtatcgaaaccccgtgctgcgattccagaagaagtacgctgtgccttttatcggaaccatctgcttcgtcctgcctaccatcattcccatgtacttctggggcgagtccctgaacaacgcctggcatatcaccctcctgcgatacattttctccatgcacactattttcctggtgaactctgtggcccatctgtggggcaaccgaccctacgacaagaacattcttcccgctgacaaccgaaccctgtctatcgccaccctcggggaggcctcccacaactaccaccacaccttcccttgggattaccggtccactgagctgggatacctgcccacgaactttaccaccaactttatcgacttcttcgcctggatcggctgggcttacgacctgaagaccacctctggagagatcatcaattctagaatccagcgaaccggcgatggaacccattctcgatctaagaagaacatctccacccaggacgagtaa

SEQ ID NO: 55 - DNA, 1042 bp, Grapholita molesta
atgcctcccgagtccaagaacgtgcccatccagcagaactttcgaaagccccttgagttcctgccccgaaagtacgacgtcgtgtacgagaacgtgttcctgcacatcgccggccatatctcggccgcctacggactgtacctgtgctttaccgttgctaagtggcagactatcgccctggccttcgtgtggtaccatctgggaaagatcggaatcatctgcggagctcacagactgtggtctcaccgatgctacaaggccaagatgcctctgcacatcatcctgatgatctgcaactgtattggatttgaaaacaccgccatcaattgggttcgaaaccaccgaatgcaccacaagcactctgacaccgacggcgatccccacaactctaaccgaggtgccttcttctcccacatcggctggctgtgcgtgcgaaagcatcctgaaactcgaaactgtaaggtggacatgtctgatatttactctaaccccgtgctggtgttccagaagcgctacaagtatcccctggtgggcttcctgtgctatggacttcccaccttcatccccatgtacttctggggcgaaactctggtgaccgcttggcacgtgaacatcctgcggtacttcctgtcgatgaacgccgtcttcctggtgaattcccttgcccatctgtacggcaacaagccctacgacatttccatctgcccccggcagtctcccttcgtgtctctcctgaccatcggagagggctttcacaactaccaccacaccttcccttgggactaccgagccgccgagctgggcaataactaccttaacgtgggaaagtgggtgatcgacttcttcgccatgattggatgggcctacgacctgaagactgtgcccgatgagaccattaagcgacgaatgaagcgaactggagacggaaccaactgctggggatggggcgacaaggacatgactcgagaggaccgagacatcgccaaaattatttaccccgagtctatttctaaggaagagcgagacattatcgccatgttaa

SEQ ID NO: 56 - DNA, 1026 bp, Grapholita molesta
atgacccctgacgctcgagaggaaaccctgctcgaggacgacggctacgtgcgactggtggctccccaggcctcttctggaaagcaagaggcctcctacctgaacattgccatgtactcgttcttccacaccgctggcgcctacggcctgtacctggccgccaccgaggccaagtgggctaccatcttcctggccttcatcctgcacgaggccgccatcctgggcgtgaccgccggtgctcaccgactgtgggctcaccgaacctacaaggccaagctgcccctgcagatcttcctgatgctgctgcactctttcgcctgccgatactctgccttccactgggcccgagatcaccgactccaccacaagtactctgacaccgacggcgaccctcacaacgcctctcgaggcctgttcttctctcacatcggctggctgatgttcaagaagcaccccgaggctaagaagcgaatgggcaagatcgacgtgtctgacctgaaggccaacaaggtcctgatgtttcagaagaagtactctatccccttcatcggcaccatctgcttcattctgcccactctgatccctatgtacttctgggacgagactttctcttctgccttcttcgtgaccgtgctgcgagtgatcgtgtctctgcacgtgacctttctggtgaactccttcgctcacgccttcggcaaccgaccttacgaccggtacatcaagccctctcagtctatccccatctctctggctaccctcggcgagggctaccacaactaccaccacgcattcccttgggactacaaggcttctgagttcggcaacaccatcggcaacttcactactatggtgatcaacttcttcgcctggctcggctgggcttacgacctgaagtctgtgggcgccgacatgatcgccaagaagaaggaaaagaccggcgacggcaccaacctgtggggctggggcgacaaggacatgtctaaggaacacaaggaaatcgccaacgtcacttacaaggaaaagtag

SEQ ID NO: 57 - DNA, 1017 bp, Helicoverpa zea
atggcccagtcttaccagtctaccaccgtgctgtctgaggaaaaggaactgaccctgcagcacctggtgcctcaggcttcgccccgaaagtaccagatcgtgtaccccaacctgatcaccttcggctactggcacattgccggcctgtacggactgtacctgtgcttcacctctgccaagtgggccaccatcctgttctcttacattctgttcgtgctggccgagatcggcatcaccgctggcgcccaccgactgtgggctcacaagacctacaaggccaagctgcccctcgagatcctgctgatggtgttcaactctatcgccttccagaactctgccatcgactgggtgcgagatcaccgactgcaccacaagtactctgacaccgacgctgaccctcacaacgcctctcgaggcttcttctactctcacgtcggctggctgctggtgcgaaagcaccccgaggtgaagaagcgaggcaaggaactcaacatgtctgacatctacaacaaccccgtgctgcgattccagaagaagtacgctatccccttcatcggcgccgtgtgcttcgctctgcccactatgatccccgtgtacttctggggcgagacttggtctaacgcctggcacatcaccatgctgcggtacatcatgaacctgaacgtgaccttcctggtgaactctgctgcccacatctggggcaacaagccctacgacgccaagattctgcccgctcagaacgtggccgtgtctgtggccaccggcggagagggcttccacaactaccaccacgtgttcccttgggactaccgagccgccgagctgggcaacaactctctgaacctgaccaccaagttcatcgacctgttcgccgccatcggctgggcctacgacctcaagaccgtgtccgaggacatgatcaagcagcgaatcaagcgaaccggcgacggcaccgacctctggggccacgagcagaactgcgacgaggtgtgggacgtgaaggacaagtcctcttaa

SEQ ID NO: 58 - DNA, 1014 bp, Lobesia botrana
atggtgcctcgagccgcttcggaggagaccgaccttaaggaggctacccagcttgagccccgaaagtacgagatcgtgtacactaacgtgatctacttcacctattggcatatcgccggactgtacggtctgtacctgtgttttacctccgctaaatgggagaccatcgtgttcgcttgggcttggtatgtgctcggagagctgggagtgattgccggcgctcatagattgtgggcccaccgaacctacaaggcaaagatgcccctgcagatcatcctgatgctgtttaattgtatcggttttcagaacaccgctaccgattgggttcgagatcaccgagtgcatcacaagcactctgacaccgacgccgacccccataactctcagcgaggcttctttttctctcacgtgggctggctgctgacccgaaagcatcggctggtgaaggagaagggagaagctgttgacatgactgatatctactctaaccctgttttaagattccagaagaagtactctctccccctgatcggcactctttgctttgctctgcccaccctgctgcccgcttactgctggggagaggccgtcggcaccgcttggaacattaacctgctgcgatactgtcttaacctgaacggaaccttcctgactaactccgccgctcacaagtttggctctaagccctatgacaagaccattcctcccacccagaacttgctggtgtctttcatgactctgggagaaggatttcataattaccaccacgtcttctcgtgggactaccgagctgctgagcttggcaacacctacctgaacatgaccactatctttatcgacttcttcgctcttattggatgggcctacgacctgaagaccgttcctgaggatgttattaaaaagcgaatggcccgaactggagatggtactaacctgtggggttggggagacaaggacatgaccaaggaggacgtggtggacaccgagatacgattccacgctaagaaggagtaa

SEQ ID NO: 59 - DNA, 1059 bp, Lobesia botrana
atgcctccaaacgtgaccgacgtgaacggcgtgctgttcgagtctgacgccgctactcccgacctggctctggctacccctcctgtgcagaaggccgacaacacccctcgagtctacgtgtggcgaaacatcatcctgttcggctacctgcacattgccgctctgtacggcggatacctgttcctggtgtctgctaagtggcagaccgacatcttcgcctacctgctgtacgtggcctctggcctgggcatcaccgctggcgcccaccgactgtgggctcacaagtcttacaaggccaagtggcccctgcgagtgatcctgaccatcttcaacaccattgccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagctgaagcgaaagggcaagggcctcgacctgtctgacctgtacgctgatcccatcctgcgattccagaagaagtactacctgattctgatgcccctggcctgcttcatcatgcccactctgatccccgtgtacttctggggcgagacttggaccaacgccttcttcgtggctgccctgttccgatacgccttcatcctgaacatcacctggctggtgaactctgctgcccacaagtggggcgacaagccctacgacaagaccatcaagccctctgagaacctgtctgtgtctctgttcgccttcggcgagggcttccacaactaccaccacacattcccttgggactacaagaccgccgagctgggcaaccaccgactcaacttcactaccaagttcatcaactttttcgccaagctcggctgggcctacgacatgaagaccgtgtctgacgagatcgtgcagaagcgagtccagcgaaccggcgacggctctcaccacctgtggggctggggagacaaggaccagtctaaggacgaggtggacgccgccatccgaatcaaccccaaggacgactag

SEQ ID NO: 60 - DNA, 1026 bp, Manducta sexta
atggctcccaacttcggcaacgaggtgtcgtcgcccatcgtggccgaggaatcttacgagaagctgatccctcctcaggctgctccccgaaagtacaagtacctgtacgccaacatgatctacttcacctactggcacattgccggcctgtacggcatctacctggccatcaccaccgccaagtgggccaccatcatcctggcctacctgctgttcgtggccggcgagatcggcatcaccgctggcgcccaccgactgtgggctcacaagtcttacaaggccaagctgcccctgcagatcctgctgatgctgttcaactctaccgccttccagaactctgtgatcacctgggtgaaggaccaccgaatgcaccacaagtactctgacaccgacgctgaccctcacaacgctacccgaggcttcttctactctcacgtcggctggctgatggtgaagcgacaccccgaggccatcaagcgaggcaagtctctggacatgtctgacatctacaacaaccccgtgctgaagttccagaagaagtacgctattcccctgattaccaccgtggccttcgtgctgcccactattatccctatgtacttctgggacgagtctttcaacgtggcctggcacatgaccatgctgcggtacatcatcaacctgaacaccatctttctggtgaactctgtcgctcacatgtggggctacaagccctacgacaagaacattgctcccactcagaactacattgccaccttcgctaccctcggcgagggcttccacaactaccaccacgcattcccttgggactaccgagcctctgagctgggcaacaactacctgaacctgactaccaagttcatcgactttttcgcctggatcggctgggcctacgacctcaagaccgtgcctgaggacctgctgcaaaagcgaatggaacgaaccggcgacggcaccaacctgtggggccgaggcgataagaacatgaagaaggactacgtcaagtctaccgacgtgcacgagtaa

SEQ ID NO: 61 - DNA, 990 bp, Ostrinia nubilalis
atggtcccctacgccactaccgccgacggccaccccgaaaaggacgagtgcttcgaggacaacgagatcaagtctaattctctacctaagctggagattctgtactttaacgttatgaccttcaccttcctgcacctgtctgctctgtacggtctgtacctgggcttcacctctgtgaagtgggctaccatcggactgggcatcattttctacttcttcgccgagatcggaattaccgccggagcccatcgactgtggtctcaccgatcttacaaggccaagttgcccttggagatcctgctgatggtgttcaactccatggccttccagaacaccgccctgtcttgggctcgagaccatcgagttcaccacaagtgccccgacaccaacggagaccctcataacgctaatcgaggattcttctactcccacgttggctggttgatgaccaagaagtctgacgaggtgattaagcagggaaagctgtgtgacgtggctgacctgtattctaaccccgttctgcgattccagaagaagtacgctgttcctttcattggaactctgtgcttcgtcctgcccaccctcatccccatgtacttctggggagagaccctgaacaatgcttggcatttcaacatgttccgatacgtgattaacctgaacgccaccttctgcgtgaactctgtcgtgcacaagtggggatacaagccctacgacaagaacatctgtcccacccagaacgtgctgctgaacttggctgttctgggtgaggcctttcataactaccaccatgtgttcccttgggactaccgagccgctgagctgggtaatcagaagatgaaccccactactctgttcattgatttctttgcctggattggctgggcctacgaccttaagactgcctccaaggagatgatcaagtctcgatctgaacgaaccggtgacggaaccgacctgtggggtcactcggccgacaagctgaagtaa

SEQ ID NO: 62 - DNA, 1020 bp, Pectinophora gossypiella
atggctcccaacaccaagacctctgagtctctgctcaagacccctgaggacctgaagtcctctgaggacaccaagcagaagctggtccacctgaacctgctgaccttcggctacggccacctggccggcctgtacggactgtacctgtgcttcacctctgccaagtggcctaccatcttcctgaactacgtgctgctgatcatctctgagctgggcgtgaccgctggcgcccaccgactgtgggctcacaagacctacaaggccaagctgcccctgcagatcctgctgatgatcttcaactctgtggccttccagaacaccgctaccgactgggtgcgagatcaccgaatgcaccacaagtactctgacaccgacgctgaccctcacaactctaaccgaggcttcttctactctcacgtcggctggctgctgatgcgaaagcaccccgaggtcaagcgactcggcaagggcatcgagatgcgagacatctactctaaccccgtgctgcgattccagaagcgatacgctctgcccgtgatcggagccgcctgcttcattctgcccaccgtgattcccgtgtactgctggggcgagactctgtctaactcttggcacgtgaccattttccgatacgtggtgaacctgaacgtgaccttcctggtgaactctgccgctcacatctggggcaacaagccctacgacaagaccctgcgacctgctcagaacatccccgtggctctgctcaccctcggcgagggcttccacaactaccaccacgtgttcccttgggactaccgaaccgccgagctgggcaacaacaagctcaacgtgactaccttcttcatcaactttttcgcctggatcggctgggcctacgacctcaagaccgctcctgacgacatcatccgaaagcgagtcgagcgaaccggcgacggcaccaacatgtggggctggggagacaaggacatgtctaaggacgaccgagagtctgtgaagatcctgggcgagaagttcctgtaa

SEQ ID NO: 63 - DNA, 1107 bp, Pelargonium hortorum
atgggcgtgctgctgaacatctgttcgtctcccttccccgtggtggcctctgccgcctctacctctatctctaaggtgaaccacatccgaaaggtgggcgtgaccggcgtgatggctccccagaagatcgagatcttcaagtctatggaggagtggggcaagcacaacatcctgcccctggccaagcccgtcgagaagtcttggcagcctaccgacttcctgcccgacccctcgtctgagggcttcatggaggagtacaacgccttcaaggagcgaactcgagagctgcccgacgagtacttcgtggtgctggccggcgacatgatcaccgaggaggccctgcccacctaccagaccctggtgaaccgacccgacgaggtggccgacgagactggccactctgagtctccctgggccgtgtggtcccgagcctggaccgccgaggagaaccgacacggcgacctgctgaacaagtacctgtacctgtctggcaagctggacatgcgacaggtcgagaagaccatccagtacctgatcgctctgggccaggacatcggcaccgagaagaacccctaccacctgttcatctacacctcgttccaggagcgagccaccttcatctctcacgccaacaccgccaagctggcccagcagcacggcgacaagcagctggctcagatctgcggcaccattgccgccgacgagaagcgacatgagactgcctacacccgaatcgtggacaagctgttcgagctggaccccgacgagactatgtcttgcctggcccacatgatgaagcgaaagatcaccatgcccgcccacctgatgcgagatggccgagatccccacctgttccagcacttctctgtggtggcctcccgaaccggcgtgtacaccgtgatggactacatcaacatcctcgagcacttcgtcgagaagtggaacatcgagaagatcaccgccggactgtctgacaagggccgagaggcccaggactacgtgtgcaagctgggcgagcgactgcgaaaggtcgaggagcgagctcatcagcgagtggtgcaggccgaccccatccccttctcttggatcttcgaccgaaaggtgtaa

SEQ ID NO: 64 - DNA, 1050 bp, Plutella xylostella
atgtgccccaagtctcccgagcaggcctctgtggtcgaggaacgagaggaaaagtctgccgagttcgagaagctgatcgctccccaggctggaccccgaaagttcaacatcgtgtacttcaacctgctgaccttcggctactggcacctggctggcgcctacggcctgtacctgtgcttcacctctgccaagttcgccaccgtgctgttcgccatcctgacctacaccgccgctgagatcggcatcaccgctggcgcccaccgactgtggtcccacaaggcctacaaggccaagctgcccctgcagatcatcctgatgaccttcaacaccctggccttccagaactctgccatcgagtgggtgcgagatcaccgactgcaccacaagtactctgacaccgacgctgaccctcacaacgctacccgaggcttcttctactctcacgtcggctggctgctggtgcgaaagcactctgaggtgaagaagcgaggcaagaccatcgacatgtctgatatgtactctaaccccgtgctggctttccaaaagcggtacatcgtgccctgggtgattctggtgaccttcctgctgcctaccatcattcccgtctacctgtggaacgagtctctgtggacctcttggcacgtgaccatgctgcgatacgtggccaacctgaacgccaccttcctggtgaactctgctgcccacctgtggggctacaagccctacgacaagaacatcatgcccgctcagaacatctctgtgtctctggccaccttcggcgagggcttccacaactaccaccacgtgttcccttgggactacaaggctgccgagctgggcaacaaccgatacaacctgactaccaagttcatcgactttttcgcctggatcggatgggcctacgacctgaagtctgtgtctgaggaactggtggtgaaccgaatgcgacgaaccggcgacggctctaacctctggggctggggcgacaaggacatgaccgaagaggaccgaaacggcgccctgctcaccaccaagtccgtggtcgagtag

SEQ ID NO: 65 - DNA, 1191 bp, Ricinus communis
atggccctgaagctgaaccccttcctgtctcaaacccagaagctgccctctttcgccctgcctcccatggcctctacccgatctcccaagttctacatggcctccaccctgaagtctggctctaaggaggtcgagaacctgaagaagcccttcatgcctccccgagaggtgcacgtgcaggtcacccactctatgcctccccagaagatcgagatcttcaagtctctggacaactgggccgaggagaacatcctggtccacctgaagcccgtcgagaagtgctggcagccccaggacttcctgcccgaccccgcctctgacggcttcgacgagcaggtccgagagctgcgagagcgagccaaggagatccccgacgactacttcgtggtgctggtgggcgacatgatcaccgaggaggccctgcccacctaccagaccgccctgaaccgaggcgacggcgtgcgagatgagactggcgcctctcccacctcttgggccatctggacccgagcctggaccgctgaggagaaccgacacggcgacctgctgaacaagtacctgtacctgtctggccgagtggacatgcgacagatcgagaagaccatccagtacctgatcggctctggcatggacgtgcgagtcgagaactctccctacctgctgttcatctacacctcgttccaggagcgagccaccttcatctctcacggcaacaccgcccgacaggccaaggagcacggcgacatcaagctggcccagatctgcggcaccattgccgccgacgagaagcgacacgagactgcctacaccaagatcgtcgagaagctgttcgagatcgaccccgacggcaccgtgctggccttcgccgacatgatgcgaaagaagatctctatgcccgcccacctgatgtacgacggccgagatgacaacctgttcgaccacttctctgccgtggctcagcgactgggcgtgtacaccgccaaggactacgccgacatcctcgagtttctggtgggccgatggaaggtggacaagctgaccggcctgtctgccgagggccagaaggcccaggactacgtctgccgactgcctccccgaatccgacgactcgaggagcgagctcagggccgagctaaggaggctcccaccatgcccttctcttggatcttcgaccgacaggtgaagctgtaa

SEQ ID NO: 66 - DNA, 1533 bp, Saccharomyces cerevisiae
atgcccacctctggcaccaccatcgagctgattgacgatcagttccccaaggacgactctgcctcttctggcatcgtggacgaggtggacctgaccgaggccaacatcctggccaccggcctgaacaagaaggctccccgaatcgtcaacggcttcggctctctgatgggctctaaggaaatggtgtctgtcgagttcgacaagaagggcaacgagaagaagtctaacctggaccgactgctcgagaaggacaaccaggaaaaggaagaggccaagaccaagatccacatctctgagcagccctggactctgaacaactggcaccagcacctgaactggctgaacatggtgctggtctgcggcatgcccatgatcggctggtacttcgccctgtctggcaaggtgcccctgcacctcaacgtgtttctgttctctgtgttctactacgccgtcggcggcgtgtctatcaccgccggctaccaccgactgtggtcccaccgatcttactctgctcactggcccctgcgactgttctacgccatcttcggctgcgcctctgtcgagggctctgccaagtggtggggccactctcaccgaatccaccaccgatacaccgacactctgcgagatccctacgacgcccgacgaggcctgtggtactctcacatgggctggatgctgctgaagcccaaccctaagtacaaggcccgagccgacatcaccgacatgactgacgactggaccatccgattccagcaccgacattacatcctgctgatgctgctcaccgccttcgtgatccccactctgatctgcggctacttcttcaacgactacatgggcggcctgatctacgccggcttcatccgagtgttcgtgattcagcaggctaccttctgtatcaactctctggcccactacatcggcacccagcctttcgacgaccgacgaacccctcgagacaactggatcaccgccattgtgaccttcggcgagggctaccacaacttccaccacgagttccccaccgactaccgaaacgccatcaagtggtatcagtacgaccccaccaaggtgattatctacctgacctctctggtcggcctggcctacgacctgaagaagttctctcagaacgccatcgaggaagccctgatccagcaagagcagaagaagatcaacaagaagaaggcaaagatcaactggggacccgtgctgaccgacctgcctatgtgggacaagcagaccttcctggccaagtccaaggaaaacaagggcctcgtcatcatctctggcattgtgcacgacgtgtctggctacatttctgagcaccccggtggcgagactctgatcaagaccgctctcggcaaggacgccaccaaggccttctctggcggagtgtaccgacattctaacgccgctcagaacgtgctggccgacatgcgagtggccgtgatcaaggaatctaagaactctgccatccgaatggcctctaagcgaggcgagatctacgagactggcaagttcttttag

SEQ ID NO: 67 - DNA, 1017 bp, Spodoptera exigua
atggcccagactatccagaccaccaccattctggagcagaaggaggaaaagaccgtgaccctcctggtgccccaagccggtaagcgaaagttcgagttcgtctaccagaaccttattactttcgcttactggcacatcgctggtctgtacggcctctacctgtgcttcacctccgccaagtgggctaccatccttttctccttcatcctgttcgtgatcgctgagattggtatcaccgccggtgctcaccgattatggtcccacaagtcttacaaggtcaagctgcctctggagattctgctgatggtcatgaactccattgcttttcagaacaccgtcattgactgggtccgagaccaccgactgcatcacaagtactccgacaccgacgctgacccccacaacgcctcccgaggcttcttctactctcacattggttggctgtttgttcgaaagcaccccgaggtcaagaagcgaggcaaggagctggatatgtccgacatctacaacaaccctgtcttgcgattccagcggaagtacgccgttcccttcattggagctgtctgcttcggcctgcctacccttattcctgtgtactgctggggcgagtcctggactaacgcctggcatatcaccatgctgcgatacatcatgaacctgaacgccactttcctggtgaactccgccgctcacatttacggcaagcgaccctacgacaagaagattcttcctgcccagaacatcggcgtctccatcgccaccttcggtgaaggttttcacaactaccaccacgtgttcccctgggactaccgagctgctgagctgggcaacaacggtctgaacctgactaccaagttcatcgacttcttcgcctggattggatgggcctacgatctgaagaccgtctccaaggagatgatcaagcagcgatccaagagaaccggcgacggcactaacctttggggtttggaggacaaggacacccctgaaaacctgaagaatatcaagggagagtaa

SEQ ID NO: 68 - DNA, 1017 bp, Spodoptera littoralis
atggcccagtgcgtccagaccaccaccatcctcgagcagaaggaagagaaaaccgtgaccctcctggtgccccaggccggaaagcgaaagttcgagatcgtgtacttcaacattattaccttcgcttactggcacattgccggactttacggcctgtacctgtgcttcacctctaccaagtgggccaccgttcttttctctttcttcctgttcgtggtggctgaggttggagttaccgccggctctcaccgactttggtctcataagacctacaaggccaagctccccctgcagattcttcttatggtgatgaactctctggcttttcaaaatactgttatcgactgggtgcgagaccatcggctgcaccataagtactctgacaccgacgctgatccccacaacgcttctcgaggattcttctactctcacgtgggatggcttctggtgcgaaagcaccccgacgtcaagaagcgaggaaaggagatcgacatttctgacatctacaacaatcccgtgcttcgattccagaagaagtacgctatccccttcattggcgccgtgtgtttcgtgctgcccaccctcatccctgtttacggctggggagagacctggaccaacgcctggcatgtggccatgctgcgatacattatgaacctgaacgtgactttcctggtgaactctgccgctcacatttacggcaagcgaccctacgataagaagatccttccctctcagaacatcgccgtctctatcgccaccttcggagagggatttcacaactaccaccacgtgttcccctgggattaccgagctgctgagctgggaaacaactctctgaacttccctaccaagtttattgacttcttcgcctggattggatgggcctacgacctgaagaccgtgtctaaggagatgatcaagcagcgatcgaagcgaaccggcgacggcacaaacctgtggggactggaggacgtcgacactcccgaggaccttaagaacaccaagggcgagtaa

SEQ ID NO: 69 - DNA, 1017 bp, Spodoptera littoralis; identical to SEQ ID NO: 68

SEQ ID NO: 70 - DNA, 1017 bp, Spodoptera litura
atggctcagaccatccagaccaccaccatcctcgagcagaaggaggagaagaccgtgaccctgctggtccctcaggctggtaagagaaagttcgagatcgtctatttcaacctggtctcttttgcctactggcatatcgctggcctgtacggtctttacctgtgcttcacctccgccaagtgggccaccattctgttctccttcttcctgttcgtcgtcgccgaggttggagttaccgccggagctcaccgactgtggtcccacaagacttacaaggccaagctcccccttcagatccttctgatggtcatgaactccctggccttccagaataccgccattgactgggtccgagaccaccggcttcaccataagtactccgacaccgatgccgaccctcataacgcctcccgaggcttcttctactcccacatcggctggctgttcgtgcgaaagcaccccgatgtcaagaagcgaggtaaggagattgacatctccgacatctacaataaccccgtcctgagattccagaagaagtacgccatccctttcattggcgctgtctgcttcgccctgcccacccttatccccgtctatggttggggcgagacctggaccaacgcctggcacgtcgccatgctgcgatacatcatgaacctcaatgttaccttcctggtcaactctgccgcccacatctacggaaagcgaccctacgacaagaagatccttccctctcagaacatcgccgtctccattgccacctttggtgagggtttccacaactaccaccacgtgttcccttgggattaccgagctgccgagctgggcaacaactgcctgaacttcaccaccaagttcattgacttcttcgcttggatcggatgggcctacgacctcaagacggtctccaaggagatgatcaagcagcgatccaagcgaactggcgacggaactaacctgtggggcctggaggacgtcgatactcctgaggacctgaagaacaccaagggcgagtaa

SEQ ID NO: 71 - DNA, 1062 bp, Spodoptera litura
atggctcccaacatctctgaggacgtgaacggcgtgctgttcgagtctgacgccgctactcccgacctggctctggcccgacctcctgtgcagaaggccgacaacaagcccaagcagctggtgtggcgaaacatcatcctgttcgcctacctgcacctggccgctctgtacggcggctacctgttcctgttctctgccaagtggcagaccgacatcttcgcctacatcctgtacgtgatctctggcctgggcatcaccgctggcgcccaccgactgtgggctcacaagtcttacaaggctaagtggcccctgaaggtgatcctgatcatcttcaacaccgtggccttccaggacgccgccatggactgggcccgagatcaccgaatgcaccacaagtactctgagactgacgctgaccctcacaacgctacccgaggcttcttcttctctcacatcggctggctgctggtgcgaaagcaccccgacctgaaggaaaagggcaagggcctcgacatgtctgacctgctggctgaccccgtgctgcgattccagaagaagtactacctgctgctgatgcccctggcctgcttcgtcatgcccaccgtgattcccgtgtacctgtggggcgagacttggaccaacgccttcttcgtggccgccatgttccgatacgccttcattctgaacgtgacctggctggtgaactctgctgcccacaagtggggcgacaagccctacgacaagtctatcaagccctctgagaacatgtctgtggccatgttcgccctcggcgagggattccacaactaccaccacacattcccttgggactacaagaccgccgagttcggcaacaacaagctgaacttcactaccgccttcatcaactttttcgccaagatcggctgggcttacgacatgaagaccgtgtctgaggacatcgtgaagaaccgagtgaagcgaaccggcgacggctctcaccacctctggggctggggcgacgagaaccagcctaaggaagagatcgaggccgccatccgaatcaaccccaaggacgactaa

SEQ ID NO: 72 - DNA, 1044 bp, Thaumetopoea pityocampa
atggcccccaacacccgagagaacgagactatctacgatgaggtggaacacaagctggagaagctggtgcctccccaggccggcccgtggaactacaagatcgtgtacctcaacctgttaaccttctcctactggctgatcgctggcgcttacggcctgtacctgtgtttcacctctgccaagtgggccaccatcatctttgagttcatcctgttcttcttcgctgagatgggcatcaccgccggtgctcaccgactgtggacccacaagtcctacaaggccaagctgcccctggagatttttctgatggtgctcaactctgtggcttttcagaacactgctactgattgggtgcgagaccaccgactccatcacaagtattcggacactgacgccgacccccacaacgccgcccgaggactgttcttctctcacgtgggatggctgcttgtccgaaaacacgacgaggtcaagaagcgaggcaagttcaccgacatgtctgacatctacaacaaccctgtgctgaagttccagaagaagtacgccatcccctttatcggagccgtgtgtttcatcctgcccaccgtgattcctatgtacttctggggcgagtctctgaacaacgcttggcatatttgtatcttgcgatacgccatgaacttaaacgttaccttctctgtgaactctctagcccatatctggggtaacaagccctacgacaaggacatcaagcccgcacagaacttcggcgtgaccctggccacctttggtgagggcttccacaactaccaccacgtgtttccctgggactatagaacctctgagctcggcgacaacaagtttaacttcaccaccaagttcatcaatttcttcgagagaattggactggcctacgacctgaagactgtgtccgacgacgtgatcgcccagcgagctaagcgaactggagatggcacccacctgtgggactgtgccgataagaacaacaacgacgtggtgcaaaccaaggcccagattgacaccctctgtaccaagcacgagtaa

SEQ ID NO: 73 - DNA, 909 bp, Tribolium castaneum
atgttcctgcgaaccatcacctctaagttctactctgaccagatcgtgtggcgaaacgtgttcctgctgctgatcctgcacatcatctccctgcaaggctggtacttcgtgctgaccaccaccaactggcccactctgatctacggcttcatcttcggcgccctgaccggccagggaatcaagctgggagcccaccgactgtgggctcaccgatgctacaaggccaagctgcccctgcgaatcttcctgtgcttcctgcagaccgtgactctgcagaaccctctgtacgagtgggtgcgagatcaccaggtgcaccacaagtacaccgacaccaacgctgaccctctgaacgctacccgaggcttcttcttctctcacatgggctggctgctggtgcgaaagcaccccaacgtgatcgccaagggcaagaccctggacctgtctgacctggaagaggaccccgtggtgatgttccagaagaagtactacaagatcattgcccctgtgctgaccctggctatccccgctctgatcccctggtactttttcggcgaggacctgtacctgtcttgggtgaccacctgtgtgctgccctacttcatcaccctgcactctacctgggccgtgaactctgtggcccacatctggggcaccaagccttacaacaagaacattctgcccaccgagaacattgccgtggccattgtggcctacggcgaaggctggcacaactaccaccacgtgttcccttgggactacaaggctgccgagctgggcaactaccgacctaacctgtctaccgccttcatcgacttcatggccaagatcgcctgggcctacgacctgaagtctgtgtctcccgagatgctgcgaaagcgaaagatgcgaaccggcgactgcgactactaa

SEQ ID NO: 74 - DNA, 1170 bp, Tribolium castaneum
atggctcccaacctgctgggcaactctaccctgttcctggccgagactaactctgctgagcccatccagatcatctctaagcccggcctgcaggacgtgctgccccaggtgaagccccagatctcttctcgatcttctgtgtctcagtaccgatggcagatcgtgtggcgaaacgtgctgatcttcatctacctgcacattgccggcatctacggcctgtactacgccattgctcaggcccagtggaagaccctgctgtggggctacctggtgatcctggcctctggcatcggcgtgaccgctggcgcccaccgactgtgggctcaccgaacctacaaggccaagctgcccctgcgaatctacctggccttctgccagaccgtggctctgcagaacgacatctacgagtgggtgcgagatcaccgagtgcaccacaagttcaccgacaccgacgctgaccctcacaactctaaccgaggcttcttcttctctcacatgggctggctgctggtgaagaagcacaaggacgttttcgtgaagggcaagaccgtggacatgtctgacgtcgaggctgaccccgtggtgcgattccagcgaaagtactacatcattctgacccctatcctgaccttcgtgttccccgctatcgtgccctggtacttctggaacgagactcccaccgtgtgcttctactctgtggccatcttccgatatatcctgactctgcacggcacctggctggtgaactctgccgctcacatctggggataccgaccttacgacaagaacatcaacgccaccgagaacaagtctgtgtctattctggccttcggcgaaggatggcacaactaccaccacgtgttcccttgggactacaaggctgccgagctgggaaactaccgaatgaacttcaccaccgcctttctggacctgatgtctaagatcggccaggcctacgacctcaagactgtgtctgtggacatgatcaacaagcgacgaaagcgaaccggcgacggaaccggcctggtggacgaggaactgctcgagaacgaggacaagcaccaccaccatcacgacgactctatttggggctggggcgacaaggacatgaagcaggacgacatggacatggtccaggtgcacaacccttctcgagagaagttcgactaa

SEQ ID NO: 75 - DNA, 1050 bp, Trichoplusia ni
atggctgttatggctcaaactgttcaagaaactgctaccgttttggaagaagaagctagaactgttactttggttgctccaaaaactaccccaagaaagtacaagtacatctacaccaatttcttgaccttctcctatgctcatttggctgcattatatggtttgtacttgtgtttcacttccgctaagtgggaaactttgttgttctctttcgttttgttccacatgtccaacattggtattactgctggtgctcatagattgtggactcataagacttttaaggccaagttgccattggaaatcgtcttgatgattttcaactccttggctttccaaaacactgctattacttgggctagagaacatagattgcatcacaagtactctgatactgatgctgatccacataatgcttctagaggtttcttctactctcatgttggttggttgttggttaagaaacacccagatgttttgaagtacggtaagaccattgatatgtccgatgtttacaacaacccagtcttgaagttccaaaagaaatacgccgttccattgattggtactgtttgttttgctttgccaaccttgattccagtttactgttggggtgaatcttggaacaatgcttggcatattgctttgttcagatatatcttcaacttgaacgtcaccttcttggttaattccgctgctcatatttggggtaacaagccatatgataagtccattttgccagcccaaaacttgttggtttcttttttggcttctggtgaaggtttccataactaccatcatgtttttccatgggattacagaactgctgaattgggtaacaacttcttgaacttgaccaccttgttcattgatttctgtgcttggtttggttgggcttacgatttgaaatctgtctccgaagatatcatcaagcaaagagctaaaagaaccggtgatggttcttctggtgttatatggggttgggatgataaggatatggatagagatattaagtccaaggccaacattttctacgctaagaaagaatga

SEQ ID NO: 76 - DNA, 1449 bp, Yarrowia lipolytica
atggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttag

SEQ ID NO: 77 - protein, 458 aa, Agrotis segetum
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 78 - protein, 458 aa, Agrotis ipsilon
MAVIISREEEKLSVPEFYAGKSIFITGGTGFLGKVFIEKLLYSCPDIDKIYMLIREKKNLSIDERMTMFLDDPLFSRLKEKRPGDVEKIVLIPGDISSPNLGLSAENERILIENVSVIIHSAATIKFNEPLPIAWKINVEGTRMLMDLSRRMKRIKVFIHISTAYSNANSERAAVEEILYPAPADMDQVYQLVKDGVTEEETEILLNGLPNTYTFTKALAEHLAAEHQVHVPTVIIRPSIVGSIKDEPIRGWLCNWFGATGISVFTAKGLNRVLLGKASNIVDVIPVDYVANLVIVAGAKNGGEKSEELKIYNCCSSDCNPVTVKKILKEFIDDTIKNKSHIMPLPGWFVFTKYKWLMTLLTIIFQMIPMYLADVYRVLMGKNPRYMKLHHLVIQTRLVINFFTFHSWVMKTDRARELFGSLSPVEKHMFPWDPSGIDWTEYLQSYCYGVRHFLEKRK

SEQ ID NO: 79 - protein, 478 aa, Bicyclus anynana
MYRNVNNNYKGHSVYTNGAGSRVKSLLSSATDNTNEYQSIAECYKGQSVFITGGTGFVGKVLLEKLLYSCPGIDKVYLLVRETQGATAHQRMQKLLEEPAFSRIKEENPQAFEKVIPIVGDITQPQLGIMAENEELLIKEVSFVYHVAATTKFNETLDIAMNVNVAGTGRVLDLSKRMENIKAFVYVSTAYSNTDREVVEEVLYPAPVSLNEVHKLLKIGITDAQVKELIKGRPNTYTFTKALAENLVADNHGHVPAIIVRPSIVSSSKKEPITGWIDSWYGATFLATVTMKGFNRVFVSSYEYNLDFIPVDYVSNLIIVAAARCKCSDKVDVYNSSTSGENPLKIGAFFDDIIAYSCKHKFYDIPLPMAYLTRYRWVMFLITLLLQTLPAYIADLFLLIVGKKPRYVKLASKISAAHEVLDYFPSRTWSMSARQTTALFQSLSPSDRDQFPCDPTDIDWKEYIVTYCQGIRQFLCKS

SEQ ID NO: 80 - protein, 482 aa, Bombus lapidarius
MSHHHHHHGSNTKLNENEINEKLRNVNSIGGFYAGTGILITGGTGFVGKGLLEKLIRTCSHIAAIFILIRPKRNQTIEQRFKKIIDDPIFDGVRAQNPAIFYKIHLVEGDVTLPDLGLLQKDRDMLIENVNIVFHIAATINFHQPLDMIVNVNVKGTANIIKLCKELKHVISVVYVSTAYSNPNLSDIEEKVYTTNLDPSLVMDICDRQDKELINLIEERILKTHPNTYTFTKNLAEQTISNNSKGLTVAIVRPSIISSSLKEPFPGWLVSFAGQSGIFKNIGNGMAKVLLGRGDVISDIVPVDYVVDAIMCAAWHVTLQIDNNVKVYNCTSSARPIKLGEIVNIFVECSREIPMKNTLWYPSCTIVANRFVYNVLNILLNVLPAFAVDIFLRLRGGKPMAMNMNKYYNKLVVATSYFNSNEWSFKRDNIADMINKVNTLEDGNIVKLDLQDMVWRKYIANYLAGIKKFILKEDPKSINAAP

SEQ ID NO: 81 - protein, 468 aa, Chilo suppressalis
MEPYKSDEEIKSKLFSPVVNFYTGKSVFITGATGFLGTVLLEKLMFTCAHNIKNIYILIKPTDGQSIDEKMSKFFDSRAFERLREHNPNFRSKIIPLTGDITKKSIGLSENDIFILRKEVSVVFHSAADTSFQLSLSEAIIINTKATEELLKICKDMHQLKAFVYVSTAYSNCNRPIIDEKVYPTDVSLETVYELLEYSKSDKLIEFLFNGRPNAYTYSKALSEELVQNYGNIIPSIIIRPSIITSSIKEPYPGWLSGWNGLNQVILSGMKGYLRCWFADDSCIADTIPVDYTANVMIVSAWDAHERRLKNDKQLKVFNCCSGLQNPIDTGNMMSICLEHNKKHEKDKSKANTMFIIRKNFYVYFFYFLVLHLIPALIIDVFYFLLGREMLMCTNLKKIKRFSSILKVFCFNQFLFIDKNVRRLHQALNETDKVLFDFDVRNIQWRVYLKDFVIALKEYDKTSRTVKI

SEQ ID NO: 82 - protein, 507 aa, Cydia pomonella
MDMIDEAEARGESQIQKFLSGSTILLTGGTGFLGKLLVEKLLRTCPDIKKIYLLARPKKNKEIQKRLQEQFEDPLYERLRKQVPDFMSKIGVVEGDVGKLGLGISESDRQTVVDEVDVIFHGAATLRFNEPLRDAVFINVRGTREMMLLARACTKLKAMVHISTAYSNCTLSEIDEVFYESPIPGDKLIDLAESLDEKTINSITPGLIGDFPNTYAYTKGVAEDVLQKYSQGLPVAVVRPSIVIGTAKDPVAGWIDNVYGPTGVIVGAELGLLHVLHAAPNASASLVPGDAVAAACVAAAWSVSRAENHQAPARDAPPLYHCVCSEKAPITWSQFMSLAETHGLVVPPMQAMWYYMLTLTNSKAMYTLLALLMHWIPAYIIDGVCMVLGKKPQLRKAYTKIEQFAAVIEFFALREWRFHNNNMTRLYNELCDADKHIYDFDTSAIDWNEFFANYMKGIRVYLLKDPVSTIPESLKRHKRLKWLHYALLTVLSLLVLRLLWFFVSFLF

SEQ ID NO: 83 - protein, 455 aa, Helicoverpa armigera
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 84 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 85 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 86 - protein, 453 aa, Heliothis virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 87 - protein, 468 aa, Plutella xylostella
MKLGTFGNMAANGGSVCDFYANSTVFITGGTGFLGKVLIEKLLRSCRKIDKIYVLLRGTTNKTAQERLEDLSHEVLFEKVRQDDPNIMKKLIPVNGDLTLPNLGMNNEDYSLIVKNVSIVFHAAASIRFNLKIKEALHCNVEGTKKVIELCHKARCIKAFVHISTAYSNTDKTFVKEEVYPAPENLDKIYVIIGQNGDDTEADDLTSSRPNTYTYAKAVAEYEVLRNHGSVPTAIIRPSIVTPIWRDPTPGWLDKWSGGTPIIFGLCKGLLRCLDTQPSNVIDFIPADIAVNLSIVAAAKCHVDTPKIRVYNCCSGPTNPMTFKMLSDYCHENSIKHGLHWFPYPKIFLSTNRVFLKIVRLVLQFLPLFLADMWLKIRGKQPRFVKMLEKLTKVEKTLHFFIKNQWQFENHNVEALISTLSPSDRVQFPCDPSEICWKTHFEDYCLGIDKYLMNTSVKSANINSGMYK

SEQ ID NO: 88 - protein, 454 aa, Spodoptera exigua
MVVLTSKEKSNMSVADFYAGKSVFITGGTGFLGKVFIEKLLYSCPDIDKIYMLIREKKGQSIRERLTKIVDDPLFNRLKEKRPGDLDKIVLIPGDVTVPGLGISDENEAILIDKVSVVIHSAATVKFNEPLETAWNVNVEGTRMIMALSRKMKRIEIFIHISTAYTNTNRAVVDEVLYPPPADINEVHQYVKNGITEEETEKILNGRPNTYTFTKALTEHLVAENQAYMPTIIVRPSIVGAIKDDPIRGWLANWYGATGLSVFTAKGLNRVIYGQSSHVVDLIPVDYVANLVIVAGAKTYRSNEVTIYNSCSSSCNPITMERLVGLFIDDTVKHNSYVMPLPGWYVYSNYRWLVYLVTIIFQMIPAYLADIGRRLLGKNPRYYKLQSLVAQTQEAVHFFTSHTWEIKSKRTSELFASLSHTDQRIFPCDAKKIDWTDYITDYCSGVRQFLEKKK

SEQ ID NO: 89 - protein, 450 aa, Spodoptera frugiperda
MSKANCSNVASFYAGKSVFITGGTGFVGKTLIEKLLYSCSGIDKIYVLVRDKYGKQASERLARVTSTPVFDRLKTSKSEELKKIVVISGDITRENLGLQEDVLKTLENEVSVVFHLAATVAFKLPLKDAMRINVNGTENVIELCRRMKKLQAFVHVSTAFTNSDRKEINEMVYPMPINLEEARHVAYTYSHDDVIVSAFMGKKPNTYTFSKAFAEQQVMKQCEDLPTAIIRPSIVMSSVKEPCPGWIDTWNGSTGLFVGMPAGILRVVLGRGSNVTDLVPVDIVTNLIVVAATECQKSKEIKVYNCCTGTSNPITCDEATTISRKVALKYSLNELPWPFLMFTPSVFLYTLITFLLQIVPAYLIDLWCILTGRKATQIKLQRRLKKVVDAVKFFLLNEWKFSDDNIRDLLKTMSPVDKEIFNFDVKTINWETCIEDYILGARKYLLKSEK

SEQ ID NO: 90 - protein, 454 aa, Spodoptera littoralis
MVVLTSKEKSNMSVADFYAGKSVFITGGTGFLGKVFIEKLLYSCPDIDKIYMLIREKKGQSIRERLTKIVDDPLFNRLKDKRPGDLGKIVLIPGDITVPGLGISEENETILTEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYKWLVFLVTVIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 91 - protein, 454 aa, Spodoptera litura
MVVLTSKEKSNMSVADFYAGKSVFITGGTGFLGKVFIEKLLYSCPDIDKIYMLIREKKGQSIRERLTKIVDDPLFNRLKDKRPGDLGKIILIPGDITVPGLGISEENETILIEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYRWLVFLVTLIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 92 - protein, 515 aa, Tyta alba
MVSIPEYYEGKNILLTGATGFMGKVLLEKLLRSCPKVKAVYVLVRHKAGQTPEARIEEITNCKLFDRLRDEQPDFKAKIIVITSELTQPELDLSEPIKEKLIERINIIFHCAATVRFNETLRDAVQLNVTATQQLLFLAQRMKNLEVFMHVSTAYAYCNRKQIEEIVYPPPVDPKKLIDSLEWMDDGLVNDITPKLIGDRPNTYTYTKALAEYVVQQEGAKLNTAIIRPSIVGASWKEPFPGWIDNFNGPSGLFIAAGKGILRTMRASNSAVADLVPVDVVVNTTLAAAWYSGVNRPRNVMIYNCTTGGTNPFHWGEVGYHINLNFKINPLENAVRHPNCSLQSNPLLHQYWTAVSHTMPAFLLDLLLRLTGHKPWMMKTITRLHKAMMLLEYFTSNSWIWNTENMTMLMNQLNPEDKKTFNFDVRQLHWAEYMENYCMGTKKYVLNEEMSGLPAARKHLNKLRNIRYGFNTVLVILIWRIFIARSQMARNIWYFVVSLCYKFLSYFRASSTMRY

SEQ ID NO: 93 - protein, 453 aa, Trichoplusia ni
MIAVTSHENEASIVDFYEGKSVFITGGTGFLGKVFIEKLLYCCPGLVKIYMLIREKKGLTIKERIDRFVDDSLFDRLKTQRPKDLDKIVLIPGDITAPSLGISQEQENILIEEVSVIIHSAATIKFNAPLEEAWSINVDGTRRILDLCRNMKKVEVFLHISTAYTNINRNVIEEVLYPPPADIKELEKLDKHNVTEKQTMKLLNGFPNTYTFTKSLTEHLVAENCAYMPTIIVRPSVVAAVLEDPIKGWLENWYGATGISVFTAKGLNRVFLGHKNIIVDVIPADYVANLVIIAGARNNKSKELKIYNCCSSGCNPLTIGNLLGAFIDDAIRHKTYAMPLPGWFIFTRYRLVATIVTFIFQVVPAYLADLYRRIIGKKPKYTKLLYLVVQTRVALDFFSSNSWVMKADRMRELYASLSNNDKRMFPCDPTHINWPEYLSDYGAGVRRFLEKRN

SEQ ID NO: 94 - DNA, 1377 bp, Agrotis segetum
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 95 - DNA, 1377 bp, Agrotis ipsilon
atggccgtgatcatctcccgagaggaagagaagctgtctgtccccgagttctacgccggcaagtctatcttcattaccggcggcaccggattcctcggcaaggtgttcatcgagaagctgctctactcttgccccgacatcgacaagatctacatgctgatccgagagaagaagaacctctctatcgacgagcgaatgaccatgttcctggacgaccctctgttctctcgactgaaggaaaagcgacccggcgacgtcgagaagatcgtgctgatccccggcgacatctcttctcccaacctgggcctgtctgccgagaacgaacgaatcctgatcgagaacgtgtctgtgatcatccactctgccgccaccatcaagttcaacgagcccctgcctatcgcctggaagatcaacgtcgagggcacccgaatgctgatggacctgtctcgacgaatgaagcgaatcaaggtgtttatccacatctctaccgcctactctaacgccaactctgagcgagccgccgtggaagagattctgtaccccgctcctgccgacatggaccaggtgtaccagctcgtgaaggacggcgtgaccgaggaagaaaccgagatcctgctgaacggactgcccaacacctacaccttcaccaaggctctggccgagcacctggccgctgagcaccaggtgcacgtgcccaccgtgattattcgaccctctatcgtgggctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagaacggcggcgagaagtctgaggaactgaagatctataactgctgttcttctgactgcaaccccgtgaccgtgaagaagatcctgaaggaattcatcgacgacaccattaagaacaagtctcacatcatgcctctgcctggctggttcgtgttcaccaagtacaagtggctgatgaccctgctgaccatcatcttccagatgatccccatgtacctggccgacgtgtaccgagtcctgatgggcaagaaccctcggtacatgaagctgcaccacctggtcattcagacccgactggtgatcaacttcttcaccttccactcttgggtgatgaagaccgatcgagcccgagagctgttcggctctctgtctcccgttgagaagcacatgttcccttgggacccctctggcatcgactggaccgagtacctgcagtcttactgctacggcgtgcgacacttcctcgagaagcgaaagtag

SEQ ID NO: 96 - DNA, 1437 bp, Bicyclus anynana
atgtaccgaaacgtgaacaacaactacaagggccactctgtgtacaccaacggcgctggctctcgagtgaagtctctgctgtcctctgccaccgacaacaccaacgagtaccagtctatcgccgagtgctacaagggacagtccgtgttcatcaccggcggcaccggcttcgtcggcaaggtgctgctcgagaagctgctgtactcttgccccggcatcgacaaggtgtacctgctggtgcgagagactcagggcgccaccgctcaccagcgaatgcagaagctcctggaagaacccgccttctcgcgaatcaaggaagagaaccctcaggccttcgagaaggtgatccccatcgtgggcgacatcacccagcctcagctgggcatcatggccgagaacgaggaactgctgattaaggaagtgtctttcgtctaccacgtggccgccaccaccaagttcaacgagactctggacattgccatgaacgtgaacgtggccggaaccggacgagtgctggacctgtctaagcgaatggaaaacatcaaggccttcgtgtacgtgtctaccgcctactctaacaccgaccgagaggtggtggaagaggtgctgtaccccgctcctgtgtctctgaacgaggtgcacaagctgctgaagatcggcatcaccgacgctcaggtgaaggaactgatcaagggacgacccaacacctacaccttcaccaaggctctggctgagaacctggtggccgacaaccacggacacgtgcccgccatcatcgtgcgaccctctatcgtgtcctcgtctaagaaggaacccatcaccggatggatcgactcttggtacggcgccaccttcctggccaccgtgaccatgaagggcttcaaccgagtgttcgtgtcgtcttacgagtacaacctggacttcatccccgtggactacgtgtccaacctgatcatcgtggccgctgctcgatgcaagtgctctgacaaggtggacgtgtacaactcttctacctccggcgaaaaccctctgaagattggcgccttcttcgacgacatcattgcctactcttgcaagcacaagttctacgacatccctctgcctatggcctacctgactcgataccgatgggtcatgttcctgatcaccctgctgctgcagaccctgcctgcctatatcgccgacctgttcctgctgatcgtgggcaagaagccccgatacgtcaagctggcctctaagatctctgccgctcacgaggtcctggactacttcccctctcgaacctggtctatgtctgcccgacagaccaccgctctgttccagtctctgtctccctcggaccgagatcagtttccttgcgaccccaccgacatcgactggaaggagtacatcgtcacctactgccagggaatccgacagttcctgtgcaagtcttaa

SEQ ID NO: 97 - DNA, 1449 bp, Bombus lapidarius
atgtctcaccaccaccatcaccacggctctaacaccaagctgaacgagaacgagatcaacgagaagctgcgaaacgtgaactctatcggcggcttctacgccggcaccggcatcctgatcaccggcggcaccggattcgtcggcaagggcctgctcgagaagctcatccgaacctgctctcacattgccgccatcttcatcctgattcgacccaagcgaaaccagaccatcgagcagcgattcaagaagatcatcgacgaccccatcttcgacggcgtgcgagcacagaaccccgccattttctacaagatccacctggtcgagggcgacgtgaccctgcctgacctgggcctgctgcaaaaggaccgagacatgctgattgagaacgtgaacatcgtgttccacattgctgccaccatcaacttccaccagcctctggacatgatcgtgaacgtcaacgtgaagggcaccgccaacatcatcaagctgtgcaaggaactgaagcacgtgatctctgtggtgtacgtgtctaccgcctactctaaccccaacctgtctgacatcgaggaaaaggtctacactaccaacttggacccctctctggtgatggacatctgcgaccgacaggacaaggaactcatcaacctgatcgaggaacgaatcctcaagactcaccccaacacctacaccttcaccaagaacctggccgagcagaccatctctaacaactctaagggcctgaccgtggccatcgtgcgaccctctatcatctcttcgtccctgaaggaaccctttcctggctggctggtgtctttcgccggccagtctggcatcttcaagaacatcggcaacggcatggccaaggtgctgctcggacgaggcgacgtcatctccgacatcgtgcccgtggactacgtggtggacgccatcatgtgcgccgcctggcacgtcaccctgcagatcgacaacaacgtcaaggtgtacaactgcacctcttctgctcgacccattaagctgggcgagattgtgaacatcttcgtcgagtgttctcgagagatccccatgaagaacaccctgtggtatccctcttgcaccatcgtggccaaccgattcgtgtacaacgtgctgaacatcctgctgaacgtgctgcccgccttcgccgtggacatcttcctgcgactgcgaggcggcaagcccatggctatgaacatgaacaagtactacaacaagctggtggtcgccacctcttacttcaactctaacgagtggtctttcaagcgagacaacattgccgacatgatcaacaaggtgaacaccctcgaggacggcaacatcgtgaagctggacctgcaggacatggtgtggcgaaagtacattgccaactacctggccggcatcaagaagttcattctgaaggaagatcccaagtctatcaacgctgctccctaa

SEQ ID NO: 98 - DNA, 1407 bp, Chilo suppressalis
atggaaccctacaagtctgacgaggaaatcaagtctaagctgttctctcccgtggtgaacttctacaccggcaagtctgtgttcattaccggcgctaccggcttcctgggcaccgtgctgctcgagaagctgatgttcacctgtgctcacaacatcaagaacatctacatcctgatcaagcccaccgacggccagtctatcgacgagaagatgtctaagttcttcgactctcgagccttcgagcgactgcgagagcacaaccccaacttccgatctaagatcattcccctgaccggcgacatcaccaagaagtctatcggcctgtctgagaacgacatcttcattctgcgaaaggaagtgtctgtcgttttccactctgccgccgacacctctttccagctgtctctgtctgaggccatcatcatcaacaccaaggccaccgaggaactgctgaagatctgcaaggacatgcaccagctgaaggctttcgtgtacgtgtctaccgcctactctaactgcaaccgacctatcattgacgagaaggtgtaccctaccgacgtgtctctcgagactgtgtacgagctgctggaatactctaagtctgacaagctgatcgagttcctgttcaacggacgacccaacgcctacacctactccaaggctctgtcggaggaactggtgcagaactacggcaacatcatcccctctatcatcattcgaccctccatcatcacctcttctatcaaggaaccctatcctggctggctgtctggctggaacggcctgaaccaggtgatcctgtctggcatgaagggctacctccgatgctggttcgccgacgactcttgtatcgccgacactatccccgtggactacaccgccaacgtgatgatcgtgtctgcctgggacgcccacgagcgacgactgaagaacgacaagcagctcaaggtgttcaactgctgctctggcctgcagaaccccatcgacaccggaaacatgatgtctatctgcctcgagcacaacaagaagcacgagaaggacaagtccaaggccaacaccatgttcatcatccgaaagaacttttacgtgtacttcttctacttcctggtgctgcatctgatccccgctctgattatcgacgtgttctactttctgctgggccgagagatgctgatgtgtaccaacctgaagaagatcaagcgattctcttcgatcctgaaggtgttctgcttcaaccagtttctgttcatcgacaagaacgtccgacgactgcaccaggctctgaacgagactgacaaggtgctgttcgacttcgacgtgcgaaacatccagtggcgagtgtacctgaaggacttcgtgatcgccctgaaggaatacgacaagacctctcgaaccgtgaagatttag

SEQ ID NO: 99 - DNA, 1524 bp, Cydia pomonella
atggacatgatcgacgaggccgaggctcgaggcgagtctcagatccagaagttcctgtctggctctaccatcctgctgaccggcggaaccggcttcctgggcaagctgctggtcgagaagctgctgcgaacctgtcctgacatcaagaagatctacctgctggctcgacccaagaagaacaaggaaatccagaagcgactgcaagagcagttcgaggaccctctgtacgagcgactccgaaagcaggtccccgacttcatgtctaagatcggcgtggtcgagggcgacgtgggaaagctcggcctgggcatctctgagtctgaccgacagaccgtggtggacgaggtggacgtgatcttccacggcgctgctaccctgcgattcaacgagcccctgcgagatgccgtgttcatcaacgtgcgaggcacccgagagatgatgctgctggcccgagcctgcaccaagctgaaggccatggtgcacatctctaccgcctactctaactgcaccctgtctgagattgacgaggtgttctacgagtctcccattcctggcgacaagctgatcgacctggccgagtctctggacgaaaagaccatcaactctatcacccctggcctgatcggcgacttccccaacacctacgcctacaccaagggcgtcgccgaggacgtgctgcagaagtactctcagggactgcccgtggccgtggtgcgaccctctatcgtgatcggcaccgctaaggaccccgtcgccggctggatcgacaacgtgtacggtcccaccggtgtgattgtgggtgctgagctgggcctgctgcacgtgctccacgctgctcccaacgcctctgcctctctggtgcccggtgacgctgtggctgctgcttgcgtggctgctgcttggtctgtgtctcgagccgagaaccatcaggctcccgctcgagatgcccctcctctgtaccactgcgtgtgctctgagaaggctcccatcacctggtcgcagttcatgtctctggccgagactcacggcctggtggtgcctccaatgcaggccatgtggtactacatgctgaccctgaccaactctaaggccatgtacaccctgctcgccctgctgatgcactggatccccgcctacatcatcgacggcgtgtgcatggtgctgggcaagaagccccagctgcgaaaggcttacaccaagatcgagcagtttgccgccgtgatcgagttcttcgctctgcgagagtggcgattccacaacaacaacatgacccgactgtacaacgagctgtgcgacgccgacaagcacatctacgacttcgacacctctgccatcgactggaacgagttctttgccaactacatgaagggcatccgagtgtacctgctgaaggaccctgtgtctactatccctgagtctctgaagcgacacaagcgactgaagtggctgcactacgccctgctcaccgtgctgtctctgctggtgctgcgactgctgtggttcttcgtgtctttcctgttttag

SEQ ID NO: 100 - DNA, 1368 bp, Helicoverpa armigera
atggtggtcctgacctctaaggagactaagccctccgtggccgagttctacgctggcaagtctgtcttcatcaccggcggaaccggtttcctgggcaaggtcttcattgagaagctgctgtactcctgtcccgacatcggcaacatctacatgctgatccgagagaagaagggactgtctgtgtccgagcgaattaagcacttcctggacgaccccctgttcacccgactgaaggagaagcgacccgccgacctggagaagatcgtgctgattcccggagacatcaccgctcccgacctgggtattacctctgagaacgagaagatgctgatcgagaaggtgtctgtcatcattcactccgccgctaccgtcaagttcaacgagcccctgcccaccgcctggaagatcaacgtggagggaacccgaatgatgctggctctgtctcgacgaatgaagcgaattgaggtcttcatccacatttccaccgcctacaccaacaccaaccgagaggtggtggacgagatcctgtaccctgctcctgctgacattgaccaggtgcaccgatacgtcaaggacggtatctctgaggaagagactgagaagattctgaacggccgacccaacacctacaccttcaccaaggccctgaccgagcacctggtggctgagaaccaggcttacgtgcccaccatcattgtccgaccctccgtggtcgccgctatcaaggacgagcccattaagggatggctgggtaactggtacggagctaccggactgaccgtgttcaccgctaagggtctgaaccgagtcatctacggccactcttccaacatcgtggacctgattcccgtggactacgtcgccaacctggtcattgccgctggcgctaagtcttccaagtccaccgagctgaaggtgtacaactgttgctcttccgcctgcaaccccatcaccattggaaagctgatgtctatgttcgccgaggacgctatcaagcagaagtcctacgctatgcccctgcccggttggtacatcttcaccaagtacaagtggctggtcctgctgctgaccattctgttccaggtcatccccgcctacattaccgacctgtaccgacacctgatcggcaagaacccccgatacattaagctgcagtctctggtcaaccagacccgatcttccattgacttcttcacctctcactcctgggtcatgaaggctgaccgagtccgagagctgttcgcctctctgtcccccgctgacaagtacctgttcccctgtgaccccaccgacatcaactggacccactacattcaggactactgctggggagtgcgacacttcctggagaagaagtcctacgagtag

SEQ ID NO: 101 - DNA, 1371 bp, Helicoverpa assulta
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 102 - DNA, 1362 bp, Heliothis subflexa
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 103 - DNA, 1362 bp, Heliothis virescens
atggttgtcttgacctccaaagaaactaagccatctgttgctgaattttacgctggtaagtctgttttcattactggtggtactggtttcttgggtaaggttttcattgaaaagttgttgtactcctgcccagatatcgttaacatctacatgttgatcagagaaaagaagggtttgtccgtttccgaaagaatcaagcaatttttggatgaccctttgttcaccagattgaaggacaaaagaccagctgatttggaaaagatcgttttgattccaggtgatattaccgctccagatttgggtattactgctgctaacgaaaagatgttgatcgaaaaggtttccgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagtttaccaatacgtcaaagaaggtatctccgaagaagataccgaaaaaatcttgaacggtagaccaaacacttacactttcactaaggctttgaccgaacatttggttgctgaaaatcaagcttacgttccaaccattatcgttagaccatcagttgttgctgccattaaggatgaaccattgaaaggttggttgggtaattggtttggtgctacaggtttgactgtttttactgctaagggtttgaacagagttatctacggtcattccaactacatcgttgatttgatcccagttgattacgttgccaacttggttattgctgctggtgctaaatctaacacctcttctgaattgaaggtctacaactgttgttcctcatcatgtaacccagttaagatcggtactttgatgtctatgtttgctgatgatgccatcaagcaaaagtcttatgctatgccattgccaggttggtacatttttactaagtacaagtggttggtcttgttgttgaccttcttgttccaagttattccagcctacattaccgatttgtcaagacacttggttggtaagagtccaagatatatcaagttgcaatccttggtcaatcaaaccagatcctccattgatttcttcaccaatcattcttgggttatgaaggccgatagagtcagagaattatacgcttctttgtctccagcagataagtacttgtttccatgtgatccagttaacatcaactggacccaatacttgcaagattactgttggggtgttagaaacttcttggaaaaaaagacttaa

SEQ ID NO: 104 - DNA, 1407 bp, Plutella xylostella
atgaagctgggcaccttcggcaacatggccgccaacggcggctctgtgtgcgacttctacgccaactctaccgtgttcattaccggcggcaccggattcctcggcaaggtgctgatcgagaagctgctgcgatcttgccgaaagatcgacaagatctacgtcctgctgcgaggcaccaccaacaagaccgctcaagagcgactcgaggacctgtctcacgaggtcctgttcgagaaggtgcgacaggacgaccccaacatcatgaagaagctgatccccgtcaacggcgacctgactctgcccaacctgggcatgaacaacgaggactactctctgatcgtgaagaacgtgtctatcgtgttccacgccgctgcctctatccgattcaacctgaagatcaaggaagccctgcactgcaacgtcgagggcaccaagaaggtgatcgagctttgccacaaggcccgatgcatcaaggccttcgtgcacatctctaccgcctactctaacaccgacaagaccttcgtgaaggaagaggtgtaccccgctcctgagaacctggataagatctatgtgatcatcggccagaacggcgacgacaccgaggccgacgacctgacctcttctcgacccaacacctacacctacgccaaggccgtggccgagtacgaggtgctgcgaaaccacggatctgtgcccaccgccatcattcgaccctctatcgtgacccctatctggcgagatcccactcctggctggctggacaagtggtctggcggcacccctatcatcttcggcctgtgcaagggcctgctccgatgcctggacacccagccttctaacgtgatcgacttcatccccgccgacattgccgtgaacctgtccatcgtggccgctgccaagtgccacgtggacacccctaagatccgagtgtacaactgctgctctggccctacaaaccccatgaccttcaagatgctgtctgactactgccacgagaactctatcaagcacggcctgcactggttcccttatcctaagatcttcctgtctaccaaccgagtgttcctgaagattgtgcgactggtgctgcagttcctgcctctgttcctggccgacatgtggctgaagattcgaggcaagcagccccgattcgtgaagatgctggaaaagctgaccaaggtggaaaagaccctgcacttcttcatcaagaaccagtggcagttcgagaaccacaacgttgaggccctgatctctaccctgtctccttcggaccgagtgcagttcccttgcgacccctctgagatctgctggaagacccacttcgaggattactgcctgggcatcgacaagtacctgatgaacacctctgtgaagtctgccaacatcaactctggcatgtacaagtag

SEQ ID NO: 105 - DNA, 1365 bp, Spodoptera exigua
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggaaaagcgacccggcgacctggacaagattgtgctgatccccggcgacgtgaccgtgcctggcctgggcatctctgacgagaacgaggccatcctgatcgacaaggtgtctgtggtgatccactctgccgccaccgtgaagttcaacgagcccctcgagactgcctggaacgtgaacgtcgagggcacccgaatgatcatggccctgtctcgaaagatgaagcgaatcgagatcttcatccacatctctaccgcctacaccaacaccaaccgagccgtggtggacgaggtgctgtaccctcctcctgccgacatcaacgaggtgcaccagtacgtgaagaacggcatcaccgaggaagagactgagaagatcctgaacggacgacccaacacctacaccttcaccaaggctctgaccgagcacctggtggccgagaaccaggcctacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccagtcctctcacgtggtggatctgattcccgtggactacgtggccaacctggtgatcgtggctggcgccaagacctaccgatctaacgaggtgactatctacaactcttgttcttcttcttgcaaccccatcaccatggaacgactggtgggcctgttcattgacgacaccgtcaagcacaactcttacgtgatgcccctgcctggctggtacgtgtactctaactaccgatggctggtgtacctggtgaccatcatcttccagatgatccccgcctacctggccgacatcggccgacgactgctgggcaagaaccctcggtactacaagctgcagtctctggtggctcagacccaagaggccgtgcacttcttcacctctcacacctgggagatcaagtctaagcgaacctctgagctgttcgcctctctgtctcataccgaccagcgaatcttcccatgcgacgccaagaagatcgactggaccgactacatcaccgactactgctctggcgtgcgacagttcctggaaaagaagaagtag

SEQ ID NO: 106 - DNA, 1353 bp, Spodoptera frugiperda
atgtctaaggccaactgctctaacgtggcctcgttctacgccggcaagtctgtgttcatcaccggcggcaccggcttcgtgggcaagaccctgatcgagaagctgctgtactcttgctctggcatcgacaagatctacgtgctggtgcgagacaagtacggcaagcaggcctctgagcgactggcccgagtgacctctactcccgtgttcgaccgactcaagacctctaagtctgaggaactgaagaagatcgtcgtcatctctggcgacatcacccgagagaacctgggcctgcaagaggacgtgctcaagaccctcgagaacgaggtgtctgtggtgttccacctggccgccaccgtggccttcaagctgcccctgaaggacgccatgcgaatcaacgtgaacggcaccgagaacgtgatcgagctgtgccgacgaatgaagaagctgcaggccttcgtccacgtgtctaccgccttcaccaactctgaccgaaaggaaatcaacgagatggtgtaccccatgcctatcaacctggaagaggcccgacacgtggcctacacctactctcacgacgacgtgatcgtgtctgccttcatgggaaagaagcccaacacctacaccttctccaaggccttcgccgagcagcaggtcatgaagcagtgcgaggacctgcctaccgccatcattcgaccctctatcgtgatgtcctctgtgaaggaaccctgtcctggctggatcgacacctggaacggctctaccggcctgttcgtgggaatgcccgctggcatcctgcgagtggtgctcggacgaggatctaacgtgaccgacctggtgcctgtggacatcgtgaccaacctgatcgtggtggccgctaccgagtgccagaagtctaaggaaatcaaggtctacaactgctgcaccggcacttctaaccccatcacctgtgacgaggctaccaccatctctcgaaaggtggccctgaagtactctctgaacgagctgccctggcctttcctgatgtttaccccttctgtgttcctgtataccctgatcaccttcctgctgcagatcgtgcccgcctacctgatcgacctgtggtgcatcctgaccggccgaaaggctacccagatcaagctgcagcgacgacttaagaaggtggtcgacgccgtcaagttctttctgctgaacgagtggaagttctctgacgacaacattcgagatctgcttaagaccatgtctcccgtggacaaggaaatcttcaacttcgacgtcaagaccatcaactgggagacttgcatcgaggactacatcctgggagcccgaaagtacctgctgaagtctgagaagtag

SEQ ID NO: 107 - DNA, 1365 bp, Spodoptera littoralis
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggaaagatcgtgctgatccccggcgacatcaccgtgcctggcctgggcatctctgaggaaaacgagactatcctgaccgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactacaagtggctggtgttcctggtgaccgtgatcttccaggtgatccccgcctacctgggcgacatcggccgacgactgctgggcaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 108 - DNA, 1365 bp, Spodoptera litura
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggcaagatcattctgatccccggcgacatcaccgtgcctggcctgggcatctctgaggaaaacgagactatcctgatcgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactaccgatggctggtgttcctggtgaccctgatcttccaggtgatccccgcctacctgggcgacatcggccgacgactgctgggaaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 109 - DNA, 1557 bp, Tyta alba
atggtgtctatccccgagtactacgagggcaagaacatcctgctgaccggcgccaccggcttcatgggcaaggtgctgctcgagaagctgctgcgatcttgccccaaggtgaaggccgtgtacgtgctggtgcgacacaaggccggacagacccctgaggctcgaatcgaggaaatcaccaactgcaagctgttcgaccgactgcgagatgagcagcccgacttcaaggccaagatcatcgtgatcacctctgagctgacccagcctgagctggacctgtctgagcccatcaaggaaaagctgatcgagcgaatcaacatcatcttccactgcgccgccaccgtgcgattcaacgagactctccgagatgccgtgcagctgaacgtgaccgctactcagcagctcctgttcctggctcagcgaatgaagaacctggaagtgttcatgcacgtgtctaccgcctacgcctactgcaaccgaaagcagatcgaagagatcgtgtaccctcctccagtggaccccaagaagctgattgactctctcgagtggatggacgacggcctggtgaacgacatcacccctaagctcatcggcgaccgacctaacacctacacttacaccaaggctctggccgagtacgtggtgcagcaagagggcgccaagctgaacaccgccatcattcgaccctctatcgtgggcgcctcttggaaggaaccctttcctggctggatcgacaacttcaacggcccctctggcctgttcattgccgccggaaagggcatcctgcgaaccatgcgagcctctaactctgccgtggccgacctggtgcctgtggacgtggtggtgaacaccactctggccgctgcctggtactctggcgtgaaccgacctcgaaacgtgatgatctacaactgcaccaccggcggcactaaccccttccactggggcgaagtgggctaccacatcaacctgaacttcaagatcaaccctctcgagaacgccgtgcgacaccccaactgttctctgcagtctaaccctctgctccatcagtactggaccgccgtgtctcacaccatgcctgcctttctgctggacctcctgctgcgactgaccggacacaagccctggatgatgaagaccatcactcgactgcacaaggccatgatgctcctcgagtacttcacctccaactcttggatctggaacaccgagaacatgaccatgctgatgaaccagctgaaccccgaggacaagaagaccttcaacttcgacgtgcgacagctgcactgggctgagtacatggaaaactactgcatgggcaccaagaagtacgtcctgaacgaggaaatgtctggactgcccgctgccagaaagcacctgaacaagctgcgaaacatccgatacggcttcaacaccgtgctggtcatcctgatctggcgaatcttcattgcccgatctcagatggcccgaaacatctggtacttcgtggtgtctctgtgctacaagttcctgtcttacttccgagcctcttctaccatgcgatactctaagctgtag

SEQ ID NO: 110 - DNA, 1362 bp, Trichoplusia ni
atgatcgccgtgacctctcacgagaacgaggcctctatcgtggacttctacgagggcaagtctgtgttcattaccggcggcaccggattcctcggcaaggtgttcatcgagaagctgctgtactgctgtcccggcctggtgaagatctacatgctgatccgagagaagaagggcctgaccatcaaggaacgaattgaccgattcgtggacgactctctgttcgaccgactcaagacccagcgacctaaggacctggacaagatcgtgctgatccccggcgacatcactgccccttctctgggcatctctcaagagcaagagaacatcctgatcgaagaggtgtctgtgatcatccactctgccgccaccatcaagttcaacgcccctctggaagaggcctggtctatcaacgtggacggcacccgacgaatcctggacctgtgccgaaacatgaagaaggtcgaggttttcctgcacatctctaccgcctacaccaacatcaaccgaaacgtgatcgaggaagtgctctaccctcctccagccgacattaaggaactggaaaagctggacaagcacaacgtgaccgagaagcagaccatgaagctgctcaacggcttccccaacacctacaccttcaccaagtctctgaccgagcacctggtggccgagaactgcgcctacatgcccaccatcatcgtgcgaccctctgtggtggccgccgtgctcgaggaccccatcaagggctggctggaaaactggtacggcgccaccggcatctccgtgttcaccgccaagggactgaaccgagtgttcctgggccacaagaacattattgtggacgtgattcccgccgactacgtggccaacctggtgatcattgccggcgctcgaaacaacaagtccaaggaactcaagatctataactgctgttcttctggctgcaaccctctgaccatcggcaacctgctgggcgccttcatcgacgacgccatccgacacaagacctacgctatgcccctgcctggctggttcatcttcacccgataccgactggtggctaccatcgtgaccttcatcttccaggtggtgcccgcctacctggccgacctgtaccgacgaattatcggcaagaagcccaagtacaccaagctgctctacctggtggtgcagacccgagtggccctggacttcttctcctctaactcttgggtgatgaaggccgaccgaatgcgagagctgtacgcctctctgtctaacaacgacaagcgaatgttcccttgcgaccccactcacatcaactggcccgagtacctgtctgactacggcgctggcgtgcgacgattcctcgagaagcgaaactag

SEQ ID NO: 111 - protein, 547 aa, Drosophila grimshawi
MGDSTLKLPGAAAAPLKPPAVAALSVSSLQLPSSAPQKLTSAASNGNVSGSATGNPRNKCALKPGYSLMSWIRLCNSGADLSGTAGRVVPVSRTELARHNQVNDAWMAIRGKVFNVTRYMDYHPGGVDELMRGVGRDATKLFDEVHAWVNYPQLLGKCYVGPLKDTVASKKATDAELLRLPPPSAPAELKPRFDWIQQRRELTLYFYTRRWANPGVLLKRKTAEELEVRVQLAEIWHLYNFKLHSGIIWPPKALRVSPETGKIELVLSKQEEQPWSSYGSHEASKTSGAPEEELLDFMVQHCSHFNHDSFELCLESVEQELLLHLPVGYHLDIEVPHNGERVQRSYTPVPHNCLLAEAYSSSSSPNSQRFLIKHYENGIVSSQLNQLESGGNLQLTLPRGNFNLAQLEAHRHILLLAAGSGLTPMLSLLRPLLERHTNRIERLQLLYFNKTQADIWWKDKLLALQSSDERFLCQHILSQEVEEQPPTPNQNTRICPELLAPLFEQQQPLGERFSYVAICGPTGFNTAALDSLRRLEFKLTNVHVFQG

SEQ ID NO: 112 - protein, 535 aa, Drosophila melanogaster
MSDPSLKPPASNQPLRPPQLMAPAGSGGGLQLPVTAPQKLNSNGSSASGSATGNPRNKCALKPGYSLMNWIRLCNSGADLSGTGGRLVPVSRTELARHNKIDDAWMAIRGRVFNVTRYMDFHPGGVDELMRGVGRDATKLFDEVHAWVNYPQLLGKCYVGPLKDNETKPAKESPQITNVILQPPEIVPRFDWIQQRSELTLIFYTRSLANPGLVVRRKDLQQLSVRVLVQHNWHSFDFQLTNNVEWPPKLAKIGSETGKIELVFVKEEAEPWPTYGTHVSNRLDSSRLHDETFEYEVVHSKDFNHDSFELCLQSVGQKVLMVLPAGYHVDIEVPLEGRVIQRSYTPVDHTYLRLENIRSSRSECLHFLIKRYPNGPVSSHLQKLETGSRVHWSAPRGSFQLSDLTAHRNILLLAAGSGLTPILSLIQPILKRNTNRIESLQLLYFNKTNEDIWLKEKLHELHTDDERFSCTNYVSQSEDNPQRIALELLAPLFQKNQPERCTYVLICGPSGFNTAALDILSQLDVKANQIHVFRG

SEQ ID NO: 113 - protein, 521 aa, Homo sapiens
MLNVPSQSFPAPRSQQRVASGGRSKVPLKQGRSLMDWIRLTKSGKDLTGLKGRLIEVTEEELKKHNKKDDCWICIRGFVYNVSPYMEYHPGGEDELMRAAGSDGTELFDQVHRWVNYESMLKECLVGRMAIKPAVLKDYREEEKKVLNGMLPKSQVTDTLAKEGPSYPSYDWFQTDSLVTIAIYTKQKDINLDSIIVDHQNDSFRAETIIKDCLYLIHIGLSHEVQEDFSVRVVESVGKIEIVLQKKENTSWDFLGHPLKNHNSLIPRKDTGLYYRKCQLISKEDVTHDTRLFCLMLPPSTHLQVPIGQHVYLKLPITGTEIVKPYTPVSGSLLSEFKEPVLPNNKYIYFLIKIYPTGLFTPELDRLQIGDFVSVSSPEGNFKISKFQELEDLFLLAAGTGFTPMVKILNYALTDIPSLRKVKLMFFNKTEDDIIWRSQLEKLAFKDKRLDVEFVLSAPISEWNGKQGHISPALLSEFLKRNLDKSKVLVCICGPVPFTEQGVRLLHDLNFSKNEIHSFTA

SEQ ID NO: 114 - protein, 671 aa, Spodoptera litura
MADSPEMEQKPTISNTHRVSLPTVLCTPESTDDNSDISIKVNSIISSDSKMSLPSNLGTPTSPLRLSSPLSLPSSPCVKNSPQSTNSLPNPNPKPMTSSTASNKTVVNSGSATGNPRNKCALQPGHSLMDWIRLGNSGKDLTGVGGRMRPVTPAELSTHNTTNDAWLAIRGRVYNITHYLPYHPGGPEELMRGAGMDATQLFDKVHPWVNYDSLLAKCLVGPLRFDLPDAEELFDTSNPSPKSDRLREPSKAQELVRKSMENLANCITPVRKKITKGDDNSKGSPPSKIMQSLVQSADLPMSISRRAASSPVKKTDKSPDSPLPLRHDWIQTSTKLTISVYTGHLANPGGCARITEGFLLIEVATNGWLRTLKIKPEAKLKEPLQLRVFAESGKIEITALKAEPSVWKGCGEVTLGAASRVSSPRTVECRVMEVSRVSHDTSLLSVCPRAGPVVVPLGHHVRVHRRVEGSECVRSYTPVGEGWGPPDGTEIFSALRLAVKRYDSGVLSPHLTALKVGDLITLSGPYGNFQLQKLKTVKSLYLIAAGTGITPMLGLIKFMMSRSNPRCERTHLIFFNKTEADILFLENFEEMAKEDDRLKITHVLSDASSSWSGHKGRIRDELLTEILGQVKCDDKCSHYSCLCGPTEFTYAGLDLLKKHGFKDDCIHAFMG

SEQ ID NO: 115 - DNA, 1644 bp, Drosophila grimshawi
atgggcgactctaccctgaagctgcccggtgccgccgctgctcccctgaagcctcctgccgtggccgctctgtctgtgtcctctctgcagctgccctcttcggctccccagaagctgacctctgccgcctctaacggcaacgtgtctggctctgccactggcaaccctcgaaacaagtgcgctctgaagcccggctactctctgatgtcttggatccgactgtgcaactctggcgccgacctgtctggcaccgccggacgagtggtgcccgtgtctcgaaccgagctggcccgacacaaccaggtgaacgacgcctggatggccattcgaggcaaggtgttcaacgtgacccggtacatggactaccatcctggcggcgtggacgagctgatgcgaggcgtgggccgagatgccaccaagctgttcgacgaggtgcacgcctgggtgaactaccctcagctgctgggcaagtgctacgtgggacccctgaaggacaccgtggcctctaagaaggccaccgacgccgagctgctgcgactgcctcctccatctgctcccgctgagctgaagccccgattcgactggattcagcagcgacgagagctgaccctgtacttctacacccgacgatgggctaaccccggcgtgctgctgaagcgaaagaccgccgaggaactcgaggtgcgagtgcagctggccgagatctggcacctgtacaacttcaagctgcattctggcatcatctggcctccaaaggctctgcgagtgtctcccgagactggcaagatcgagctggtgctgtctaagcaagaggaacagccctggtcctcttacggctctcacgaggcctccaagacctctggcgctcccgaggaagaactgctggacttcatggtgcagcactgctctcacttcaaccacgactcgttcgagctgtgcctcgagtctgtcgagcaagagctgctcctgcatctgcccgtgggctaccacctggacatcgaggtgccccacaacggcgagcgagtccagcgatcttacacccctgtgcctcacaactgcctgctggctgaggcctactcttcttcctcttcgcccaactctcagcgattcctgatcaagcactacgagaacggaatcgtgtcctcgcagctgaaccagctggaatctggcggcaacctgcagctcaccctgcctcgaggaaacttcaacctggctcagctcgaggcccaccgacacatcctgctgctggccgctggctctggactgacccctatgctgtctctgctccgacctctgctcgagcgacacaccaaccgaatcgagcgactgcagctcctgtactttaacaagacccaggccgacatctggtggaaggacaagctgctcgccctgcagtcctctgacgagcgatttctgtgccagcacatcctgtctcaagaggtggaagaacagcctcctactcctaaccagaacacccgaatctgccccgagcttctggctcccctgttcgagcagcagcagcccctgggcgagcgattctcttacgtggccatctgcggccccaccggcttcaacaccgccgctctcgactccctgcgacgactcgagttcaagctgaccaacgtccacgtgttccagggctag

SEQ ID NO: 116 - DNA, 1608 bp, Drosophila melanogaster
atgtctgacccctcgctgaagcctcctgcctctaaccagcctctgcgacctcctcagctgatggctcccgccggatctggcggcggactgcagctgcccgtgaccgctcctcagaagctgaactctaacggatcttctgcctctggctctgccactggcaaccctcgaaacaagtgcgctctgaagcccggctactctctgatgaactggatccgactgtgcaactctggcgccgacctgtctggcaccggcggacgactggtgcccgtgtctcgaaccgagctggcccgacacaacaagatcgacgacgcctggatggccattcgaggccgagtgttcaacgtgacccggtacatggactttcaccccggtggcgtggacgagctgatgcgaggcgtgggccgagatgccaccaagctgttcgacgaggtgcacgcctgggtgaactaccctcagctgctgggcaagtgctacgtgggacccctgaaggacaacgagactaagcccgccaaggaatctccccagatcaccaacgtgatcctgcagcctcctgagatcgtgccccgattcgactggattcagcagcgatctgagctgaccctgatcttctacacccgatctctggctaaccccggcctggtggtgcgacgaaaggacctgcagcagctgtctgtgcgagtgctggtgcagcacaactggcactcgttcgacttccagctgaccaacaacgtcgagtggcctcctaagctggccaagatcggctctgagactggcaagatcgagctggtgttcgtgaaggaagaggctgagccctggcctacctacggcacccacgtgtctaaccgactggactcttctcgactgcacgacgagactttcgagtacgaggtggtgcactctaaggacttcaaccacgactctttcgagctgtgcctgcagtctgtgggccaaaaggtgctgatggtgctgcccgctggctaccacgtggacatcgaggtgcctctcgagggacgagtgatccagcgatcttacacccctgtggaccacacctacctgcgactcgagaacatccgatcttcccgatctgagtgcctgcacttcctgatcaagcgataccccaacggacccgtgtcctctcacctccagaagctcgagactggatctcgagtgcactggtctgctccccgaggctcgttccagctgtccgacctgaccgctcaccgaaacatcctgctgctggctgccggctccggactgacccctatcctgtctctgattcagcccatcctgaagcgaaacaccaaccgaatcgagtctctgcagctcctgtacttcaacaagaccaacgaggacatctggctgaaggaaaagctgcacgagctgcacaccgacgacgagcgattctcttgcaccaactacgtgtctcagtctgaggacaaccctcagcgaatcgctctcgagctgctcgctcccctgttccagaagaaccagcctgaacgatgcacctacgtgctgatctgcggcccctctggcttcaacaccgccgctctggacattctgtctcagctggacgtgaaggccaaccagatccacgtgttccgaggctag

SEQ ID NO: 117 - DNA, 1566 bp, Homo sapiens
atgctgaacgtgccctctcagtctttccccgctcctcgatctcagcagcgagtggcctctggcggccgatctaaggtgcccctgaagcagggccgatctctgatggactggatccgactgaccaagtctggcaaggacctgaccggcctgaagggccgactgatcgaggtgaccgaggaagaactgaagaagcacaacaagaaggacgactgctggatctgcatccgaggcttcgtgtacaacgtgtctccctacatggaataccatcctggcggcgaggacgagctgatgcgagccgccggatctgacggcaccgagctgttcgaccaggtgcaccgatgggtgaactacgagtctatgctgaaggaatgcctggtgggccgaatggccatcaagcccgccgtcctgaaggactaccgagaggaagagaagaaggtcctgaacggcatgctgcccaagtctcaggtgactgacaccctggccaaggaaggcccttcttacccctcttacgactggttccagaccgactctctggtgaccattgccatctacaccaagcagaaggacatcaacctggactctatcatcgtggaccaccagaacgactcgttccgagccgagactatcatcaaggactgcctgtacctgatccacatcggcctgtctcacgaggtgcaagaggacttctctgtgcgagtggtcgagtctgtgggcaagatcgagatcgtgctgcagaagaaggaaaacacctcttgggactttctgggacaccctctgaagaaccataactctctgattccccgaaaggacaccggcctgtactaccgaaagtgccagctgatctctaaggaagatgtgacccacgacacccgactgttctgcctgatgctgcctccttcgactcatctgcaggtccccatcggccagcacgtgtacctgaagctgcccatcaccggaaccgagattgtgaagccctatactcccgtgtctggctctctgctgtctgagttcaaggaacccgtgctgcctaacaacaagtacatctactttctgatcaagatctaccccaccggactgttcacccctgagctggaccgactgcagatcggcgacttcgtgtctgtctcttcgcccgagggcaacttcaagatctccaagttccaagagcttgaggacctgttcctgctggccgctggcaccggcttcacccctatggtgaagatcctgaactacgccctgaccgacattccctcgctgcgaaaggtgaagctgatgttcttcaacaagaccgaggacgacatcatctggcgatcccagctcgagaagctggccttcaaggacaagcgactggacgtcgagttcgtcctgtctgctcccatctctgagtggaacggcaagcagggacacatctctcccgctctgctctccgagttcctgaagcgaaacctggacaagtccaaggtgctggtgtgcatctgtggccccgtgcctttcaccgagcagggcgtgcgactgctgcacgacctgaacttctctaagaacgagatccactctttcaccgcctag

SEQ ID NO: 118 - DNA, 2016 bp, Spodoptera litura
atggccgactctcccgagatggaacagaagcccaccatctctaacacccaccgagtgtctctgcccaccgtgctgtgcacccctgagtctaccgacgacaactctgacatctctatcaaggtgaactctatcatctcttctgactctaagatgtcgctgccctctaacctgggaacccctacctctcctctgcgactgtcgtcgcccctctctctgccctcttcgccctgcgtgaagaactctccccagtctaccaactctctgcctaaccctaacccaaagcctatgacctcttctaccgcctctaacaagaccgtggtgaactccggctctgccactggcaaccctcgaaacaagtgcgctctgcagcccggccactctctgatggactggatccgactgggcaactctggcaaggacctgaccggcgtcggcggacgaatgcgacccgtgactcccgccgagctgtctacccacaacaccaccaacgacgcctggctggccatccgaggccgagtgtacaacatcacccactacctgccttaccatcctggcggacccgaggaactgatgcgaggcgccggaatggacgctacccagctgttcgacaaggtgcacccttgggtgaactacgactctctgctggccaagtgcctggtgggacccctgcgattcgacctgcctgacgctgaggaactgtttgacacttctaacccctcgcctaagtctgaccgactgcgagagccctctaaggcccaagagctggtgcgaaagtctatggaaaacctggccaactgcatcacccctgtgcgaaagaagatcaccaagggcgacgataactctaagggctcgcctccttcgaagatcatgcagtctctggtgcagtctgctgacctgcctatgtctatctctcgacgagccgcttcttcgcccgtgaagaagaccgacaagtctcctgactctcccctgcctctccgacacgactggattcagacctctaccaagctgaccatctctgtgtacaccggccacctggctaaccccggtggctgcgcccgaattaccgagggcttcctgctgatcgaggtcgccaccaacggctggctgcgaaccctgaagatcaagcccgaggccaagctgaaggaacccctgcagctgcgagtgttcgccgagtctggcaagatcgagatcaccgctctgaaggctgagccctctgtgtggaagggctgcggcgaggtgaccctgggagccgcctctcgagtgtcctcgcctcgaaccgtcgagtgccgagtgatggaagtgtcccgagtgtcccacgacacctcgctgctgtctgtgtgtccccgagctggccccgtggtggtgcccctgggccaccacgtgcgagtgcaccgacgagtcgagggctctgagtgcgtgcgatcttacacccctgtcggcgaaggctggggccctcctgacggcaccgagatcttctcggccctgcgactggccgtgaagcgatacgactctggcgtgctgtctccccacctgactgccctgaaggtgggcgacctgattaccctgtctggcccctacggcaacttccagctgcaaaagctcaagactgtgaagtctctgtacctgattgccgccggaaccggaatcacccctatgctgggcctgatcaagttcatgatgtctcgatctaaccctcgatgcgagcgaacccacctgattttcttcaacaagactgaggccgacatcctgttcctcgagaacttcgaggaaatggccaaggaggacgaccgactgaagattacccacgtgctgtccgacgcctcttcttcttggtctggccacaagggacgaatccgagatgagctgctgaccgagatcctcggacaggtgaagtgcgacgacaagtgctctcactactcttgcctgtgcggccctaccgagtttacctacgccggcctggacctgctgaagaagcacggcttcaaggacgactgcatccacgccttcatgggctag

SEQ ID NO: 119 - protein, 608 aa, Yarrowia lipolytica
MSDDKHTFDFIIVGGGTAGPTLARRLADAWISGKKLKVLLLESGPSSEGVDDIRCPGNWVNTIHSEYDWSYEVDEPYLSTDGEERRLCGIPRGHCLGGSSCLNTSFVIRGTRGDFDRIEEETGAKGWGWDDLFPYFRKHECYVPQGSAHEPKLIDFDTYDYKKFHGDSGPIKVQPYDYAPISKKFSESLASFGYPYNPEIFVNGGAPQGWGHVVRSTSNGVRSTGYDALVHAPKNLDIVTGHAVTKILFEKIGGKQTAVGVETYNRAAEEAGPTYKARYEVVVCCGSYASPQLLMVSGVGPKKELEEVGVKDIILDSPYVGKNLQDHLICGIFVEIKEPGYTRDHQFFDDEGLDKSTEEWKTKRTGFFSNPPQGIFSYGRIDNLLKDDPVWKEACEKQKALNPRRDPMGNDPSQPHFEIWNAELYIELEMTQAPDEGQSVMTVIGEILPPRSKGYVKLLSPDPMENPEIVHNYLQDPVDARVFAAIMKHAADVATNGAGTKDLVKARWPPESKPFEEMSEEWETYVRDKSHTCFHPCGTVKLGGANDKEAVVDERLRVKGVDGLRVADVSVLPRVPNGHTQAFAYAVGEKAADLILADIAGKDLRPRI

SEQ ID NO: 120 - protein, 720 aa, Yarrowia lipolytica
MSETDHLLAAEPVAEYPQYTPWPNSRKSVDTEFSATSWIYDLVLWIFTACFDIFFREIRPRGAFRIPRKGPVLFVAAPHANQFVDPVILMNQVKQEAGRRISFLVAEKSMRRAAVGRMARSMNSIPVVRAQDNAKKGEGKIYVDAEDPTKIHGIGTQFTKQCEVRGLVVCSSSVGSIDVAEIVSDTLLIARKEFKGPKAKEALKESNGGITYKYADYVNQATVYRSVFDKLHHGGCVGIFPEGGSHDRTELLPLKAGVAIMALGALAEDPSCGVRIVPCGLNYFHAHKFRSRAVVEFGSPIAIPPDLVEKYKAGGEAKREAVKTVLDITAAGLKSVTVQVQDFDTLMLIQAIRRLYRPPGKKIPLPMVVELNRRLVYAYNHYKDDPRIEEMKQEIRKYNKFLQAMGLKDHQVEKARISKIEILGRLLYRSIKLVFLSIGCLPGLLLFSPIFIISKSISKTKAKEALKASSVKIKANDVVATWKVLVAMGLTPVLYILYSLVGSVVIRKLDLISWFPTILLPGLVLSIIITTSYAALAMGEAGMDIFKSLRPLALALNPSTKNSLLKLQNERKRLVLKSSELVTSLGPELFPDFPENSILQGSDKFEDEENYENEKRSHSRSTSATSLSAMSEGDGDELVREVRKGASYFPVSTISEDEDQAISRVGSEASLADIPLFGMSRSQSGASLSEASTHGSSTGADAEEAKTEVTRRIALAMEEK

SEQ ID NO: 121 - protein, 518 aa, Yarrowia lipolytica
MSTFDWESIVPATPLDQIPGDIQRLRKGFRSGKTLDLNYRLDQIRNLHYVLRDNVEAIKDAVYKDLGRPKHETDLCEVGFLWGEFNNVVANLKKWAADEDVKTNLQYSISSPKIRKRPLGNVLIISPWNYPFMLTVSPLIGALAAGNTVAVKFSEMAPHTSKIVGDLCTKALDPDVFQAIQGGVPVVTKTLEQKFDKIMYGNHTVGKIIATAANKYLTPVILELGGKSPVFVTKNCKNIKLAAKRALWGKVVNAGQTCVAPDYVIVEPEVEQEFIDACKYWINEFYSGKIDQYNPDFAKIATPNHWNRLTSMLSKSKGEIITGGNTDEKTRFIAPTVVAKVPDNDSLMEDEIFGPLLPILTARSVEEGIKYVHENHDTPLAMYVFTDKASEGDYIQSQINSGGLIFNDTLIHVGCVQAPFGGVGMSGYGAYHGEDSFLAFTHRQTYLNQPKLLEPLQDVRYAPYTKTKRSMVKNLLLVGPIFPRTGSVYPNVLIRIFRKIWFWVLIVAIGAAGAKALL

SEQ ID NO: 122 - protein, 529 aa, Yarrowia lipolytica
MTTTATETPTTNVTPTTSLPKETASPGGTASVNTSFDWESICGKTPLEEIESDISRLKKTFRSGKTLDLDYRLDQIRNLAYAIRDNENKIRDAIKADLKRPDFETMAAEFSVQMGEFNYVVKNLPKWVKDEKVKGTSMAYWNSSPKIRKRPLGSVLVITPWNYPLILAVSPVLGAIAAGNTVALKMSEMSPNASKVIGDIMTAALDPQLFQCFFGGVPETTEILKHRWDKIMYTGNGKVGRIICEAANKYLTPVELELGGKSPVFVTKHCSNLEMAARRIIWGKFVNGGQTCVAPDYVLVCPEVHDKFVAACQKVLDKFYPNNSAESEMAHIATPLHYERLTGLLNSTRGKVVAGGTFNSATRFIAPTIVDGVDANDSLMQGELFGPLLPIVKAMSTEAACNFVLEHHPTPLAEYIFSDNNSEIDYIRDRVSSGGLVINDTLIHVGCVQAPFGGVGDSGNGGYHGKHTFDLFSHSQTVLRQPGWVEMLQKKRYPPYNKSNEKFVRRMVVPSPGFPREGDVRGFWSRLFN

SEQ ID NO: 123 - protein, 377 aa, Yarrowia lipolytica
MWGSSHAFAGESDLTLQLHTRSNMSDNTTIKKPIRPKPIRTERLPYAGAAEIIRANQKDHYFESVLEQHLVTFLQKWKGVRFIHQYKEELETASKFAYLGLCTLVGSKTLGEEYTNLMYTIRDRTALPGVVRRFGYVLSNTLFPYLFVRYMGKLRAKLMREYPHLVEYDEDEPVPSPETWKERVIKTFVNKFDKFTALEGFTAIHLAIFYVYGSYYQLSKRIWGMRYVFGHRLDKNEPRIGYEMLGLLIFARFATSFVQTGREYLGALLEKSVEKEAGEKEDEKEAVVPKKKSSIPFIEDTEGETEDKIDLEDPRQLKFIPEASRACTLCLSYISAPACTPCGHFFCWDCISEWVREKPECPLCRQGVREQNLLPIR

SEQ ID NO: 124 - protein, 679 aa, Cydia pomonella
MADSPPEDNTPTRNGSLKISLPTVLCTPESSENSDILCKTKTFISSESKLSLPSNLGTPTSPLRLSSPLSLPTSPTPYMKITQPQPEKTVAATQMNSPPTTIVQQTGPKQPLVNTGSATGNPRNKCALQPGHSLMDWIRLGNSGKDLTGVGGRMRPVSPDELAAHNTRNDAWLAIRGRVYNITHYLPYHPGGPEELMRGAGIDATQLFDKVHPWVNYDSLLAKCLVGPLRLDRPDAEELFGPSTPSPKSENRLREPSKAQELVRKSMENLANCITPVRKKMSAKSEENIKGSPPSKIMQSLIQSSDLPVSISRRAAASPVKTSEKSDIAPSPLRFDWIQTSTKLTLSIYTGPLANPGGCARIIEGILFVEVATNGWVRTLKLVPEAKLLESLQLRVFAESGKIEVTAHKAEPGVWKTCGEAMAGAASRVSTPRSVSCRVARMLRVSHDTVLLSLAAPAQALVAPLGHHVRVHRTISDVECVRSYTPVGEGWGPDAERDSAIHLAVKGYDTGALSPYLTALQPGDEVTVSGPYGNFQLHTLKGVKEIYFVAAGTGITPMLGLLKFMLPRSNPRCERIHLLFFNKTEEDILFRENFEEIAREDDRFTVTHVLSDAGPSWSGHKGRVTTNLLTQVIGKSFLKCRDQCTHFACVCGPTEFTHTAIDLLKKLDMKDSCIHAFMG

SEQ ID NO: 125 - DNA, 2040 bp, Cydia pomonella
atggccgactctccacctgaggacaacacccctactcgaaacggctctctgaagatctctctgcccaccgtgctgtgcacccctgagtcctctgagaactctgacatcctgtgcaagaccaagaccttcatctcttctgagtctaagctgtctctgccctctaacctgggaacccctacctctcctctgcgactctcttctcccctgtcgctgcccacctctcctacaccttacatgaagatcacccagcctcagcctgaaaagaccgtggccgccactcagatgaactccccacctaccaccatcgtgcagcagaccggacctaagcagcccctggtgaacaccggctctgccactggcaaccctcgaaacaagtgcgctctgcagcccggccactctctgatggactggatccgactgggcaactctggcaaggacctgaccggcgtcggcggacgaatgcgacccgtgtctcccgacgagctggccgctcacaacacccgaaacgacgcctggctggccatccgaggccgagtgtacaacatcacccactacctgccttaccatcctggcggacccgaggaactgatgcgaggcgccggaatcgacgctacccagctgttcgacaaggtgcacccttgggtgaactacgactctctgctggccaagtgcctggtgggacccctgcgactggaccgacctgacgctgaggaactgttcggcccctctactccctcgcctaagtctgagaaccgactgcgagagccctctaaggcccaagagctggtgcgaaagtctatggaaaacctggccaactgcatcacccctgtgcgaaagaagatgtctgccaagtctgaggaaaacatcaagggatctccaccttctaagatcatgcagtctctgattcagtcctccgacctgcctgtgtctatttctcgacgagccgccgcttctcccgtcaagacctccgagaagtctgatatcgccccttcgcctctgcgattcgactggattcagacctctaccaagctgaccctgtctatctacaccggacctctggctaaccccggtggctgcgcccgaatcatcgagggcatcctgttcgtcgaggtcgccaccaacggctgggtgcgaaccctgaagctggtgcccgaggccaagctgctcgagtctctgcagctgcgagtgttcgccgagtctggcaagatcgaggtgactgcccacaaggctgagcccggcgtgtggaagacctgcggcgaggccatggctggcgctgcctctcgagtgtctacccctcgatctgtgtcttgccgagtggcccgaatgctgcgagtctcccacgacaccgtcctgctgtccctggctgctcccgctcaggccctggtggctcccctgggccaccacgtgcgagtgcaccgaaccatctctgacgtcgagtgcgtgcgatcttacacccctgtcggcgaaggctggggacccgacgccgagcgagactctgccatccacctggccgtgaagggctacgacaccggcgctctgtctccctacctgactgccctgcagcctggcgacgaggtgaccgtgtctggcccctacggcaacttccagctgcacaccctgaagggcgtgaaggaaatctacttcgtcgccgctggcaccggaatcacccctatgctgggcctgctgaagttcatgctgccccgatctaaccctcgatgcgagcgaatccacctcctgttcttcaacaagaccgaagaggacattctgttccgagagaacttcgaggaaatcgcccgagaggacgaccgattcaccgtgactcacgtgctgtctgacgctggcccctcttggtctggccacaagggacgagtgaccaccaacctgctgacccaggtgatcggcaagtctttcctgaagtgccgagatcagtgcacccacttcgcctgcgtgtgcggccccaccgagtttacccacaccgccatcgacctgctcaagaagctggacatgaaggactcttgcatccacgccttcatgggctag

SEQ ID NO: 126 - protein, 346 aa, Antheraea pernyi
MAPCTEKEDELITCEGSIEKLIAPQANKRKHQIVYMNLFTFGYWYIAGLYGLYLFFTSTKWSTIILNIFLIIASGLGITVGAHRLWAHKCFKAKLPLQIILMILNTLAFQNTAITWVRDHRMHHKFTDTDADPHNATRGFFFSHVGWLLVKKHPELMKRSKHIDMSDIYGNPVLTFQKKYAFPLVATFTFILPTVVPIYFWDESLNNAWHATVFRYAYNLNITFLVNSAAHMWGYRPYDKNIQPTQNIFTTLCTFGEGFHNYHHTFPWDYRAAELGNNYLNLSTKVIDFFAWIGWAYDLKTVPADIVNSRAKRTGDGTDLWGGNHKHTLDEKENRELDDYVHKSAF

SEQ ID NO: 127 - protein, 329 aa, Argyrotaenia velutinana
MAPNAEDIETNMPETEENWETLVAPQAAPRKYQIVYKSLLTFGYGHLAGLYGLYLCFTSAKWQTIGLAIILHAMAILGITAGAHRLWTHRAYKATVPLQIILIIFNSLSFQNSAFTWIRDHRLHHKYSDTDGDPHNATRGFFYSHIGWLLVRKHPEVMKRGRMTEMSDIYSNPIIMFQKNYAIPFIGTVCFVLPTIIPMYFWGETLNNAWHITVLRYIFSLNCIFLVNSAAHLYGYKPYDKNILPAENKAASIASFGEAFHNYHHVFPWDYRASELGNITMNWTIYFIDFFAWIGWAYDLKTASDETIKSRIKRTGDGTDFSGQQIYAR

SEQ ID NO: 128 - protein, 351 aa, Bombus lapidarius
MAPNITSSPTGVLFEGETLEETSRVIDAPKTKFKTQIVWRNVIIFTYLHISAVYGLYLAFTSAKWATLLFAFFLHVYSALGITAGAHRLWAHRSYKAKWPMQLILIIGNTIAFQDSAIDWARDHRLHHKYSETNADPHNAKRGFFFAHIGWLLCKKHPDIRAKGKGIDMSDLKNNPILTFQKKYYTILMPLLCFIVPTMIPVYCWNETWGNGYFIPAILRYVYTLNMTWLVNSAAHMFGNKPYDKYINPVENKMVAITALGEGWHNYHHVFPWDYKTAELGNYKVNVTTLFIDVCSKLGLAYDMKIVPQDLVQKRVERTGDGSHNVWGWGDKDQTQQDRDVTMVVNLKKDH

SEQ ID NO: 129 - protein, 339 aa, Bombyx mori
MEAKQNNLAPTLEEEAQFEKLIAPQASDRKHEIIYANLITFAYGHISALYGLYLCFSSAKWATIIMAYVILIAAEVGVTAGAHRLWTHRAYKAKRPLQIILMVMNSFAFQNSAITWIRDHRMHHRYSDTDADPHNATRGFFYSHIGWLLVRKHPEVKRRGKTIDMSDIYSNPVLVFQKKYAIPFIGAVCFVIPTLVPIYFWGETLTNAWHITLLRYIISLHVTFLVNSAAHLWGTRAYDKRIFPAQNLIVSLLAVGEGFHNYHHVFPWDYRTAELGNNYLNLTTKFIDFFAWLGWAYDLKSVPDSAVQSRAARTGDGTNSWGWPEEDANEDILKQTPPL

SEQ ID NO: 130 - protein, 345 aa, Chilo supprealis
MAPNSIQNDEGLGVEVQPESLTPPKLTQIRHKILYVNLTYFLYWHLAAPYGIYLCFTSVKWETIIFGFVVFLAAELGVTAGAHRLWSHKSYKAKRPLEIILMLFNSMAFQNSIIHWARDHRLHHKYCDTDADPYNASRGFFYSHVGWLLTKKNKEVLKRGKTIDASDLFNNPVLQFQKKYAIPVFALCCFILPTAIPMYVWGETLNNSWHANLLRYTINLNVTFLVNSAAHIWGYRPYDKSIYPAQNVYVSLVTLGEGFHNYHHVFPWDYKTAELGNNKLNVTTWFINFFTKLGWAYDLKTVSNEIVRARAMRTGDGKDLWGWNDKDITDMDKEHTDILYSKTED

SEQ ID NO: 131 - protein, 374 aa, Drosophila ananassae
MPPNSKESTDKCTGLASEVPGTVDDGSSKQTGVLFEADVETNDGDLAVSTVEFKRAEKRKLELVWRNIILFAYVHLAALYGGYLMFTQAKLATTIFAAGLYICGMLGITGGAHRLWAHRSYKAKWPLRLILIVFNTIAFQDAAYHWARDHRVHHKFSETDADPHNATRGFFFSHVGWLLTKKHPDVKAKGKILDLSDLHADPILMFQKKHYFVLMPLACFILPTIIPVLCWNESLTCGWLVATMFRWCFQLNMTWLVNSAAHKFGGRPYDKNMNPSENAYVATMTFGEGWHNYHHVFPWDYKTSEWGNRLNMTARFIDLFAKIGWAYDLKSVAPDTVKRRVWRTGDGSHELWGWGDKDLTPEDAKDVLFVDKMN

SEQ ID NO: 132 - protein, 344 aa, Diatraea saccharalis
MAPNLIKNEGAFDVDIKPDELQPPKLSKIKHKILYVNLIYFLYWHLAAPYGLYLCCTSAKWATIIFAFVMFLAAELGITAGAHRLWSHKSYKAKLPLEIILMLFNSIAFQNSIIHWARDHRLHHKYSDTDADPHNASRGFFYSHVGWLITKKNKEVLKRGKNIDVSDLYNNPVLQFQRKYAVPVFASFCFILPTLIPMYLWGESFKNSWYVNLLRYVINLNVTFLVNSAAHKWGYKPYEKNINPAQNVYVSFATLGEGFHNYHHIFPWDYKTAELGNNKLNVTTWFINFFAKIGWAYDLKTVSNEMVRARAMRTGDGNDLWGWDDEDITDKDKEHTDILNGKTD

SEQ ID NO: 133 - protein, 358 aa, Drosophila yakuba
MAPYEPSSNSSKQTGVLFEGDAETADGDLTSDRFQLKRAENRRLDLVWRNIILFALVHLSALYGLYSIFTKAKLATTLFAAGLYIIGMLGVTAGAHRLWAHRTYKAKWPLRLILVIFNTVAFQDAAYHWARDHRVHHKYSETDADPHNATRGFFFSHVGWLLCKKHPDIKEKGKGLDLSDLRADPILMFQRKHYYILMPLACFVLPTVIPMVYWNETLASSWFVASMFRWCFQLNMTWLVNSAAHQFGNRPYDKTMNPTQNAYVSAFTFGEGWHNYHHSFPWDYKTAEWGRYSLNITTAFIDMFAKIGWAYDLKTVAPDMIQRRVLRTGDGSHELWGWGDKDLTPEDARNVLFVDKRS

SEQ ID NO: 134 - protein, 342 aa, Cadra cautella
MVPYKGSSTVLTEDQPQFEKLVAPQAGPRKYEIVYRNLITFGYWHLAAIYGLYLCFTTAKWATIIFALFLYTIAEIGITAGAHRLWTHRAYKAKLPLQILLMVMNSIAFQDTALTWCRDHRMHHRYSDTDADPHNATRGFFYSHVGWLLVKKHPEVKARGKYIPLDDLLNNPVLRFQKKYAVPVVGTLCFLMPTFVPVYFWGETISTAWHINFLRYVMNLNMTFLVNSAAHMFGNKPYDRTLVPVQNVAVSFATFGEGFHNYHHTYPWDYRTAELGNNKLNITTHFIDFFAWIGWAYDLKTIPQDAIEKRMIRTGDGTDMWGFGEKRFRKEVDNSNETYSDF

SEQ ID NO: 135 - protein, 350 aa, Lobesia botrana
MVPRLEVNTGVKTVDEEQLPSRDENKPRISKLVKINIVSFGYLHIAALYGLYLCFTSAKWATIGLAFFLLVVGEIGITAGAHRLWSHRTYKANRALEILLIIMNSVAFQNSAIHWIRDHRLHHKYSDTNADPHNASRGFFYSHIGWLLVRKHSDVINKGKMIDMSDIYNNPVLTFQKKYAIPFIGTICFGLPTLIPMYCWGESLNNAWHINIMRYVLCLNITFLVNSAAHFFGQKPYDKNILPGQNILVSILAVGEGFHNYHHVFPWDYRTAELGNNMLNLTTKFIDFFAWLGWAYELKTVSTNAIDLRAKRTGDGTNLWGWGDKHMTQEEIEASDILFVNKENEKTHRH

SEQ ID NO: 136 - protein, 367 aa, Ostrinia furnacalis
MADIDATNHYDLQDKSEDDKPEDTEGELTIVGTDYSYTHRIIWPIVMVYVVMHIGAITGLLLVLGGNVKIASIIWAVFYSLVATEGAHMGAHRCFSHRAFKAKPLLKVILLIMQTISGQHSTYIWCRDHRQHHRYSDTDGDPHNSKRGMFYCHVGWLMTSRHPLCKKLRKTIDMSDLQQDPLVMFQYRYFRSLFFTFGFLLPVWVPMHFFQESFTNAVFVCFFLRYVYALHVTYFINSLAHKYGTRPYDKTIQPVETWFVSLLSLGEGWHNYHHAYPWDYKAAEIGMPLNSTASLIRLCASLGLAYDLKSVDPETLNKRIMNKGDGTYEVKYLQEHVTAIGPLHPLNPSYRGTCPDPEIKLKVRMKP

SEQ ID NO: 137 - protein, 344 aa, Plodia interpunctella
MVPNKGSSVVLTENAEPQFKKLVAPQAGPRKYQIVYRNLLTFGYWHLSAIYGLYLCFTSAKWATIIFAFFLYMFAEIGITAGAHRLWTHRAYKAKLPLQILLIIMNSIAFQDTALTWCRDHRMHHRYSDTDADPHNATRGFFYSHVGWLLVKKHPEVKTRGKYIPLDDLRNNPVLRFQKKYAIPFVGTLCFLMPTFVPVYFWGESISTAWHINLLRYVTNLNVTFLVNSAAHLIGNKPYDRTLASVQNIPVSIATFGEGFHNYHHTYPWDYRTAELGNNRLNVTTKFIDFFAWLGWAYDLKTVPQEAVEKRMARTGDGTDLWGYGEKKPVKDVKDNSDETYYDF

SEQ ID NO: 138 - protein, 320 aa, Tribolium castaneum
MSTLETQVQIVWRNVILFIYLHVAAIYGLYFTFTAAKWPTILFTYFLTIISTQGTGAGVHRLWSHRSYKAKLPLRILLCIYQTHCLQNHIYEWVRDHRAHHKFSDTDADPHNSTRGFFFSHMGWLLVRKHPQVKIKGKLIDLSDLEEDQVVMFQKKYYLILAPFFAFLLPAWVPWYFWGEDLHVSWCVASMLRYALSLHGTWLVNSAAHMWGTRPYDRNIKATETKVVSYITNGERFHNYHHTFPWDYKAAELGSYWGNWSTAFIDFMAKIGWAYDLKIIPPELVEKRAKRTGECTHKVWGWGDKDIDKEEIEIVERNRG

SEQ ID NO: 139 - protein, 352 aa, Yponomeuta padella
MPPNSANSEPLLHDEVKEEQLEKLVAPQAGPRKFQIVYPNLITFGYGHISLLYGAYLMFTFAKWQTIVFAHVMIILSGFGITAGAHRLWAHRTYKAKMPLQILLMLLNTLAFQNTAMDWVRDHRLHHKYSDTDADPHNATRGFFYSHVGWLLVRKHKEVKRRGKLIDLSDIENNPVLSFQKKYAIPLVSLVTFVMPTVIPMYFWGETLNNAWHVATLYRYVFVLNVTFLVNSAAHYWGCKPYEKSILPSQNHFVSLFALGEGFHNYHHVFPWDYRTAELGNNTLNPTTKFIDFFAWIGWAYDLKAVPDDMINNRKSRTGDGSSLWGWGDKDQPKEEMNAAIIKYPDENDKDK

SEQ ID NO: 140 - DNA, 1041 bp, Antheraea pernyi
atggctccctgcaccgagaaggaagatgagctgatcacctgtgagggctctatcgagaagctgatcgctccccaggccaacaagcgaaagcaccagatcgtgtacatgaacctgttcaccttcggctactggtacattgccggcctgtacggactgtacctgttcttcacctctaccaagtggtctactattatcctgaacatcttcctgatcattgcctctggcctgggcatcaccgtgggagcccaccgactgtgggctcacaagtgcttcaaggccaagctgcccctgcagatcatcctgatgattctgaacaccctggccttccagaacaccgccatcacctgggtgcgagatcaccgaatgcaccacaagttcaccgacaccgacgctgaccctcacaacgctacccgaggcttcttcttctctcacgtcggctggctgctggtgaagaagcaccccgagctgatgaagcgatctaagcacatcgacatgtctgacatctacggcaaccccgtgctgacctttcagaagaagtacgctttccctctggtcgccaccttcaccttcattctgcccaccgtggtgcctatctacttctgggacgagtctctgaacaacgcctggcacgccaccgtgttccgatacgcctacaacctgaacattacctttctggtgaactctgccgctcacatgtggggctaccgaccttacgacaagaacattcagcccactcagaacattttcaccactctgtgtaccttcggcgagggcttccacaactaccaccacacattcccttgggactaccgagccgccgagctgggcaacaactacctgaacctgtctaccaaggtgatcgacttcttcgcctggatcggctgggcctacgacctcaagaccgtgcctgccgacatcgtgaactctcgagccaagcgaaccggcgacggcaccgacctctggggcggcaaccacaagcacaccctggatgagaaggaaaaccgagagctggacgactacgtgcacaagtctgccttttag

SEQ ID NO: 141 - DNA, 990 bp, Argyrotaenia velutinana
atggctcccaacgccgaggatattgaaaccaacatgcccgagacagaggagaactgggagactttggttgctccgcaggcagctcctcgaaaataccagatcgtctacaaaagtctactgacattcggttacggccatctggctgggctctatggcctgtacctgtgctttacgtcagccaagtggcagactattggtcttgccattattctccatgctatggccattctcggaatcactgcaggagcccacagactgtggacccaccgagcttacaaggctactgtgcctctgcaaatcattctcattatcttcaactccttgtctttccaaaactctgcattcacgtggatcagggaccatcgcctccaccacaagtacagcgacaccgatggtgatcctcacaatgcgacgagaggtttcttctactctcatattggatggctgctggtgcgaaagcatcctgaggtcatgaagcgaggccgaatgaccgagatgtctgatatctactccaaccccatcatcatgttccagaagaattatgctattcctttcattggcaccgtgtgttttgttctcccaacgatcatccccatgtacttctggggcgaaaccctcaataacgcatggcacatcactgtgttgcggtacattttctccctgaactgcatttttctggtcaattcggcagctcacctgtatggatacaagccctacgacaagaacattcttccagcggaaaacaaggccgcttccatcgcctcgtttggcgaggcctttcacaactaccaccatgtattcccctgggactaccgagcgtctgaacttggaaacatcaccatgaactggacaatctactttattgacttctttgcctggattggctgggcctatgaccttaagaccgcctccgacgagactatcaaatcgcgtataaagcgtactggagatggaaccgactttagcggccagcagatttatgcccggtga

SEQ ID NO: 142 - DNA, 1056 bp, Bombus lapidarius
atggctcccaacatcacctcttctcccaccggcgtgctgttcgagggcgagactctggaagagacttctcgagtgatcgacgctcccaagaccaagttcaagacccagatcgtgtggcgaaacgtgatcatcttcacctacctgcacatctctgccgtgtacggcctgtacctggccttcacctctgccaagtgggctaccctgctgttcgccttcttcctgcacgtgtactctgccctgggcatcaccgctggcgcccaccgactgtgggctcaccgatcttacaaggccaagtggcccatgcagctgatcctgatcatcggcaacaccattgccttccaggactctgccatcgactgggcccgagatcaccgactgcaccacaagtactctgagactaacgctgaccctcacaacgccaagcgaggcttcttcttcgcccacatcggctggctgctgtgcaagaagcaccccgacatccgagccaagggcaagggcatcgacatgtctgacctgaagaacaaccccatcctgaccttccagaagaagtactacaccattctgatgcccctgctgtgcttcatcgtgcccaccatgattcccgtgtactgctggaacgaaacctggggcaacggctacttcatccccgccatcctgcgatacgtgtacaccctgaacatgacctggctggtgaactctgccgctcacatgttcggcaacaagccctacgacaagtacatcaaccccgtcgagaacaagatggtggccattaccgctctcggcgaaggctggcacaactaccaccacgtgttcccttgggactacaagaccgccgagctgggcaactacaaggtgaacgtgaccactctgttcatcgacgtgtgctctaagctgggcctcgcctacgacatgaagatcgtgccccaggacctggtgcagaagcgagtcgagcgaaccggcgacggctctcacaacgtgtggggctggggcgacaaggaccagactcagcaggaccgagatgtgaccatggtggtcaacctcaagaaggaccactag

SEQ ID NO: 143 - DNA, 1020 bp, Bombyx mori
atggaggccaagcagaacaatctggcacccaccctggaggaggaggcacagttcgagaagctgatcgcccctcaggcctccgatcgcaagcacgagatcatctacgccaacctgatcaccttcgcctatggccacatctctgccctgtacggcctgtatctgtgctttagctccgccaagtgggccacaatcatcatggcctacgtgatcctgatcgcagcagaagtgggagtgaccgcaggagcccacaggctgtggacacaccgcgcctataaggccaagcggcccctgcagatcatcctgatggtcatgaactccttcgcctttcagaattctgccatcacatggatcagggaccaccgcatgcaccacaggtactctgacaccgatgccgacccacacaacgccacacgcggcttcttttatagccacatcggatggctgctggtgcggaagcacccagaggtgaagcggagaggcaagaccatcgatatgtctgacatctacagcaatcctgtgctggtgttccagaagaagtatgccatcccattcatcggcgccgtgtgctttgtgatcccaacactggtgcccatctacttttggggcgagaccctgacaaacgcctggcacatcaccctgctgagatatatcatcagcctgcacgtgacattcctggtgaattccgccgcacacctgtggggaaccagggcatacgataagagaatctttcccgcccagaacctgatcgtgtccctgctggcagtgggagagggcttccacaattaccaccacgtgtttccttgggattataggacagccgagctgggcaacaattacctgaatctgaccacaaagtttatcgacttctttgcctggctgggctgggcctatgatctgaagtccgtgcctgactctgccgtgcagagcagggcagcaagaaccggcgacggaacaaacagctggggatggccagaggaggatgccaatgaggacatcctgaagcagaccccccctctgtga

SEQ ID NO: 144 - DNA, 1038 bp, Chilo supprealis
atggctcccaactctatccagaacgacgaaggcctgggcgtcgaggtgcagcccgagtctctgacccctcctaagctgacccagatccgacacaagatcctgtacgtgaacctgacctacttcctgtactggcacctggctgctccctacggcatctacctgtgcttcacctctgtgaagtgggagactatcatcttcggcttcgtggtgttcctggccgccgagctgggcgtgaccgctggcgcccacagactgtggtcccacaagtcttacaaggccaagcgacccctcgagatcatcctgatgctgttcaactctatggccttccagaactctatcatccactgggcccgagatcaccgactgcaccacaagtactgcgacaccgacgctgacccctacaacgcctctcgaggcttcttctactctcacgtcggctggctgctgaccaagaagaacaaggaagtcctgaagcgaggcaagaccatcgacgcctctgacctgttcaacaaccccgtgctgcagttccagaagaagtacgctatccccgttttcgccctgtgctgcttcattctgcccaccgctattcccatgtacgtgtggggcgagactctgaacaactcttggcacgccaacctgctgcgatacaccatcaacctgaacgtgaccttcctggtgaactctgccgctcacatctggggctaccgaccttacgacaagtctatctaccccgctcagaacgtgtacgtgtctctggtgaccctcggcgagggcttccacaactaccaccacgtgttcccttgggactacaagaccgctgagctgggaaacaacaagctgaacgtcactacctggttcatcaactttttcaccaagctcggctgggcctacgacctcaagaccgtgtctaacgagatcgtgcgagcccgagccatgcgaaccggcgacggcaaggacctgtggggctggaacgacaaggacatcaccgacatggacaaggaacacactgacatcctgtactctaagaccgaggactag

SEQ ID NO: 145 - DNA, 1125 bp, Drosophila ananassae
atgcctccgaactctaaggaatctaccgacaagtgcaccggcctggcctctgaggtgcccggcaccgtggacgacggatcttctaagcagaccggcgtgctgttcgaggccgacgtcgagactaacgacggcgacctggccgtgtctaccgtcgagttcaagcgagccgagaagcgaaagctggaactggtgtggcgaaacatcatcctgttcgcctacgtgcacctggccgctctgtacggcggctacctgatgttcacccaggccaagctggccaccaccatcttcgccgctggcctgtacatctgcggcatgctgggcatcaccggcggagcccaccgactgtgggctcaccgatcttacaaggctaagtggcccctgcgactgatcctgatcgtgttcaacaccattgccttccaggacgccgcctaccactgggcccgagatcaccgagtgcaccacaagttctctgagactgacgctgaccctcacaacgctacccgaggcttcttcttctctcacgtcggctggctgctgaccaagaagcaccccgacgtcaaggccaagggcaagatcctggacctgtctgacctgcatgctgaccccattctgatgttccaaaagaagcactacttcgttctgatgcccctggcctgcttcattctgcccaccatcattcccgtgctgtgctggaacgagtctctgacctgtggctggctggtcgccaccatgttccgatggtgcttccagctgaacatgacctggctggtgaactctgccgctcacaagttcggcggacgaccctacgacaagaacatgaacccctctgagaacgcctacgtcgctaccatgaccttcggcgaaggctggcacaactaccaccacgtgttcccttgggactacaagacctctgagtggggcaaccgactcaacatgaccgctcgattcatcgacctgttcgctaagatcggctgggcctacgacctgaagtctgtggctcccgacaccgtgaagcgacgagtctggcgaaccggcgacggctctcacgagctgtggggctggggcgacaaggacctgactcctgaggacgccaaggacgtgctgtttgtggacaagatgaactag

SEQ ID NO: 146 - DNA, 1035 bp, Diatraea saccharalis
atggctcccaacctgatcaagaacgagggcgccttcgacgtggacatcaagcccgacgagctgcagcctcctaagctgtctaagatcaagcacaagatcctgtacgtgaacctgatctacttcctgtactggcacctggctgctccctacggcctgtacctgtgctgcacctctgccaagtgggccaccatcatcttcgccttcgtgatgttcctggccgccgagctgggcatcaccgctggcgcccaccgactgtggtcccacaagtcttacaaggccaagctgcccctcgagatcatcctgatgctgttcaactctatcgccttccagaactctatcatccactgggcccgagatcaccgactgcaccacaagtactctgacaccgacgctgaccctcacaacgcctctcgaggcttcttctactctcacgtcggctggctgatcaccaagaagaacaaggaagtcctgaagcgaggcaagaacatcgacgtgtctgacctgtacaacaaccccgtgctgcagttccagcgaaagtacgctgtgcccgtgttcgcctctttctgcttcattctgcccactctgatccccatgtacctctggggcgagtctttcaagaactcctggtacgtcaacctcctgcgatacgtgatcaacctgaacgtgaccttcctggtgaactctgccgctcacaagtggggctacaagccctacgagaagaacatcaaccccgctcagaacgtgtacgtgtctttcgctaccctcggcgagggcttccacaactaccaccacatctttccatgggactacaagaccgctgagctgggaaacaacaagctgaacgtcactacctggttcatcaactttttcgccaagatcggctgggcctacgacctcaagaccgtgtctaacgagatggtgcgagcccgagccatgcgaaccggcgacggcaacgacctgtggggctgggacgacgaggacattaccgacaaggacaaggaacacaccgacatcctgaacggcaagaccgactag

SEQ ID NO: 147 - DNA, 1077 bp, Drosophila yakuba
atggctccctacgagccctcttcgaactcttctaagcagaccggcgtgctgttcgagggcgacgccgagactgccgacggcgacctgacctctgaccgattccagctgaagcgagccgagaaccgacgactggacctggtgtggcgaaacatcatcctgttcgccctggtgcacctgtctgccctgtacggcctgtactctatcttcaccaaggccaagctggccaccactctgttcgctgccggactgtacatcatcggcatgctgggcgtgaccgctggcgcccaccgactgtgggctcaccgaacctacaaggctaagtggcccctgcgactgatcctggtgatcttcaacaccgtggccttccaggacgccgcctaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggcaagggcctcgacctgtccgacctgcgagctgaccccatcctgatgttccagcgaaagcactactacattctgatgcccctggcctgcttcgtgctgcccaccgtgattcccatggtgtactggaacgagactctggcctcttcctggttcgtggcctctatgttccgatggtgcttccagctcaacatgacctggctggtgaactctgccgctcaccagttcggcaaccgaccttacgacaagactatgaaccccactcagaacgcctacgtgtctgccttcaccttcggcgaaggctggcacaactaccaccactcgtttccctgggactacaagaccgccgagtggggccgatactctctgaacatcaccaccgccttcatcgacatgttcgccaagatcggctgggcctacgacctcaagaccgtggctcccgacatgatccagcgacgagtgctgcgaaccggcgacggctctcacgagctgtggggctggggcgacaaggacctgactcctgaggacgcccgaaacgtcctgttcgtggacaagcgatcttag

SEQ ID NO: 148 - DNA, 1029 bp, Cadra cautella
atggtgccctacaagggatcttctaccgtgctgaccgaggaccagcctcagttcgagaagctggtggctccccaggctggaccccgaaagtacgagatcgtgtaccgaaacctgatcaccttcggctactggcacctggccgccatctacggcctgtacctgtgcttcaccaccgccaagtgggccactattatcttcgccctgttcctgtacaccattgccgagatcggcatcaccgctggcgcccaccgactgtggacccaccgagcctacaaggccaagctgcccctgcagatcctgctgatggtgatgaactctatcgccttccaggacaccgctctgacctggtgtcgagatcaccgaatgcaccaccgatactctgacaccgacgctgaccctcacaacgctacccgaggcttcttctactctcacgtcggctggctgctggtgaagaagcaccccgaggtcaaggcccgaggcaagtacattcccctggacgacctgctgaacaaccccgtgctgcgattccaaaagaagtacgctgtccccgtcgtgggcaccctgtgctttctgatgcccaccttcgtgcccgtgtacttctggggcgagactatctctaccgcctggcacatcaactttctgcgatacgtgatgaacctgaacatgacctttctggtgaactctgccgctcacatgttcggcaacaagccctacgaccgaactctggtgcccgtgcagaacgtggccgtgtctttcgccaccttcggagagggcttccacaactaccaccacacttacccctgggactaccgaaccgccgagctgggcaacaacaagctgaacatcaccactcacttcatcgactttttcgcctggatcggctgggcctacgacctcaagaccattcctcaggacgccatcgagaagcgaatgatccgaaccggcgacggcaccgacatgtggggcttcggtgagaagcgatttcgaaaggaagtggacaactctaacgagacttactctgacttttag

SEQ ID NO: 149 - DNA, 1053 bp, Lobesia botrana
atggtgccccgcctagaagtaaatactggggtgaaaacagtggatgaagagcaattgccgtcaagggatgagaacaaaccgagaataagcaaactagtaaaaataaacatagtcagctttggatatctacatatagctgcactttatggattatatttatgcttcacatcggctaaatgggctacaattgggctcgctttctttttgctggtcgtcggtgaaatcggcatcacggcaggcgcccacaggttatggtcccacagaacctacaaagccaacagggcgttagaaatattactcatcattatgaactccgtggccttccaaaactcggccatccactggattcgggaccacagattacaccataaatacagcgacactaatgccgacccccacaatgcatccagaggatttttctattcacatataggctggttgctagtcaggaaacactctgacgtcataaacaaaggaaagatgattgacatgtctgatatatacaacaatccagttttaacattccaaaagaaatatgccattcccttcatcggcaccatatgcttcggtctacctaccctgataccgatgtactgctggggagagtctctgaacaacgcttggcacatcaacataatgcgatatgtcctgtgcctcaacattactttcctagtgaacagtgccgcgcatttcttcggtcagaaaccgtatgacaagaacattttaccgggacagaatatacttgtttcaatactcgctgttggcgaaggatttcataattaccaccacgtctttccgtgggactatcgcacggcagaactaggcaacaacatgttgaacttaacaacgaagtttatagacttcttcgcttggcttggttgggcgtacgagttaaaaactgtttcaactaatgcgattgatttaagagctaagaggactggcgacggaaccaacttgtggggttggggcgacaagcatatgacgcaagaagaaattgaggctagtgacattttgtttgttaacaaagaaaacgagaaaactcataggcattaa

SEQ ID NO: 150 - DNA, 1104 bp, Ostrinia furnacalis
atggccgacatcgacgccaccaaccactacgacctgcaggacaagtctgaggacgacaagcccgaggacaccgagggcgagctgaccatcgtgggcaccgactactcttacacccaccgaatcatctggcccatcgtgatggtgtacgtggtgatgcacatcggcgccatcaccggcctgctgctggtgctcggcggcaacgtgaagatcgcctctatcatctgggccgtgttctactctctggtggccactgagggcgcccacatgggagcccaccgatgcttctctcaccgagccttcaaggctaagcccctgctgaaggtgatcctgctgatcatgcagaccatctctggccagcactctacctacatctggtgccgagatcaccgacagcaccaccgatactccgacaccgacggtgaccctcacaactctaagcgaggcatgttctactgccacgtcggctggctgatgacctctcgacaccctctgtgcaagaagctgcgaaagaccatcgacatgtctgacctgcagcaggaccctctggtgatgttccagtaccgatactttcgatctctgttcttcaccttcggctttctgctgcccgtgtgggtgcctatgcacttcttccaagagtctttcaccaacgccgtgttcgtgtgcttcttcctgcgatacgtgtacgccctgcacgtgacctacttcatcaactccctggctcacaagtacggcacccgaccttacgacaagaccattcagcccgtggaaacctggttcgtgtctctgctgtctctcggcgaaggctggcacaactaccaccacgcttacccctgggactacaaggccgccgagatcggcatgcccctgaactctaccgcctctctgatccgactgtgcgcctctctcggcctggcttacgacctgaagtctgtggaccccgagactctgaacaagcgaatcatgaacaagggcgacggcacctacgaggtgaagtacctgcaagagcacgtcaccgccatcggacccctgcatcctctgaacccctcttaccgaggcacctgtcctgatcctgagatcaagctgaaggtccgaatgaagccctag

SEQ ID NO: 151 - DNA, 1035 bp, Plodia interpunctella
atggtgcccaacaagggatcttctgtggtgctgaccgagaacgctgagccccagttcaagaagctggtcgctccccaggctggaccccgaaagtaccagatcgtgtaccgaaacctgctgaccttcggctactggcacctgtctgccatctacggcctgtacctgtgcttcacctctgccaagtgggctactattatcttcgccttcttcctgtacatgttcgccgagatcggcatcaccgctggcgcccaccgactgtggacccaccgagcctacaaggccaagctgcccctgcagatcctgctgatcatcatgaactctatcgccttccaggacaccgctctgacctggtgccgagatcaccgaatgcaccaccgatactctgacaccgacgctgaccctcacaacgctacccgaggcttcttctactctcacgtcggctggctgctggtgaagaagcaccccgaggtcaagacccgaggcaagtacattcccctggacgacctgcgaaacaaccccgtgctgcgattccagaagaagtacgctatccccttcgtgggcaccctgtgctttctgatgcccaccttcgtgcccgtgtacttctggggcgagtctatctctaccgcctggcacatcaacctgctgcgatacgtgaccaacctgaacgtgaccttcctggtgaactctgccgctcacctgatcggcaacaagccctacgaccgaactctggcctctgtgcagaacatccccgtgtctatcgccaccttcggcgagggcttccacaactaccaccacacttacccctgggactaccgaaccgccgagctgggcaacaaccgactgaacgtcactaccaagttcatcgactttttcgcctggctcggctgggcctacgacctcaagaccgtgcctcaagaggccgtcgagaagcgaatggcccgaaccggcgacggcaccgacctgtggggctacggcgaaaagaagcccgtcaaggacgttaaggacaactctgacgagacttactacgacttttag

SEQ ID NO: 152 - DNA, 963 bp, Tribolium castaneum
atgtctaccctcgagactcaggtgcagatcgtgtggcgaaacgtgatcctgttcatctacctgcacgtggccgccatctacggcctgtacttcaccttcaccgccgccaagtggcccactatcctgtttacctacttcctgaccatcatctctacccaaggcaccggcgctggcgtgcaccgactgtggtcccaccgatcttacaaggccaagctgcccctgcgaatcctgctgtgcatctaccagactcactgcctgcagaaccacatctacgagtgggtgcgagatcatcgagcccaccacaagttctctgacaccgacgctgaccctcacaactctacccgaggcttcttcttctctcacatgggctggctgctggtgcgaaagcaccctcaggtgaagattaagggcaagctgatcgacctgtctgacctggaagaggaccaggtggtgatgttccagaagaagtactacctgattctggctcccttcttcgccttcctgctgcctgcctgggtgccctggtacttctggggcgaagatctgcacgtgtcttggtgcgtggcctctatgctgcgatacgccctgtctctgcacggcacctggctggtgaactctgccgctcacatgtggggcacccgaccttacgaccgaaacatcaaggccaccgagactaaggtggtgtcttacatcaccaacggcgagcgattccacaactaccaccacacattcccttgggactacaaggccgccgagctgggctcttactggggcaactggtctaccgccttcatcgacttcatggccaagatcggctgggcctacgacctgaagatcatccctcctgagctggtcgagaagcgagccaagcgaaccggcgagtgcacccacaaggtgtggggctggggcgacaaggacatcgacaaggaagagatcgagattgtcgagcgaaaccgaggctaa

SEQ ID NO: 153 - DNA, 1059 bp, Yponomeuta padella
atgcctccaaactctgccaactctgagcccctgctgcacgacgaggtgaaggaagaacagctcgagaagctggtggctccccaggctggaccccgaaagttccagatcgtgtaccccaacctgatcaccttcggctacggccacatctctctgctgtacggcgcctacctgatgttcaccttcgccaagtggcagaccatcgtgttcgcccacgtgatgatcattctgtctggcttcggcatcaccgctggcgcccaccgactgtgggctcaccgaacctacaaggccaagatgcccctgcagatcctgctgatgctgctgaacaccctggccttccagaacaccgccatggactgggtgcgagatcaccgactgcaccacaagtactctgacaccgacgctgaccctcacaacgctacccgaggcttcttctactctcacgtcggctggctgctggtgcgaaagcacaaggaagtgaagcgacgaggcaagctgatcgacctgtctgacatcgagaacaaccccgtgctgtctttccagaagaagtacgctattcccctggtgtctctggtgaccttcgtgatgcccaccgtgattcctatgtacttctggggcgagactctgaacaacgcctggcacgtggctaccctgtaccgatacgtgttcgtgctgaacgtgaccttcctggtgaactctgccgctcactactggggctgcaagccctacgagaagtctattctgccctctcagaaccacttcgtgtctctgttcgccctcggcgagggattccacaactaccaccacgtgttcccttgggactaccgaaccgccgagctgggcaacaacactctgaaccccactaccaagttcatcgactttttcgcctggatcggctgggcctacgacctgaaggctgtgcccgacgacatgatcaacaaccgaaagtctcgaaccggcgacggatcttctctgtggggctggggcgacaaggaccagcctaaggaagagatgaacgccgccatcatcaagtaccccgacgagaacgacaaggataagtag

SEQ ID NO: 154 - protein, 543 aa, Amyelois transitella
MAAKCEIGVSQFFAGKHVFITGATGFMGKVLVERLLSTCADVGRIYLLMRSKKDHSPEQRLQELKASQAFDNLRQSQPSQLEKLRIVAGDIMLPGLGISQEGLEDLQEVSVVFHSAATVKFNEALKIAIEQNLMSVIRLLEICDRLPHMQAFVHVSTAYSNPELSTVEERVYPPPVPLDYLLDLAAEDSPLLDNVESFISPKPNSYTFTKAMAEAAVQRHGCQYYPIAIVRPTIVISSLKHPFPGWLENANGPTGVIFGAGRGLLRVFRCRQSALADLLPVDIGIDTLIAAAWEVASDRPPSPRVYNCSISENPTTWGDLKRAINLHTRSQPQCGAVWYPGLHTAENGFLYKILEVLLQTTPLYIGEILVRILGYSHKRLNLIRVSTQMKNMQNAMKYFALREWRFQSDNVRRLRERMSDVDKRIYNLDPCSIVWDEHYSNYVKGVRLYLLREKELDQPAAKKRLKKLYYLHYGLSVFMPFLLFRAFVRRKHRQNILEVLFAILSIFNAIFRRSTQNILYIYKVSLTFLNDNVFLNKVFNESS

SEQ ID NO: 155 - protein, 521 aa, Amyelois transitella
MVARRESVSAPVIPQFYAGRSIFITGATGFMGKVLIERILATCPDVDRLYLLVRHKKEVTPERRLQQLIESPVFDPIRASNPSQLDKLSMLAGDITRPDLGMTQESLMRLQEVSIVFHSAATLKFDEAMMMAVEQNVLSVIRLLEICDRLPKMQAFVHVSTAYSNAELSEVEEHVYPPKVPLEKLLAIAESVPDELMTEITPKFIAPKPNTYTFTKSMAEHVVQQHGNKGYPVAIFRPTIVISSVKHPFPGWIENLNGPSGVVAAAGKGLLHVFCCRGDAHADMLPVDVAIDTLIAAAWETAIDKPNEARVYNCTTRENPTKWIDFEHALRKYLVEYPLDKAFWYPSGAAIDNTYAQKAMELFTQTVPLHIVAYTVRLLGIKMQMNLITVSHRLQAMNKVLKFFAQREWVFHNENVKRLRNRLTPQDAAIYNLDPKSFNWDEHYCNFIKGTRKYLLKEKEQDLEEARKHVRRMFYVHYSVLLFVVVLLLRFALNNNFVRQFIYGVVRTLTAAFGAVFSKIV

SEQ ID NO: 156 - protein, 523 aa, Amyelois transitella
MASCVGGAFHAQEYLPVADFYADKSIFVTGGTGFMGKVLVEKLLRSCPKIKKIYLLMRPKRGQDVSSRLTELTQSPLFETLRKERPSELSKIVPIVGDITEPELGISPADQAMLCQKVSVVFHSAATVKFDEKLKLSVTINMLGTQQLVQLCHRMLGLEALVHVSTAYCNCERERVEETVYAPPAHPEHVVTLVQTLPDDLVDRITPDLVGDRPNTYTFTKALAEDMLIKECGNLPVAIVRPSIVLSSLREPVKGWVDNWNGPNGIIAAVGKGVFRSMLGTGARVADLVPVDTVINLMIVCAWRTHLRRGEGVVVYNCCTGQQNPITWQRFVKTSFKYMRKHPFSEVVWFPGGDITNNRLKHNILSLLQHKAPAIVMDLVSRASGNKPVMVRVQNKLEKAAACLEYFTTRQWAFSDDNVQALCSALSVEDRRIFDFNVRNIDWDAYIESYVLGIRRFLFKESPDTLPKSRALLRRLHIVHTLTQVTTVFLLWRFLFSQSSTLRSIWRRILEFITRIFRLLAIA

SEQ ID NO: 157 - protein, 461 aa, Chrysodeixis includens
MFASTDGGRSDEETPQQHESIADFYAGKSVFITGVTGFLGKAYLEKLLYSCKDIDKVFVLIRNKRGDDVGKRIEKLLDSSVFSRLRSHRPEDLKKITPVYGDIDKSELGLSLADQERLIKEVSVVFHVAASVNLDADLKTSLVTNYFGTTHVLKLCHRMKKLKVFVYVSTAYCNTTVKVLEEKVYPLPVELDEVVKIMEQPHLDSNRVKKLLNGRPNTYALSKALAEHYIAENHGDIPVIFIRPSIVTSSAREPAPGWADSFQGATALITACWKRVNRVIYGNGDNIIDLIPVDYVSNLSIVAAAKVKSTTEIAVYNSCTSSIKPMTLKGIAEQIERVNTENKQGSIFLPTIFFTSSWLFVIMLTFVLQLLPSFIADLFLYITGNKPMYMKIQSKVLHGRHILNYYTNDSWVFKSDSSQRLQDSLSPADKRLFPCCPSDIKWDEYFNTYFCGIDNYLLKRK

SEQ ID NO: 158 - protein, 522 aa, Cydia pomonella
MVAPRDRRGALIPQFFAGRSVLITGATGFVGKVLVERLLSTCPDISTLHLLLRVKKGATPSERLKELKESQVFDVVRQRNPSQLDKLRVLEGDVAAPGLGLAADAALTLQDVSVVFHSAATLKFDEELRKAVDQNVRSVMRLLELCDKLPNMQAFIHVSTAYSNAELSSVEERVYPPPAPLEAVLALTDHVPDPTLAGITHQYIAPKPNTYTFTKALAETVVERHGNTGYPISIFRPTIVISAQKHPFPGWIENFNGPSGVVVATGKGLLHVFPTKCGVHTDFLPVDIAVDTLIAVAWETAVDHSKEVRVYNCSTGANPTTFEDFERNLRRETTRHPLDGALWYPSGTAVQTKWARTLLEFLLQTVPLHLAEYTARMIGLKTPVNLITANKRLTAMTDALEFFATREFNFECERVRALHHRLSPEDKKIYNLDVMSINWDDHYADFVKGTRKYLLNEKEQDLEKARRHMHKMWFLHKVLKLVTLLLFLRILVQNRYARSFVYGTLRLLLSLFGAVYHRLVHA

SEQ ID NO: 159 - protein, 512 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 160 - protein, 522 aa, Manducta sexta
MVPRPVSPYAREPQIPQFYAGKCIFITGATGFMGKVLIERLLWTCPDIGKLYLLMRQKREVSPEKRLSQLKQSQVFDNIREKCPSQLDKLYIIHGDTTEPNLGISLDSMSLLNEVSIVFHGAATLKFDEVLGKSVDQNVRSVMRLLDICDRLPNIQAFIHVSTAYCNADLITVEEKVYTSPVSLDQVLALTDSVPDELMAEITDKFIAPKPNTYTFTKALAENAVEQHRNRGYPVAIFRPTIVISALQHPFPGWIENLNGPSGVVVGAGKGLLHVFCCKNSAKADLLPVDIAIDTLIAVAWETALDKLSEVRVYNCSTIDNPVTWREFETTIRRELRIHPFDKAFWYPSGTVIESKYLQKAIALLTQTLPLHLAEYTVRLFGIKTRLSFITVNQRLCAMSDVLQFFSLREWIFKKDNVRRLQARLSPRDAEIYNLDPQTINWTEHFINFVKGTRKYLLKEKDQDIGEAKRHIRRMYYVHHGFILFLLVLVFRYAMQNPIIRSIVYGTLKLLFSAFNAAYGRI

SEQ ID NO: 161 - protein, 462 aa, Ostrinia furnacalis
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLIFKMSLTAAVNINTKPTEQLIAICKKMRRNPIFIYVSSAYSNVNEQIIDEKVYSTGVPLETIYDTLDAKNTRLMDIFLDKRPNTYTYSKALAEVVVEKEFDESAAIVRPSIIASSIREPIPGWLSGSHGFPRVVEAACKGLLLRWHGDGTVAFDVIPVDHVANLIIAAAWESNERRLMGNKGVKVYNCCSGLRNPIDVSTVMNTCIKYRKYFGTRTMSIITPRFIMKKNYFLYKLLYFTYHTIPAAIIDGFFWLTGRTPMMLNTLHKLRKLSSVLEYFTLHQFLFLDSNVRGLLRRMESTDRQTFNFDVTEIEWEPYLQNFVRGIANNYNYSM

SEQ ID NO: 162 - protein, 535 aa, Plodia interpunctella
MRLVPSALDVVLAAERRLVPIHEATLKGDSEVQKFYAGSVVLVTGGSGFLGKQLIEKLFRTCDVKKLYILLRAKKGKSIGERLDYILRNPVYDTLRETRPWFADRIEAVEGDIASYRLGVADHMWDRLVDEVTVVFHGAATINFADPLQLATNINVKGTLEMLEFGKACKNLRSYVHISTAYNQALKNNVSCDILEEFPKAPLSPTTLVDLANRTETKVLDEMFHKYLAPEYPNTYAYTKAVAEEAVRTMAGDLPICVVRPSIVIPAYTEPSAGWVDKNSIYGASGIIMGLGLGVIHTLISDPDIKIDIIPVDIVNNAVIVAGWETARRREQGETDTKIYTVGSSNRNHITWRFLTNVFDNEGRKHLSSQAVWYAFAVQTKSKHLYLALAWLLHFIPGYIVDGVLMIRGKKPQVSKIYRLLNTMATIFSFFTLRGWNFKDDNLLQLYKSLSKTDQQIFNMDIATLDMKEVIYLWHIGLRRFYLKDKLNDQEKSMKKQFFLRIITYTFIPIYLYFLFKISCFVFYGLYYLIFSYLI

SEQ ID NO: 163 - protein, 531 aa, Plodia interpunctella
MTVNTRPSTNMESNIISERNYVRDCTGIDVNMNEKRLTSVQQFYNGKNVLITGATGFLGKILVEKLLRCCPGVENLYLLVRQKKGKDIYTRIDEIFDDPVFDLLKEQHPKFRHKVVVVPADCEVAGLGLTLTDRQMLTEKVNIIFHSAATVKFDEHLRAALNTNVRAPLHLLRLARDMKDLDVLMHISTAYSNSHLPHIEERFYPCEADCEQLHRMIDKLTDGEIDALLPTILGAWPNTYTFTKALAEKELRENAGDLPVGIFRPAIVTSTYKEPINNWLDNMYGPTGVAVGTATGILRTLQCDPNVTADLVPVDHVVNCLVVAAQKVNAAYQTSTPPREPPIFNYVSSTENRMTWGDFMRQNIARLDKTPFSNAIWYLSLRLTRSGARHRVYSLLLHLLPAALADALAFCLGKKPKMLKVYRKVHKLSSVLSYFCTTELTFCNRNTRQLWESTSDEDKKIFPFSMRDVNWDEFFEEYIVGMRRHLFKDGDDTLPQARIKWKRLYYLHQIVKILFCALAVYALWSIFLRLW

SEQ ID NO: 164 - protein, 459 aa, Spodoptera exigua
MTYRQINEFDAEKFTAAAVPTSYVSVPDFYAGKSIFITGGTGFLGKVFLEKLLYSCKDVETVYILIREKKGKTPQQRVEDLFNKPIFSRLKQKDSQCMKKVTAIIGDLSEPGLGISKDDEELLLQKVSVVFHVAANVQFYKEFKEIINTNVGGTKYVLQLCQRIKDIKAFVHISTAYCHTDQKVLEERIYPPPAELSEVLKFLQQPQHDKKQIKELFKKQPNSYTFAKALAETYIAENCGRVPTIIIRPSIISASLKEPLPGWVDSWNGATGLITASYNGANRVLLGEGGNFLDLIPVDFVANLAIVAAAKCTSSLKVYNCCSSGCNPLTLKQLVSHMNNVGFDKNVSIIFTNNKASLSTLTFFLQTTPSFTADMFLRVTGKSPRYMKIQSKLTIARNALNFFTCHSWVMKADNSRRLYASLSLHDRHTFPCDPTDIDWKKYITIYIEGINQFLMKKRS

SEQ ID NO: 165 - protein, 520 aa, Spodoptera exigua
MVPRPAPQFPTPPLIPEYFAGREVLITGATGFMGKVLVERLLWTCPDIGRLHLLMRHKRDVAPDKRLALLKQSQVFDVVRERCPQQLDKLCMVPGDVTKRRFGFDQSALNQLNQVSVVFHSAATLKFDEPLSVAAEQNVRPVLTLMDICDQLPNMQVLVHVSTAYSNAELAEVEERVYPAPVTPEHLLALVDALPASMLQEITPRLIAPKPNTYTFTKAVAESAVSERAVTARYACAIFRPTIVVSSLRHPFPGWIENLNGPSGVVAGAGKGLLRVLRCGAQRRADMMPVDICIDTLIAVAWETGIDNLREARVYQCASSSHAATWGQFRERMLRLVREHPFDNVLWYPYGVICENTVVQKVLEAVLQTAPLCVAHCVARACGLKQKPSLWTACKRLQAMNQALQFFATRHWSFRTTRVQQLAHRLHPDDQKLYNLRPETIDWEQHCVDFVKGARRYLLRERDDDIHAARRRMRILYLIHKATLLFAIFTMCRLTIRTAPAILWGVAALTRQRNKSALLS

SEQ ID NO: 166 - protein, 526 aa, Trichoplusia ni
MASCLSGGHYVPGSQEYVPVAEFYADKSVFVTGGTGFMGKVLVEKLLRSCPKIKKIYLLMRPKRGQDVASRLTELTQSPLFETLRRERPQELNKIVPIVGDITEPELGISAADQTMLCQKVSVVFHSAATVKFDEKLKLSVTINMLGTQQLVQLCHRMLSLEALVHVSTAYCNCERERVEETVYAPPAHPEHVVTLVQTLPDELVDRITPDLVGDRPNTYTFTKALAEDMLIKESGNLPVSIVRPSIVLSSLREPVKGWVDNWNGPNGIIAAVGKGIFRTMLGTGTKVADLVPVDTVINLMIVCAWRTHLRRGEGVVVYNCCTGQQNPITWQRFVKTSFKYMRKHPFNEVVWYPGGDITSNRLWHGALSLLQHRAPAALMDLAASASGKKPMMVRVQNKLEKAAACLEYFTTRQWAFADDNVQALCSSLSPEDRRTFDFNVRNINWDAYIESYVLGIRRFLFKESPDTLPQSRAVLRRLHIVHILTQVATVFFLWRFLFSRSNALRNLWRRVLELLTRAARLLAIA

SEQ ID NO: 167 - protein, 449 aa, Yponomeuta rorellus
MVQLKEDSVAAFYAEKSIFITGGTGFLGKVLIEKLLYSCKAVDQIYVLIRKKKDQTPSERIAQLLESELFSRLRKDDPSALKKVVPVVGDLTMPNLGLSAAVEDLIVSKVTVIFHVAATVKFNERMKNALVNNVEATREVINLCHRLEKVDAFIHVSTAYSNTDQKVVEERVYPPPAPLSEVYAFVKNYGDDMDIIQNLLNGRPNTYTYTKALAEDIVLKEHGGIPTAIIRPSIVLSVLKEPIPGWLDNWNGPTGLLHASSQGVHCSMLGSGSNVADLIPVDIVTNLMIVVASRCRKSNGLKVYNSCSGTTNPITYQAFTKMFLDSCISRGWNKVPFPLLIFVKWAFLNRVLKFLLVIVPFFLIDVYLRFFGKPNYMRMITYTKKAEDLMTFFTSHEWQFKDGNVRDLINMMSPEDRKIFYCDPEEIQWKPYFDDYCVGVFKYLLKRKV

SEQ ID NO: 168 - DNA, 1632 bp, Amyelois transitella
atggccgccaagtgcgagatcggcgtgtctcagttcttcgccggcaagcacgtgttcatcaccggcgccaccggcttcatgggcaaggtgctggtcgagcgactgctgtctacctgcgccgacgtgggccgaatctacctgctgatgcgatctaagaaggaccactctcctgagcagcgactgcaagagctgaaggcctctcaggccttcgacaacctgcgacagtctcagccctctcagctcgagaagctgcgaatcgtggccggcgacatcatgctgcccggcctgggcatctctcaagagggcctcgaggacctgcaagaggtgtctgtggtgttccactctgccgccaccgtgaagttcaacgaggccctgaagatcgccatcgagcagaacctgatgtctgtgatccgactgctcgagatctgcgaccgactgcctcacatgcaggctttcgtgcacgtgtctaccgcctactctaaccccgagctgtccaccgtcgaggaacgagtgtaccctcctcctgtgcctctggactacctgctcgacctggccgccgaggactctcccctgctggacaacgtcgagtctttcatctctcccaagcctaactcttacaccttcaccaaggccatggctgaggccgccgtgcagcgacacggctgccagtactaccccattgccatcgtgcgacccaccatcgtgatctcttcgctgaagcacccctttccaggctggctcgagaacgccaacggacccaccggcgtgattttcggagccggccgaggcctgctgcgagtgttccgatgccgacagtctgccctggccgacctgctgcctgtggacatcggcatcgacaccctgatcgccgctgcctgggaagtcgcctctgaccgacctccttcgcctcgagtgtacaactgctctatctctgagaaccccaccacctggggcgacctgaagcgagccatcaacctgcacacccgatctcagccccagtgtggcgccgtgtggtatcccggactgcataccgccgagaacggcttcctgtacaagatcctcgaggtgctgctgcagaccactcctctgtacatcggcgagatcctggtgcgaatcctgggctactctcacaagcgactgaacctgatccgagtgtctacccagatgaagaacatgcagaacgccatgaagtactttgccctgcgagagtggcgattccagtctgacaacgtgcgacgactgcgagagcgaatgtctgacgtggacaagcgaatctacaacctggatccttgctctatcgtgtgggacgagcactactccaactacgtgaagggcgtgcgactgtacctcctccgagagaaggaactggaccagcctgccgccaagaagcgactcaagaagctctactacctgcactacggcctgtctgtgttcatgccctttctgctgttccgagccttcgtccgacgaaagcaccgacagaacattctcgaggtcctgttcgccatcctgtctatcttcaacgccatcttccgacgatcgacccagaacatcctgtacatctacaaggtgtctctgaccttcctgaacgacaacgtgttcctgaacaaggtgtttaacgagtcctcttag

SEQ ID NO: 169 - DNA, 1566 bp, Amyelois transitella
atggtggcccgacgagagtctgtgtctgctcccgtgattccccagttctacgccggacgatctatcttcatcaccggcgccaccggcttcatgggcaaggtgctgatcgagcgaatcctggccacctgtcctgacgtggaccgactgtacctgctggtgcgacacaagaaggaagtcacccctgagcgacgactgcagcagctgattgagtctcccgtgttcgaccccatccgagcttctaacccctctcagctggacaagctgtctatgctggccggcgacatcacccgacctgacctgggcatgacccaagagtctctgatgcgactgcaagaggtgtctatcgtgttccactctgccgccactctgaagttcgacgaggccatgatgatggccgtcgagcagaacgtgctgtctgtgatccgactgctcgagatctgcgaccgactgcctaagatgcaggccttcgtgcacgtgtctaccgcctactctaacgccgagctgtctgaggtcgaggaacacgtgtaccctccaaaggtgcccctcgagaagctgctggccattgccgagtctgtccccgacgagctgatgaccgagatcacccctaagttcattgctcccaagcctaacacctacaccttcaccaagtctatggccgagcacgtggtgcagcagcacggcaacaagggctaccccgtggccatcttccgacctaccatcgtgatctcttccgtgaagcacccctttccaggctggatcgagaacctgaacggcccctctggcgtggtggctgctgccggcaagggcctgctgcacgtgttctgctgccgaggcgacgcccacgccgacatgctgcccgtggacgtggccattgacaccctgatcgccgctgcctgggagactgccatcgacaagcccaacgaggcccgagtgtacaactgcaccactcgagagaaccccaccaagtggatcgacttcgagcacgccctgcgaaagtacctggtcgagtaccctctggacaaggccttctggtatccctctggtgccgctatcgacaacacctacgctcaaaaggccatggaactgttcacccagaccgtgcctctgcacatcgtggcctacaccgtgcgactgctgggcatcaagatgcagatgaacctgatcaccgtgtctcaccgactgcaggccatgaacaaggtcctgaagttcttcgcccagcgagagtgggtgttccacaacgagaacgtgaagcgactgcgaaaccgactgacccctcaggacgccgccatctacaacctggatcctaagtctttcaactgggacgagcactactgcaacttcatcaagggcacccgaaagtatctgctgaaggaaaaggaacaggacctggaagaggcccgaaagcacgtgcgacgaatgttctacgtgcactactctgtgctgctgttcgtggtggtcctgctgctgcgattcgccctgaacaacaacttcgtgcgacagttcatctacggcgtggtgcgaaccctgaccgctgccttcggcgccgtgttctctaagatcgtgtag

SEQ ID NO: 170 - DNA, 1572 bp, Amyelois transitella
atggcctcttgcgtcggcggagccttccacgctcaagagtacctgcctgtggccgacttctacgccgacaagtctatcttcgtgaccggcggcaccggcttcatgggcaaggtgctggtcgagaagctgctgcgatcttgccccaagatcaagaagatctacctgctgatgcgacccaagcgaggccaggacgtgtcctctcgactgaccgagctgacccagtctcctctgttcgagactctgcgaaaggaacgaccctctgagctgtctaagatcgtgcccatcgtgggcgacatcaccgagcctgagctgggcatctctcccgccgaccaggccatgctgtgccaaaaggtgtctgtggtgttccactctgccgccaccgtgaagttcgacgagaagctcaagctgtctgtgaccatcaacatgctgggcacccagcagctggtgcagctgtgccaccgaatgctgggactcgaggccctggtgcacgtgtctaccgcctactgcaactgcgagcgagagcgagtcgaggaaaccgtgtacgcccctcctgctcaccccgagcacgtggtgaccctggtccagactctgcccgacgacctggtggaccgaatcacccctgacctggttggcgaccgacctaacacctacaccttcaccaaggctctggccgaggacatgctgatcaaggaatgcggcaacttgcccgtggccatcgtgcgaccctctatcgtgctgtcctctctgcgagagcccgtgaagggctgggtcgacaactggaacggccccaacggcatcattgccgccgtcggcaagggcgtgttccgatctatgctcggaaccggcgctcgagtggccgatctggtgcccgtggacaccgtgatcaacctgatgatcgtgtgcgcctggcgaacccacctccgacgaggcgagggcgtcgtggtgtacaactgctgcaccggccagcagaaccccatcacctggcagcgattcgtcaagacctctttcaagtacatgcgaaagcacccattctctgaggtggtgtggttccccggtggcgacattaccaacaaccgactgaagcacaacatcctgtctctgctccagcacaaggctcccgccatcgtgatggacctggtgtctcgagcctctggcaacaagcccgtgatggtgcgagtgcagaacaagctcgagaaggccgctgcctgcctcgagtacttcaccactcgacagtgggccttctctgacgacaacgtgcaggccctgtgctctgccctgtctgtcgaggaccgacgaatcttcgacttcaacgtgcgaaacatcgactgggacgcctacatcgagtcttacgtgctgggaatccgacgattcctgttcaaggaatctcccgacactctgcccaagtcgcgagccctgctgcgacgactgcacatcgtgcacaccctgactcaggtgaccaccgtgtttctgctgtggcgatttctgttctctcagtcgtctaccctgcgatctatctggcgacgaattctcgagttcatcacccgaattttccgactgctggccattgcctag

SEQ ID NO: 171 - DNA, 1386 bp, Chrysodeixis includens
atgttcgcctctaccgacggcggacgatctgacgaggaaacccctcagcagcacgagtctatcgccgacttctacgccggcaagtctgtgttcattaccggcgtgaccggcttcctcggcaaggcctacctcgagaagctgctgtactcttgcaaggacatcgacaaggtgttcgtgctgatccgaaacaagcgaggcgacgacgtgggcaagcgaattgagaagctcctggactcctctgtgttctctcgactgcgatctcaccgacctgaggacctgaagaagatcacccctgtctacggcgacattgacaagtctgagctgggcctgtctctggccgaccaagagcgactgatcaaggaagtgtctgtcgttttccacgtggccgcctctgtgaacctggacgccgacctcaagacctctctggtgaccaactacttcggcaccactcacgtgctgaagctgtgccaccgaatgaagaagctgaaggttttcgtctacgtgtctaccgcctactgcaacaccaccgtgaaggtgctggaagagaaggtgtaccctctgcctgtcgagctggacgaggtggtgaagatcatggaacagccccacctggactctaaccgagtcaagaagttgctgaacggacgacccaacacctacgctctgtctaaggccctggccgagcactacattgccgagaaccacggtgacatccccgtgattttcattcgaccctctatcgtgacctcttctgctcgagagcccgctcctggctgggccgactctttccagggcgccaccgctctgatcaccgcctgctggaagcgagtgaaccgagtgatctacggcaacggcgacaacatcatcgatctgatccccgtggactacgtgtccaacctgtctatcgtggccgctgccaaggtgaagtctaccaccgagatcgccgtgtacaactcttgcacctcttctatcaagcccatgactctgaagggaatcgccgagcagatcgagcgagtcaacaccgagaacaagcagggctctatcttcctgcctactattttcttcacctcctcttggctgttcgtgatcatgctgaccttcgtgctgcagctgctcccctcttttattgccgacctgttcctgtacatcaccggcaacaagcctatgtacatgaagatccagtctaaggtgctgcacggccgacacatcctgaactactacaccaacgactcttgggtgttcaagtctgactcttctcagcgactgcaggactctctgtctcccgccgacaagcgactgttcccctgctgtccctctgacatcaagtgggacgagtactttaacacctacttttgcggcatcgacaactacctgctcaagcgaaagtag

SEQ ID NO: 172 - DNA, 1569 bp, Cydia pomonella
atggtggctccccgagatcgacgaggcgctctgattccccagttcttcgccggacgatctgtgctgatcaccggcgccaccggcttcgtcggcaaggtgctggtcgagcgactgctgtctacctgtcctgacatctctaccctgcatctgctgctgcgagtgaagaagggcgctaccccttctgagcgactgaaggaactcaaggaatctcaggtgttcgacgtggtgcgacagcgaaacccctctcagctggacaagctgcgagttcttgagggtgacgttgctgctcctggtcttggtcttgctgctgacgctgctcttactcttcaggacgtgtctgtggtgttccactctgccgccactctgaagttcgacgaggaactgcgaaaggccgtggaccagaacgtgcgatctgtgatgcgactgctcgagctgtgcgacaagctccccaacatgcaggccttcatccacgtgtctaccgcctactctaacgccgagctgtcctctgtcgaggaacgagtgtaccctcctcctgctcctctggaagccgtgctggccctgaccgaccacgtgcctgatcctactctggccggcatcacccaccagtacattgctcccaagcctaacacctacaccttcaccaaggctctggccgagactgtggttgagcgacacggcaacactggctaccccatctctatcttccgacctaccatcgtgatctctgcccagaagcacccctttccaggctggatcgagaacttcaacggcccctctggcgtggtcgtggccaccggcaagggcctgctgcacgtgttccccaccaagtgcggcgtgcacaccgactttctgcccgtggacattgccgtggacaccctgatcgccgtggcctgggagactgccgtcgaccactctaaggaagtgcgagtctacaactgctctaccggcgctaaccccaccaccttcgaggacttcgagcgaaacctgcgacgagagactacccgacatcctctggacggcgccctgtggtatccctctggcaccgccgtgcagaccaagtgggctcgaaccctgctcgagttcctgctgcagaccgtgcctctgcacctggccgagtacaccgctcgaatgatcggcctcaagacccctgtgaacctgattaccgccaacaagcgactgaccgccatgaccgacgctctcgagttctttgctacccgagagttcaacttcgagtgtgagcgagtgcgagccctgcaccaccgactgtcgcctgaggacaagaagatctacaacctggacgtgatgtctatcaactgggacgaccactacgccgacttcgtgaagggcacccgaaagtacctgctgaacgagaaggaacaggacctcgagaaggcccgacgacacatgcacaagatgtggtttctgcacaaggtcctgaagctggtgaccctgctgctgttcctgcgaatcctggtgcagaaccgatacgcccgatctttcgtgtacggcaccctccgactgctgctctcgctgttcggcgccgtgtaccatcgactggtgcacgcctag

SEQ ID NO: 173 - DNA, 1009 bp, Marinobacter algicola
atgtgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcaggccacccagcagcagcagaacggtgcatccgcttcgggcgttctggagcagcttagaggcaagcatgtcttgattaccggtactacaggatttctgggaaaggtggttctggagaagctgatccgaaccgtgcctgacatcggtggtattcatctgctgattagaggcaacaagagacatcctgctgccagagaaagattcttgaacgaaatcgcctcttcctctgtgttcgagcggcttagacatgacgacaacgaagcctttgagactttcctggaggagcgtgtgcactgcatcaccggagaagtgaccgagtcgagatttggccttactcctgagcggttccgagcccttgctggccaagtggatgccttcatcaattccgccgcctctgttaacttcagagaggagctggacaaggcactcaagatcaacaccctgtgtctggagaacgtggctgctctggccgaacttaactccgctatggcagtgatccaagtttccacctgttacgtgaacggcaagaactctggacagatcaccgagtccgttatcaagcccgctggcgaatccatccccagatccacagatggctactacgagatcgaggagctggtccaccttctgcaagacaagatc

SEQ ID NO: 174 - DNA, 1569 bp, Manducta sexta
atggtgccccgacctgtgtctccctacgctcgagagccccagattcctcagttctacgccggcaagtgcatcttcatcaccggcgccaccggcttcatgggcaaggtgctgatcgagcgactgctgtggacctgtcctgacatcggcaagctgtacctgctgatgcgacagaagcgagaggtttctcccgagaagcgactgtctcagctgaagcagtctcaggtgttcgacaacatccgagagaagtgcccctctcagctggacaagctctacatcatccacggcgacaccaccgagcctaacctgggcatctctctggactctatgtctctgctgaacgaggtgtctatcgtgttccacggcgctgctaccctgaagttcgacgaggtgctgggcaagtctgtggaccagaacgtgcgatctgtgatgcgactgctcgacatctgcgaccgactgcctaacatccaggccttcatccacgtgtctaccgcctactgcaacgccgacctgatcaccgtggaagagaaggtctacacctctcctgtgtcgctggaccaggtgctggccctgaccgactctgtgcccgacgagctgatggccgagatcaccgacaagttcattgctcccaagcctaacacctacaccttcaccaaggctctggccgagaacgccgtcgagcagcaccgaaaccgaggctaccccgtggccatcttccgacctaccatcgtgatctctgccctgcagcacccctttccaggctggatcgagaacctgaacggcccctctggcgtggtggtcggagccggcaagggcctgctgcacgtgttctgctgcaagaactctgccaaggccgacctgctgcctgtggacattgccatcgacaccctgattgccgtggcctgggagactgccctcgacaagctgtctgaggtgcgagtgtacaactgctctaccatcgacaaccccgtgacctggcgagagttcgagactaccatccgacgagagctgcgaattcaccccttcgacaaggccttctggtatccctctggcaccgtgatcgagtctaagtacctgcagaaggctatcgccctgctgacccagactctgcccctgcacctggccgagtacaccgtgcgactgttcggcatcaagacccgactgtctttcattaccgtgaaccagcgactgtgcgccatgtctgacgtgctgcagttcttctcgctgcgagagtggatcttcaagaaggacaacgtccgacgactgcaggctcgactgtcgccccgagatgccgagatctacaacctggatcctcagaccatcaactggaccgagcacttcatcaacttcgtgaagggcacccgaaagtatctgctgaaggaaaaggaccaggacatcggcgaggccaagcgacacatcagacgaatgtactacgtgcaccacggcttcatcctgtttctgctggtgctggtgttccgatacgccatgcagaaccccatcatccgatccatcgtgtacggcaccctgaagctgctgttctctgccttcaacgccgcctacggccgaatctga

SEQ ID NO: 175 - DNA, 1389 bp, Ostrinia furnacalis
atgtctgccaacaccatggaaaccgacgagcagttcacctacaactctcccatcgtgaacttctactctggcaagtctgtgttcgtgaccggcgctaccggcttcctgggcaccgtgctggtcgagaagctgctgttctcttgcaagggcatcaacaacatctacatcctgatcaagcagaccgaggacctgaccatcgaggcccgaatcctgaactacctgaactctaaggccttccaccgagtgaagaacactaaccccgagctgatgaagaagatcatccccatctgcggcaacctcgaggacaagaacctgggcatctctgactctgacatgaagaccctgctggaagaggtgtctatcgtgttccacgtggccgccaagctgatcttcaagatgtctctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctaccggcgtgcccctcgagactatctacgacaccctggacgccaagaacacccgactgatggacatcttcctggacaagcgacccaacacctacacctactccaaggctctggccgaggtggtcgtcgagaaggaattcgacgagtctgccgccatcgtgcgaccctctatcattgcctcttctattcgagagcccattcctggctggctgtctggctctcacggcttccctcgagtggtcgaggccgcctgcaagggcctgctgctccgatggcacggcgacggcaccgtggccttcgacgtgatccccgtggaccacgtcgccaacctgatcattgccgccgcttgggagtctaacgagcgacgactcatgggcaacaagggcgtgaaggtctacaactgctgctctggcctgcgaaaccctatcgacgtgtctaccgtgatgaacacctgtatcaagtaccgaaagtacttcggcacccgaaccatgtctatcatcacccctcgattcattatgaagaagaactacttcctgtacaagctgctgtacttcacctaccacactatccccgctgctattatcgacggcttcttctggctgaccggacgaacccctatgatgctgaacaccctgcacaagctccgaaagctgtcctctgtgctcgagtactttaccctgcaccagtttctgtttctggactccaacgtgcgaggactgctgcgacgaatggaatctaccgaccgacagaccttcaacttcgacgtcaccgagatcgagtgggagccctacctgcagaacttcgtgcgaggcattgccaacaactacaactattctatgtag

SEQ ID NO: 176 - DNA, 1608 bp, Plodia interpunctella
atgcgactggtgccctctgctctggacgtggtgctggccgccgagcgacgactggtccccatccacgaggctaccctgaagggcgactctgaggtgcagaagttctacgccggctctgtggtcctggtgaccggcggctctggcttcctgggcaagcagctgatcgagaagctgttccgaacctgcgacgtgaagaagctgtacatcctgctgcgagccaagaagggcaagtctatcggcgagcgactggactacattctgcgaaaccccgtgtacgacaccctgcgagagactcgaccctggttcgccgaccgaatcgaggccgtcgagggcgacattgcctcttaccgactgggcgtcgccgaccatatgtgggaccgactggtggacgaggtgaccgtggtgttccacggcgctgccaccatcaacttcgctgaccctctgcagctggccaccaacatcaacgtgaagggcaccctcgagatgctcgagttcggcaaggcctgcaagaacctccgatcttacgtgcacatctctaccgcctacaaccaggctctgaagaacaacgtgtcttgcgacatcctggaagagttccccaaggctcccctgtctcctaccactctggtggacctggccaaccgaaccgagactaaggtgctggacgagatgttccacaagtacctggcacctgagtaccccaacacctacgcctacaccaaggccgtggccgaagaggccgtgcgaaccatggccggcgacctgcctatctgcgtggtgcgaccctctatcgtgatccccgcctacactgagccctctgccggctgggtcgacaagaactctatctacggcgcctctggcatcatcatgggcctcggcctgggcgtgattcacaccctgatctctgaccccgacatcaagatcgacatcatccccgtggacatcgtgaacaacgccgtgatcgtggccggatgggagactgcccgacgacgagagcagggcgagactgacaccaagatctacaccgtgggatcttctaaccgaaaccacatcacctggcgatttctgaccaacgtgttcgacaacgagggccgaaagcacctgtcctctcaggccgtgtggtacgccttcgccgtgcagaccaagtctaagcacctctacctggctctggcctggctgctgcacttcatccccggctacatcgtggacggcgtgctgatgatccgaggcaagaagccccaggtttctaagatctaccgactgctgaacactatggccaccatcttctcgttcttcaccctccgaggctggaacttcaaggacgacaacctgctgcagctgtacaagtctctgtctaagaccgaccagcagatcttcaacatggacattgctaccctggacatgaaggaagtgatctacctctggcacattggcctgcgacgattctacctgaaggacaagctgaacgaccaagagaagtctatgaagaagcagttcttcctgcgaatcatcacctacaccttcattcctatctacctgtacttcctgttcaagatctcttgcttcgtgttctacggcctgtactacctgatcttctcttacctgatttga

SEQ ID NO: 177 - DNA, 1596 bp, Plodia interpunctella
atgaccgtgaacacccgaccttctaccaacatggaatctaacatcatctctgagcgaaactacgtgcgagactgcaccggcatcgacgtgaacatgaacgagaagcgactgacctctgtgcagcagttctacaacggcaagaacgtgctgattaccggcgctaccggcttcctgggcaagatcctggtcgagaagctgctccgatgctgtcccggcgtcgagaacctgtacctgctggtgcgacagaagaagggcaaggacatctacacccgaatcgacgagatcttcgacgaccccgtgttcgacctgctgaaggaacagcaccccaagttccgacacaaggtggtggtggtccccgccgactgcgaggtggccggcctgggactgaccctgaccgaccgacagatgctgaccgagaaggtgaacatcattttccactctgccgccaccgtgaagttcgacgagcacctccgagccgctctgaacaccaacgtgcgagcccctctgcacctcctgcgactggcccgagacatgaaggacctggacgtcctgatgcacatctctaccgcctactctaactctcatctgccccacatcgaggaacgattctacccctgcgaggccgactgtgagcagctgcaccgaatgatcgacaagctgaccgacggcgagatcgacgctctgctgcccaccatcctcggcgcttggcccaacacctacaccttcaccaaggctctggccgagaaggaactgcgagagaacgccggcgacctgcctgtgggcatcttccgacctgccatcgtgacctctacctacaaggaacccatcaacaactggctggacaacatgtacggccccactggcgtggccgtgggcaccgccaccggaatcctgcgaaccctgcagtgcgaccccaacgtgaccgccgacctggtgcctgtggaccacgtcgtgaactgcctggtggtggccgctcagaaggtcaacgccgcctaccagacctccacacctcctcgagagcctccaatcttcaactacgtgtcctctaccgagaaccgaatgacctggggcgacttcatgagacagaacattgcccgactggacaagacccctttctctaacgccatctggtatctgtctctgcgactcacccgatctggcgcccgacaccgagtgtactccctgctgctgcatctgctgcctgccgctctggctgacgccctggccttctgcctgggaaagaagcccaagatgctgaaggtctaccgaaaggtgcacaagctgtcctctgtgctgtcttacttctgtaccaccgagctgactttctgcaaccgaaacactcgacagctgtgggagtctacctctgacgaggacaagaagattttccccttctctatgcgggacgtgaactgggacgagttctttgaagagtacatcgtgggcatgcgacgacacctgttcaaggacggtgacgacaccctgcctcaggctcgaatcaagtggaagcgactctactacctgcaccagatcgtgaagatcctgttctgtgccctggccgtgtacgccctgtggtctatctttctgcgactgtggtag

SEQ ID NO: 178 - DNA, 1380 bp, Spodoptera exigua
atgacctaccgacagatcaacgagttcgacgccgagaagttcaccgccgctgctgtgcccacctcttacgtgtctgtgcccgacttctacgccggcaagtctatcttcattaccggcggcaccggattcctcggcaaggtgttcctcgagaagctgctgtactcttgcaaggacgtcgagactgtgtacatcctgatccgagagaagaagggcaagacccctcagcagcgagtcgaggacctgttcaacaagcccatcttctcgcgactgaagcaaaaggactctcagtgcatgaagaaggtgaccgccatcatcggcgacctgtctgagcccggcctgggcatctctaaggacgacgaggaactgctgctgcaaaaggtgtctgtggtgttccacgtggccgccaacgtgcagttctacaaggaattcaaggaaatcatcaacaccaacgtcggaggcaccaagtacgtgctgcagctgtgccagcgaatcaaggacatcaaggccttcgtgcacatctctaccgcctactgccacaccgaccagaaggtgctggaagaacgaatctaccctcctcctgccgaactgtctgaggtgctgaagttcctgcagcagccccagcacgacaagaagcagatcaaggaactgtttaagaagcagcccaactcttacaccttcgccaaggctctggccgagacttacattgccgagaactgcggacgagtgcccaccatcatcattcgaccctctattatctctgcctctctgaaggaacccctgcctggctgggtcgactcttggaacggcgccaccggcctgatcaccgcctcttacaacggcgctaaccgagtgctgctcggcgaaggcggcaacttcctggacctgattcctgtggacttcgtggccaacctggccatcgtggccgctgccaagtgcacctcttcgctgaaggtgtacaactgctgttcttctggctgcaaccctctgactctgaagcagctggtgtctcacatgaacaacgtgggcttcgacaagaacgtgtctatcatctttaccaacaacaaggcctctctgtctaccctgacattcttcctgcagaccactccttcttttaccgccgacatgttcctgcgagtgaccggaaagtctccccgatatatgaagatccagtctaagctgaccattgctcgaaacgccctgaacttcttcacctgtcactcttgggtgatgaaggccgacaactctcgacgactgtacgcttctctgtctctgcacgaccgacacactttcccttgcgaccccaccgacatcgactggaagaagtacatcaccatctacatcgagggaatcaaccagttcctgatgaagaagcgatcttag

SEQ ID NO: 179 - DNA, 1563 bp, Spodoptera exigua
atggtgccccgacctgctcctcagttccccacacctcctctgatccccgagtacttcgccggacgagaggtgctgatcaccggcgccaccggcttcatgggcaaggtgctggtcgagcgactgctgtggacctgtcctgacatcggccgactgcatctgctgatgcgacacaagcgagatgtggctcccgacaagcgactggccctgctgaagcagtctcaggtgttcgacgtggtgcgagaacgatgtccccagcagctggacaagctgtgcatggtgcccggcgacgtgaccaagcgacgattcggcttcgaccagtctgccctgaaccagctcaaccaggtgtctgtggtgttccactctgccgccactctgaagttcgacgagcccctgtctgtggccgccgagcagaacgtgcgacccgtgctgaccctgatggacatctgcgaccagctgcctaacatgcaggtcctggtgcacgtgtctaccgcctactctaacgccgagctggccgaggtcgaggaacgagtgtaccccgctcctgtgactcccgagcacctcctggctctggtggacgctctgcccgcctctatgctgcaagagatcacccctcgactgatcgctcccaagcctaacacctacaccttcaccaaggccgtggccgagtctgccgtgtctgagcgagccgtgaccgctcgatacgcctgcgccatcttccgacctaccatcgtggtgtcctcgctgcgacacccctttccaggctggatcgagaacctgaacggcccctctggcgtggtggctggcgccggaaagggcctgctgcgagtgctccgatgtggcgcccagcgacgagccgacatgatgcccgtcgacatctgcatcgacaccctgatcgccgtggcctgggagactggaatcgacaacctgcgagaggcccgagtctaccagtgcgcctcttcttctcacgccgccacctggggccagttccgagagcgaatgctgcgactggtccgagagcaccccttcgacaacgtgctgtggtatccctacggcgtgatctgcgagaacaccgtggtgcaaaaggtgctcgaggccgtgctgcagaccgctcctctgtgcgtggcccactgtgtggcccgagcctgcggcctgaagcagaagccctctctctggaccgcctgtaagcgactgcaggccatgaaccaggctctgcagttcttcgctacccgacactggtctttccgaactacccgagtgcagcagctcgcccaccgactgcaccccgacgaccagaagctgtacaacctccgacctgaaaccatcgactgggagcagcactgcgtggacttcgtgaagggcgctcgacgatacctgcttcgagagcgagatgacgacatccacgctgcccgacgacgaatgcgaatcctgtacctgatccacaaggctaccctgctgttcgccattttcaccatgtgccgactgaccatccgaactgctcccgccatcctgtggggcgtcgccgctctgacccgacagcgaaacaagtctgctctgctgtcttag

SEQ ID NO: 180 - DNA, 1581 bp, Trichoplusia ni
atggcctcttgcctgtctggcggccactacgtgcccggctctcaagaatacgtccccgtggccgagttctacgccgacaagtctgtgttcgtgaccggcggcaccggcttcatgggcaaggtgctggtcgagaagctgctgcgatcttgccccaagatcaagaagatctacctgctgatgcgacccaagcgaggccaggacgtggcctctcgactgaccgagctgacccagtctcctctgttcgagactctgcgacgagagcgaccccaagagctgaacaagatcgtgcccatcgtgggcgacatcaccgagcctgagctgggcatctctgccgccgaccagaccatgctgtgccaaaaggtgtctgtggtgttccactccgccgccaccgtgaagttcgacgagaagctcaagctgtctgtgaccatcaacatgctgggcacccagcagctggtgcagctgtgccaccgaatgctgtctctcgaggccctggtgcacgtgtctaccgcctactgcaactgtgagcgagagcgagtcgaggaaaccgtgtacgcccctcctgctcaccccgagcacgtggtgaccctggtccagactctgcccgacgagctggtggaccgaatcacccctgacctggtgggcgaccgacctaacacctacaccttcaccaaggctctggccgaggacatgctgatcaaggaatctggcaacctgcctgtgtctatcgtgcgaccctctattgtgctgtcctctctgcgagagcccgtgaagggctgggtcgacaactggaacggccccaacggcatcattgccgccgtcggcaagggcatcttccgaaccatgctcggaaccggcaccaaggtggccgatctggtgcccgtggacaccgtgatcaacctgatgatcgtgtgcgcctggcgaacccacctccgacgaggcgagggcgtcgtggtgtacaactgctgcaccggccagcagaaccccatcacctggcagcgattcgtcaagacctctttcaagtacatgcgaaagcaccccttcaacgaggtggtgtggtatcccggcggagacatcacctctaaccgactgtggcacggcgccctgtctctgctccagcatcgagcccctgccgctctgatggacctggccgcctctgcctctggcaagaagcccatgatggtgcgagtgcagaacaagctcgagaaggccgctgcctgcctcgagtacttcaccactcgacagtgggccttcgccgacgacaacgtgcaggccctgtgttcttcgctgtctcctgaggaccgacgaaccttcgacttcaacgtgcgaaacatcaactgggacgcctacatcgagtcttacgtgctgggaatccgacgattcctgtttaaggaatctcccgacactctgccccagtctcgagccgtgctgcgacgactgcacatcgtgcacattctgacccaggtggctaccgtgttctttctgtggcgatttctgttctctcgatctaacgccctgcgaaacctgtggcgacgagtgctcgagctgctgacccgagccgctcgactgctggccattgcctag

SEQ ID NO: 181 - DNA, 1350 bp, Yponomeuta rorellus
atggtccaattgaaagaagattccgttgctgctttttacgccgaaaagtctattttcattactggtggtactggtttcttgggtaaggttttgattgaaaagttgttgtactcctgcaaggccgttgatcaaatctacgttttgatcagaaagaagaaggaccaaaccccatctgaaagaattgcccaattattggaatccgaattattctccagattgagaaaggatgatccatccgctttgaaaaaggttgttccagttgttggtgatttgaccatgcctaatttgggtttgtctgctgctgttgaagatttgattgtctctaaggttaccgttatcttccatgttgctgctactgttaagttcaacgaaagaatgaagaacgccttggttaacaacgttgaagctactagagaagtcatcaacttgtgccatagattggaaaaggttgatgccttcattcatgtttctaccgcttactctaataccgatcaaaaggtcgttgaagaaagagtttatccaccaccagctccattgtctgaagtttatgcttttgttaagaactacggtgacgacatggatatcatccaaaatttgttgaacggtagaccaaacacttacacctacacaaaagctttggctgaagatatcgtcttgaaagaacatggtggtattccaaccgctattatcagaccatctatcgttttgtccgttttaaaagaacctatcccaggttggttggataattggaatggtccaactggtttgttgcatgcttcttcacaaggtgttcattgctctatgttgggttctggttctaatgttgccgatttgattccagttgatatcgtcactaacttgatgatcgttgttgcttctagatgcagaaagtctaatggtttgaaggtctacaactcttgttctggtactaccaacccaattacttaccaagctttcaccaagatgttcttggattcctgtatttctagaggttggaacaaggttccattccctttgttgatttttgtcaagtgggctttcttgaacagagtcttgaagttcttgttggttatcgtcccattcttcttgatcgatgtctacttgagatttttcggtaagccaaactacatgagaatgatcacctacaccaagaaagctgaagatttgatgaccttcttcacctctcatgaatggcaattcaaggatggtaatgtcagagacttgatcaacatgatgtctccagaagatagaaagatcttctactgcgatcctgaagaaattcaatggaagccatacttcgatgattactgcgttggtgtctttaaatacttgttgaagagaaaggtttaa

SEQ ID NO: 182 - protein, 679 aa, Cydia pomonella; identical to SEQ ID NO: 124

SEQ ID NO: 183 - protein, 669 aa, Agrotis segetum
MNTMDGSPGRENQKSTRKISLPTVLCTPEPSDDRSDMKVNSFMSSDSKMSLPTNLGTPTSPLRLSSPISLPTSPCVKTSQTSSANSLPNPNPKPVTSSAATNNIVNSGSATGNPRNKCALQPGHSLMDWIRLGNSGKDLTGVGGRIRPVSPAELSTHNTLNDAWLAIRGRVYNITHYLPYHPGGPEELMRGAGMDATQLFDKVHPWVNYDSLLAKCLVGPLRFELPDAEELFDTSNPSPKSDRLREPSKAQELVRKSMENLANCITPVRKKITKGDDNTKGSPPSKIMQSLVQSADLPMSISRRAASSPVKKTEKSPDSPLPLRYDWIQTSTKLTIYVYTGHLANPGGCARITEGFLLIEVATNGWLRTLKIKPEAKLKEPLQLRVFAESGKIEITALKVDPSVWKGCGDVSVGVASHVSSPRSVECRVMEVSRVSHDTSLLSVCPRAGPVVVPLGHHVRVHRRIEGTECIRSYTPVGEGWGPADGTEVFSALRLAVKRYDSGALSPHLTALKVGDLITLSGPYGNFQLQKLKTVKSLYLIAAGTGVTPMLGLIRFMISRSNPRCERTHLLFFNKTEEDILFRENFEEMMKEDDRLKITHVLSEGSSSWSGHRGRIRDELLAETIGNVKCDDKCSHYACLCGPTEFTYAGLDLLKKHGFKDDCIHAFMG

SEQ ID NO: 184 - protein, 578 aa, Bombus terrestris
MKKSLSKNQRKSDDTETKDIRGKMEEIDVKSSSSTNSMTNNLALLPSGVGRMKQLQGAKMVNQTGSSSSGSATGNPRNKTALAPGHSLMDWIRLGNSGVDLTGVGGVPRVVTLSELATHNKQDDAWIAIRGIVFNVTRYMDFHPGGVNELMRGVGKDATKLFENVHAWVNYQSILQKCVVGRLSRGSVSGTTSSPTENPVSSTSNCSSATLNLITSCTTHSVSQENVSDASSLNIKMDWRQTSDSLTLSYQTTRYYPGLCYQLSRINDSKLMYKLVFASGSITHELELTAEIQWPPVCIKNFETREITFTFMKQKQELWKSYGEQMVCREMNRDNRTYREYQVLTNKQLSKLVHLLVVRAKDFLQIVPIGRHVEAMLNVMGTEVSRLYTPVPPCLHPDDMAPNYKSDCLCFMIKKYPNGALSPSITALQIGETLRLSNALGDFTVESCDGYSINHMIAGGTGLTAMLGIIQRALARRSVKTINLLNFNKDEDNMFYVKELEKASADNKLKVTHILSQAGDAWTGRRGTLSDDLLQDLVGTSNPDAFVYTCGPQEFIQLAKNSLQKLNWKPFQMYEFDD

SEQ ID NO: 185 - protein, 678 aa, Lobesia botrana
MADPPAEDNTPSRNGSLKISIPTVLCTPESSENSDILCKTKSLLSSESKLSLPSNLGTPTSPLRLSSPLSLPASPTPFMKVTQPPPEKVTSGQMNSPPSTIVLQSGPKPPLANTGSATGNPRNKCALQPGHSLMDWIRLGNSGKDLTGVGGRMRPVSPDELASHNSQNDAWLAIRGRVYNITHYLPYHPGGPEELMRGAGIDATQLFDKVHPWVNYDSLLAKCLVGPLRLERPDAEELFGPSTPSPKTENRLREPSKAQELVRKSMENLANCITPVRKKISAKSEESVKGSPPSKIMQSLIQSSDLPVSISRRAAANPVKTSEKTDVAPSPLRFDWIQTSTKLTLSIYTGPLVNPGVCARIIEGILFVEVATNGWVRTLKLVPEAKLQDSLQLRVFAESGKIEVVAQKVEPGVWKGCGDAVSGAASHVPTPRSVVCRVSRVTRVSHDTVLLSLATPAQALVAPLGHHVRVHRTISDVECVRSYTPVGEGWTSDGERDSAIHLAVKGYETGALSPHLVALQPDDEVTVSGPYGNFELQKLRGVKEICFVAAGTGITPMLGLLKFILPRSNPRCERVHLLFFNKTEEDILFRQNFEEIAKEDDRFSVTHVLSDASSSWSGRRGRITSDLLTQVVGKDSLKCREQCVHFACVCGPTDFTHTTIDLLKKLHMKDSCIHAFMG

SEQ ID NO: 186 - DNA, 2040 bp, Cydia pomonella; identical to SEQ ID NO: 125

SEQ ID NO: 187 - DNA, 2010 bp, Agrotis segetum
atgaacaccatggacggctctcccggccgagagaaccagaagtctacccgaaagatctctctgcccaccgtgctgtgtacccctgagccttctgacgaccgatctgatatgaaggtgaactctttcatgtcctctgactctaagatgtccctgcctaccaacctgggaacccctacctctcctctgcgactgtcctctcctatttctctgcctacctcgccttgcgtcaagacctctcagacctcttctgccaactcgctgcccaaccctaaccctaagcctgtgacctcttccgccgccaccaacaacatcgtgaactctggctctgccactggcaaccctcgaaacaagtgcgctctgcagcccggccactctctgatggactggatccgactgggcaactctggcaaggacctgaccggcgtcggcggacgaattcgacccgtgtctcccgccgagctgtctacccacaacaccctgaacgacgcctggctggccatccgaggccgagtgtacaacatcacccactacctgccttatcaccccggtggacccgaggaactgatgcgaggcgccggaatggacgctacccagctgttcgacaaggtgcacccctgggtgaactacgactctctgctggccaagtgcctggtgggacccctgcgattcgagctgcccgacgctgaggaactgtttgacacttctaacccctcgcctaagtctgaccgactgcgagagccctctaaggcccaagagctggtgcgaaagtctatggaaaacctggccaactgcatcacccctgtgcgaaagaagatcaccaagggcgacgacaacaccaagggatctccaccttctaagatcatgcagtctctggtgcagtctgccgacctgcctatgtctatctctcgacgagccgcctcttcgcccgtgaagaagaccgagaagtctcctgactctcccctgcctctgcgatacgactggattcagacctccaccaagctgaccatctacgtgtacaccggccacctggctaaccctggcggctgcgcccgaattaccgagggcttcctgctgatcgaggtggctaccaacggctggctgcgaaccctgaagatcaagcccgaggccaagctgaaggaacccctgcagctgcgagtgttcgccgagtctggcaagatcgagatcaccgctctgaaggtggacccctctgtgtggaagggctgcggcgacgtgtctgtgggcgtcgcctctcacgtgtcctcgcctcgatctgtcgagtgccgagtgatggaagtgtctcgagtgtctcacgacacctcgctgctgtctgtgtgtccccgagctggccccgtggtggtgcccctgggccaccacgtgcgagtgcaccgacgaatcgagggcaccgagtgcatccgatcttacacccctgtcggcgaaggctggggacccgccgacggcaccgaggtgttctctgccctgcgactggccgtgaagcgatacgattctggcgctctgtctccccacctgactgccctgaaggtcggcgacctgatcaccctgtctggcccctacggcaacttccagctgcaaaagctcaagaccgtcaagtctctgtacctgattgccgccggaaccggcgtgacccctatgctgggcctgatccgattcatgatctctcgatctaaccctcgatgcgagcgaacccacctcctgttcttcaacaagaccgaagaggacatcctgtttcgagagaacttcgaggaaatgatgaaggaggacgaccgactcaagatcacccacgtgctgtctgagggatcttcttcttggtctggacaccgaggacgaatccgagatgagctgctggccgagactatcggcaacgtgaagtgtgacgacaagtgctctcactacgcctgcctgtgcggccccaccgagtttacctacgccggcctggacctgctgaagaagcacggcttcaaggacgactgcatccacgccttcatgggctag

SEQ ID NO: 188 - DNA, 1737 bp, Bombus terrestris
atgaagaagtccctgtctaagaaccagcgaaagtctgacgacaccgagactaaggacatccgaggcaagatggaagagatcgacgtgaagtcctcttcttctaccaactctatgaccaacaacctggctctgctgccctctggcgtgggccgaatgaagcagctgcagggcgccaagatggtgaaccagaccggatcttcttcttctggatctgccactggcaaccctcgaaacaagaccgctctggctcccggccactctctgatggactggatccgactgggcaactccggcgtggacctgaccggcgtcggaggcgtgccccgagtggtgaccctgtctgagctggctacccacaacaagcaggacgacgcctggatcgccatccgaggaatcgtgttcaacgtgacccggtacatggactttcaccccggtggcgtgaacgagctgatgcgaggcgtcggcaaggacgccaccaagctgttcgagaacgtgcacgcctgggtgaactaccagtctatcctgcagaagtgcgtcgtcggacgactgtctcgaggctctgtgtctggcaccacctcttcgcccactgagaaccccgtgtcctctacctctaactgttcttctgctaccctgaacctgatcacctcttgcaccactcactctgtgtcccaagagaacgtgtctgacgcctcttcgctgaacatcaagatggattggcgacagacctctgactctctcaccctgtcttaccagaccactcggtactaccccggcctgtgctaccagctgtctcgaatcaacgactctaagctgatgtacaagctggtgttcgcctctggatctatcacccacgagctggaactgaccgccgagatccagtggcctcctgtgtgcatcaagaacttcgagactcgagagatcaccttcacctttatgaagcagaagcaagagctttggaagtcttacggcgagcagatggtgtgccgagagatgaaccgagacaaccgaacctaccgagagtaccaggtgctgactaacaagcagctgtctaagctggtgcacctcctggtggtgcgagccaaggacttcctgcagatcgtgcccatcggccgacacgtcgaggccatgctgaacgtgatgggcaccgaggtgtctcgactgtacacccctgtgcctccatgtctgcaccccgacgacatggctcccaactacaagtctgactgcctgtgcttcatgatcaagaagtaccccaacggcgctctgtctccctctatcaccgctctgcagattggcgagactctgcgactgtctaacgccctgggcgacttcaccgttgagtcttgcgacggctactctatcaaccacatgattgccggcggaaccggcctgaccgccatgctgggcatcatccagcgagccctggctcgacgatctgtcaagaccatcaacctgctgaacttcaacaaggacgaggacaacatgttctacgtgaaggaactcgagaaggcctctgccgacaacaagctgaaggtgacccacatcctgtctcaggccggcgacgcttggaccggccgacgaggcaccctctctgacgacctgctgcaggacctggtgggcacttctaaccccgacgccttcgtgtacacctgtggaccccaagagttcatccagctggccaagaactctctgcagaagctgaactggaagcccttccagatgtacgagttcgacgactag

SEQ ID NO: 189 - DNA, 2037 bp, Lobesia botrana
atggctgaccctccagccgaggacaacaccccttctcgaaacggctctctgaagatctctatccccaccgtgctgtgcacccctgagtcctctgagaactctgacatcctgtgcaagaccaagtctctgctgtcctccgagtctaagctgtctctgccctctaacctgggaacccctacctctcctctgcgactctcttctcccctgtcgctgcccgcttctcccactcctttcatgaaggtgacccagcctccacctgagaaggtcacctctggacagatgaactctcctccttctaccatcgtgctgcagtctggccccaagcctcctctggccaacaccggctctgccactggcaaccctcgaaacaagtgcgctctgcagcccggccactctctgatggactggatccgactgggcaactctggcaaggacctgaccggcgtcggcggacgaatgcgacccgtgtctcccgacgagctggcctctcacaactctcagaacgacgcctggctggccatccgaggccgagtgtacaacatcacccactacctgccttaccatcctggcggacccgaggaactgatgcgaggcgccggaatcgacgctacccagctgttcgacaaggtgcacccttgggtgaactacgactctctgctggccaagtgcctggtgggacccctgcgactcgagcgacccgacgctgaggaactgttcggcccctctactccctcgcctaagaccgagaaccgactgcgagagccctctaaggcccaagagctggtgcgaaagtctatggaaaacctggccaactgcatcacccctgtgcgaaagaagatctccgccaagtctgaggaatctgtgaagggctcgcctccttcgaagatcatgcagtctctgattcagtcctccgacctgcctgtgtctatttctcgacgagccgctgctaaccccgtcaagacctctgaaaagaccgacgtggccccttcgcctctgcgattcgactggattcagacctctaccaagctgaccctgtctatctacaccggacctctggtgaaccccggcgtgtgcgcccgaatcatcgagggcatcctgttcgtcgaggtcgccaccaacggctgggtgcgaaccctgaagctggtgcccgaggccaagctgcaggactctctccagctgcgagtgttcgccgagtctggcaagatcgaggtggtggcccaaaaggtcgagcccggtgtctggaagggttgcggtgacgccgtgtccggtgctgcctctcacgtgcccactcctcgatctgtggtgtgccgagtgtctcgagtcacccgagtgtcccacgacaccgtcctgctgtccctggctacccctgctcaggccctggtggctcccctgggacaccacgtgcgagtgcaccgaaccatctctgacgtcgagtgcgtgcgatcttacacccctgtcggcgaaggctggacctctgacggcgagcgagactctgccatccacctggccgtgaagggatacgagactggcgctctgtctccccacctcgtggccctgcagcctgacgacgaggtgaccgtgtctggaccctacggcaacttcgagctgcaaaagctgcgaggcgtgaaggaaatctgcttcgtcgccgctggcaccggaatcacccctatgctgggcctgctgaagttcattctgccccgatctaaccctcgatgcgagcgagtgcatctgctgttcttcaacaagaccgaagaggacattctgttccgacagaacttcgaggaaatcgccaaggaggacgaccgattctctgtgacccacgtgctgtctgacgcctcttcttcttggtctggccgacgaggccgaatcacctctgacctgctgacccaggtggtcggcaaggactccctgaagtgccgagagcagtgcgtgcacttcgcctgcgtgtgcggccccaccgacttcacccacaccaccatcgacctgctcaagaagctgcacatgaaggactcttgcatccacgccttcatgggctag

SEQ ID NO: 190 - protein, 130 aa, Mortierella alpina
MAELKSFTLADLSQHTTKDSLYLAIHGKVYDCTGFIDEHPGGEEVLIDEAGRDATESFEDVGHSDEARDIMSKLLVGEFKTDSSEKPKAKSPSSSTPRPIPAAEPSDSGSLQYVLALAVVAGCVIWKVLL

SEQ ID NO: 191 - protein, 298 aa, Mortierella alpina
MTLSNPAIAAASGVILAGAYLIDPSALPFVAAGVAATWARVLFKKTAVKTPPMDPKEYRKFKLVDKVHCSPNTAMYKFALPHEDDLLNLPIGQHISIMANINGKDISRSYTPTSSSDDVGHFVLCIKSYPQGNISKMFSELSIGDSINARGPKGQFSYTPNMCRAIGMIAGGTGLTPMLQIIRAIVKNPEDKTQVNFIFANVTEEDIILKAELDLLSQKHPQFKVYYVLNNAPEGWTGGVGFVNADMIKEHMPAPAADIKVLLCGPPPMVSAMSKITQDLGYDKVNAVSKLPDQVFKF

SEQ ID NO: 192 - protein, 298 aa, Mortierella alpina
MSSSNPPVTATSRAFIVGASLIDSTMIPFVVASVGALLACLFFKKGTLRLPPLHAKEYRKFTLVEKIVLSPNTATYKFALPSNDDVLNLPIGQHVTVMANINGKDISRSYTPTSGSEDLGYFVLCIKSYPQGNISGMISRLAIGDQINVRGPKGHFSYIPNMCRAIGMIAGGTGITPMLQLIRSITKNPEDKTLVTLLFANVTEQDILLRAELDLLSKVHPQFKVCYVLSRASEDWTGARGYINAEMIKEHLPAPAADIKILLCGPQPMVSSMVKITQDLGYEKANAISKPHDQVFKF

(19) United States
(12) Patent Application Publication
(10) Pub. No.: US 2025/0320528 A1
(43) Pub. Date: Oct. 16, 2025

(54) IMPROVED METHODS AND CELLS FOR INCREASING ENZYME ACTIVITY AND PRODUCTION OF INSECT PHEROMONES

(71) Applicant: FMC AGRICUL TURAL SOLUTIONS A/S, Harboøre (DK)

(72) Inventors: Irina Borodina, Harboøre (DK); Carina Holkenbrink, Harboøre (DK); Kanchana Rueksomtawin Kildegaard, Harboøre (DK); Karolis Petkevicius, Harboøre (DK); Leonie Wenning, Harboøre (DK)

(21) Appl. No.: 18/559,825

(22) PCT Filed: May 10, 2022
(86) PCT No.: PCT/EP2022/062641

§ 371 Date: Nov. 9, 2023

(30) Foreign Application Priority Data
May 10, 2021 (EP) 21173017.1

(51) Int. Cl. C12P 7/04; A01N 31/02; C12N 9/02; C12P 7/54; C12P 7/6409

(52) U.S. Cl. CPC C12P 7/04; A01N 31/02; C12N 9/0008; C12N 9/004; C12N 9/0071; C12P 7/54; C12P 7/6409; C12Y 102/0105; C12Y 106/02002; C12Y 114/19001; C12Y 114/19005

(57) ABSTRACT

Herein are provided methods for increasing enzymatic activity of desaturases and fatty acyl reductases by co-expressing NAD(P)H cytochrome b5 reductases (EC 1.6.2.2, Ncb5or), as well as to methods for production of compounds comprised in pheromones, in particular moth pheromones, such as saturated and desaturated fatty alcohols, and saturated and desaturated fatty alcohol acetates and fatty acids, and derivatives thereof, in a cell.

### TECHNICAL FIELD

The present invention relates to methods for increasing enzymatic activity, as well as to methods for production of compounds comprised in pheromones, in particular moth pheromones, such as saturated and desaturated fatty alcohols, saturated and desaturated fatty acids and saturated and desaturated fatty alcohol acetates, and derivatives thereof, in a cell.

### BACKGROUND

Integrated Pest Management (IPM) is expected to play a major role for both increasing the crop yield and for minimizing environmental impact and enabling organic food production. IPM employs alternative pest control methods, such as mating disruption using pheromones, mass trapping using pheromones, beneficial insects, etc. Pheromones constitute a group of diverse chemicals that insects (like other organisms) use to communicate with individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides. The biological production of pheromones for use pest control is advantageous over chemical synthesis in respect to price, specificity, and environmental impact.

Type I pheromones of the moth order Lepidoptera are unsaturated fatty alcohols, aldehydes, or acetates of 10 to 18 carbon chain length. The receptors in male moth antennae are selective for pheromones with a specific chain length, desaturation at a specific carbon in right stereoisomery (Z or E confirmation of double bond), and terminal functional group (Tupec, Bucek, Valterova, & Pichova, 2017). Several biosynthetic enzymes contribute to pheromone production, including fatty acyl-CoA desaturases and fatty acyl-CoA reductases. The desaturases introduce double-bonds into a fatty acyl-CoA. They are thought to be integral membrane proteins receiving electrons from NADH supplied by cytochrome b5 reductase and cytochrome b5. The fatty acyl reductases convert saturated or desaturated fatty acyl-CoA's into saturated or desaturated fatty alcohols. These enzymes are also integral membrane proteins but are thought to receive electrons directly from NADPH.

Besides the “classic” single-domain membrane-bound cytochrome b5 reductase (CytB5Red) and cytochrome b5 (CytB5), another flavoheme reductase named NAD(P)H cytochrome b5 oxidoreductase (Ncb5or; also known as cytochrome b5 reductase 4 or Cyb5R4) is highly conserved in the animal kingdom. Ncb5or enzymes are distinct from classic CytB5Red/CytB5 pairs, as Ncb5ors contain three domains: a cytochrome b5-like domain, a cytochrome b5 reductase-like domain, and CHORD-SGT1 (CS domain) (Deng, et al., 2010) (see FIG. 1). The CS domain occurs in many diverse proteins and was proposed to be involved in protein-protein interaction (Benson, et al., 2019; Zhu, et al., 2004). The Ncb5or CytB5Red-like domain contains multiple insertions and deletions in comparison with CytB5Red (Benson et al., 2019). The same is true for the CytB5-like domain (FIG. 2), (Benson et al., 2019). Ncb5ors are believed to have a fundamentally different mechanism for electron transfer compared to the CytB5Red/CytB5 system (Benson et al., 2019). Furthermore, they have an unusual ability to utilize both NADH and NADPH (Benson et al., 2019).

Soluble Ncb5or have been mainly studied in mice or human cells line where knockouts of Ncb5or have led to a reduction in Δ9 desaturation (Zambo, et al., 2020; Larade, et al., 2008). To date, there are to the best of our knowledge no descriptions of Ncb5or genes or their functions in insects.

### SUMMARY

The invention is as defined in the claims.

Herein is provided a cell expressing:
i) a first enzyme or group of enzymes capable of converting a fatty acyl-CoA to a compound selected from a desaturated fatty alcohol, a saturated fatty alcohol, a desaturated fatty alcohol acetate and a desaturated fatty acyl-CoA; and
ii) a heterologous NAD(P)H cytochrome b5 oxidoreductase (Ncb5or);
whereby the cell is capable of producing the compound with a higher titer and/or purity compared to a cell expressing the first group of enzymes but no heterologous Ncb5or when cultivated in the same conditions.

Also provided herein is a cell expressing:
i) a first enzyme or group of enzymes capable of converting a fatty acyl-CoA to a compound selected from a desaturated fatty alcohol, a saturated fatty alcohol, a desaturated fatty alcohol acetate, a desaturated fatty acyl-CoA and a desaturated fatty acid; and
ii) a heterologous NAD(P)H cytochrome b5 oxidoreductase (Ncb5or);
whereby the cell is capable of producing the compound with a higher titer and/or purity compared to a cell expressing the first group of enzymes but no heterologous Ncb5or when cultivated in the same conditions.

Further provided herein is a method for increasing the activity of at least one enzyme selected from the group consisting of desaturases and fatty acyl CoA reductases (FARs), said method comprising the steps of:
a. providing a desaturase capable of introducing at least one double bond in a fatty acyl-CoA, thereby converting at least part of said fatty acyl-CoA to a desaturated fatty-acyl-CoA; and/or
b. providing a FAR capable of converting at least part of said desaturated fatty acyl-CoA to a desaturated fatty alcohol, thereby producing said desaturated fatty alcohol; and
c. contacting said desaturase and/or FAR with an Ncb5or, thereby increasing the activity of said desaturase and/or FAR compared to the activity of said desaturase and/or FAR in the absence of said Ncb5or, wherein the activity is measured under the same conditions;
wherein the increase in activity is measured by measuring the concentration of product formed by the desaturase and/or the FAR.

Also provided herein is a method for production of a compound selected from a desaturated fatty alcohol, a saturated fatty alcohol, a desaturated fatty alcohol acetate, and a desaturated fatty acyl-CoA in a cell, said method comprising the steps of:
a. providing a cell and incubating said cell in a medium; and
b. expressing in said cell a first enzyme or group of enzymes capable of converting a fatty acyl-CoA to said compound, thereby converting at least part of said fatty acyl-CoA to said compound; and
c. expressing in said cell an Ncb5or;
d. optionally, recovering said compound.

Further provided herein is a method for increasing the titer and/or purity of a compound selected from a desaturated fatty alcohol, a saturated fatty alcohol, a desaturated fatty alcohol acetate, and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the titer and/or purity of said compound compared to the titer and/or purity from a cell not expressing said Ncb5or in the same conditions;
c. optionally, recovering said compound.

Also provided herein is a system of nucleic acid constructs comprising nucleic acids encoding an Ncb5or and:
a. a desaturase capable of introducing at least one double bond in a fatty acyl-CoA; and/or
b. a fatty acyl CoA reductase FAR capable of converting at least part of a desaturated fatty acyl-CoA to a desaturated fatty alcohol.

Further provided herein is a kit of parts comprising:
a. a cell as provided in the present application;
b. a nucleic acid system as provided in the present application, wherein said construct is for modifying a cell;
c. instructions for use; and
d. optionally, the cell to be modified.

Also provided herein is the use of an Ncb5or in a method for increasing the activity of one or more enzymes.

Further provided herein is a desaturated fatty alcohol, a saturated fatty alcohol, a desaturated fatty alcohol acetate, a saturated fatty alcohol acetate, a desaturated fatty aldehyde, a desaturated fatty acid and/or a saturated fatty aldehyde obtainable by the methods of the present application.

Also provided herein is the use of a desaturated fatty alcohol, a saturated fatty alcohol, a desaturated fatty alcohol acetate, a saturated fatty alcohol acetate, a desaturated fatty aldehyde, a desaturated fatty acid and/or a saturated fatty aldehyde obtainable by the methods of the present application.

Further provided herein is a method of monitoring the presence of pest or disrupting the mating of pest, said method comprising the steps of:
a. producing a desaturated fatty alcohol and optionally a desaturated fatty alcohol acetate and/or a desaturated fatty aldehyde according to the methods of the present application; and
b. formulating said fatty alcohol and optionally said fatty alcohol acetate and/or said desaturated fatty aldehyde as a pheromone composition; and
c. employing said pheromone composition as an integrated pest management composition.

Also provided herein is a fermentation broth containing the yeast cell according to the present application.

Further provided herein is a fermentation system or a catalytic system comprising the yeast cell according to the present application.

Also provided herein is a device, such as a pheromone dispenser, for diffusing a pheromone composition, said pheromone composition comprising the desaturated fatty alcohol and/or the desaturated fatty alcohol acetate and/or the desaturated fatty aldehyde obtainable by the methods of the present application.

Further provided herein is a method for producing at least 1 mg/L of a desaturated fatty alcohol, a saturated fatty alcohol, a desaturated fatty alcohol acetate, a saturated fatty alcohol acetate, a desaturated fatty aldehyde and/or a saturated fatty aldehyde in a cell, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

Also provided herein is a method for increasing the purity of a compound selected from a desaturated fatty alcohol, a desaturated fatty acid and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the production of said compound compared to the production from a cell not expressing said Ncb5or in the same conditions;
   wherein the purity of said compound is the ratio or percentage of said compound in relation to all compounds within the same compound group produced by the cell, such as the percentage of said desaturated fatty alcohol in relation to all desaturated fatty alcohols produced by the cell, such as the percentage of desaturated fatty acid in relation to all fatty acids produced by the cell, and/or such as the percentage of desaturated fatty acyl-CoA in relation to all fatty acyl-CoA produced by the cell.

### DESCRIPTION OF FIGURES

FIG. 1. A) Schematic to visualize the domain prediction for classic cytochrome B5, cytochrome B5 reductase and Ncb5or. B) Amino acid alignment and domain prediction of multiple Ncb5ors and classic cytochrome B5 and cytochrome B5 reductase. Domains were predicted using the Batch CD-search tool (https://www.ncbi.nlm.nih.gov/Structure/bwrpsb/bwrpsb.cgi).

FIG. 2. Amino acid alignment of predicted cytochrome B5 domains from different Ncb5ors and classic cytochrome B5 proteins; DmCytB5, Drosophila melanogaster cytochrome B5 (Uniprot ID Q9V4N3); HsCytB5typeB, Homo sapiens cytochrome B5 (Uniprot ID 043169); MaCytB5, Mortierella alpina cytochrome B5 (NCBI ID Q9Y706.1); Remaining sequences can be found in the sequence list.

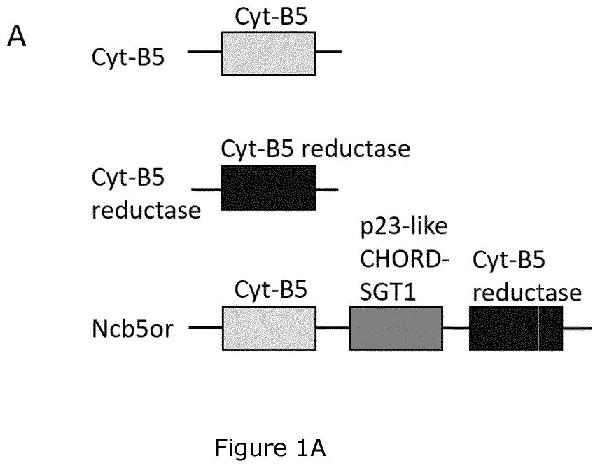

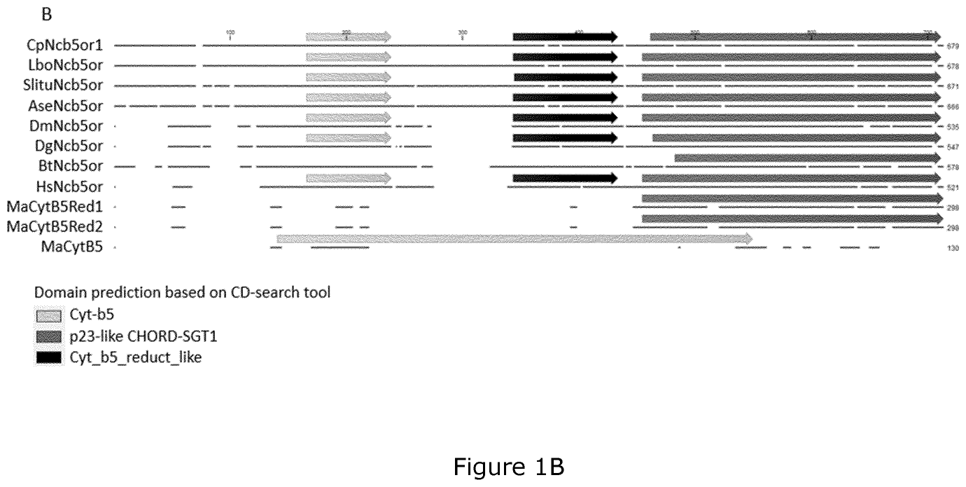

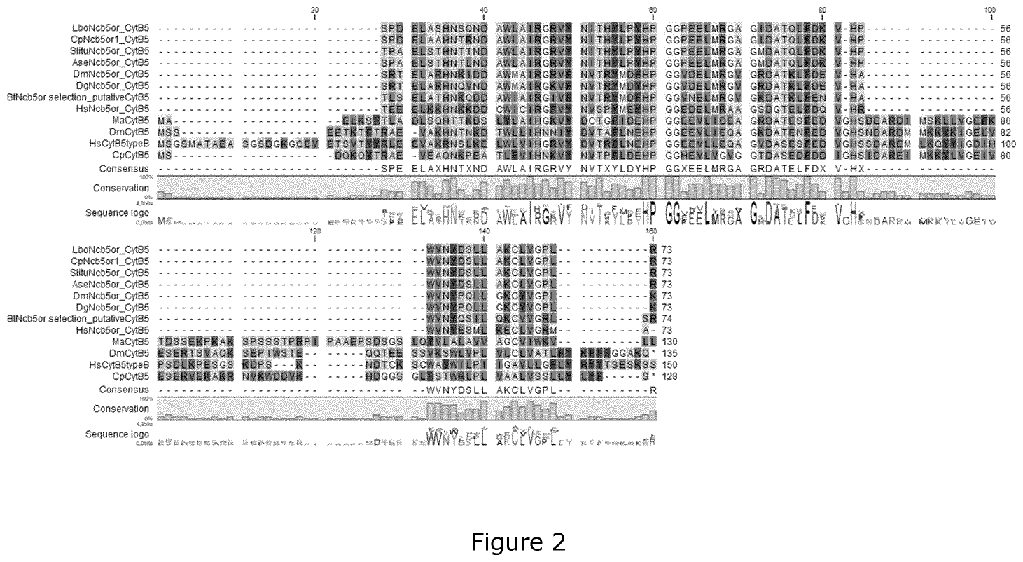

### DETAILED DESCRIPTION

### Definitions

Biopesticide: the term ‘biopesticide’ is a contraction of ‘biological pesticide’ and refers to several types of pest management intervention: through predatory, parasitic, or chemical relationships. In the EU, biopesticides have been defined as “a form of pesticide based on micro-organisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present invention relates more particularly to biopesticides comprising natural products or naturally occurring substances. They are typically created by growing and concentrating naturally occurring organisms and/or their metabolites including bacteria and other microbes, fungi, nematodes, proteins, etc. They are often considered to be important components of integrated pest management (IPM) programmes, and have received much practical attention as substitutes to synthetic chemical plant protection products (PPPs). The Manual of Biocontrol Agents (2009: formerly the Biopesticide Manual) gives a review of the available biological insecticide (and other biology-based control) products.

Cloud concentration: the term will herein be used to refer to the concentration of a surfactant, in particular non-ionic, or a glycol solution, in a solution above which, at a given temperature, a mixture of said surfactant and said solution starts to phase-separate, and two phases appear, thus becoming cloudy. For example, the cloud concentration of a surfactant in an aqueous solution at a given temperature is the minimal concentration of said surfactant which, when mixed with the aqueous solution, gives rise to two phases. The cloud concentration can be obtained from the manufacturer of the surfactant, or it may be determined experimentally, by making a dosage curve and determining the concentration at which the mixture phase separates.

Cloud point: The cloud point of a surfactant, in particular non-ionic, or a glycol solution, in a solution, for example an aqueous solution, is the temperature at which a mixture of said surfactant and said solution, for example said aqueous solution, starts to phase-separate, and two phases appear, thus becoming cloudy. This behavior is characteristic of non-ionic surfactants containing polyoxyethylene chains, which exhibit reverse solubility versus temperature behavior in water and therefore “cloud out” at some point as the temperature is raised. Glycols demonstrating this behavior are known as “cloud-point glycols”. The cloud point is affected by salinity, being generally lower in more saline fluids.

Desaturated: the term “desaturated” will be herein used interchangeably with the term “unsaturated” and refers to a compound containing one or more double or triple carbon-carbon bonds.

Derived from: the term when referring to a polypeptide or a polynucleotide derived from an organism means that said polypeptide or polynucleotide is native to said organism, i.e. that it is naturally found in said organism.

Ethoxylated and propoxylated C₁₆-C₁₈ alcohol-based antifoaming agent: the term refers to a group of polyethoxylated, non-ionic surfactants which comprise or mainly consist of ethoxylated and propoxylated alcohols in C₁₆-C₁₈, for example CAS number 68002-96-0, also termed C₁₆-C₁₈ alkyl alcohol ethoxylate propoxylate or C₁₆-C₁₈ alcohols ethoxylated propoxylated polymer.

Extractant: the term “extractant” as used herein refers to a non-ionic surfactant such as an antifoaming agent which facilitates recovery of hydrophobic compounds produced in a fermentation, in particular a polyethoxylated surfactant selected from: a polyethylene polypropylene glycol, a mixture of polyether dispersions, an antifoaming agent comprising polyethylene glycol monostearate such as simethicone and ethoxylated and propoxylated C₁₆-C₁₈ alcohol-based antifoaming agents and combinations thereof.

Fatty acid: the term “fatty acid” refers to a carboxylic acid having a long aliphatic chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Most naturally occurring fatty acids are unbranched. They can be saturated, or desaturated.

Fatty alcohol acetate: the term refers to an acetate having a fatty carbon chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty acyl acetates can be saturated or desaturated.

Fatty acyl-CoA: the term will herein be used interchangeably with “fatty acyl-CoA ester”, and refers to compounds of general formula R—CO—SCoA, where R is a fatty carbon chain having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. The fatty carbon chain is joined to the —SH group of coenzyme A by a thioester bond. Fatty acyl-CoAs can be saturated or desaturated, depending on whether the fatty acid which it is derived from is saturated or desaturated.

Fatty alcohol: the term “fatty alcohol” refers herein to an alcohol derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty alcohols can be saturated or desaturated.

Fatty aldehyde: the term refers herein to an aldehyde derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty aldehydes can be saturated or desaturated.

Functional variant: the term refers herein to functional variants of an enzyme, which retain at least some of the activity of the parent enzyme. Thus, a functional variant of an acyl-CoA oxidase, a desaturase, an alcohol-forming fatty acyl-CoA reductase, an alcohol dehydrogenase, an aldehyde-forming fatty acyl-CoA reductase, an acetyltransferase, or an NAD(P)H cytochrome b5 oxidoreductase (Ncb5or) can catalyse the same conversion as the acyl-CoA oxidase, the desaturase, the alcohol-forming fatty acyl-CoA reductase, the alcohol dehydrogenase, the aldehyde-forming fatty acyl-CoA reductase, or the acetyltransferase, respectively, from which they are derived, although the efficiency of reaction may be different, e.g. the efficiency is decreased or increased compared to the parent enzyme or the substrate specificity is modified.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous Δ9 desaturase” when applied to Saccharomyces cerevisiae refers to a Δ9 desaturase which is not naturally present in a wild type S. cerevisiae cell, e.g. a Δ9 desaturase derived from Drosophila melanogaster.

Identity/homology: the terms identity and homology, with respect to a polynucleotide (or polypeptide), are defined herein as the percentage of nucleic acids (or amino acids) in the candidate sequence that are identical or homologous, respectively, to the residues of a corresponding native nucleic acids (or amino acids), after aligning the sequences and introducing gaps, if necessary, to achieve the maximum percent identity/similarity/homology, and considering any conservative substitutions according to the NCIUB rules (http://www.chem.qmul.ac.uk/iubmb/misc/naseq.html; NC-IUB, Eur J Biochem (1985)) as part of the sequence identity. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given homology between two sequences implies that the identity between these sequences is at least equal to the homology; for example, if two sequences are 70% homologous to one another, they cannot be less than 70% identical to one another—but could be sharing 80% identity.

Increased activity: the term “increased activity” may herein refer to an increase in activity of a given peptide, such as a protein or an enzyme. The increase in activity can be measured using methods known in the art, such as for example using enzyme assays to measure the increase in activity of an enzyme. In some cases, the increase in activity results in higher production of the compound or compounds which the enzyme is generating, i.e. the product. Thus, increased activity of an enzyme may be measured by measuring the amount, such as the concentration, of said product. If an enzyme has increased activity, the concentration of product will be higher compared the concentration of product generated in similar or identical conditions by the same enzyme which does not have increased activity, e.g. the parent enzyme or unmodified enzyme. If the enzyme with increased activity is expressed inside a cell, the product can be measured as the product titer, i.e. the amount of product said cell has produced, and can be compared to the titer or amount of the same product obtained in similar or identical conditions from a cell expressing the parent or unmodified enzyme but otherwise having an identical or similar genotype as the cell expressing the enzyme with increased activity.

Native: the term “native” when referring to a polypeptide, such as a protein or an enzyme, or to a polynucleotide, shall herein be construed to refer to a polypeptide or a polynucleotide which is naturally present in a wild type cell.

Pest: as used herein, the term ‘pest’ shall refer to an organism, in particular an animal, detrimental to humans or human concerns, in particular in the context of agriculture or livestock production. A pest is any living organism which is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

Pheromone: pheromones are naturally occurring compounds. Lepidopteran pheromones are designated by an unbranched aliphatic chain (between 9 and 18 carbons, such as 9, 10, 11, 12, 13, 14, 15, 16, 17 or 18 carbon atoms) ending in an alcohol, aldehyde or acetate functional group and containing up to 3 double bonds in the aliphatic backbone. Thus, desaturated fatty alcohols, desaturated fatty aldehydes and desaturated fatty alcohol acetates are typically comprised in pheromones. Pheromone compositions may be produced chemically or biochemically, for example as described herein. Pheromones thus comprise desaturated fatty alcohols, desaturated fatty aldehydes and/or desaturated fatty alcohol acetates, such as can be obtained by the methods and cells described herein.

Purity: the term “purity” as used herein refers to the ratio or percentage of a compound in relation to all compounds within the same compound group produced by the cell. For example, the purity of a specific desaturated fatty alcohol is the percentage of said desaturated fatty alcohol in relation to all desaturated fatty alcohols produced by the cell; the purity of a fatty acid is the percentage of said fatty acid in relation to all fatty acids produced by the cell; and the purity of a desaturated fatty-acyl CoA is the percentage of said desaturated fatty acyl-CoA in relation to all fatty acyl-CoA produced by the cell.

Reduced activity: the term “reduced activity” may herein refer to a total or a partial loss of activity of a given peptide, such as a protein or an enzyme. In some cases, peptides are encoded by essential genes, which cannot be deleted. In these cases, activity of the peptide can be reduced by methods known in the art, such as down-regulation of transcription or translation, inhibition of the peptide. In other cases, the peptide is encoded by a non-essential gene, and the activity may be reduced or it may be completely lost, e.g. as a consequence of a deletion of the gene encoding the peptide. In order to reduce, whether partially or totally, the activity of a given peptide, methods known in the art include not only mutations in the genes encoding said peptide, but also mutation of genes encoding regulatory factors involved in transcription or translation of the gene encoding said peptide, e.g. mutation of transcription factor genes or of transcription repressor genes resulting in increased or decreased expression of said transcription factors or repressors, which in turn reduce transcription levels from the gene encoding the peptide; truncation or mutation of the native promoter of the gene, for example to remove transcription factor binding sites or to render them inaccessible to said transcription factors; replacement of the native promoter with a weaker promoter, leading to reduced transcription of the coding sequence encoding the peptide; truncation or mutation of the native terminator of the gene, or replacement of the native terminator of the gene with another terminator sequence; mutation of the Kozak sequence. Other methods involve regulation at the RNA level, and include RNA interference systems such as Dicer or Argonaute, RNA silencing methods, introduction of CRISPR/Cas systems resulting in targeted RNA degradation. Regulation at the protein level is also envisaged, e.g. by using inhibitors or protein degradation sequences. The listed methods may be inducible, i.e. they may be activated in a transient manner as known in the art.

Saturated: the term “saturated” refers to a compound which is devoid of double or triple carbon-carbon bonds.

Specificity: the specificity of an enzyme towards a given substrate is the preference exhibited by this enzyme to catalyse a reaction starting from said substrate. In the present disclosure, a desaturase and/or a fatty acyl-CoA reductase having a higher specificity towards tetradecanoyl-CoA (myristoyl-CoA) than towards hexadecanoyl-CoA (palmitoyl-CoA) preferably catalyse a reaction with tetradecanoyl-CoA than with hexadecanoyl-CoA as a substrate. Methods to determine the specificity of a desaturase or a fatty acyl-CoA reductase are known in the art. For example, specificity of a given desaturase in a given cell expressing it can be determined by incubating said cell in a solution comprising methyl myristate for up to 48 hours, followed by extraction and esterification of the products with methanol. The profiles of the resulting fatty acid methyl esters can then be determined by GC-MS. Desaturases with higher specificity towards myristoyl-CoA and low specificity towards palmitoyl-CoA, for example, will result in higher concentration of (Z)9-C14:Me than (Z)9-C16:Me. For example, specificity of a given reductase in a given cell can be determined by incubating cells that express said reductase in a solution comprising methyl ester of (Z)9-myristate for up to 48 hours, followed by extraction and analysis of the resulting fatty alcohols by GC-MS. Reductases with higher specificity towards (Z)9-C14:CoA and low specificity towards (Z)9-C16:CoA will result in higher concentration of (Z)9-C14:OH than (Z)9-C16:OH.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

The present inventors have discovered that NAD(P)H cytochrome b5 oxidoreductases (Ncb5ors) are enzymes which increase the activity of other enzymes, in particular membrane-bound enzymes localised in the cell membrane. Ncb5ors can thus, when expressed in cells engineered to produce compounds such as desaturated and saturated fatty alcohols, desaturated and saturated fatty aldehydes, and desaturated and saturated fatty alcohol acetates, the production of which relies on such membrane-bound enzymes, significantly improve production of these compounds. In other words, Ncb5ors significantly increase the activity of certain enzymes, such as of fatty acyl desaturases and of reductases such as fatty acyl-CoA reductases and cytochrome P450.

Herein are disclosed cells capable of producing compounds such as the ones listed above. Said cells express: a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous Ncb5or; whereby the cells are capable of producing the compound with a higher titer compared to a cell expressing the first group of enzymes but no heterologous Ncb5or when cultivated in the same conditions. Preferably, the first enzyme or group of enzymes are heterologous enzymes, i.e. are not naturally expressed in the cell.

In one embodiment, the first enzyme or group of enzymes may comprise or consist of one or more desaturase capable of converting a fatty acyl-CoA to a desaturated fatty acyl-CoA, whereby the cell is capable of producing a desaturated fatty acyl-CoA with a higher titer compared to a cell expressing said one or more desaturase but no heterologous Ncb5or when cultivated in the same conditions.

In another embodiment, the first enzyme or group of enzymes comprises or consists of one or more fatty acyl reductase (FAR) capable of converting a saturated or desaturated fatty acyl-CoA to a saturated or desaturated fatty alcohol, respectively, whereby the cell is capable of producing a saturated or desaturated fatty alcohol with a higher titer compared to a cell expressing said one or more FAR but no heterologous Ncb5or when cultivated in the same conditions.

In yet another embodiment, the first enzyme or group of enzymes comprises or consists of one or more fatty acyl reductase (FAR) and one or more desaturase capable of converting a fatty acyl-CoA to a desaturated fatty alcohol, whereby the cell is capable of producing a desaturated fatty alcohol with a higher titer compared to a cell expressing said one or more FAR and said one or more desaturase but no heterologous Ncb5or when cultivated in the same conditions.

The cell may further express an acetyltransferase, whereby said cell is capable of converting the desaturated or the saturated fatty alcohol to a desaturated or a saturated fatty alcohol acetate, respectively, whereby the cell is capable of producing a desaturated or a saturated fatty alcohol acetate with a higher titer compared to a cell expressing the first group of enzymes and the acetyltransferase but no heterologous Ncb5or when cultivated in the same conditions.

Preferably, the first enzyme or group of enzymes are native to insect species. In some embodiments, the first enzyme or group of enzymes are heterologous desaturases and reductases. Such cells produce desaturated fatty alcohols, saturated fatty alcohols, and desaturated fatty alcohol acetates, i.e. produce pheromone compounds with a higher titer compared to cells expressing the same heterologous desaturase and reductase but not expressing a heterologous Ncb5or.

The desaturase introduces at least one double bond in an acyl-CoA, which is then converted into the corresponding alcohol by the action of the reductase. This desaturated fatty alcohol can then further be converted into a desaturated fatty alcohol acetate and/or a desaturated fatty aldehyde, as detailed herein.

### Desaturase

In the present invention, the terms ‘fatty acyl-CoA desaturase’, ‘desaturase’, ‘fatty acyl desaturase’ and ‘FAD’ will be used interchangeably. The term generally refers to an enzyme capable of introducing at least one double bond in E/Z confirmations in an acyl-CoA having a chain length of 8, 9, 10, 11, 12, 13, 14, 15, 16, 17, 18, 19, 20, 21 or 22 carbon atoms. The double bond may be introduced in any position. For example, a desaturase introducing a double bond in position 9 is termed Δ9 desaturase.

### Desaturases Catalyse the Reaction:

Fatty acyl-CoA+2 ferrocytochrome b5+O(2)+2H(+)<=>desaturated fatty acyl-CoA+2 ferricytochrome b5+2H(2)O

The heterologous desaturase may be native to any type of organism. In some embodiments, the heterologous desaturase is native to a plant, such as such as Ricinus communis or Pelargonium hortorum. In another embodiment, the heterologous desaturase is native to an insect, such as of the Diptera, the Coleoptera, or the Lepidoptera order, such as of the genus Agrotis, Antheraea, Argyrotaenia, Amyelois, Bombus, Bombyx, Cadra, Chauliognathus, Chilo, Choristoneura, Cydia, Dendrophilus, Diatraea, Drosophila, Ephestia, Epiphyas, Grapholita, Helicoverpa, Lampronia, Lobesia, Manducta, Ostrinia, Pectinophora, Plodia, Plutella, Thalassiosira, Thaumetopoea, Tribolium, Trichoplusia, Spodoptera or Yponomeuta, such as Agrotis segetum, Antheraea pernyi, Argyrotaenia velutiana, Amyelois transitella, Bombus lapidarius, Bombyx mori, Cadra cautella, Chauliognathus lugubris, Chilo supprealis, Choristoneura parallela, Choristoneura rosaceana, Cydia pomonella, Dendrophilus punctatus, Diatraea saccharalis, Drosophila ananassae, Drosophila melanogaster, Drosophila virilis, Drosophila yakuba, Ephestia elutella, Ephestia kuehniella, Epiphyas postvittana, Grapholita molesta, Helicoverpa assulta, Helicoverpa zea, Lampronia capitella, Lobesia botrana, Manducta sexta, Ostrinia furnacalis, Ostrinia nubilalis, Pectinophora gossypiella, Plodia interpunctella, Plutella xylostella, Spodoptera exigua, Spodoptera littoralis, Spodoptera litura, Thalassiosira pseudonana, Thaumetopoea pityocampa, Tribolium castaneum, Trichoplusia ni, or Yponomeuta padella.

In some embodiments, the cell is capable of expressing a first enzyme or group of enzymes which comprises or consists of a desaturase. In one embodiment, the cell is capable of expressing at least one heterologous Δ5 desaturase. In another embodiment, the cell is capable of expressing at least one heterologous Δ6 desaturase. In another embodiment, the cell is capable of expressing at least one heterologous Δ7 desaturase. In another embodiment, the cell is capable of expressing at least one heterologous Δ8 desaturase. In another embodiment, the cell is capable of expressing at least one heterologous Δ9 desaturase. In another embodiment, the cell is capable of expressing at least one heterologous Δ10 desaturase. In another embodiment, the cell is capable of expressing at least one heterologous Δ11 desaturase. In another embodiment, the cell is capable of expressing at least one heterologous Δ12 desaturase. In another embodiment, the cell is capable of expressing at least one heterologous Δ13 desaturase. In another embodiment, the cell is capable of expressing at least one heterologous Δ14 desaturase. In another embodiment, the cell is capable of expressing at least one heterologous Δ15 desaturase. In another embodiment, the cell is capable of expressing at least one heterologous Δ16 desaturase. In another embodiment, the cell is capable of expressing at least one heterologous Δ17 desaturase. In another embodiment, the cell is capable of expressing at least one heterologous Δ18 desaturase. In another embodiment, the cell is capable of expressing at least one heterologous Δ19 desaturase. In another embodiment, the cell is capable of expressing at least one heterologous Δ20 desaturase. In another embodiment, the cell is capable of expressing at least one heterologous Δ21 desaturase. In preferred embodiments, the desaturase is a Δ9 desaturase or a Δ11 desaturase.

The gene encoding the heterologous desaturase may be codon-optimized for the cell, as is known in the art. Methods to determine whether the desaturase is expressed in the cell are known to the person of skill in the art, and include for instance detection of a given product from a given substrate, as detailed herein above and as illustrated in the examples below.

The skilled person will know, depending on which desaturated fatty alcohol is desired, which kind of desaturase to use. For example, for the production of a fatty alcohol desaturated in position 11, a Δ11 desaturase is preferably used. If a fatty alcohol desaturated in position 9 is desired, a Δ9 desaturase may be used, such as a Δ9 desaturase having at least 60% identity to a Drosophila desaturase such as a Drosophila Δ9 desaturase, for example the Δ9 desaturase from Drosophila melanogaster as set forth in SEQ ID NO: 14 or a Δ9 desaturase having at least 60% identity thereto, or to a Spodoptera desaturase such as a Spodoptera Δ9 desaturase, for example the Δ9 desaturase from Spodoptera litura as set forth in SEQ ID NO: 33 or a Δ9 desaturase having at least 60% identity thereto.

In some embodiments, the desaturase is a desaturase selected from the group of desaturases set forth in SEQ ID NOs: 1 to 38 and SEQ ID NOs: 126 to 139, or variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity a desaturase selected from the group of desaturases set forth in SEQ ID NOs: 1 to 38.

In one embodiment, the heterologous desaturase is an Agrotis desaturase. In one embodiment, the desaturase is an Agrotis segetum desaturase, such as the desaturase as set forth in SEQ ID NO: 1 (Desat19). In some embodiments, the desaturase is a variant of an Agrotis desaturase, a variant of an Agrotis segetum desaturase or a variant of the desaturase as set forth in SEQ ID NO: 1 (Desat19), having at least 60% identity thereto.

In one embodiment, the heterologous desaturase is an Amyelois desaturase. In one embodiment, the desaturase is an Amyelois transitella desaturase, such as the desaturase as set forth in SEQ ID NO: 3 (Desat17), as set forth in SEQ ID NO: 2 (Desat16) or as set forth in SEQ ID NO: 4 (Desat18). In some embodiments, the desaturase is a variant of an Amyelois desaturase, a variant of an Amyelois transitella desaturase or a variant of the desaturase as set forth in SEQ ID NO: 3 (Desat17), as set forth in SEQ ID NO: 2 (Desat16) or as set forth in SEQ ID NO: 4 (Desat18), having at least 60% identity thereto.

In one embodiment, the heterologous desaturase is an Antheraea desaturase. In one embodiment, the desaturase is an Antheraea pernyi desaturase, such as the desaturase as set forth in SEQ ID NO: 126 (Desat72). In some embodiments, the desaturase is a variant of an Antheraea desaturase, a variant of an Antheraea pernyi desaturase or a variant of the desaturase as set forth in SEQ ID NO: 126 (Desat72), having at least 60% identity thereto.

In one embodiment, the heterologous desaturase is an Argyrotaenia desaturase. In one embodiment, the desaturase is an Argyrotaenia velutinana desaturase, such as the desaturase as set forth in SEQ ID NO: 127 (Desat76). In some embodiments, the desaturase is a variant of an Argyrotaenia desaturase, a variant of an Argyrotaenia velutinana desaturase or a variant of the desaturase as set forth in SEQ ID NO: 127 (Desat76), having at least 60% identity thereto.

In one embodiment, the heterologous desaturase is a Bombus desaturase. In one embodiment, the desaturase is a Bombus lapidarius desaturase, such as the desaturase as set forth in SEQ ID NO: 128 (Desat75). In some embodiments, the desaturase is a variant of a Bombus desaturase, a variant of a Bombus lapidarius desaturase or a variant of the desaturase as set forth in SEQ ID NO: 128 (Desat75), having at least 60% identity thereto.

In one embodiment, the heterologous desaturase is a Bombyx desaturase. In one embodiment, the desaturase is a Bombyx mori desaturase, such as the desaturase as set forth in SEQ ID NO: 129 (Desat78). In some embodiments, the desaturase is a variant of a Bombyx mori desaturase, a variant of a Bombyx mori desaturase or a variant of the desaturase as set forth in SEQ ID NO: 129 (Desat78), having at least 60% identity thereto.

In one embodiment, the heterologous desaturase is a Cadra desaturase. In one embodiment, the desaturase is a Cadra cautella desaturase, such as the desaturase as set forth in SEQ ID NO: 134 (Desat70). In some embodiments, the desaturase is a variant of a Cadra desaturase, a variant of a Cadra cautella desaturase or a variant of the desaturase as set forth in SEQ ID NO: 134 (Desat70), having at least 60% identity thereto.

In one embodiment, the heterologous desaturase is a Chauliognathus desaturase. In one embodiment, the desaturase is a Chauliognathus lugubris desaturase, such as the desaturase as set forth in SEQ ID NO: 5 (Desat25). In some embodiments, the desaturase is a variant of a Chauliognathus desaturase, a variant of a Chauliognathus lugubris desaturase or a variant of the desaturase as set forth in SEQ ID NO: 5 (Desat25), having at least 60% identity thereto.

In one embodiment, the heterologous desaturase is a Chilo desaturase. In one embodiment, the desaturase is a Chilo supprealis desaturase, such as the desaturase as set forth in SEQ ID NO: 6 (Desat47) or SEQ ID NO: 130 (Desat44). In some embodiments, the desaturase is a variant of a Chilo desaturase, a variant of a Chilo supprealis desaturase or a variant of the desaturase as set forth in SEQ ID NO: 6 (Desat47) or SEQ ID NO: 130 (Desat44), having at least 60% identity thereto.

In one embodiment, the heterologous desaturase is a Choristoneura desaturase. In one embodiment, the desaturase is a Choristoneura parallela desaturase, such as the desaturase as set forth in SEQ ID NO: 7 (Desat36), or a Choristoneura rosaceana desaturase, such as the desaturase as set forth in SEQ ID NO: 8 (Desat35). In some embodiments, the desaturase is a variant of a Choristoneura desaturase, a variant of a Choristoneura parallela desaturase, a Choristoneura rosaceana desaturase or a variant of the desaturase as set forth in SEQ ID NO: 7 (Desat36), of the desaturase as set forth in SEQ ID NO: 8 (Desat35), having at least 60% identity thereto.

In one embodiment, the heterologous desaturase is a Cydia desaturase. In one embodiment, the desaturase is a Cydia pomonella desaturase, such as the desaturase as set forth in SEQ ID NO: 9 (Desat4), the desaturase as set forth in SEQ ID NO: 10 (Desat2) or the desaturase as set forth in SEQ ID NO: 11 (Desat1). In some embodiments, the desaturase is a variant of a Cydia desaturase, a variant of a Cydia pomonella desaturase or a variant of the desaturase as set forth in SEQ ID NO: 9 (Desat4), the desaturase as set forth in SEQ ID NO: 10 (Desat2) or the desaturase as set forth in SEQ ID NO: 11 (Desat1), having at least 60% identity thereto.

In one embodiment, the heterologous desaturase is a Dendrolimus desaturase. In one embodiment, the desaturase is a Dendrolimus punctatus desaturase, such as the desaturase as set forth in SEQ ID NO: 12 (Desat40). In some embodiments, the desaturase is a variant of a Dendrolimus desaturase, a variant of a Dendrolimus punctatus desaturase or a variant of the desaturase as set forth in SEQ ID NO: 12 (Desat40), having at least 60% identity thereto.

In one embodiment, the heterologous desaturase is a Diatraea Z11 desaturase. In one embodiment, the desaturase is a Diatraea saccharalis Z11 desaturase, such as the Z11 desaturase as set forth in SEQ ID NO: 132 (Desat63). In some embodiments, the desaturase is a variant of a Diatraea Z11 desaturase, a variant of a Diatraea saccharalis Z11 desaturase or a variant of the Z11 desaturase as set forth in SEQ ID NO: 132 (Desat63), having at least 60% identity thereto.

In one embodiment, the heterologous desaturase is a Drosophila desaturase. In one embodiment, the desaturase is a Drosophila virilis desaturase, for example such as Desat61 as set forth in SEQ ID NO: 15. In one embodiment, the desaturase is a Drosophila ananassae desaturase, for example such as the desaturase set forth in SEQ ID NO: 131 (Desat60). In one embodiment, the desaturase is a Drosophila melanogaster desaturase, for example such as the desaturase set forth in SEQ ID NO: 14 (Desat24). In one embodiment, the desaturase is a Drosophila grimshawi desaturase, for example such as the desaturase set forth in SEQ ID NO: 13 (Desat59). In one embodiment, the desaturase is a Drosophila yakuba desaturase, for example such as the desaturase set forth in SEQ ID NO: 133 (Desat56). In some embodiments, the desaturase is a variant of a Drosophila desaturase, a variant of a Drosophila anannasae desaturase, a variant of a Drosophila virilis desaturase, a variant of a Drosophila melanogaster desaturase, a variant of a Drosophila grimshawi desaturase, a variant of a Drosophila yakuba desaturase, such as a variant of the desaturase as set forth in SEQ ID NO: 131 (Desat60), of the desaturase as set forth in SEQ ID NO: 14 (Desat24), of the desaturase as set forth in SEQ ID NO: 15 (Desat61) or of the desaturase as set forth in SEQ ID NO: 13 (Desat59), of the desaturase as set forth in SEQ ID NO: 133 (Desat56), having at least 60% identity thereto.

In one embodiment, the heterologous desaturase is an Epiphyas desaturase. In one embodiment, the desaturase is an Epiphyas postvittana desaturase, such as the desaturase as set forth in SEQ ID NO: 16 (Desat33). In some embodiments, the desaturase is a variant of an Epiphyas desaturase, a variant of an Epiphyas postvittana desaturase or a variant of the desaturase as set forth in SEQ ID NO: 16 (Desat33), having at least 60% identity thereto.

In one embodiment, the heterologous desaturase is a Grapholita desaturase. In one embodiment, the desaturase is a Grapholita molesta desaturase, such as the desaturase as set forth in SEQ ID NO: 17 (Desat31) or as set forth in SEQ ID NO: 18 (Desat55). In some embodiments, the desaturase is a variant of a Grapholita desaturase, a variant of a Grapholita molesta desaturase or a variant of the desaturase as set forth in SEQ ID NO: 17 (Desat31) or as set forth in SEQ ID NO: 18 (Desat55), having at least 60% identity thereto.

In one embodiment, the heterologous desaturase is a Helicoverpa desaturase. In one embodiment, the desaturase is a Helicoverpa zea desaturase, such as Desat51 as set forth in SEQ ID NO: 19. In some embodiments, the desaturase is a variant of a Helicoverpa desaturase, a variant of a Helicoverpa zea desaturase or a variant of Desat51 as set forth in SEQ ID NO: 19, having at least 60% identity thereto.

In one embodiment, the heterologous desaturase is a Lobesia desaturase. In one embodiment, the desaturase is a Lobesia botrana desaturase, such as Desat30 (SEQ ID NO: 20), Desat71 (SEQ ID NO: 135) or Desat43 (SEQ ID NO: 21). In some embodiments, the desaturase is a variant of a Lobesia desaturase, a variant of a Lobesia botrana desaturase or a variant of the desaturase as set forth in SEQ ID NO: 20 (Desat30), as set forth in SEQ ID NO: 135 (Desat71) or as set forth in SEQ ID NO: 21 (Desat43), having at least 60% identity.

In one embodiment, the heterologous desaturase is a Manducta desaturase. In one embodiment, the desaturase is a Manducta sexta desaturase, such as the desaturase as set forth in SEQ ID NO: 22 (Desat52). In some embodiments, the desaturase is a variant of an Manducta desaturase, a variant of an Manducta sexta desaturase or a variant of the desaturase as set forth in SEQ ID NO: 22 (Desat52), having at least 60% identity thereto.

In one embodiment, the heterologous desaturase is an Ostrinia desaturase. In one embodiment, the desaturase is an Ostrinia nubilalis desaturase, such as the desaturase as set forth in SEQ ID NO: 23 (Desat32). In one embodiment, the desaturase is an Ostrinia furnacalis desaturase, such as the desaturase as set forth in SEQ ID NO: 136 (Desat77). In some embodiments, the desaturase is a variant of an Ostrinia desaturase, a variant of an Ostrinia nubilalis desaturase, a variant of an Ostrinia furnacalis desaturase, or a variant of the desaturase as set forth in SEQ ID NO: 23 (Desat32) or as set forth in SEQ ID NO: 136 (Desat77), having at least 60% identity thereto.

In one embodiment, the heterologous desaturase is a Pectinophora desaturase. In one embodiment, the desaturase is a Pectinophora gossypiella desaturase, such as the desaturase as set forth in SEQ ID NO: 24 (Desat48). In some embodiments, the desaturase is a variant of a Pectinophora desaturase, a variant of a Pectinophora gossypiella desaturase or a variant of the desaturase as set forth in SEQ ID NO: 24 (Desat48), having at least 60% identity thereto.

In one embodiment, the heterologous desaturase is a Pelargonium desaturase. In one embodiment, the desaturase is a Pelargonium hortorum desaturase, such as the desaturase as set forth in SEQ ID NO: 25 (Desat22). In some embodiments, the desaturase is a variant of a Pelargonium desaturase, a variant of a Pelargonium hortorum desaturase or a variant of the desaturase as set forth in SEQ ID NO: 25 (Desat22) having at least 60% identity thereto.

In one embodiment, the heterologous desaturase is a Plodia desaturase. In one embodiment, the desaturase is a Plodia interpunctella desaturase, such as the desaturase as set forth in SEQ ID NO: 137 (Desat65). In some embodiments, the desaturase is a variant of a Plodia desaturase, a variant of a Plodia interpunctella desaturase or a variant of the desaturase as set forth in SEQ ID NO: 137 (Desat65) having at least 60% identity thereto.

In one embodiment, the heterologous desaturase is a Plutella desaturase. In one embodiment, the desaturase is a Plutella xylostella desaturase, such as the desaturase as set forth in SEQ ID NO: 26 (Desat45). In some embodiments, the desaturase is a variant of a Plutella desaturase, a variant of a Plutella xylostella desaturase or a variant of the desaturase as set forth in SEQ ID NO: 26 (Desat45) having at least 60% identity thereto.

In one embodiment, the heterologous desaturase is a Ricinus desaturase. In one embodiment, the desaturase is a Ricinus communis desaturase, such as Desat23 as set forth in SEQ ID NO: 27. In some embodiments, the desaturase is a variant of a Ricinus desaturase, a variant of a Ricinus communis desaturase or a variant of Desat23 as set forth in SEQ ID NO: 27, having at least 60% identity thereto.

In one embodiment, the heterologous desaturase is a Saccharomyces desaturase. In one embodiment, the desaturase is a Saccharomyces cerevisiae desaturase, such as the desaturase as set forth in SEQ ID NO: 28 (Desat42). In some embodiments, the desaturase is a variant of a Saccharomyces desaturase, a variant of a Saccharomyces cerevisiae desaturase or a variant of the desaturase as set forth in SEQ ID NO: 28 (Desat42), having at least 60% identity thereto.

In one embodiment, the heterologous desaturase is a Spodoptera desaturase. In one embodiment, the desaturase is a Spodoptera littoralis desaturase, such as the desaturase as set forth in SEQ ID NO: 31 (Desat20), or a Spodoptera litura desaturase, such as the desaturase as set forth in SEQ ID NO: 32 (Desat38) or as set forth in SEQ ID NO: 33 (Desat26), or a Spodoptera exigua desaturase, such as the desaturase as set forth in SEQ ID NO: 29 (Desat37). In some embodiments, the desaturase is a variant of a Spodoptera desaturase, a variant of a Spodoptera littoralis desaturase, a variant of a Spodoptera litura desaturase, a variant of a Spodoptera exigua desaturase or a variant of the desaturase as set forth in SEQ ID NO: 31 (Desat20), as set forth in SEQ ID NO: 32 (Desat38), as set forth in SEQ ID NO: 33 (Desat26), or as set forth in SEQ ID NO: 29 (Desat37), having at least 60% identity thereto.

In one embodiment, the heterologous desaturase is a Thaumetopoea desaturase. In one embodiment, the desaturase is a Thaumetopoea pityocampa desaturase, such as the desaturase as set forth in SEQ ID NO: 34 (Desat34). In some embodiments, the desaturase is a variant of a Thaumetopoea desaturase, a variant of a Thaumetopoea pityocampa desaturase or a variant of the desaturase as set forth in SEQ ID NO: 34 (Desat34), having at least 60% identity thereto.

In one embodiment, the heterologous desaturase is a Tribolium desaturase. In one embodiment, the desaturase is a Tribolium castaneum desaturase, such as the desaturase as set forth in SEQ ID NO: 35 (Desat28), as set forth in SEQ ID NO: 138 (Desat27), or as set forth in SEQ ID NO: 36 (Desat29). In some embodiments, the desaturase is a variant of a Tribolium desaturase, a variant of a Tribolium castaneum desaturase or a variant of the desaturase as set forth in SEQ ID NO: 35 (Desat28), as set forth in SEQ ID NO: 138 (Desat27) or as set forth in SEQ ID NO: 36 (Desat29), having at least 60% identity thereto.

In one embodiment, the heterologous desaturase is a Trichoplusia desaturase. In one embodiment, the desaturase is a Trichoplusia ni desaturase, such as the desaturase as set forth in SEQ ID NO: 37 (Desat21). In some embodiments, the desaturase is a variant of a Trichoplusia desaturase, a variant of a Trichoplusia ni desaturase or a variant of the desaturase as set forth in SEQ ID NO: 37 (Desat21), having at least 60% identity thereto.

In one embodiment, the heterologous desaturase is a Yarrowia desaturase. In one embodiment, the desaturase is a Yarrowia lipolytica desaturase, such as the desaturase as set forth in SEQ ID NO: 38 (Desat69). In some embodiments, the desaturase is a variant of a Yarrowia desaturase, a variant of a Yarrowia lipolytica desaturase or a variant of the desaturase as set forth in SEQ ID NO: 38 (Desat69), having at least 60% identity thereto.

In one embodiment, the heterologous desaturase is an Yponomeuta desaturase. In one embodiment, the desaturase is an Yponomeuta padella desaturase, such as the desaturase as set forth in SEQ ID NO: 139 (Desat73). In some embodiments, the desaturase is a variant of an Yponomeuta desaturase, a variant of an Yponomeuta padella desaturase or a variant of the desaturase as set forth in SEQ ID NO: 139 (Desat73), having at least 60% identity thereto.

It will be understood that a variant desaturase having at least 60% identity to a given desaturase as above may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the desaturase, or more.

### Nucleic Acids Encoding Desaturases

In some embodiments, the heterologous desaturase is encoded by a nucleic acid having at least 60% identity to a nucleic acid selected from the group of desaturases set forth in SEQ ID NOs: 39 to 76 and SEQ ID NOs: 140 to 153, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous desaturase is encoded by a nucleic acid having at least 60% identity to the nucleic acid encoding an Agrotis segetum desaturase, as set forth in SEQ ID NO: 39. In one embodiment, the heterologous desaturase is encoded by a nucleic acid having at least 60% identity to the nucleic acid encoding an Amyelois transitella desaturase, as set forth in SEQ ID NO: 40, SEQ ID NO: 41 or SEQ ID NO: 42. In one embodiment, the heterologous desaturase is encoded by a nucleic acid having at least 60% identity to the nucleic acid encoding an Antheraea pernyi desaturase, as set forth in SEQ ID NO: 140. In one embodiment, the heterologous desaturase is encoded by a nucleic acid having at least 60% identity to the nucleic acid encoding an Argyrotaenia velutinana desaturase, as set forth in SEQ ID NO: 141. In one embodiment, the heterologous desaturase is encoded by a nucleic acid having at least 60% identity to the nucleic acid encoding a Bombus lapidarius desaturase, as set forth in SEQ ID NO: 142. In one embodiment, the heterologous desaturase is encoded by a nucleic acid having at least 60% identity to the nucleic acid encoding a Bombyx mori desaturase, as set forth in SEQ ID NO: 143. In one embodiment, the heterologous desaturase is encoded by a nucleic acid having at least 60% identity to the nucleic acid encoding a Cadra cautella desaturase, as set forth in SEQ ID NO: 148. In one embodiment, the heterologous desaturase is encoded by a nucleic acid having at least 60% identity to the nucleic acid encoding a Chauliognathus lugubris desaturase, as set forth in SEQ ID NO: 43. In one embodiment, the heterologous desaturase is encoded by a nucleic acid having at least 60% identity to the nucleic acid encoding a Chilo supprealis desaturase, as set forth in SEQ ID NO: 44 or SEQ ID NO: 144. In one embodiment, the heterologous desaturase is encoded by a nucleic acid having at least 60% identity to the nucleic acid encoding a Choristoneura parallela desaturase, as set forth in SEQ ID NO: 45. In one embodiment, the heterologous desaturase is encoded by a nucleic acid having at least 60% identity to the nucleic acid encoding a Choristoneura rosaceana desaturase, as set forth in SEQ ID NO: 46. In one embodiment, the heterologous desaturase is encoded by a nucleic acid having at least 60% identity to the nucleic acid encoding a Cydia pomonella desaturase, as set forth in SEQ ID NO: 47, in SEQ ID NO: 48 or in SEQ ID NO: 49. In one embodiment, the heterologous desaturase is encoded by a nucleic acid having at least 60% identity to the nucleic acid encoding a Dendrolimus punctatus desaturase, as set forth in SEQ ID NO: 50. In one embodiment, the heterologous desaturase is encoded by a nucleic acid having at least 60% identity to the nucleic acid encoding a Diatraea saccharalis desaturase, as set forth in SEQ ID NO: 146. In one embodiment, the heterologous desaturase is encoded by a nucleic acid having at least 60% identity to the nucleic acid encoding a Drosophila virilis desaturase, as set forth in SEQ ID NO: 53. In one embodiment, the heterologous desaturase is encoded by a nucleic acid having at least 60% identity to the nucleic acid encoding a Drosophila ananassae desaturase, as set forth in SEQ ID NO: 145. In one embodiment, the heterologous desaturase is encoded by a nucleic acid having at least 60% identity to the nucleic acid encoding a Drosophila melanogaster desaturase, as set forth in SEQ ID NO: 52. In one embodiment, the heterologous desaturase is encoded by a nucleic acid having at least 60% identity to the nucleic acid encoding a Drosophila yakuba desaturase, as set forth in SEQ ID NO: 147. In one embodiment, the heterologous desaturase is encoded by a nucleic acid having at least 60% identity to the nucleic acid encoding a Drosophila grimshawi desaturase, as set forth in SEQ ID NO: 51. In one embodiment, the heterologous desaturase is encoded by a nucleic acid having at least 60% identity to the nucleic acid encoding an Epiphyas postvittana desaturase, as set forth in SEQ ID NO: 54. In one embodiment, the heterologous desaturase is encoded by a nucleic acid having at least 60% identity to the nucleic acid encoding a Grapholita molesta desaturase, as set forth in SEQ ID NO: 55 or SEQ ID NO: 56. In one embodiment, the heterologous desaturase is encoded by a nucleic acid having at least 60% identity to the nucleic acid encoding a Helicoverpa zea desaturase, as set forth in SEQ ID NO: 57. In one embodiment, the heterologous desaturase is encoded by a nucleic acid having at least 60% identity to the nucleic acid encoding a Lobesia botrana desaturase, as set forth in SEQ ID NO: 58, in SEQ ID NO: 149 or in SEQ ID NO: 59. In one embodiment, the heterologous desaturase is encoded by a nucleic acid having at least 60% identity to the nucleic acid encoding a Manducta sexta desaturase, as set forth in SEQ ID NO: 60. In one embodiment, the heterologous desaturase is encoded by a nucleic acid having at least 60% identity to the nucleic acid encoding an Ostrinia nubilalis desaturase, as set forth in SEQ ID NO: 61. In one embodiment, the heterologous desaturase is encoded by a nucleic acid having at least 60% identity to the nucleic acid encoding an Ostrinia furnacalis desaturase, as set forth in SEQ ID NO: 150. In one embodiment, the heterologous desaturase is encoded by a nucleic acid having at least 60% identity to the nucleic acid encoding a Pectinophora gossypiella desaturase, as set forth in SEQ ID NO: 62. In one embodiment, the heterologous desaturase is encoded by a nucleic acid having at least 60% identity to the nucleic acid encoding a Pelargonium hortorum desaturase, as set forth in SEQ ID NO: 63. In one embodiment, the heterologous desaturase is encoded by a nucleic acid having at least 60% identity to the nucleic acid encoding a Plodia interpunctella desaturase, as set forth in SEQ ID NO: 151. In one embodiment, the heterologous desaturase is encoded by a nucleic acid having at least 60% identity to the nucleic acid encoding a Plutella xylostella desaturase, as set forth in SEQ ID NO: 64. In one embodiment, the heterologous desaturase is encoded by a nucleic acid having at least 60% identity to the nucleic acid encoding a Ricinus communis desaturase, as set forth in SEQ ID NO: 65. In one embodiment, the heterologous desaturase is encoded by a nucleic acid having at least 60% identity to the nucleic acid encoding a Saccharomyces cerevisiae desaturase, as set forth in SEQ ID NO: 66. In one embodiment, the heterologous desaturase is encoded by a nucleic acid having at least 60% identity to the nucleic acid encoding a Spodoptera exigua desaturase, as set forth in SEQ ID NO: 67. In one embodiment, the heterologous desaturase is encoded by a nucleic acid having at least 60% identity to the nucleic acid encoding a Spodoptera littoralis desaturase, as set forth in SEQ ID NO: 68, or in SEQ ID NO: 69. In one embodiment, the heterologous desaturase is encoded by a nucleic acid having at least 60% identity to the nucleic acid encoding a Spodoptera litura desaturase, as set forth in SEQ ID NO: 70 or in SEQ ID NO: 71. In one embodiment, the heterologous desaturase is encoded by a nucleic acid having at least 60% identity to the nucleic acid encoding a Thaumetopoea pityocampa desaturase, as set forth in SEQ ID NO: 72. In one embodiment, the heterologous desaturase is encoded by a nucleic acid having at least 60% identity to the nucleic acid encoding a Tribolium castaneum desaturase, as set forth in SEQ ID NO: 73, in SEQ ID NO: 152 or in SEQ ID NO: 74. In one embodiment, the heterologous desaturase is encoded by a nucleic acid having at least 60% identity to the nucleic acid encoding a Trichoplusia ni desaturase, as set forth in SEQ ID NO: 75. In one embodiment, the heterologous desaturase is encoded by a nucleic acid having at least 60% identity to the nucleic acid encoding a Yarrowia lipolytica desaturase, as set forth in SEQ ID NO: 76. In one embodiment, the heterologous desaturase is encoded by a nucleic acid having at least 60% identity to the nucleic acid encoding a Yponomeuta padella desaturase, as set forth in SEQ ID NO: 76. Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to the given nucleic acid, or more.

### Co-Expression of a Plurality of Heterologous Desaturases

The present cells may express at least one heterologous desaturase. In some embodiments, the cell expresses one heterologous desaturase. It may however be desirable to express several heterologous desaturases, such as at least two heterologous desaturases, which may be identical or different. Alternatively, it may be desirable to express several copies of the nucleic acid encoding the at least one heterologous desaturases, such as at least two copies, at least three copies or more. In other embodiments, the cell expresses at least two heterologous desaturases, for example three heterologous desaturases.

The cell to be modified may express a native desaturase, which may have a negative impact on the production of desaturated fatty alcohol and/or desaturated fatty alcohol acetate. Accordingly, if the cell to be modified expresses such a native desaturase, the organism may preferably be modified so that activity of the native desaturase is reduced or absent.

To ensure lack of activity of a native desaturase, methods known in the art can be employed. The gene encoding the native desaturase may be deleted or partly deleted in order to ensure that the native desaturase is not expressed. Alternatively, the gene may be mutated so that the native desaturase is expressed but lacks activity, e.g. by mutation of the catalytic site of the enzyme. Alternatively, translation of mRNA to an active protein may be prevented by methods such as silencing RNA or siRNA.

Alternatively, the cell may be incubated in a medium comprising an inhibitor which inhibits activity of the native desaturase. A compound inhibiting transcription of the gene encoding the native desaturase may also be provided so that transcription is inactivated when said compound is present. Other methods are known in the art may be employed.

Inactivation of the native desaturase may thus be permanent or long-term, i.e. the modified cell exhibits reduced or no activity of the native desaturase in a stable manner, or it may be transient, i.e. the modified cell may exhibit activity of the native desaturase for periods of time, but this activity can be suppressed for other periods of time.

### Increased C14 Specificity

Many desirable pheromone compounds have a carbon chain length of 14. It may thus be of interest to direct the reaction towards the production of C14 compounds. In some embodiments, the cell disclosed herein expresses a desaturase having a higher specificity towards tetradecanoyl-CoA than towards hexadecanoyl-CoA and/or an acyl-CoA reductase having a higher specificity towards desaturated tetradecanoyl-CoA than towards desaturated hexadecanoyl-CoA. In other words, the desaturase is more specific for substrates having a carbon chain length of 14 than for substrates having a chain length of 16. Examples of yeast cells expressing such desaturases are disclosed in WO 2018/109167.

Expression of such desaturases (and of any of the reductases described herein below) in the cell increases the fraction of total desaturated fatty alcohols having a carbon chain length of 14, particularly compared to the fraction of total desaturated fatty alcohols having a carbon chain length of 16. Desaturases which have the required specificity are in particular desaturases native to Drosophila, Spodoptera, Choristneura species, such as desaturases native to Drosophila melanogaster, Drosophila grimshawi, Drosophila virilis, Spodoptera litura, Choristoneura parallela or Choristoneura rosaceana, for example as set forth in SEQ ID NOs: 1 to 38, or variants thereof having at least 60% identity thereto.

In some embodiments, the desaturase is selected from the groups consisting of:
i) a Δ9 desaturase having at least 60% identity to the Δ9 desaturase from Drosophila melanogaster as set forth in SEQ ID NO: 14;
ii) a desaturase having at least 60% identity to the Δ9 desaturase from Drosophila grimshawi as set forth in SEQ ID NO: 13;
iii) a desaturase having at least 60% identity to the Δ9 desaturase from Drosophila virilis as set forth in SEQ ID NO: 15;
iv) a Δ9 desaturase having at least 60% identity to the Δ9 desaturase from Spodoptera litura as set forth in SEQ ID NO: 33;
v) a Δ11 desaturase having at least 60% identity to the Δ11 desaturase from Choristoneura parallela as set forth in SEQ ID NO: 7;
vi) a Δ11 desaturase having at least 60% identity to the Δ11 desaturase from Choristoneura rosaceana as set forth in SEQ ID NO: 8;
   and functional variants thereof having at least 60% identity thereto, 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto.

These desaturases have been found to preferentially catalyse desaturation of C14 substrates when expressed in a yeast cell.

In such cells, the ratio of desaturated tetradecanoyl-CoA to desaturated hexadecanoyl-CoA is of at least 0.1, such as at least 0.2, such as at least 0.3, such as at least 0.4, such as at least 0.5, such as at least 0.75, such as at least 1, such as at least 2, such as at least 3, such as at least 4, such as at least 5, such as at least 6, such as at least 7, such as at least 8, such as at least 9, such as at least 10, such as at least 12.5, such as at least 15, or more.

In some embodiments, the titre of desaturated fatty alcohols is of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, the titre of desaturated fatty alcohol having a chain length of 14 is of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, or more.

In some embodiments, desaturated fatty alcohols are yielded comprising at least 1% of a desaturated fatty alcohol having a chain length of 14, such as at least 1.5%, such as at least 2%, such as at least 2.5%, such as at least 3%, such as at least 3.5%, such as at least 4%, such as at least 4.5%, such as at least 5%, such as at least 7.5%, such as at least 10%, or more.

How to test whether a given desaturase has the required specificity can be done as described herein.

### Fatty Acyl-CoA Reductase

The terms ‘fatty acyl-CoA reductase’ and ‘FAR’ will be used herein interchangeably.

The term ‘heterologous FAR’ refers to a FAR which is not naturally expressed by the organism, such as by the cell.

FARs catalyze the two-step reaction:

$$Acyl - CoA + 2 NADPH \text{<=>} CoA + Alcohol + 2 NADP ( + )$$

wherein in a first step, the fatty acyl-CoA is reduced to a fatty aldehyde, before the fatty aldehyde is further reduced into a fatty alcohol in a second step. The fatty acyl-CoA may be a desaturated or a saturated fatty acyl-CoA.

The FARs capable of catalyzing such reaction are alcohol-forming fatty acyl-CoA reductases with an EC number 1.2.1.84.

Ncb5ors can increase the activity of FARs. In some embodiments, the first enzyme or group of enzymes comprises or consists of one or more FARs.

The FAR may be heterologous to the cell disclosed herein. In some embodiments, the FAR is preferably native to an insect such as an insect of the Lepidoptera order, such as of the genus Lepidoptera order, such as of the genus Agrotis, Amyelois, Bicyclus, Bombus, Chilo, Chrysodeixis, Cydia, Helicoverpa, Heliothis, Manducta, Ostrinia, Plodia, Plutella, Spodoptera, Trichoplusia, Tyta or Yponomeuta, such as Agrotis segetum, Amyelois transitella, Bicyclus anynana, Bombus lapidaries, Chilo suppressalis, Chrysodeixis includes, Cydia pomonella, Helicoverpa armigera, Helicoverpa assulta, Heliothis virescens, Heliothis subflexa, Manducta sexta, Ostrinia furnacalis, Plodia interpunctella, Plutella xylostella, Spodoptera exigua, Spodoptera frugiperda, Spodoptera littoralis, Spodoptera litura, Trichoplusia ni, Tyta alba, Yponomeuta rorellus, or a functional variant having at least 60% identity thereto. In some embodiments, the FAR is native to a bacteria, such as of the genus Marinobacter, such as Marinobacter algicola.

In one embodiment, the heterologous FAR is an Agrotis FAR. In one embodiment, the FAR is an Agrotis segetum FAR, such as the FAR as set fort in SEQ ID NO: 77 (FAR12). In some embodiments, the FAR is an Agrotis ipsilon FAR, such as the FAR as set fort in SEQ ID NO: 78 (FAR18). In some embodiments, the FAR is a variant of an Agrotis FAR, a variant of an Agrotis segetum FAR, such as the FAR as set forth in SEQ ID NO: 77 (FAR12), a variant of an Agrotis ipsilon FAR, such as the FAR as set forth in SEQ ID NO: 78 (FAR18) or a variant thereof, having at least 60% identity thereto.

In one embodiment, the heterologous FAR is an Amyelois FAR. In one embodiment, the FAR is an Amyelois transitella FAR, such as the FAR as set fort in SEQ ID NO: 154 (FAR33), SEQ ID NO: 155 (FAR34) or SEQ ID NO: 156 (FAR35). In some embodiments, the FAR is a variant of an Amyelois FAR, a variant of an Amyelois transitella FAR, such as the FAR as set forth in SEQ ID NO: 77 (FAR12), a variant of an Amyelois transitella FAR, such as the FAR as set forth in SEQ ID NO: 154 (FAR33), SEQ ID NO: 155 (FAR34) or SEQ ID NO: 156 (FAR35) or a variant thereof, having at least 60% identity thereto.

In one embodiment, the heterologous FAR is a Bicyclus FAR. In one embodiment, the FAR is a Bicyclus anynana FAR, such as the FAR as set fort in SEQ ID NO: 79 (FAR11). In some embodiments, the FAR is a variant of a Bicyclus FAR, a variant of a Bicyclus anyana FAR or a variant of the FAR set forth in SEQ ID NO: 79 (FAR11), having at least 60% identity thereto.

In one embodiment, the heterologous FAR is a Bombus FAR. In one embodiment, the FAR is a Bombus lapidarius FAR, such as the FAR as set fort in SEQ ID NO: 80 (FAR14). In some embodiments, the FAR is a variant of a Bombus FAR, a variant of a Bombus lapidarius FAR or a variant of the FAR set forth in SEQ ID NO: 80 (FAR14), having at least 60% identity thereto.

In one embodiment, the heterologous FAR is a Chilo FAR. In one embodiment, the FAR is a Chilo suppressalis FAR, such as the FAR as set fort in SEQ ID NO: 81 (FAR13). In some embodiments, the FAR is a variant of a Chilo FAR, a variant of a Chilo suppressalis FAR or a variant of the FAR set forth in SEQ ID NO: 81 (FAR13), having at least 60% identity thereto.

In one embodiment, the heterologous FAR is a Chrysodeixis FAR. In one embodiment, the FAR is a Chrysodeixis includens FAR, such as the FAR as set fort in SEQ ID NO: 157 (FAR47). In some embodiments, the FAR is a variant of a Chrysodeixis FAR, a variant of a Chrysodeixis includens FAR or a variant of the FAR set forth in SEQ ID NO: 157 (FAR47), having at least 60% identity thereto.

In one embodiment, the heterologous FAR is a Cydia FAR. In one embodiment, the FAR is a Cydia pomonella FAR, such as the FAR as set fort in SEQ ID NO: 82 (FAR23). In some embodiments, the FAR is a variant of a Cydia FAR, a variant of a Cydia pomonella FAR or a variant of the FAR set forth in SEQ ID NO: 82 (FAR23), having at least 60% identity thereto.

In one embodiment, the heterologous FAR is a Helicoverpa FAR. In one embodiment, the FAR is a Helicoverpa armigera FAR. In one embodiment, the FAR is a Helicoverpa armigera FAR, such as the FAR as set fort in SEQ ID NO: 83 (FAR1). In one embodiment, the FAR is a Helicoverpa assulta FAR, such as the FAR as set fort in SEQ ID NO: 84 (FAR6). In some embodiments, the FAR is a variant of a Helicoverpa FAR, a variant of a Helicoverpa armigera FAR, a variant of a Helicoverpa armigera FAR, a variant of a Helicoverpa assulta FAR such as a variant of the FAR set forth in SEQ ID NO: 83 (FAR1) or of the FAR set forth in SEQ ID NO: 84 (FAR6), having at least 60% identity thereto.

In one embodiment, the heterologous FAR is a Heliothis FAR. In one embodiment, the FAR is a Heliothis subflexa FAR, such as the FAR as set fort in SEQ ID NO: 85 (FAR4). In one embodiment, the FAR is a Heliothis virescens FAR, such as the FAR as set fort in SEQ ID NO: 86 (FAR5). In some embodiments, the FAR is a variant of a Heliothis FAR, a variant of a Heliothis subflexa FAR, a variant of a Heliothis virescens FAR, a variant of the FAR set forth in SEQ ID NO: 85 (FAR4), or of the FAR as set forth in SEQ ID NO: 86 (FAR5), having at least 60% identity thereto.

In one embodiment, the heterologous FAR is a Manducta FAR. In one embodiment, the FAR is a Manducta sexta FAR, such as the FAR as set fort in SEQ ID NO: 160 (FAR43). In some embodiments, the FAR is a variant of a Manducta FAR, a variant of a Manducta sexta FAR or a variant of the FAR set forth in SEQ ID NO: 160 (FAR43), having at least 60% identity thereto.

In one embodiment, the heterologous FAR is a Marinobacter FAR. In one embodiment, the FAR is a Marinobacter algicola FAR, such as the FAR as set fort in SEQ ID NO: 159 (FAR42). In some embodiments, the FAR is a variant of a Marinobacter FAR, a variant of a Marinobacter algicola FAR or a variant of the FAR set forth in SEQ ID NO: 159 (FAR42), having at least 60% identity thereto.

In one embodiment, the heterologous FAR is an Ostrinia FAR. In one embodiment, the FAR is an Ostrinia furnacalis FAR, such as the FAR as set fort in SEQ ID NO: 161 (FAR44). In some embodiments, the FAR is a variant of an Ostrinia FAR, a variant of an Ostrinia furnacalis FAR or a variant of the FAR set forth in SEQ ID NO: 161 (FAR44), having at least 60% identity thereto.

In one embodiment, the heterologous FAR is a Plodia FAR. In one embodiment, the FAR is a Plodia interpunctella FAR, such as the FAR as set fort in SEQ ID NO: 162 (FAR28) or in SEQ ID NO: 163 (FAR30). In some embodiments, the FAR is a variant of a Plodia FAR, a variant of a Plodia interpunctella FAR or a variant of the FAR set forth in SEQ ID NO: 162 (FAR28) or in SEQ ID NO: 163 (FAR30), having at least 60% identity thereto.

In one embodiment, the heterologous FAR is a Plutella FAR. In one embodiment, the FAR is a Plutella xylostella FAR, such as the FAR as set fort in SEQ ID NO: 87 (FAR27). In some embodiments, the FAR is a variant of a Plutella FAR, a variant of a Plutella xylostella FAR or a variant of the FAR set forth in SEQ ID NO: 87 (FAR27), having at least 60% identity thereto.

In one embodiment, the heterologous FAR is a Spodoptera FAR. In one embodiment, the FAR is a Spodoptera exigua FAR, such as the FAR as set fort in SEQ ID NO: 88 (FAR16). In one embodiment, the FAR is a Spodoptera frugiperda FAR, such as the FAR as set fort in SEQ ID NO: 89 (FAR22). In one embodiment, the FAR is a Spodoptera littoralis FAR, such as the FAR as set fort in SEQ ID NO: 90 (FAR15). In one embodiment, the FAR is a Spodoptera litura FAR, such as the FAR as set fort in SEQ ID NO: 91 (FAR19). In some embodiments, the FAR is a variant of a Spodoptera FAR, a variant of a Spodoptera exigua FAR, a variant of a Spodoptera frugiperda FAR, a variant of a Spodoptera littoralis FAR, a variant of a Spodoptera littura FAR, a variant of the FAR set forth in SEQ ID NO: 88 (FAR16), a variant of the FAR set forth in SEQ ID NO: 89 (FAR22), a variant of the FAR set forth in SEQ ID NO: 90 (FAR15), a variant of the FAR set forth in SEQ ID NO: 91 (FAR19), having at least 60% identity thereto.

In one embodiment, the heterologous FAR is a Tyta FAR. In one embodiment, the FAR is a Tyta alba FAR, such as FAR25 as set fort in SEQ ID NO: 92. In some embodiments, the FAR is a variant of a Tyta FAR, a variant of a Tyta alba FAR or a variant of FAR25 as set forth in SEQ ID NO: 92, having at least 60% identity thereto.

In one embodiment, the heterologous FAR is a Trichoplusia FAR. In one embodiment, the FAR is a Trichoplusia ni FAR, such as FAR38 as set fort in SEQ ID NO: 93. In some embodiments, the FAR is a variant of a Trichoplusia FAR, a variant of a Trichoplusia ni FAR or a variant of FAR38 as set forth in SEQ ID NO: 93, having at least 60% identity thereto.

In one embodiment, the heterologous FAR is a Trichoplusia FAR. In one embodiment, the FAR is a Trichoplusia ni FAR, such as FAR41 as set fort in SEQ ID NO: 166. In some embodiments, the FAR is a variant of a Trichoplusia FAR, a variant of a Trichoplusia ni FAR or a variant of FAR41 as set forth in SEQ ID NO: 166, having at least 60% identity thereto.

In one embodiment, the heterologous FAR is a Yponomeuta FAR. In one embodiment, the FAR is a Yponomeuta rorellus FAR, such as FAR8 as set fort in SEQ ID NO: 167.

In some embodiments, the FAR is a variant of a Yponomeuta FAR, a variant of a Yponomeuta rorellus FAR or a variant of FAR8 as set forth in SEQ ID NO: 167, having at least 60% identity thereto.

It will be understood that a variant FAR having at least 60% identity to a given FAR as above may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the FAR, or more.

### Nucleic Acids Encoding FARs

In some embodiments, the heterologous FAR is encoded by a nucleic acid having at least 60% identity to a nucleic acid selected from the group of desaturases set forth in SEQ ID NOs: 94 to 110 and SEQ ID NOs: 168-181, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity, such as 100% identity thereto.

In one embodiment, the heterologous FAR is encoded by a nucleic acid having at least 60% identity to the nucleic acid encoding a FAR from Agrotis segetum, as set forth in SEQ ID NO: 94. In one embodiment, the heterologous FAR is encoded by a nucleic acid having at least 60% identity to the nucleic acid encoding a FAR from Agrotis ipsilon, as set forth in SEQ ID NO: 95. In one embodiment, the heterologous FAR is encoded by a nucleic acid having at least 60% identity to the nucleic acid encoding a FAR from Amyelois transitella, as set forth in SEQ ID NO: 168, SEQ ID NO: 169 or SEQ ID NO: 170. In one embodiment, the heterologous FAR is encoded by a nucleic acid having at least 60% identity to the nucleic acid encoding a FAR from Bicyclus anynana, as set forth in SEQ ID NO: 96. In one embodiment, the heterologous FAR is encoded by a nucleic acid having at least 60% identity to the nucleic acid encoding a FAR from Bombus lapidarius, as set forth in SEQ ID NO: 97. In one embodiment, the heterologous FAR is encoded by a nucleic acid having at least 60% identity to the nucleic acid encoding a FAR from Chilo suppressalis, as set forth in SEQ ID NO: 98. In one embodiment, the heterologous FAR is encoded by a nucleic acid having at least 60% identity to the nucleic acid encoding a FAR from Chrysodeixis includens, as set forth in SEQ ID NO: 171. In one embodiment, the heterologous FAR is encoded by a nucleic acid having at least 60% identity to the nucleic acid encoding a FAR from Cydia pomonella, as set forth in SEQ ID NO: 99 or SEQ ID NO: 172. In one embodiment, the heterologous FAR is encoded by a nucleic acid having at least 60% identity to the nucleic acid encoding a FAR from Helicoverpa armigera, as set forth in SEQ ID NO: 100. In one embodiment, the heterologous FAR is encoded by a nucleic acid having at least 60% identity to the nucleic acid encoding a FAR from Helicoverpa assulta, as set forth in SEQ ID NO: 101. In one embodiment, the heterologous FAR is encoded by a nucleic acid having at least 60% identity to the nucleic acid encoding a FAR from Heliothis subflexa, as set forth in SEQ ID NO: 102. In one embodiment, the heterologous FAR is encoded by a nucleic acid having at least 60% identity to the nucleic acid encoding a FAR from Heliothis virescens, as set forth in SEQ ID NO: 103. In one embodiment, the heterologous FAR is encoded by a nucleic acid having at least 60% identity to the nucleic acid encoding a FAR from Marinobacter algicola, as set forth in SEQ ID NO: 173. In one embodiment, the heterologous FAR is encoded by a nucleic acid having at least 60% identity to the nucleic acid encoding a FAR from Manducta sexta, as set forth in SEQ ID NO: 174. In one embodiment, the heterologous FAR is encoded by a nucleic acid having at least 60% identity to the nucleic acid encoding a FAR from Ostrinia furnacalis, as set forth in SEQ ID NO: 175. In one embodiment, the heterologous FAR is encoded by a nucleic acid having at least 60% identity to the nucleic acid encoding a FAR from Plodia interpunctella, as set forth in SEQ ID NO: 176 or SEQ ID NO: 177. In one embodiment, the heterologous FAR is encoded by a nucleic acid having at least 60% identity to the nucleic acid encoding a FAR from Plutella xylostella, as set forth in SEQ ID NO: 104. In one embodiment, the heterologous FAR is encoded by a nucleic acid having at least 60% identity to the nucleic acid encoding a FAR from Spodoptera exigua, as set forth in SEQ ID NO: 105, SEQ ID NO: 178 or SEQ ID NO: 179. In one embodiment, the heterologous FAR is encoded by a nucleic acid having at least 60% identity to the nucleic acid encoding a FAR from Spodoptera frugiperda, as set forth in SEQ ID NO: 106. In one embodiment, the heterologous FAR is encoded by a nucleic acid having at least 60% identity to the nucleic acid encoding a FAR from Spodoptera littoralis, as set forth in SEQ ID NO: 107. In one embodiment, the heterologous FAR is encoded by a nucleic acid having at least 60% identity to the nucleic acid encoding a FAR from Spodoptera litura, as set forth in SEQ ID NO: 108. In one embodiment, the heterologous FAR is encoded by a nucleic acid having at least 60% identity to the nucleic acid encoding a FAR from Tyta alba, as set forth in SEQ ID NO: 109. In one embodiment, the heterologous FAR is encoded by a nucleic acid having at least 60% identity to the nucleic acid encoding a FAR from Trichoplusia ni, as set forth in SEQ ID NO: 110 or SEQ ID NO: 180. In one embodiment, the heterologous FAR is encoded by a nucleic acid having at least 60% identity to the nucleic acid encoding a FAR from Yponomeuta rorellus, as set forth in SEQ ID NO: 181. Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to the given nucleic acid, or more.

### Co-Expression of a Plurality of FARs

The present cells may express at least one heterologous FAR. In some embodiments, the cell expresses one heterologous FAR. It may however be desirable to express several heterologous FARs, such as at least two heterologous FARs, which may be identical or different. Alternatively, it may be desirable to express several copies of the nucleic acid encoding the at least one heterologous FAR, such as at least two copies, at least three copies or more. In other embodiments, the cell expresses at least two heterologous FARs, for example three heterologous FARs.

For example, the cell may express two copies of FAR1 or a variant thereof; or one copy of FAR1 and one copy of FAR5; or two copies of FAR1, one copy of FAR5 and one copy of FAR4.

### Desaturases and FARs

Any of the above FARs can be expressed together with any desaturase, in particular any of the desaturases described herein.

In some embodiments, the cell expresses:
an Agrotis FAR, such as an Agrotis segetum FAR, for example FAR12 as set forth in SEQ ID NO: 77, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
an Agrotis FAR, such as an Agrotis ispsilon FAR, for example FAR18 as set forth in SEQ ID NO: 78, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Bicyclus FAR, such as a Bicyclus anynana FAR, for example FAR11 as set forth in SEQ ID NO: 79, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Bombus FAR, such as a Bombus lapidarius FAR, for example FAR14 as set forth in SEQ ID NO: 80, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Chilo FAR, such as a Chilo suppressalis FAR, for example FAR13 as set forth in SEQ ID NO: 81, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Cydia FAR, such as a Cydia pomonella FAR, for example FAR23 as set forth in SEQ ID NO: 82, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Helicoverpa FAR, such as a Helicoverpa assulta FAR, for example FAR6 as set forth in SEQ ID NO: 83 or a Helicoverpa armigera FAR, for example FAR1 as set forth in SEQ ID NO: 82; and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Heliothis FAR, such as a Heliothis subflexa FAR, for example FAR4 as set forth in SEQ ID NO: 85 or a Heliothis virescens FAR, for example FAR5 as set forth in SEQ ID NO: 86; and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Plutella FAR, such as a Plutella xylostella FAR, for example FAR27 as set forth in SEQ ID NO: 87, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Spodoptera FAR, such as a Spodoptera exigua FAR, for example FAR16 as set forth in SEQ ID NO: 88, a Spodoptera frugiperda FAR, for example FAR22 as set forth in SEQ ID NO: 89, a Spodoptera littoralis FAR, for example FAR15 as set forth in SEQ ID NO: 90, or a Spodoptera litura FAR, for example FAR19 as set forth in SEQ ID NO: 91, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Tyta FAR, such as a Tyta alba FAR, for example FAR25 as set forth in SEQ ID NO: 92, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

In other embodiments, the cell expresses:
a Trichoplusia FAR, such as a Trichoplusia ni FAR, for example FAR38 as set forth in SEQ ID NO: 93, and/or
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and
an Ncb5or as detailed below,
   or variants thereof having at least 60% identity thereto.

The term “variant thereof having at least 60% identity” in relation to a given enzyme shall be understood to refer to variants having 60% identity or more to said enzyme, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the enzyme, or more.

### NAD(P)H Cytochrome b5 Oxidoreductase

While expression of one or more heterologous desaturases and/or one or more reductases can result in the production of desaturated fatty alcohols, saturated fatty alcohols, desaturated fatty alcohol acetates, and/or saturated fatty alcohol acetates, the inventors have found that introduction of an additional enzyme in the cell appears to have a positive effect on the activity of the desaturase and/or reductase, as it results in an increase in titer. This additional enzyme is an NAD(P)H cytochrome b5 oxidoreductase, and is naturally found in a number of insects, including of the Lepidoptera order.

The term ‘NAD(P)H cytochrome b5 oxidoreductase’ and ‘Ncb5or’ will be used herein interchangeably. The term ‘heterologous Ncb5or’ refers to an Ncb5or which is not naturally expressed by the organism, such as by the cell.

Ncb5or is also known as cytochrome b5 reductase 4 and is an oxidoreductase acting on NADH or NADPH, with a heme protein as acceptor. It contains three functional domains similar to cytochrome b5, cytochrome b5 reductase and CHORD-SGT1 (Deng, et al., 2010). Ncb5ors catalyze the reaction:

$$2 {Fe}^{3 +} + NAD ( P ) H \text{<=>} 2 {Fe}^{2 +} + H + + NAD ( P ) +$$

Ncb5ors capable of catalyzing such reaction have EC number 1.6.2.2.

The cell disclosed herein expresses a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous NAD(P)H cytochrome b5 oxidoreductase (Ncb5or); whereby the cell is capable of producing the compound with a higher titer compared to a cell expressing the first group of enzymes but no heterologous Ncb5or when cultivated in the same conditions.

In one embodiment, the first enzyme or group of enzymes may consist of one or more desaturase capable of converting a fatty acyl-CoA to a desaturated fatty acyl-CoA, whereby the cell is capable of producing a desaturated fatty acyl-CoA with a higher titer compared to a cell expressing said one or more desaturase but no heterologous Ncb5or when cultivated in the same conditions.

In another embodiment, the first enzyme or group of enzymes consists of one or more fatty acyl reductase (FAR) capable of converting a fatty acyl-CoA to a saturated fatty alcohol, whereby the cell is capable of producing a saturated fatty alcohol with a higher titer compared to a cell expressing said one or more FAR but no heterologous Ncb5or when cultivated in the same conditions.

In yet another embodiment, the first enzyme or group of enzymes consists of one or more fatty acyl reductase (FAR) and one or more desaturase capable of converting a fatty acyl-CoA to a desaturated fatty alcohol, whereby the cell is capable of producing a desaturated fatty alcohol with a higher titer compared to a cell expressing said one or more FAR and said one or more desaturase but no heterologous Ncb5or when cultivated in the same conditions.

The cell may further express an acetyltransferase, whereby said cell is capable of converting the desaturated or the saturated fatty alcohol to a desaturated or a saturated fatty alcohol acetate, respectively, whereby the cell is capable of producing a desaturated or a saturated fatty alcohol acetate with a higher titer compared to a cell expressing the first group of enzymes and the acetyltransferase but no heterologous Ncb5or when cultivated in the same conditions.

In a preferred embodiment, the production of said desaturated fatty alcohol, saturated fatty alcohol, desaturated fatty alcohol acetate, saturated fatty alcohol acetate, and a desaturated fatty acyl-CoA is increased in said organism compared to the production of said desaturated fatty alcohol, saturated fatty alcohol, desaturated fatty alcohol acetate, saturated fatty alcohol acetate, and a desaturated fatty acyl-CoA in an organism cultivated in the same conditions and not expressing said heterologous Ncb5or. In other words, expression of a heterologous Ncb5or increases the production of said desaturated fatty alcohol, saturated fatty alcohol, desaturated fatty alcohol acetate, saturated fatty alcohol acetate, and a desaturated fatty acyl-CoA in a cell expressing said first enzyme or group of enzymes. Thus, expression of a heterologous Ncb5or increases the activity of said first enzyme or group of enzymes, such as said heterologous desaturase and/or of said heterologous FAR compared to the activity of said heterologous desaturase and/or said heterologous FAR in the absence of said heterologous Ncb5or, when tested in identical or similar conditions, wherein the activity is measured e.g. by measuring the titer of product formed by the heterologous desaturase and the heterologous FAR.

Further provided herein is the use of an Ncb5or in a method for increasing the activity of one or more enzymes.

In one embodiment, the one or more enzymes is one or more membrane-bound enzymes. The skilled person knows how to determine whether an enzyme is membrane-bound. For instance, fluorescent markers can be used, to determine whether an enzyme fused to a fluorescent marker colocalises with a protein which is known to be found in the membrane.

In one embodiment, the one or more enzymes is selected from the group consisting of desaturases and fatty acyl reductases, such as the desaturases and fatty acyl reductases presented herein in the sections “Desaturase” and “Fatty acyl-CoA reductase”, respectively.

In one embodiment, the increase in activity of said of one or more enzymes, for example a desaturase and/or a FAR as described herein, is at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, such as at least 30-fold, such as at least 40-fold, such as at least 50-fold; wherein the increase in activity of said of one or more enzymes is compared to the activity of said one or more enzymes in the absence of said Ncb5or, wherein the activity is measured under the same conditions, wherein the increase is measured by measuring the concentration of product formed by the one or more enzymes.

In one embodiment, the increase in activity of said of one or more enzymes, for example a desaturase and/or a FAR as described herein, is at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, such as at least 30-fold, such as at least 40-fold, such as at least 50-fold; wherein the increase in activity of said of one or more enzymes is compared to the activity of said one or more enzymes in the absence of said Ncb5or, wherein the activity is measured under the same conditions, wherein the increase is measured by measuring the concentration of product formed by the desaturase and/or the FAR.

The Ncb5ors disclosed herein may be any type of Ncb5or. In some embodiments, the Ncb5or is native to a plant, an insect or a mammal. In some embodiments, the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa. In preferred embodiments, the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.

In some embodiments, the Ncb5or is an Ncb5or selected from Table 5.

In some embodiments, the Ncb5or is an Ncb5or selected from the group of Ncb5ors as set forth in SEQ ID NOs: 111 to 114, SEQ ID NO: 124 or SEQ ID NOs: 182 to 185, or variants thereof having at least 60% identity to an Ncb5or selected from the group of Ncb5ors set forth in SEQ ID NOs: 111 to 114, SEQ ID NO: 124 or SEQ ID NOs: 182 to 185.

In one embodiment, the Ncb5or is a Cydia Ncb5or. In one embodiment, the Ncb5or is a Cydia pomonella Ncb5or. In some embodiments, the Ncb5or is a variant of a Cydia Ncb5or as set forth in SEQ ID NO: 124 (CpoNcb5or1) or SEQ ID NO: 182 (CpNcb5or), or a variant thereof having at least 60% identity thereto.

In one embodiment, the Ncb5or is a Drosophila Ncb5or. In one embodiment, the Ncb5or is a Drosophila melanogaster Ncb5or. In one embodiment, the Ncb5or is a Drosophila virilis Ncb5or. In some embodiments, the Ncb5or is a variant of a Drosophila Ncb5or, a variant of a Drosophila melanogaster Ncb5or, a variant of a Drosophila grimshawi Ncb5or, a variant of the Ncb5or as set forth in SEQ ID NO: 112 (DmNcb5or), or a variant of the Ncb5or as set forth in SEQ ID NO: 111 (DgNcb5or), having at least 60% identity to the Ncb5or set forth in SEQ ID NO: 112 or SEQ ID NO: 111.

In one embodiment, the Ncb5or is a Homo Ncb5or. In one embodiment, the Ncb5or is a Homo sapiens Ncb5or. In some embodiments, the Ncb5or is a variant of a Homo Ncb5or, a variant of a Homo sapiens Ncb5or or a variant of the Ncb5or as set forth in SEQ ID NO: 113 (HsNcb5or), having at least 60% identity to the Ncb5or set forth in SEQ ID NO: 113.

In one embodiment, the Ncb5or is a Lobesia Ncb5or. In one embodiment, the Ncb5or is a Lobesia botrana Ncb5or, such as set forth in SEQ ID NO: 189 (LboNcb5or). In some embodiments, the Ncb5or is a variant of a Lobesia Ncb5or such as set forth in SEQ ID NO: 189, having at least 60% identity thereto.

In some embodiments, the Ncb5or is a Bombus Ncb5or. In one embodiments, the Ncb5or is a Bombus terrestris Ncb5or, such as set forth in SEQ ID NO: 184 (BterNcb5or), or a variant thereof having at least 60% identity thereto.

In one embodiment, the Ncb5or is a Spodoptera Ncb5or. In one embodiment, the Ncb5or is a Spodoptera litura Ncb5or. In some embodiments, the Ncb5or is a variant of a Spodoptera Ncb5or, a variant of a Spodoptera litura Ncb5or or a variant of the Ncb5or as set forth in SEQ ID NO: 114 (SlitNcb5or), having at least 60% identity to the Ncb5or set forth in SEQ ID NO: 114.

A variant of an Ncb5or refers to a functional variant of an Ncb5or, which retains at least some or all of the Ncb5or activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

### Nucleic Acids Encoding Ncb5ors

In some embodiments, the heterologous Ncb5or is encoded by a nucleic acid having at least 60% identity to an Ncb5or selected from the group of Ncb5ors set forth in SEQ ID NOs: 115 to 118. Such nucleic acids may be introduced in a cell as described herein, or may be comprised within a vector such as a plasmid, as is known in the art.

In one embodiment, the heterologous Ncb5or is encoded by a nucleic acid having at least 60% identity to a nucleic acid encoding an Agrotis segetum Ncb5or, such as the nucleic acid sequence as set forth in SEQ ID NO: 187. In one embodiment, the heterologous Ncb5or is encoded by a nucleic acid having at least 60% identity to a nucleic acid encoding a Bombus terrestris Ncb5or, such as the nucleic acid sequence as set forth in SEQ ID NO: 188. In one embodiment, the heterologous Ncb5or is encoded by a nucleic acid having at least 60% identity to a nucleic acid encoding a Cydia pomonella Ncb5or, such as the nucleic acid sequence as set forth in SEQ ID NO: 125 or SEQ ID NO: 182. In one embodiment, the heterologous Ncb5or is encoded by a nucleic acid having at least 60% identity to the nucleic acid encoding the Ncb5or from Drosophila grimshawi as set forth in SEQ ID NO: 115. In one embodiment, the heterologous Ncb5or is encoded by a nucleic acid having at least 60% identity to the nucleic acid encoding the Ncb5or from Drosophila melanogaster as set forth in SEQ ID NO: 116. In one embodiment, the heterologous Ncb5or is encoded by a nucleic acid having at least 60% identity to the nucleic acid encoding the Ncb5or from Homo sapiens as set forth in SEQ ID NO: 117. In one embodiment, the heterologous Ncb5or is encoded by a nucleic acid having at least 60% identity to a nucleic acid encoding a Lobesia botrana Ncb5or, such as the nucleic acid sequence as set forth in SEQ ID NO: 185. In one embodiment, the heterologous Ncb5or is encoded by a nucleic acid having at least 60% identity to the nucleic acid encoding the Ncb5or from Spodoptera litura as set forth in SEQ ID NO: 118.

Herein a nucleic acid having at least 60% identity to a given nucleic acid has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

### Co-Expression of a Plurality of Ncb5ors

The present cells express at least one heterologous Ncb5or. In some embodiments, the cell expresses one heterologous Ncb5or. It may however be desirable to express several heterologous Ncb5ors, such as at least two heterologous Ncb5ors, which may be identical or different. Alternatively, it may be desirable to express several copies of the nucleic acid encoding the at least one heterologous Ncb5or, such as at least two copies, at least three copies or more. In some embodiments, the cell expresses at least two heterologous Ncb5ors, for example three heterologous Ncb5ors.

### Desaturases, FARs and Ncb5ors

Any of the above Ncb5ors can be expressed in the cell together with any combination of desaturase and reductase described herein. The Ncb5ors listed herein below can thus be used to increase the activity of any of the FARs and/or desaturases listed herein below, not only in vivo, but also in vitro.

In some embodiments, the cell expresses a Cydia Ncb5or, such as a Cydia pomonella Ncb5or, for example CpoNcb5or1 (SEQ ID NO: 124) or CpNcb5or (SEQ ID NO: 182); and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
or variants thereof having at least 60% identity thereto.

In other embodiments, the cell expresses a Drosophila Ncb5or, such as a Drosophila grimshawhi Ncb5or, for example DgNcb5or as set forth in SEQ ID NO: 111; and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
   or variants thereof having at least 60% identity thereto.

In other embodiments, the cell expresses a Drosophila Ncb5or, such as a Drosophila melanogaster Ncb5or, for example DmNcb5or as set forth in SEQ ID NO: 112; and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
   or variants thereof having at least 60% identity thereto.

In other embodiments, the cell expresses a Homo Ncb5or, such as a Homo sapiens Ncb5or, for example HsNcb5or as set forth in SEQ ID NO: 113; and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
   or variants thereof having at least 60% identity thereto.

In other embodiments, the cell expresses a Lobesia Ncb5or, such as a Lobesia botrana Ncb5or; and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
   or variants thereof having at least 60% identity thereto.

In other embodiments, the cell expresses a Spodoptera Ncb5or, such as a Spodoptera litura Ncb5or, for example SlitNcb5or as set forth in SEQ ID NO: 114; and one or both of:
a desaturase selected from: an Agrotis desaturase, such as an Agrotis segetum desaturase, for example Desat19 as set forth in SEQ ID NO: 1; an Amyelois desaturase, such as an Amyelois transitella desaturase, for example Desat16 as set forth in SEQ ID NO: 2, Desat17 as set forth in SEQ ID NO: 3, or Desat18 as set forth in SEQ ID NO: 4; a Chauliognathus desaturase, such as a Chauliognathus lugubris desaturase, for example Desat25 as set forth in SEQ ID NO: 5; a Chilo desaturase, such as a Chilo supprealis desaturase, for example Desat47 as set forth in SEQ ID NO: 6; a Choristoneura desaturase, such as a Choristoneura parallela desaturase, for example Desat36 as set forth in SEQ ID NO: 7, or such as a Choristoneura rosaceana desaturase, for example Desat35 as set forth in SEQ ID NO: 8; a Cydia desaturase, such as a Cydia pomonella desaturase, for example Desat4 as set forth in SEQ ID NO: 9, Desat2 as set forth in SEQ ID NO: 10, or Desat1 as set forth in SEQ ID NO: 11; a Dendrolimus desaturase, such as a Dendrolimus punctatus desaturase, for example Desat40 as set forth in SEQ ID NO: 12; a Drosophila desaturase, such as a Drosophila grimshawi desaturase, for example Desat59 as set forth in SEQ ID NO: 13, or such as a Drosophila melanogaster desaturase, for example Desat24 as set forth in SEQ ID NO: 14, or such as a Drosophila virilis desaturase, for example Desat61 as set forth in SEQ ID NO: 15; or an Epiphyas desaturase, such as an Epiphyas postvittana desaturase, for example Desat33 as set forth in SEQ ID NO: 16; a Grapholita desaturase, such as a Grapholita molesta desaturase, for example Desat31 as set forth in SEQ ID NO: 17, or Desat55 as set forth in SEQ ID NO: 18; a Helicoverpa desaturase, such as a Helicoverpa zea desaturase, for example Desat51 as set forth in SEQ ID NO: 19; a Lobesia desaturase, such as a Lobesia botrana desaturase, for example Desat30 as set forth in SEQ ID NO: 20 or Desat43 as set forth in SEQ ID NO: 21; a Manducta desaturase, for example Desat52 as set forth in SEQ ID NO: 22; an Ostrinia desaturase, such as an Ostrinia nubilalis desaturase, for example Desat32 as set forth in SEQ ID NO: 23; an Pectinophora desaturase, such as an Pectinophora gossypiella desaturase, for example Desat48 as set forth in SEQ ID NO: 24; a Pelargonium desaturase, such as a Pelargonium hortorum desaturase, for example Desat22 as set forth in SEQ ID NO: 25; a Plutella desaturase, such as a Plutella xylostella desaturase, for example Desat45 as set forth in SEQ ID NO: 26; a Ricinus desaturase, such as a Ricinus communis desaturase, for example Desat23 as set forth in SEQ ID NO: 27; a Saccharomyces desaturase, such as a Saccharomyces cerevisiae desaturase, for example Desat42 as set forth in SEQ ID NO: 28; a Spodoptera desaturase, such as a Spodoptera exigua desaturase, for example Desat37 as set forth in SEQ ID NO: 29, or such as a Spodoptera littoralis desaturase, for example Desat20 as set forth in SEQ ID NO: 30 or Desat20 as set forth in SEQ ID NO: 31, or such as a Spodoptera litura desaturase, for example Desat38 as set forth in SEQ ID NO: 32 or Desat26 as set forth in SEQ ID NO: 33; a Thaumetopoea desaturase, such as a Thaumetopoea pityocampa desaturase, for example Desat34 as set forth in SEQ ID NO: 34; a Tribolium desaturase, such as a Tribolium castaneum desaturase, for example Desat28 as set forth in SEQ ID NO: 35 or Desat29 as set forth in SEQ ID NO: 36; a Trichoplusia desaturase, such as a Trichoplusia ni desaturase, for example Desat21 as set forth in SEQ ID NO: 37; a Yarrowia desaturase, such as a Yarrowia lipolytica desaturase, for example Desat69 as set forth in SEQ ID NO: 38; or combinations thereof; and/or
a FAR selected from: an Agrotis FAR, such as an Agrotis segetum FAR, for example FAR12 as set forth in SEQ ID NO: 77, or such as an Agrotis ispsilon FAR, for example FAR18 as set forth in SEQ ID NO: 78; a Bicyclus FAR, such as a Bicyclus anynana FAR, for example FAR11 as set forth in SEQ ID NO: 79; a Bombus FAR, such as a Bombus lapidarius FAR, for example FAR14 as set forth in SEQ ID NO: 80; a Chilo FAR, such as a Chilo suppressalis FAR, for example FAR13 as set forth in SEQ ID NO: 81; a Cydia FAR, such as a Cydia pomonella FAR, for example FAR23 as set forth in SEQ ID NO: 82; a Helicoverpa FAR, such as a Helicoverpa armigera FAR, for example FAR1 as set forth in SEQ ID NO: 83, or such as a Helicoverpa assulta FAR, for example FAR6 as set forth in SEQ ID NO: 84; a Heliothis FAR, such as a Heliothis virescens FAR, for example FAR5 as set forth in SEQ ID NO: 86, or such as a Heliothis subflexa FAR, for example FAR4 as set forth in SEQ ID NO: 85; a Plutella FAR, such as a Plutella xylostella FAR, for example FAR27 as set forth in SEQ ID NO: 87; a Spodoptera FAR, such as a Spodoptera exigua FAR, for example FAR16 as set forth in SEQ ID NO: 88, or a Spodoptera frugiperda FAR, for example FAR22 as set forth in SEQ ID NO: 89, or a Spodoptera littoralis FAR, for example FAR15 as set forth in SEQ ID NO: 90, or a Spodoptera litura FAR, for example FAR19 as set forth in SEQ ID NO: 91; a Tyta FAR, such as a Tyta alba FAR, for example FAR25 as set forth in SEQ ID NO: 92; and a Trichoplusia FAR, such as a Trichoplusia ni FAR, for example FAR38 as set forth in SEQ ID NO: 93;
   or variants thereof having at least 60% identity thereto.

The term “variant having at least 60% identity” in relation to a given enzyme shall be understood to refer to variants having 60% identity or more to said enzyme, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the enzyme, or more.

Methods to determine whether an Ncb5or increases the activity of a desaturase and/or a fatty acyl-CoA reductase will be apparent to the person of skill in the art in view of the present disclosure. For example, whether a given Ncb5or increases the activity of a desaturase can be determined by incubating in a solution cells comprising the fatty acyl-CoA substrate for said desaturase, wherein the cells express either (i) said Ncb5or and said desaturase; or (ii) said desaturase. After 48 hours of incubation under the same conditions, the amount (titer) of product (i.e. the amount of desaturated fatty-acyl CoA) generated by the desaturase can be determined by GC-MS. A higher titer from the cells expressing said Ncb5or compared to the cells not expressing said Ncb5or indicates that said Ncb5or increases the activity of said desaturase.

In one embodiment, the Ncb5or is DgNcb5or (SEQ ID NO: 111), the FAR is selected from the group consisting of FAR12 (SEQ ID NO: 77), FAR18 (SEQ ID NO: 78), FAR11 (SEQ ID NO: 79), FAR14 (SEQ ID NO: 80), FAR13 (SEQ ID NO: 81), FAR23 (SEQ ID NO: 82), FAR1 (SEQ ID NO: 83), FAR6 (SEQ ID NO: 84), FAR4 (SEQ ID NO: 85), FAR5 (SEQ ID NO: 86), FAR27 (SEQ ID NO: 87), FAR16 (SEQ ID NO: 88), FAR22 (SEQ ID NO: 89), FAR15 (SEQ ID NO: 90), FAR19 (SEQ ID NO: 91), FAR25 (SEQ ID NO: 92), FAR38 (SEQ ID NO: 93), FAR33 (SEQ ID NO: 154), FAR34 (SEQ ID NO: 155), FAR35 (SEQ ID NO: 156), FAR47 (SEQ ID NO: 157), FAR46 (SEQ ID NO: 158), FAR42 (SEQ ID NO: 159), FAR43 (SEQ ID NO: 160), FAR44 (SEQ ID NO: 161), FAR28 (SEQ ID NO: 162), FAR30 (SEQ ID NO: 163), FAR17 (SEQ ID NO: 164), FAR45 (SEQ ID NO: 165), FAR41 (SEQ ID NO: 166), and FAR8 (SEQ ID NO: 167), and the desaturase is selected from the group consisting of Desat19 (SEQ ID NO: 1), Desat16 (SEQ ID NO: 2), Desat17 (SEQ ID NO: 3), Desat18 (SEQ ID NO: 4), Desat25 (SEQ ID NO: 5), Desat47 (SEQ ID NO: 6), Desat36 (SEQ ID NO: 7), Desat35 (SEQ ID NO: 8), Desat4 (SEQ ID NO: 9), Desat2 (SEQ ID NO: 10), Desat1 (SEQ ID NO: 11), Desat40 (SEQ ID NO: 12), Desat59 (SEQ ID NO: 13), Desat24 (SEQ ID NO: 14), Desat61 (SEQ ID NO: 15), Desat33 (SEQ ID NO: 16), Desat31 (SEQ ID NO: 17), Desat55 (SEQ ID NO: 18), Desat51 (SEQ ID NO: 19), Desat30 (SEQ ID NO: 20), Desat43 (SEQ ID NO: 21), Desat52 (SEQ ID NO: 22), Desat32 (SEQ ID NO: 23), Desat48 (SEQ ID NO: 24), Desat22 (SEQ ID NO: 25), Desat45 (SEQ ID NO: 26), Desat23 (SEQ ID NO: 27), Desat42 (SEQ ID NO: 28), Desat37 (SEQ ID NO: 29), Desat20 (SEQ ID NO: 30), Desat20 (SEQ ID NO: 31), Desat38 (SEQ ID NO: 32), Desat26 (SEQ ID NO: 33), Desat34 (SEQ ID NO: 34), Desat28 (SEQ ID NO: 35), Desat29 (SEQ ID NO: 36), Desat21 (SEQ ID NO: 37), Desat69 (SEQ ID NO: 38), Desat72 (SEQ ID NO: 126), Desat76 (SEQ ID NO: 127), Desat75 (SEQ ID NO: 128), Desat78 (SEQ ID NO: 129), Desat44 (SEQ ID NO: 130), Desat60 (SEQ ID NO: 131), Desat63 (SEQ ID NO: 132), Desat56 (SEQ ID NO: 133), Desat70 (SEQ ID NO: 134), Desat71 (SEQ ID NO: 135), Desat77 (SEQ ID NO: 136), Desat65 (SEQ ID NO: 137), Desat27 (SEQ ID NO: 138), and Desat73, preferably the FAR is selected from the group consisting of FAR1, FAR15, FAR16, FAR12, FAR6, FAR8, FAR18, FAR38, and FAR17.

In one embodiment, the Ncb5or is DmNcb5or (SEQ ID NO: 112), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is HsNcb5or (SEQ ID NO: 113), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is SlitNcb5or (SEQ ID NO: 114), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is CpoNcb5or1 (SEQ ID NO: 124), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is CpNcb5or (SEQ ID NO: 182), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is AseNcb5or (SEQ ID NO: 183), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is BterNcb5or (SEQ ID NO: 184), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

In one embodiment, the Ncb5or is LboNcb5or (SEQ ID NO: 185), the FAR is selected from the group of FARs listed above (in the context of an Ncb5or which is DgNcb5or), preferably the FAR is selected from the group consisting of FAR1, FAR15, FAR16, FAR12, FAR6, FAR8, FAR18, FAR38, and FAR17, and the desaturase is selected from the group of desaturases listed above (in the context of an Ncb5or which is DgNcb5or).

### Cell

The present invention provides a cell which has been modified or engineered to produce desaturated and/or saturated compounds, in particular to produce desaturated and/or saturated fatty alcohols; desaturated and/or saturated fatty alcohol acetates; and/or desaturated and/or saturated fatty aldehydes. Some of these are components of pheromones, in particular of moth pheromones. The cell disclosed herein thus provides an improved platform for environment-friendly moth pheromone production.

In one embodiment, the cell described herein is capable of producing a desaturated fatty alcohol, a saturated fatty alcohol, a desaturated fatty alcohol acetate, and/or a saturated fatty alcohol acetate have a carbon chain length of 8, 9, 10, 11, 12, 13, 14, 15, 16, 17, 18, 19, 20, 21 or 22. In preferred embodiments, the carbon chain has a length of 11, 12, 13, 14, 15, 16, 17 or 18.

Accordingly, one embodiment of the present invention provides a cell expressing
i) a first enzyme or group of enzymes capable of converting a fatty-acyl CoA to a compound selected from a desaturated fatty alcohol, a saturated fatty alcohol, a desaturated fatty alcohol acetate, and a desaturated fatty acyl-CoA; and
ii) a heterologous NAD(P)H cytochrome b5 oxidoreductase (Ncb5or);
   whereby the cell is capable of producing the compound with a higher titer compared to a cell expressing the first group of enzymes but no heterologous Ncb5or when cultivated in the same conditions.

In one embodiment, the first enzyme or group of enzymes consists of one or more desaturase capable of converting a fatty acyl-CoA to a desaturated fatty acyl-CoA, whereby the cell is capable of producing a desaturated fatty acyl-CoA with a higher titer compared to a cell expressing said one or more desaturase but no heterologous Ncb5or when cultivated in the same conditions.

In one embodiment, the first enzyme or group of enzymes consists of one or more fatty acyl reductase (FAR) capable of converting a fatty acyl-CoA to a saturated fatty alcohol, whereby the cell is capable of producing a saturated fatty alcohol with a higher titer compared to a cell expressing said one or more FAR but no heterologous Ncb5or when cultivated in the same conditions.

In one embodiment, the first enzyme or group of enzymes consists of one or more fatty acyl reductase (FAR) and one or more desaturase capable of converting a fatty acyl-CoA to a desaturated fatty alcohol, whereby the cell is capable of producing a desaturated fatty alcohol with a higher titer compared to a cell expressing said one or more FAR and said one or more desaturase but no heterologous Ncb5or when cultivated in the same conditions.

In one embodiment, the cell further expresses an acetyltransferase capable of converting a desaturated or a saturated fatty alcohol to a desaturated or a saturated fatty alcohol acetate, respectively, whereby the cell is capable of producing a desaturated or a saturated fatty alcohol acetate with a higher titer compared to a cell expressing the first group of enzymes and the acetyltransferase but no heterologous Ncb5or when cultivated in the same conditions.

The cell may express any of the combinations of Ncb5or and first enzyme or group of enzymes, in particular any desaturase or FAR, described herein.

In one embodiment, the cell expresses a heterologous NAD(P)H cytochrome b5 oxidoreductase (Ncb5or) selected from the group consisting SEQ ID NOs: 111 to 114, SEQ ID NO: 124 and SEQ ID NOs: 182 to 185, a heterologous desaturase selected from the group consisting of SEQ ID NOs: 1 to 38 and SEQ ID NO: 126 to 139, and a heterologous fatty acyl CoA reductase (FAR) selected from the group consisting of SEQ ID NOs: 77 to 93 and SEQ ID NO: 154-167; for example a desaturase from Spodoptera litura (Desat38) as set forth in SEQ ID NO: 32, and a fatty acyl CoA reductase (FAR) from Helicoverpa armigera (FAR1) as set forth in SEQ ID NO: 83; or a desaturase from Lobesia botrana (Desat30) as set forth in SEQ ID NO: 20, and a fatty acyl CoA reductase (FAR) from Helicoverpa armigera (FAR1) as set forth in SEQ ID NO: 83; or a desaturase from Drosophila virilis (Desat61) as set forth in SEQ ID NO: 15, and a fatty acyl CoA reductase (FAR) from Helicoverpa armigera (FAR1) as set forth in SEQ ID NO: 83.

In one embodiment, the cell expresses:
a. a desaturase from Spodoptera litura (Desat38) as set forth in SEQ ID NO: 32;
b. a fatty acyl CoA reductase (FAR) as described herein above, such as a Helicoverpa armigera FAR, for example FAR1 as set forth in SEQ ID NO: 83, or such as a FAR from Agrotis segetum, for example FAR12 as set forth in SEQ ID NO: 77; and
c. an NAD(P)H cytochrome b5 oxidoreductase (Nc5bor) selected from the group consisting of an Ncb5or from Drosophila melanogaster such as DmNcb5or as set forth in SEQ ID NO: 112, an Ncb5or from Spodoptera litura such as SlitNcb5or as set forth in SEQ ID NO: 114, an Ncb5or from Drosophila grimshawi such as DgNcb5or as set forth in SEQ ID NO: 111, an Ncb5or from Cydia pomonella such as CpNcb5or as set forth in SEQ ID NO: 182, an Ncb5or from Agrotis segetum such as AseNcb5or as set forth in SEQ ID NO: 183, an Ncb5or from Bombus terrestris such as BterNcb5or as set forth in SEQ ID NO: 184, an Ncb5or from Lobesia botrana such as LboNcb5or as set forth in SEQ ID NO: 185, and an Ncb5or from Homo sapiens such as HsNcb5or as set forth in SEQ ID NO: 113,
   or variants thereof having at least 60% identity thereto. In some embodiments the FAR is FAR1 and the Ncb5or is DmNcb5or or SlitNcb5or; in other embodiments the FAR is FAR12 and the Ncb5or is DmNcb5or.

In other embodiments the cell expresses:
a. a desaturase from Lobesia botrana such as Desat30 as set forth in SEQ ID NO: 20;
b. a fatty acyl CoA reductase (FAR) from Helicoverpa armigera such as FAR1 as set forth in SEQ ID NO: 83; and
c. an NAD(P)H cytochrome b5 oxidoreductase (Nc5bor) selected from the group consisting of an Ncb5or from Drosophila melanogaster such as DmNcb5or as set forth in SEQ ID NO: 112, an Ncb5or from Spodoptera litura such as SlitNcb5or as set forth in SEQ ID NO: 114, an Ncb5or from Drosophila grimshawi such as DgNcb5or as set forth in SEQ ID NO: 111, an Ncb5or from Cydia pomonella such as CpNcb5or as set forth in SEQ ID NO: 182, an Ncb5or from Agrotis segetum such as AseNcb5or as set forth in SEQ ID NO: 183, an Ncb5or from Bombus terrestris such as BterNcb5or as set forth in SEQ ID NO: 184, an Ncb5or from Lobesia botrana such as LboNcb5or as set forth in SEQ ID NO: 185, and an Ncb5or from Homo sapiens such as HsNcb5or as set forth in SEQ ID NO: 113, preferably DmNcb5or or SlitNcb5or,
   or variants thereof having at least 60% identity thereto.

In other embodiments the cell expresses:
a. a desaturase from Drosophila virilis such as Desat61 as set forth in SEQ ID NO: 15;
b. a fatty acyl CoA reductase (FAR) from Helicoverpa armigera such as FAR1 as set forth in SEQ ID NO: 83; and
c. an NAD(P)H cytochrome b5 oxidoreductase (Nc5bor) selected from the group consisting of an Ncb5or from Drosophila melanogaster such as DmNcb5or as set forth in SEQ ID NO: 112, an Ncb5or from Spodoptera litura such as SlitNcb5or as set forth in SEQ ID NO: 114, an Ncb5or from Drosophila grimshawi such as DgNcb5or as set forth in SEQ ID NO: 111, an Ncb5or from Cydia pomonella such as CpNcb5or as set forth in SEQ ID NO: 182, an Ncb5or from Agrotis segetum such as AseNcb5or as set forth in SEQ ID NO: 183, an Ncb5or from Bombus terrestris such as BterNcb5or as set forth in SEQ ID NO: 184, an Ncb5or from Lobesia botrana such as LboNcb5or as set forth in SEQ ID NO: 185, and an Ncb5or from Homo sapiens such as HsNcb5or as set forth in SEQ ID NO: 113, preferably HsNcb5or or SlitNcb5or,
   or variants thereof having at least 60% identity thereto.

The term “variant thereof having at least 60% identity” in relation to a given enzyme shall be understood to refer to variants having 60% identity or more to said enzyme, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the enzyme, or more.

### Fatty Alcohols and Fatty Alcohol Acetates

The cell expressing said Ncb5or and said first enzyme or group of enzymes is capable of producing fatty alcohols and/or fatty alcohol acetates having a carbon chain length of 8, 9, 10, 11, 12, 13, 14, 15, 16, 17, 18, 19, 20, 21 or 22. In preferred embodiments, the carbon chain has a length of 11, 12, 13, 14, 15, 16, 17 or 18.

Thus, provided herein is a desaturated fatty alcohol, a saturated fatty alcohol, a desaturated fatty alcohol acetate, and/or a saturated fatty alcohol acetate obtainable according to the methods presented herein.

Further provided herein is the use of a desaturated fatty alcohol, a saturated fatty alcohol, a desaturated fatty alcohol acetate, and/or a saturated fatty alcohol acetate obtainable according to the methods presented herein. Such compounds can be used for example for monitoring the presence of pest or disrupting the presence of pest.

In some embodiments, the desaturated fatty alcohol is desaturated in at least one position, such as at least two positions. In other embodiments, the desaturated fatty alcohol is desaturated at position 3, 4, 5, 6, 7, 8, 9, 10, 11, 12, 13, 14, 15, 16, 17, 18, 19, 20 or 21. In preferred embodiments, the desaturated fatty alcohol is selected from the group of desaturated fatty alcohols consisting of (Z)-9-tetradecen-1-ol (Z9-14:OH), (Z)-9-hexadecen-1-ol (Z9-16:OH), (Z)-11-tetradecen-1-ol (Z11-14:OH), (Z)-11-hexadecen-1-ol (Z11-16:OH), and codlemone (E8,E10-dodecadien-1-ol).

In some embodiments, the desaturated fatty alcohol acetate is desaturated in at least one position, such as at least two positions. In other embodiments, the desaturated fatty alcohol acetate is desaturated at position 3, 4, 5, 6, 7, 8, 9, 10, 11, 12, 13, 14, 15, 16, 17, 18, 19, 20 or 21. In some embodiments the desaturated fatty alcohol acetate is E8,E10-dodecadienyl acetate.

In some embodiments, the cell is capable of further converting the desaturated fatty alcohol acetate or the saturated fatty alcohol acetate into a desaturated fatty aldehyde or a saturated fatty aldehyde, respectively, e.g. by expression of at least one alcohol dehydrogenase and/or at least one fatty alcohol oxidase in said cell. The alcohol dehydrogenase and/or at the fatty alcohol oxidase may be native to the cell, or may be a heterologous alcohol dehydrogenase and/or at the fatty alcohol oxidase.

Thus, further provided herein is the use of a desaturated fatty aldehyde, and/or a saturated fatty aldehyde obtainable according to the methods presented herein.

Alternatively, an acetyltransferase may catalyse the conversion of a desaturated fatty alcohol or a saturated fatty alcohol produced by the cell to the corresponding desaturated fatty aldehyde or saturated fatty aldehyde, either in vivo or in vitro after recovering the fatty alcohol. Conversion into a fatty aldehyde may also be done chemically.

Any of the desaturated fatty alcohols produced by the cell may further be converted into the corresponding fatty alcohol acetate. In preferred embodiments, (Z)-9-tetradecen-1-yl acetate (Z9-14:OAc), (Z)-9-hexadecen-1-yl acetate (Z9-16:OAc), (Z)-11-tetradecen-1-yl acetate (Z11-14:OAc), (Z)-11-hexadecen-1-yl acetate (Z11-16:OAc), and E8,E10-dodecadien-1-yl acetate (E8,E10-Z11:OAc).

In some embodiments, the product is (Z)-11-hexadecen-1-ol (Z11-16:OH). Such product can be obtained as disclosed in WO 2016/207339. For example, it may be produced by expressing a Δ11 desaturase and a FAR in a cell, such as a yeast cell or a plant cell, preferably a yeast cell, wherein said Δ11 desaturase is capable of converting hexadecanoyl-CoA to (Z)11-hexadecenoyl-CoA and said FAR is capable of converting (Z)11-hexadecenoyl-CoA to (Z)-11-hexadecen-1-ol. In particular, the following desaturases and FARs have been found to allow production of these compounds in yeast with a high titer: Amyelois transitella desaturases, such as Desat16; Spodoptera littoralis desaturases, such as Desat20; Spodoptera exigua desaturases, such as Desat37; Agrotis segetum desaturases, such as Desat19; Trichoplusia ni desaturases such as Desat21; Helicoverpa armigera FARs such as FAR1; Heliothhis subflexa FAR such as FAR4 and Helicoverpa assulta FARs such as FAR6; or variants thereof having at least 60% identity thereto.

In some embodiments, the product is codlemone (E8,E10-dodecadien-1-ol). Such product can be obtained as disclosed in application PCT/EP2020/086975 filed by same applicant on 18 Dec. 2020 and entitled “Yeast cells and methods for production of E8,E10-dodecadienyl coenzyme A, codlemone and derivatives thereof”. For example, E8,E10-dodecadien-1-ol can be produced in a cell, such as a yeast cell or a plant cell, preferably a yeast cell, said cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA). In particular, the following desaturases can be expressed in yeast in order to produce codlemone with a high titer: Desat4 alone, or Desat4 (SEQ ID NO: 9) and an additional heterologous desaturase such as Desat2 (SEQ ID NO: 10) or Desat1 (SEQ ID NO: 11) or variants thereof having at least 60% identity thereto. Said desaturases can advantageously be expressed with at least one of the following FARs: fatty acyl-CoA reductase is selected from the group consisting of FAR12 (SEQ ID NO: 77), FAR18 (SEQ ID NO: 78), FAR4 (SEQ ID NO: 85), FAR6 (SEQ ID NO: 84), FAR5 (SEQ ID NO: 86), FAR1 (SEQ ID NO: 83), FAR23 (SEQ ID NO: 82), or variants thereof having at least 60% identity thereto.

In order to improve production of desaturated fatty alcohols, saturated fatty alcohols, desaturated fatty alcohol acetates, saturated fatty alcohol acetates, desaturated fatty aldehydes, or saturated fatty aldehydes as disclosed herein, for example of E8,E10-dodecadienyl coenzyme A and optionally E8,E10-dodecadien-1-ol and derivatives thereof, it may be advantageous to introduce additional modifications in the cell in order to increase availability of the required precursors. Such modifications are disclosed in application PCT/EP2020/086975 filed by same applicant on 18 Dec. 2020 and entitled “Yeast cells and methods for production of E8,E10-dodecadienyl coenzyme A, codlemone and derivatives thereof”, and can be useful for production of a variety of desaturated compounds besides codlemone. For example, the cell may be further modified to express a heterologous cytochrome b5; and/or to express a heterologous cytochrome b5 reductase; and/or to express a haemoglobin; and/or by inactivation of native elongase(s); and/or by inactivation of native thioesterases; and/or by inactivation or modification of activity of native fatty aldehyde dehydrogenases, fatty alcohol oxidases, peroxisome biogenesis factor and/or fatty acyl synthases; and/or by expression of a heterologous thioesterase gene; and/or by expression of a fusion protein of fatty acyl synthase and of a thioesterase. In particular, expression of a heterologous cytochrome b5, of a heterologous cytochrome b5 reductase, and/or of a haemoglobin, and/or inactivation or reduction of activity of native fatty aldehyde dehydrogenases, fatty alcohol oxidase, peroxisome biogenesis facts and/or fatty acyl synthases are particularly relevant and are described in detail in application PCT/EP2020/086975.

In some embodiments, particularly embodiments where the cell is a yeast cell, the cell is further modified in order to increase availability of fatty acyl-CoAs of a given chain length by chain shortening, as disclosed in WO 2020/169389. Without being bound by theory, such modifications are expected to increase availability of substrates having a desired carbon chain length, in particular having a carbon chain length of 12, whereby production of desaturated fatty alcohols and optionally of desaturated fatty alcohol acetates and desaturated fatty aldehydes can be increased. This can achieved by reducing the activity of native acyl-CoA oxidases in a microbial production cell and by expressing specific acyl-CoA oxidases, desaturases, reductases, and acetyltransferases. Such modifications are described in detail in WO 2020/169389.

Any of the above modifications can be combined, i.e. the cell may comprise several of said modifications.

### Fatty Aldehydes

While the present disclosure provides methods for producing desaturated and saturated fatty alcohols, and desaturated and saturated fatty alcohol acetates, it may be of interest to further convert said fatty alcohols to the corresponding desaturated or saturated aldehydes. Thus in some embodiments, the method may further comprise the step of converting at least part of the fatty alcohols to fatty aldehydes, thereby producing fatty aldehydes. This can be achieved by chemical methods or by further engineering of the yeast cell.

In some embodiments, the step of converting at least part of the fatty alcohols to the corresponding aldehydes is a step of chemical conversion. The chemical conversion is based on the oxidation of fatty alcohols to the corresponding aldehydes. Methods for performing this conversion are known in the art. Preferred methods are environmentally friendly and minimize the amount of hazardous waste.

Thus in some embodiments, the chemical conversion may be metal free, avoiding toxic heavy metal based reagents such as manganese oxides, chromium oxides (Jones ox. PDC, PCC) or ruthenium compounds (TPAP, Ley-Griffith ox.). In some embodiments, the conversion does not involve reactions with activated dimethyl sulfoxide such as the Swern oxidation or the Pfitzner-Moffat type. Such reactions may involve the stereotypic formation of traces of intensively smelling organic sulfur compounds such as dimethyl sulfide which can be difficult to remove from the target product.

In some embodiments, the method comprises a Dess-Martin reaction (Yadav et al., 2004, Meyer et al., 1994). In some embodiments, the method comprises a Copper(I)/ABNO-catalysed aerobic alcohol oxidation reaction (Steves & Stahl, 2013).

In other embodiments, the chemical conversion comprises the oxidation with sodium hypochlorite under aqueous/organic two phase conditions (Okada et al., 2014; Tamura et al., 2012; Li et al., 2009). In some embodiments, the chemical oxidation can be performed with 1-chlorobenzotriazole in a medium of methylene chloride containing 25% pyridine (Ferrell and Yao, 1972).

Alternatively, the oxidation of a fatty alcohol to the corresponding fatty aldehyde can be performed enzymatically by alcohol dehydrogenases. The skilled person will know how to carry out enzymatic oxidation. For example, enzymatic oxidation can be carried out by contacting purified enzymes, cell extracts or whole cells, with the fatty alcohol.

The fatty alcohols obtainable by the cells and methods described herein can be further converted to fatty aldehydes by introducing a gene encoding an aldehyde-forming fatty acyl-CoA reductase EC 1.2.1.50 (FAR′). In this way, at least part of the fatty acyl-CoA can be converted to the corresponding fatty aldehyde by an aldehyde-forming fatty acyl-CoA reductase (FAR′). The enzymes capable of catalysing this conversion can catalyse a reduction reaction, where the fatty acyl-CoA is reduced to a fatty aldehyde.

Such enzymes are aldehyde-forming fatty acyl-CoA reductases, herein also referred to as FAR′ or “aldehyde-forming FAR′”, with an EC number 1.2.1.50. They catalyse the following reaction:

$$Fatty acyl - CoA + NADPH = fatty aldehyde + NADP + + coenzyme A .$$

### Yeast Cell

In some embodiments of the present invention, the cell provided herein is a yeast cell. Accordingly, one embodiment of the present invention provides a yeast cell expressing a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous NAD(P)H cytochrome b5 oxidoreductase (Ncb5or); whereby the yeast cell is capable of producing the compound with a higher titer compared to a yeast cell expressing the first group of enzymes but no heterologous Ncb5or when cultivated in the same conditions.

The yeast cell may be a non-naturally occurring yeast cell, for example a yeast cell which has been engineered to produce desaturated fatty alcohols, saturated fatty alcohols, desaturated fatty alcohol acetates, saturated fatty alcohol acetates, and a desaturated fatty acyl-CoAs.

In some embodiments, the cell has been modified at the genomic level, e.g. by gene editing in the genome. The cell may also be modified by insertion of at least one nucleic acid construct such as at least one vector. The vector may be designed as is known to the skilled person to either enable integration of nucleic acid sequences in the genome, or to enable expression of a polypeptide encoded by a nucleic acid sequence comprised in the vector without genome integration.

The yeast cell may be of a genus selected from Saccharomyces, Pichia, Komagataella, Yarrowia, Kluyveromyces, Candida, Rhodotorula, Rhodosporidium, Cryptococcus, Trichosporon and Lipomyces. In a preferred embodiment, the genus is Saccharomyces or Yarrowia, most preferably the genus is Yarrowia.

The yeast cell may be of a species selected from Saccharomyces cerevisiae, Saccharomyces boulardi, Pichia pastoris, Komagataella phaffi, Komagataella pastoris, Komagataella pseudopastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the yeast cell is a Saccharomyces cerevisiae cell or a Yarrowia lipolytica cell, most preferably the yeast cell is a Yarrowia lipolytica cell.

The yeast cell to be modified, which will also be referred to as the host cell, may express native enzymes which are of the same class as the enzymes which are necessary for the production of desaturated fatty alcohols, saturated fatty alcohols, desaturated fatty alcohol acetates, saturated fatty alcohol acetates, and a desaturated fatty acyl-CoAs. In some cases, however, such native enzymes may have a negative impact on the titer of desaturated fatty alcohols, saturated fatty alcohols, desaturated fatty alcohol acetates, saturated fatty alcohol acetates, and a desaturated fatty acyl-CoAs which can be obtained; the native enzymes may thus be inactivated by methods known in the art, such as gene editing. For example, the genes encoding the native enzymes having a negative impact on the titer may be deleted or mutated so as to lead to total or partial loss of activity of the native enzyme.

In some embodiments, the yeast cell has reduced activity of one or proteins as disclosed in WO 2018/109163 and in European patent 3555268. For example, the yeast cell may have a mutation resulting in reduced activity (i.e. downregulation) of Pex10, Hfd1, Hfd4, Fao1 and/or GPAT. Preferably, the yeast cell has at least one mutation resulting in reduced activity of at least Fao1 and one or more of Hfd1, Hfd4, Pex10 and/or GPAT. Such mutations may increase the production of desaturated fatty alcohol and/or desaturated fatty alcohol acetate in a yeast cell expressing a heterologous desaturase and a heterologous fatty acyl-CoA reductase.

Thus, provided here is a yeast cell expressing a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous NAD(P)H cytochrome b5 oxidoreductase (Ncb5or); whereby the yeast cell is capable of producing the compound with a higher titer compared to a yeast cell expressing the first group of enzymes but no heterologous Ncb5or when cultivated in the same conditions.

The first enzyme or group of enzymes may be a desaturase as defined in the section “Desaturase”; a FAR as defined in the section “Fatty acyl-CoA reductase”; a desaturase and a FAR desaturase as defined in the sections “Desaturase” and “Fatty acyl-CoA reductase”, respectively. The Ncb5or may be as defined in the section NAD(P)H cytochrome b5 oxidoreductase”. The yeast cell may express a heterologous Ncb5or and a heterologous desaturase; a heterologous FAR; or a heterologous FAR and a heterologous desaturase as disclosed in the section “Cell”.

In some embodiments, the genes encoding said desaturase, FAR and/or Ncb5or has been codon optimized for said yeast cell. In other embodiments, the genes encoding said desaturase, FAR and/or Ncb5or are under control of an inducible promoter. In some embodiments, the genes encoding said desaturase, FAR and/or Ncb5or are present in high copy number, and/or they are each independently comprised within the genome of the yeast cell or within a vector comprised in the yeast cell.

In some embodiments, the yeast cell comprises a system of vectors, as described in the section “Nucleic acid”.

The yeast cell according to the present invention may be comprised in a fermentation broth, a fermentation system, and/or a catalytic system. In other words, a fermentation broth, a fermentation system and/or a catalytic system may comprise the yeast cell according to the present invention.

### Plant Cell

In some embodiments of the present invention, the cell provided herein is a plant cell. Accordingly, one embodiment of the present invention provides a plant cell expressing a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous NAD(P)H cytochrome b5 oxidoreductase (Ncb5or); whereby the plant cell is capable of producing the compound with a higher titer compared to a plant cell expressing the first group of enzymes but no heterologous Ncb5or when cultivated in the same conditions.

The plant cell may be a non-naturally occurring plant cell, for example a plant cell which has been engineered to produce desaturated fatty alcohols, saturated fatty alcohols, desaturated fatty alcohol acetates, saturated fatty alcohol acetates, and a desaturated fatty acyl-CoAs.

In some embodiments, the plant cell has been modified at the genomic level, e.g. by gene editing in the genome. The plant cell may also be modified by insertion of at least one nucleic acid construct such as at least one vector. The vector may be designed as is known to the skilled person to either enable integration of nucleic acid sequences in the genome, or to enable expression of a polypeptide encoded by a nucleic acid sequence comprised in the vector without genome integration. For example, the plant cell may be modified using horizontal gene transfer, a gene gun, and/or other techniques known in the art.

In some embodiments, the plant cell is from a genus selected from the group consisting of Nicotiana and Camelina. In some embodiments, the plant cell is from a species selected from the group consisting of Nicotiana tabacum, Nicotiana benthamiana, and Camelina sativa.

The plant cell to be modified, which will also be referred to as the host cell, may express native enzymes which are of the same class as the enzymes which are necessary for the production of desaturated fatty alcohols, saturated fatty alcohols, desaturated fatty alcohol acetates, saturated fatty alcohol acetates, and a desaturated fatty acyl-CoAs. In some cases, however, such native enzymes may have a negative impact on the titer of desaturated fatty alcohols, saturated fatty alcohols, desaturated fatty alcohol acetates, saturated fatty alcohol acetates, and a desaturated fatty acyl-CoAs which can be obtained; the native enzymes may thus be inactivated by methods known in the art, such as gene editing. For example, the genes encoding the native enzymes having a negative impact on the titer may be deleted or mutated so as to lead to total or partial loss of activity of the native enzyme.

The plant cell may be part of a plant, such as a genetically modified plant. In other words, the plant cell may be part of a transgenic plant. Transgenic plants are plants expressing a transgene, i.e. plants which have been genetically engineered. Plant genomes can be engineered by physical methods or by use of Agrobacterium for the delivery of sequences.

A gene gun, or a biolistic particle delivery system, is an example of a physical plant genetic engineering method. The gene gun may be used to deliver exogenous DNA, RNA or protein to plant cells. By coating particles with a gene of interest and firing these micro-projectiles into cells using mechanical force, an integration of desired genetic information can be induced into cells. The gene gun is able to transform almost any type of plant cell and is not limited to transformation of the nucleus; it can also transform organelles including plastids and mitochondria.

Agrobacterium is a genus of Gram-negative bacteria that uses horizontal gene transfer to cause tumours in plants. Agrobacterium well known for its ability to transfer DNA between itself and plants, and has for this reason become an important tool for genetic engineering of plants. Genomes of plants can be engineered by the use of Agrobacterium for the delivery of sequences hosted in transfer-binary vectors (T-binary vectors). The transformation with Agrobacterium can be achieved in multiple ways. Protoplasts or leaf-discs can be incubated with the Agrobacterium, and whole plants generated as described below. In agroinfiltration, the Agrobacterium may be injected directly into the leaf tissue of a plant.

Many plants are pluripotent, meaning that a single cell from a mature plant can be harvested and used to form a new plant. This can be utilized when making transgenic plants. Cells which have been successfully transformed in an adult plant can be harvested and grown in plant tissue culture to generate a new plant, where the genetic modification is present in every cell of said plant.

The plant of the present disclosure is not a naturally occurring plant. Preferably, the plant disclosed herein is not obtained by essentially biological methods, but is generally obtained by targeted genome editing methods, as is known in the art.

Thus, provided here is a plant cell expressing a first enzyme or group of enzymes capable of converting a fatty acyl-CoA to a compound selected from a desaturated fatty alcohol, a saturated fatty alcohol, a desaturated fatty alcohol acetate, and a desaturated fatty acyl-CoA; and a heterologous NAD(P)H cytochrome b5 oxidoreductase (Ncb5or); whereby the plant cell is capable of producing the compound with a higher titer compared to a plant cell expressing the first group of enzymes but no heterologous Ncb5or when cultivated in the same conditions.

The first enzyme or group of enzymes may be a desaturase as defined in the section “Desaturase”; a FAR as defined in the section “Fatty acyl-CoA reductase”; a desaturase and a FAR desaturase as defined in the sections “Desaturase” and “Fatty acyl-CoA reductase”, respectively. The Ncb5or may be as defined in the section NAD(P)H cytochrome b5 oxidoreductase”. The plant cell may express a heterologous Ncb5or and a heterologous desaturase; a heterologous FAR; or a heterologous FAR and a heterologous desaturase as disclosed in the section “Cell”.

In some embodiments, the genes encoding said desaturase, FAR and/or Ncb5or has been codon optimized for said plant cell. In other embodiments, the genes encoding said desaturase, FAR and/or Ncb5or are under control of an inducible promoter. In some embodiments, the genes encoding said desaturase, FAR and/or Ncb5or are present in high copy number, and/or they are each independently comprised within the genome of the plant cell or within a vector comprised in the plant cell.

In some embodiments, the plant cell comprises a system of vectors, as described in the section “Nucleic acid”.

Method for Production of Desaturated Fatty Acyl CoAs, Desaturated Fatty Alcohols, Saturated Fatty Alcohols, Desaturated Fatty Alcohol Acetates, and/or Saturated Fatty Alcohol Acetates

Provided herein is a method for production of a compound selected from a desaturated fatty alcohol, a saturated fatty alcohol, a desaturated fatty alcohol acetate, and a desaturated fatty acyl-CoA in a cell, said method comprising the steps of:
a. providing a cell and incubating said cell in a medium; and
b. expressing in said cell a first enzyme or group of enzymes capable of converting a fatty acyl-CoA to said compound, thereby converting at least part of said fatty acyl-CoA to said compound; and
c. expressing in said cell an NAD(P)H cytochrome b5 oxidoreductase (Ncb5or);
d. optionally, recovering said compound.

In some embodiments, the methods presented herein yields said desaturated fatty alcohol, and optionally said fatty alcohol acetate and/or fatty acid with a titer of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, such as at least 55 g/L, such as at least 60 g/L, such as at least 65 g/L, such as at least 70 g/L, such as at least 75 g/L, such as at least 80 g/L, such as at least 85 g/L, such as at least 90 g/L, such as at least 95 g/L, such as at least 100 g/L, such as at least 125 g/L, such as at least 150 g/L, such as at least 175 g/L, such as at least 200 g/L, or more.

Further provided herein is a method of increasing the titer of a compound selected from a desaturated fatty alcohol, a saturated fatty alcohol, a desaturated fatty alcohol acetate, a desaturated fatty acid and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an NAD(P)H cytochrome b5 reductase (Ncb5or), thereby increasing the titer of said compound compared to the titer from a cell not expressing said Ncb5or in the same conditions;
c. optionally, recovering said compound.

In other words, provided herein is a method to increase the production of a compound selected from a desaturated fatty alcohol, a saturated fatty alcohol, a desaturated fatty alcohol acetate, a desaturated fatty acid and a desaturated fatty acyl-CoA in a cell, wherein said cell expresses a first enzyme or group of enzymes capable of converting a fatty acyl-CoA to any of said compounds, thereby converting at least part of said fatty acyl-CoA to any of said compounds; and expressing in said cell an NAD(P)H cytochrome b5 reductase (Ncb5or); wherein the production of said compound in said cell is increased compared to the production of said compound in said cell which expresses the same enzyme or first group of enzymes, but not an Ncb5or, wherein said cells are cultivated under the same conditions.

Also disclosed herein is a method for increasing the purity of a compound selected from a desaturated fatty alcohol, a desaturated fatty acid and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the production of said compound compared to the production from a cell not expressing said Ncb5or in the same conditions;
   wherein the purity of said compound is the ratio or percentage of said compound in relation to all compounds within the same compound group produced by the cell, such as the percentage of said desaturated fatty alcohol in relation to all desaturated fatty alcohols produced by the cell, such as the percentage of desaturated fatty acid in relation to all fatty acids produced by the cell, and/or such as the percentage of desaturated fatty acyl-CoA in relation to all fatty acyl-CoA produced by the cell.

The purity of desaturated fatty alcohol, desaturated fatty acid and/or desaturated fatty acyl-CoA may be increased by at least 3% compared to the purity of the same compound obtained from a cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

In one embodiment, the first enzyme or group of enzymes consists of one or more desaturase capable of converting a fatty acyl-CoA to a desaturated fatty acyl-CoA and wherein said compound is a desaturated fatty acyl-CoA. Fatty acyl-CoAs do not accumulate in the cell but their presence can be determined by determining the presence of the corresponding fatty acid.

In one embodiment, the first enzyme or group of enzymes consists of one or more fatty acyl reductase (FAR) capable of converting a fatty acyl-CoA to a saturated fatty alcohol and wherein said compound is a saturated fatty alcohol.

In one embodiment, the first enzyme or group of enzymes consists of one or more fatty acyl reductase (FAR) and one or more desaturase capable of converting a fatty acyl-CoA to a desaturated fatty alcohol and wherein said compound is a desaturated fatty alcohol.

In one embodiment, the compound is a desaturated or a saturated fatty alcohol and wherein the method further comprises the step of converting the desaturated or the saturated fatty alcohol to a desaturated or a saturated fatty alcohol acetate, respectively.

In one embodiment, the conversion of the desaturated or the saturated fatty alcohol to a desaturated or a saturated fatty alcohol acetate is performed in vitro.

In one embodiment, the conversion of the desaturated or the saturated fatty alcohol to a desaturated or a saturated fatty alcohol acetate is performed in vivo by further expressing in the cell an acetyltransferase capable of converting the desaturated or the saturated fatty alcohol to a desaturated or a saturated fatty alcohol acetate, respectively.

In one embodiment, compound is a saturated fatty alcohol or a desaturated fatty alcohol, and wherein the method further comprises a step of converting the saturated fatty alcohol or the desaturated fatty alcohol to a saturated fatty aldehyde or to a desaturated fatty aldehyde, respectively.

In one embodiment, the conversion to an aldehyde is a chemical or an enzymatic conversion.

In some embodiments, the cell is a yeast cell as described in the section “Yeast cell”. In some embodiments, the cell is a plant cell as described in the section “Plant cell”.

The desaturase, FAR and Ncb5or may be as described in the sections “Desaturase”, “Fatty acyl-CoA reductase” and “NAD(P)H cytochrome b5 oxidoreductase”, respectively.

In some embodiments, the total titer of desaturated fatty alcohols and optionally the total titer of desaturated fatty alcohol acetates and/or of desaturated fatty acids is increased. In other embodiments, the total titer of saturated fatty alcohols and optionally the total titer of saturated fatty alcohol acetates and/or of desaturated fatty acids is increased.

In some embodiments, the titer of desaturated fatty alcohol is increased at least 3% compared to the titer from a cell such as a yeast cell or a plant cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

In some embodiments, the titer of saturated fatty alcohol acetate is increased at least 3% compared to the titer from a cell such as a yeast cell or a plant cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

In some embodiments, the titer of saturated fatty alcohol acid is increased at least 3% compared to the titer from a cell such as a yeast cell or a plant cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

Further provided herein is a method for producing at least 1 mg/L of desaturated fatty alcohol, saturated fatty alcohol, desaturated fatty alcohol acetate, saturated fatty alcohol acetate, saturated fatty acid, desaturated fatty acid, desaturated fatty aldehyde, and/or saturated fatty aldehyde, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

Methods for determining the titer are known in the art.

Any of the methods disclosed herein may further comprise a step of converting the desaturated or the saturated fatty alcohol acetate into a desaturated or a saturated fatty aldehyde. This can be done as detailed above, e.g. by expression of at least one alcohol dehydrogenase and/or at least one fatty alcohol oxidase in said cell. The alcohol dehydrogenase and/or at the fatty alcohol oxidase may be native to the cell, or may be a heterologous alcohol dehydrogenase and/or at the fatty alcohol oxidase.

Any of the methods disclosed herein may further comprise a step of converting the desaturated or saturated fatty alcohol produced by the cell to the corresponding saturated or desaturated fatty aldehyde, either in vivo or in vitro after recovering the fatty alcohol. Conversion into a desaturated or saturated fatty aldehyde may also be done chemically.

In some embodiments, the cell such as a yeast cell or a plant cell is further modified in order to increase availability of fatty acyl-CoAs of a given chain length by chain shortening, as disclosed in WO 2020/169389. Without being bound by theory, such modifications are expected to increase availability of substrates having a desired carbon chain length, in particular having a carbon chain length of 12, whereby production of desaturated fatty alcohols and optionally of desaturated fatty alcohol acetates and desaturated fatty aldehydes can be increased. This can be achieved by reducing the activity of native acyl-CoA oxidases in a microbial production cell and by expressing specific acyl-CoA oxidases, desaturases, reductases, and acetyltransferases. Such modifications are described in detail in WO 2020/169389.

Other relevant modifications are available to the skilled person, some of which are detailed herein above in the section “Fatty alcohols and fatty alcohol acetates”.

Methods for Increasing the Activity of a Desaturase and/or a Fatty Acyl-CoA Reductase

Provided herein is a method for increasing the activity of at least one enzyme selected from the group consisting of desaturases and fatty acyl CoA reductases (FAR), said method comprising the steps of:
a. providing a desaturase capable of introducing at least one double bond in a fatty acyl-CoA, thereby converting at least part of said fatty acyl-CoA to a desaturated fatty-acyl-CoA; and/or
b. providing a fatty acyl CoA reductase (FAR) capable of converting at least part of said desaturated fatty acyl-CoA to a desaturated fatty alcohol, thereby producing said desaturated fatty alcohol; and
c. contacting said desaturase and/or FAR with an NAD(P)H cytochrome b5 oxidoreductase (Ncb5or), thereby increasing the activity of said desaturase and/or FAR compared to the activity of said desaturase and/or FAR in the absence of said Ncb5or, wherein the activity is measured under the same conditions;
   wherein the increase in activity is measured by measuring the concentration of product formed by the desaturase and/or the FAR.

In some embodiments, the method is performed in vitro. In other embodiments, the method is performed in vivo. In some embodiments, the method is performed in a yeast cell, for example as described herein in the section “Yeast cell” or in a plant cell as described herein in the section “Plant cell”.

The method may increase the concentration of a specific desaturated fatty alcohol, a specific saturated fatty alcohol, a specific desaturated fatty alcohol acetate and/or a specific desaturated fatty acid, a specific desaturated fatty alcohol acetate, and/or the total concentration of all desaturated fatty alcohols and/or all desaturated fatty alcohol acetates, and/or the total concentration of all saturated fatty alcohols and/or all saturated fatty alcohol acetates.

Any of the methods disclosed herein may further comprise a step of converting the saturated fatty alcohol or the desaturated fatty alcohol to a saturated fatty aldehyde or to a desaturated fatty aldehyde, respectively. This can be done as detailed above, e.g. by expression of at least one alcohol dehydrogenase and/or at least one fatty alcohol oxidase in said cell. The alcohol dehydrogenase and/or the fatty alcohol oxidase may be native to the cell, or may be a heterologous alcohol dehydrogenase and/or at the fatty alcohol oxidase.

Any of the methods disclosed herein may further comprise a step of converting the desaturated or saturated fatty alcohol produced by the cell to the corresponding desaturated or saturated fatty aldehyde, either in vivo or in vitro after recovering the fatty alcohol. Conversion into a desaturated or saturated fatty aldehyde may also be done chemically.

In some embodiments, concentration is increased at least 3% compared to the concentration in an assay performed in the absence of Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

Methods to determine whether an Ncb5or increases the activity of a desaturase and/or a FAR are known in the art. For example, whether a given Ncb5or increases the activity of a desaturase can be determined by incubating in a suitable solution (i) said desaturase, said Ncb5or and the fatty acyl-CoA substrate for said desaturase, and (ii) said desaturase and the fatty acyl-CoA substrate for said desaturase. After 1 hour of incubation under the same conditions, the amount (concentration) of product, i.e. the amount of desaturated fatty-acyl CoA generated by the desaturase, can be determined by GC-MS. If a higher concentration of products is obtained from the solution comprising both the desaturase and the Ncb5or, it shows that said Ncb5or increases the activity of said desaturase.

Accordingly, herein is also provided the use of an enzyme having Ncb5or activity to increase the activity of a different enzyme or group of enzymes, such as membrane-bound enzyme or group of enzymes.

### Recovery

It may be desirable to recover the products obtained by the methods disclosed herein. Thus the present methods may comprise a further step of recovering the desaturated fatty alcohol, the saturated fatty alcohol, the desaturated fatty alcohol acetate, the saturated fatty alcohol acetate, the desaturated fatty aldehyde, and/or the saturated fatty aldehyde produced by the present yeast cell.

In some embodiments, the method comprises a step of recovering the desaturated fatty alcohols and/or the saturated fatty alcohols. In other embodiments, the method comprises a step of recovering the desaturated fatty alcohol acetates and/or the saturated fatty alcohol acetates.

Methods for recovering the products obtained by the present invention are known in the art and may comprise an extraction with a hydrophobic solvent such as decane, hexane or a vegetable oil.

The recovered products may be modified further, for example may the desaturated fatty alcohols and/or the saturated fatty alcohols be converted to the corresponding desaturated fatty aldehydes and/or the saturated fatty aldehydes as described herein above. In embodiments where desaturated fatty aldehydes and/or saturated fatty aldehydes are directly produced in the culture medium, e.g. in vivo or by contacting the cells with the relevant enzymes, the desaturated fatty aldehydes and/or the saturated fatty aldehydes may also be recovered.

As described in application PCT/EP2020/076351 filed by same applicant on 22 Sep. 2020 and entitled “Improved methods for production, recovery and secretion of hydrophobic compounds in a fermentation”, when a fermentation system is used to cultivate the cell, in particular a yeast cell, which is capable of producing desaturated fatty alcohols, desaturated fatty alcohol acetates and/or desaturated fatty aldehydes, the addition of an extractant to the culture medium may further increase titers and extracellular secretion. In some embodiments, the medium comprises an extractant in an amount equal to or greater than its cloud concentration in an aqueous solution, wherein the extractant a non-ionic surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone and ethoxylated and propoxylated C₁₆-C₁₈ alcohol-based antifoaming agents and combinations thereof. In some embodiments:
the non-ionic surfactant is an ethoxylated and propoxylated C₁₆-C₁₈ alcohol-based antifoaming agent, such as C₁₆-C₁₈ alkyl alcohol ethoxylate propoxylate (CAS number 68002-96-0), and wherein the culture medium comprises at least 1% vol/vol of C₁₆-C₁₈ alkyl alcohol ethoxylate propoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol C₁₆-C₁₈ alkyl alcohol ethoxylate propoxylate, or more,
the non-ionic surfactant is a polyethylene polypropylene glycol, for example Kollliphor® P407 (CAS number 9003-11-6), and wherein the culture medium comprises at least 10% vol/vol of polyethylene polypropylene glycol such as Kolliphor® P407, such as at least 11% vol/vol, such as at least 12% vol/vol, such as at least 13% vol/vol, such as at least 14% vol/vol, such as at least 15% vol/vol, such as at least 16% vol/vol, such as at least 17% vol/vol, such as at least 18% vol/vol, such as at least 19% vol/vol, such as at least 20% vol/vol, such as at least 25% vol/vol, such as at least 30% vol/vol, such as at least 35% vol/vol of polyethylene polypropylene glycol such as Kolliphor® P407, or more,
the non-ionic surfactant is a mixture of polyether dispersions, such as antifoam 204, and wherein the culture medium comprises at least 1% vol/vol of a mixture of polyether dispersions such as antifoam 204, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol of a mixture of polyether dispersions such as antifoam 204, or more; and/or
the non-ionic surfactant is a non-ionic surfactant comprising polyethylene glycol monostearate such as simethicone, and wherein the culture medium comprises at least 1% vol/vol of polyethylene glycol monostearate or simethicone, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol polyethylene glycol monostearate or simethicone, or more.

In other embodiments, the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.

The recovered products, i.e. the desaturated fatty alcohols, the saturated fatty alcohols, the desaturated fatty alcohol acetates, and/or the saturated fatty alcohol acetates, may also be formulated into a pheromone composition, such as described in the section “Pheromone composition”. The composition may further comprise one or more additional compounds such as a liquid or solid carrier or substrate. Fatty aldehydes obtained from said fatty alcohols may also be comprised in such compositions.

Fatty acids can be recovered from plants by methods known in the art, e.g. after homogenisation of the leaves and recovery of the lipids by methods known in the art. The recovered lipids are hydrolyzed into free fatty acids and esterified to fatty acid alkyl ester, followed by a reduction to either fatty alcohols or fatty aldehydes.

### Nucleic Acid

It will be understood that throughout the present invention, the term ‘nucleic acid encoding an activity’ shall refer to a nucleic acid molecule capable of encoding a peptide, a protein or a fragment thereof having said activity. Such nucleic acid molecules may be open reading frames or genes or fragments thereof. A nucleic acid construct may also be a group of nucleic acid molecules, which together may encode several peptides, proteins or fragments thereof having an activity of interest. The term ‘activity’ or ‘activity of interest’ may in particular refer to one of the following activities: a desaturase as described herein, a fatty acyl-CoA reductase as described herein, and/or an NAD(P)H cytochrome b5 reductase (Ncb5or) as described herein. The nature of the one or more activity of interest will depend on the nature of the desired product one wishes to obtain with the present methods.

A system of nucleic acid constructs comprising nucleic acids encoding an NAD(P)H cytochrome b5 oxidoreductase (Ncb5or) and:
a. a desaturase capable of introducing at least one double bond in a fatty acyl-CoA; and/or
b. a fatty acyl CoA reductase (FAR) capable of converting at least part of a desaturated fatty acyl-CoA to a desaturated fatty alcohol.

In some embodiments, the desaturase is encoded by any one of the sequences set forth in SEQ ID NO: 39 to 76 and SEQ ID NOs: 140 to 153, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

In some embodiments, the fatty acyl CoA reductase (FAR) is encoded by any one of the sequences set forth in SEQ ID NO: 94 to 110 and SEQ ID NOs: 168 to 181, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

In some embodiments, Ncb5or is encoded by any one of the sequences set forth in SEQ ID NO: 115 to 118, SEQ ID NO: 125 and SEQ ID NOs: 186 to 189, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

The system disclosed herein may further comprise all elements required for expression of nucleic acids in cell, such as in a yeast cell or a plant. Such elements include but are not limited to introns and regulatory elements such as promoters, terminators, 5′UTRs, enhancers and silencers.

### Kit

Provided herein is a kit of parts for performing the present methods. The kit of parts may comprise an organism “ready to use” as described herein. In one embodiment, the organism is a yeast cell as described in the section “Yeast cell”. In one embodiments, the yeast cell is a Yarrowia cell, such as a Yarrowia lipolytica cell. In one embodiment, the organism is a plant cell as described in the section “Plant cell”. In one embodiment the plant cell is a tobacco plant cell.

In one embodiment, the kit of parts comprises a nucleic acid construct encoding the activities of interest to be introduced in the organism, such as the system of nucleic acids described in the section “Nucleic acid” herein. The nucleic acid construct may be provided as a plurality of nucleic acid constructs, such as a plurality of vectors, wherein each vector encodes one or several of the desired activities.

The kit of parts may optionally comprise the cell to be modified.

The kit of parts may also comprise instructions for use.

In some embodiments, the kit of parts comprises all or a combination of the above.

### Pheromone Composition

The present invention thus provides compounds, in particular desaturated fatty alcohols, saturated fatty alcohols, desaturated fatty alcohol acetates, and saturated fatty alcohol acetates, as well as derivatives thereof such as desaturated fatty aldehydes and saturated fatty aldehydes, and their use. In particular, the desaturated compounds obtainable using the present cells and methods are useful as components of pheromone compositions. Such pheromone compositions may be useful for integrated pest management. They can be used as is known in the art for e.g. mating disruption.

Thus, the desaturated fatty alcohols, the desaturated fatty alcohol acetate, and the desaturated fatty aldehydes obtainable by the present methods or using the present cells, such as yeast cell or plant cell, may be formulated in a pheromone composition.

Such pheromone compositions may be used as integrated pest management products, which can be used in a method of monitoring the presence of pest or in a method of disrupting the mating of pest.

Thus, provided herein is a method for monitoring the presence of pest or disrupting the mating of pest, said method comprising the steps of:
a. producing a desaturated fatty alcohol, a desaturated fatty alcohol acetate, and/or a desaturated fatty aldehyde according to any of the methods disclosed herein; and
b. formulating said fatty alcohol, fatty alcohol acetate, and/or fatty aldehyde as a pheromone composition; and
c. employing said pheromone composition as an integrated pest management composition.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. In one embodiment, said compositions are placed in a device, such as a pheromone dispenser, which diffuses the pheromone composition. The dispenser may for example release pheromones at a constant, pre-adjustable, rate. This can result in mating disruption, thereby preventing pest reproduction, or it can be used in combination with a trapping device to entrap the pests. Non-limiting examples of pests against which the present pheromone compositions can be used are: cotton bollworm (Helicoverpa armigera), striped stemborer (Chilo suppressalis), diamond back moth (Plutella xylostella), cabbage moth (Mamestra brassicae), large cabbage-heart caterpillar (Crocidolomia binotalis), European corn stalk borer (Sesamia nonagrioides), currant clearwing (Synanthedon tipuliformis) and artichoke plume moth (Platyptilia carduidactylal). Accordingly, use of the present compositions on a culture can lead to increased crop yield, with substantially no environmental impact.

The relative amounts of fatty alcohols and fatty alcohol acetates in the present pheromone compositions may vary depending on the nature of the crop and/or of the pest to be controlled; geographical variations may also exist. Determining the optimal relative amounts may thus require routine optimisation. The pheromone compositions may also comprise fatty aldehydes.

Examples of compositions used as repellents can be found in Kehat & Dunkelblum (1993) for H. armigera; in Alfaro et al. (2009) for C. suppressalis; in Eizaguirre et al. (2002) for S. nonagrioides; in Wu et al. (2012) for P. xylostella; and in Bari et al. (2003) for P. carduidactyla.

In some embodiments, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be in the form of a solution, a gel, a powder. The pheromone composition may be formulated so that it can be easily dispensed, as is known in the art.

The present mating disruption methods may be employed in fields of transgenic crops.

Also provided herein is a method for reducing or delaying the emergence of resistance to a pesticidal trait; the method may be an integrated resistance management method. Thus are disclosed preemptive and responsive methods to delay the development of resistance in a pest such as an insect, for example any of the insects listed herein, to a transgenic insecticidal crop and/or to chemical insecticide, i.e. a preemptive strategy. Also disclosed are methods to rescue one or more pest's susceptibility to transgenic insecticidal crops and/or chemical insecticides once resistance has developed, i.e. a responsive strategy. In some embodiments, the method comprises the application of a pheromone composition, such as obtained by the methods disclosed herein, to an agricultural area comprising a field population, wherein transgenic crops comprising one or more insecticidal traits such as transgenic insecticidal trais active against one of the insects listed herein, and optionally a refuge comprising crops devoid of insecticidal traits, to disrupt mating of the pest, thereby delaying the emergence of resistance to the insecticidal trait. The present compositions may thus be used in combination with any of the methods described in WO 2017/112887.

Also provided herein is a method for preventing or reducing crop damage from a pest such as an insect as listed herein. Such methods comprise applying mating disruption to a field by applying a pheromone composition as disclosed herein, and disrupting the expression of one or more target genes in one or more pests, thereby reducing or preventing crop damage in the field. Disruption of expression of one or more target genes can be achieved using RNAi, for example as described in WO 2017/205751. The present compositions may thus be used in combination with any of the methods described in WO 2017/205751.

### EXAMPLES

### Example 1—Construction of BioBricks and Plasmids

All heterologous genes were synthesized by GeneArt (Life Technologies) in codon-optimized versions for Yarrowia lipolytica. All the genes were amplified by PCR using Phusion U Hot Start DNA Polymerase (ThermoFisher) to obtain the fragments for cloning into yeast expression vectors. The primers are listed in Table 1 and the resulting DNA fragments (BioBricks) are listed in Table 2. The PCR products were separated on a 1%-agarose gel containing Midori Green Advance (Nippon Genetics Europe GmbH). PCR products of the correct size were excised from the gel and purified using the Nucleospin Gel and PCR Clean-up kit (Macherey-Nagel).

Yeast vectors with USER cassette were linearized with FastDigest SfaAI (ThermoFisher) for 2 hours at 37° C. and then nicked with Nb.BsmI (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into vectors by USER-cloning as described in (Holkenbrink, et al., 2018). The reaction was transformed into chemically competent E. coli DHα cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

Strains marked with “***” were constructed as follows. The indicated genes were amplified with gene-specific primers containing a 5′-overhang of “ACTTTTTGCAGTACUAACCGCAG” in the forward primer and a 3′-overhang of “CACGCGAU” in the reverse primer. The first “ATG” of the target gene sequence was omitted. These PCR products were cloned together with BB9454 either into integrative vectors or episomal vectors as described in (Holkenbrink, et al., 2018).

TABLE 1
Primers.

| | | NCBI | |
|---|---|---|---|
| | | accession | |
| Primer ID | Template | number | Hybridises at positions |
| PR10595 | Yali0C | NC_006069 | 1244252 . . . 1244265 |
| PR10604 | pCfB6630* | | 3458 . . . 3498 |
| PR10607 | Yali0A | NC_006067 | 483924 . . . 483945 |
| PR11110 | pCfB6681 | — | 2306 . . . 2336 |
| PR11111 | pCfB6681 | — | 5129 . . . 5152 |
| PR14148 | Yali0E | NC_006071 | 784691 . . . 784708 |
| PR14279 | Yali0C | NC_006069 | 1244254 . . . 1244265 |
| PR15781 | Yali0A | NC_006067 | 2188556 . . . 2188574 |
| PR15788 | Yali0A | NC_006067 | 456893 . . . 456922 |
| PR15789 | Yali0F | NC_006072 | 1948664 . . . 1948678 |
| PR15930 | Yali0C | NC_006069 | 1243743 . . . 1243762 |
| PR16594 | SEQ ID NO: 100 | — | 1 . . . 18 |
| PR16595 | SEQ ID NO: 100 | — | 2735 . . . 2755 |
| PR18214 | Yali0C | NC_006069 | 1243743 . . . 1243761 |
| PR18928 | Yali0C | NC_006069 | 1244252 . . . 1244265 |
| PR20762 | Yali0B | NC_006068 | 2566672 . . . 2566691 |
| PR20763 | Yali0B | NC_006068 | 2567146 . . . 2567162 |
| PR20764 | Yali0B | NC_006068 | 2567171 . . . 2567190 |
| PR20765 | Yali0B | NC_006068 | 2567645 . . . 2567662 |
| PR21723 | SEQ ID NO: 58 | — | 4 . . . 22 |
| PR21724 | SEQ ID NO: 58 | — | 997 . . . 1014 |
| PR21806 | SEQ ID NO: 70 | — | 4 . . . 21 |
| PR21807 | SEQ ID NO: 70 | — | 999 . . . 1017 |
| PR22297 | SEQ ID NO: 70 | — | 999 . . . 1017 |
| PR23138 | Yali0E | NC_006071 | 2795545 . . . 2795556 |
| PR23139 | Yali0E | NC_006071 | 2796053 . . . 2796036 |
| PR23140 | Yali0E | NC_006071 | 2795473 . . . 2795459 |
| PR23141 | Yali0E | NC_006071 | 2268152 . . . 2268140 |
| PR23166 | Yali0E | NC_006071 | 2086416 . . . 2086402 |
| PR23167 | Yali0E | NC_006071 | 2085907 . . . 2085919 |
| PR23168 | Yali0E | NC_006071 | 2086480 . . . 2086497 |
| PR23169 | Yali0E | NC_006071 | 2087001 . . . 2086986 |
| PR23176 | Yali0E | NC_006071 | 2795526 . . . 2795545 |
| PR23177 | Yali0E | NC_006071 | 2795526 . . . 2795545 |
| PR23190 | Yali0E | NC_006071 | 2086449 . . . 2086430 |
| PR23191 | Yali0E | NC_006071 | 2086430 . . . 2086449 |
| PR23632 | SEQ ID NO: 94 | — | 4 . . . 18 |
| PR23633 | SEQ ID NO: 94 | — | 1359 . . . 1377 |
| PR24187 | SEQ ID NO: 53 | — | 4 . . . 18 |
| PR24188 | SEQ ID NO: 53 | — | 1131 . . . 1146 |
| PR25051 | SEQ ID NO: 116 | — | 4 . . . 18 |
| PR25052 | SEQ ID NO: 116 | — | 1587 . . . 1608 |
| PR25053 | SEQ ID: 115 | — | 4 . . . 18 |
| PR25054 | SEQ ID: 115 | — | 4 . . . 18 |
| PR25058 | SEQ ID: 117 | — | 1545 . . . 1566 |
| PR25075 | SEQ ID NO: 118 | — | 4 . . . 20 |
| PR25076 | SEQ ID NO: 118 | — | 1998 . . . 2016 |
| PR10852 | Yali0E | NC_006071 | 784691 . . . 784713 |
| PR14620 | Yali0A | NC_006067 | 2188565 . . . 2188546 |
| PR18241 | Yali0D | NC_006070 | 2193232 . . . 2193213 |
| PR18242 | Yali0D | NC_006070 | 2193213 . . . 2193232 |
| PR18239 | Yali0C | NC_006069 | 568875 . . . 568856 |
| PR18240 | Yali0C | NC_006069 | 568856 . . . 568875 |
| PR25309 | Yali0C | NC_006069 | 1244237 . . . 1244223 |
| PR18214 | Yali0C | NC_006069 | 1243717 . . . 1243735 |
| PR26771 | SEQ ID NO: 193 | — | 1 . . . 20 |
| PR26772 | SEQ ID NO: 193 | — | 375 . . . 393 |
| PR26773 | SEQ ID NO: 194 | — | 4 . . . 20 |
| PR26774 | SEQ ID NO: 194 | — | 876 . . . 897 |
| PR26775 | SEQ ID NO: 195 | — | 4 . . . 25 |
| PR26776 | SEQ ID NO: 195 | — | 875 . . . 897 |
| PR23004 | Yali0C | NC_006069 | 825834 . . . 825853 |
| PR13338 | Yali0C | NC_006069 | 826765 . . . 826747 |
| PR25312 | Yali0C | NC_006069 | 1244237 . . . 1244223 |
| PR25393 | | CP092955.1 | 221105 . . . 221090 |
| PR25394 | | CP092955.1 | 220687 . . . 220703 |
| PR25395 | | CP092960.1 | 204326 . . . 204346 |
| PR25396 | | CP092960.1 | 205023 . . . 204999 |
| PR26805 | SEQ ID NO: 186 | — | 1 . . . 19 |
| PR26806 | SEQ ID NO: 186 | — | 2022 . . . 2040 |
| PR26807 | SEQ ID NO: 40 | | 1 . . . 20 |
| PR26808 | SEQ ID NO: 40 | | 936 . . . 981 |
| PR26809 | SEQ ID NO: 53 | | 1 . . . 20 |
| PR26810 | SEQ ID NO: 53 | | 1128 . . . 1146 |
| PR26811 | SEQ ID NO: 100 | | 1 . . . 20 |
| PR26812 | SEQ ID NO: 100 | | 1350 . . . 1368 |

*Holkenbrink et al., 2018

TABLE 2
DNA fragments (BioBricks) obtained by PCR
using the indicated template and primers.

| Bio | | | Forward | Reverse |
|---|---|---|---|---|
| Brick ID | Description | Template | primer | primer |
| BB1135 | Vector backbone | pCfB6681* | PR11110 | PR11111 |
| BB1631 | Regions downstream of Y. | pCfB4586* | PR14148 | PR15781 |
| | lipolytica pex20 and lip2 | | | |
| BB1635 | Promoter for gRNA | pCfB4589* | PR10607 | PR15788 |
| | expression | | | |
| BB1636 | Terminator region for gRNA | pCfB4589* | PR15789 | PR10604 |
| | expression | | | |
| BB1688 | region upstream of Y. | pCfB3465* | PR14279 | PR15930 |
| | lipolytica tef1 | | | |
| BB1740 | Helicoverpa armigera fatty | pCfB5390** | PR16594 | PR16595 |
| | acyl reductase | | | |
| BB2093 | Y. lipolytica tef1 promoter | pCfB3516** | PR10595 | PR18214 |
| BB2311 | region upstream of | ST4840** | PR20762 | PR20763 |
| | Fas2pl1220 | | | |
| BB2312 | region downstream of | ST4840** | PR20764 | PR20765 |
| | Fas2pl1220 | | | |
| BB2313 | Up- and downstream | BB2311, | PR20762 | PR20765 |
| | genomic region of | BB2312 | | |
| | Fas2p^{I1220} | | | |
| BB2693 | Lobesia botrana desaturase | SEQ ID NO: 58 | PR21723 | PR21724 |
| BB2719 | region upstream of Y. | ST4840** | PR18928 | PR18214 |
| | lipolytica tef1 | | | |
| BB7935 | Spodoptera litura | SEQ ID NO: 70 | PR21806 | PR21807 |
| | desaturase | | | |
| BB8250 | region upstream of Y. | pBP7948 | PR10595 | PR22297 |
| | lipolytica tef1 and | | | |
| | desaturase from | | | |
| | Spodoptera litura | | | |
| BB8665 | Genomic region upstream | ST4840** | PR23139 | PR23138 |
| | of integration site | | | |
| BB8666 | Genomic region | ST4840** | PR23140 | PR23141 |
| | downstream of integration | | | |
| | site | | | |
| BB8679 | Genomic region upstream | ST4840** | PR23167 | PR23166 |
| | of integration site | | | |
| BB8680 | Genomic region | ST4840** | PR23168 | PR23169 |
| | downstream of integration | | | |
| | site | | | |
| BB8971 | Drosophila virilis | SEQ ID NO: 53 | PR24187 | PR24188 |
| | desaturase | | | |
| BB9373 | CDS of Drosophila | SEQ ID NO: 116 | PR25051 | PR25052 |
| | melanogaster Ncb5or | | | |
| BB9377 | CDS of Spodoptera litura | SEQ ID: 118 | PR25075 | PR25076 |
| | Ncb5or | | | |
| BB8816 | Agrotis segetum fatty acyl | SEQ ID: 94 | PR23632 | PR23633 |
| | reductase | | | |
| BB9374 | CDS of Drosophila | SEQ ID: 115 | PR25053 | PR25054 |
| | grimshawi Ncb5or | | | |
| BB9376 | CDS of Homo sapiens | SEQ ID: 117 | PR25075 | PR25058 |
| | Ncb5or | | | |
| BB8955 | regions downstream of Y. | pCfB4586* | PR10852 | PR14620 |
| | lipolytica pex20 and lip2 | | | |
| BB9454 | Y. lipolytica tef1 promoter | ST4840** | PR25309 | PR18214 |
| BB10104 | M. alpina cytochrome B5 | SEQ ID: 193 | PR26771 | PR26772 |
| BB10105 | M. alpina cytochrome B5 | SEQ ID: 194 | PR26773 | PR26774 |
| | reductase 1 | | | |
| BB10106 | M. alpina cytochrome B5 | SEQ ID: 195 | PR26775 | PR26776 |
| | reductase 2 | | | |
| BB9713 | Y. lipolytica gpd promoter | ST4840** | PR23004 | PR13338 |
| BB9457 | Y. lipolytica tef1 promoter | ST4840** | PR25312 | PR18214 |
| BB9510 | S. cerevisiae tef1 promoter | S. cerevisiae | PR25393 | PR25394 |
| | | CEN.PK102-5B | | |
| BB9511 | S. cerevisiae tdh3 promoter | S. cerevisiae | PR25395 | PR25396 |
| | | CEN.PK102-5B | | |
| BB10118 | C. pomonella Ncb5or | SEQ ID: 186 | PR26805 | PR26806 |
| BB10119 | A. transitella desaturase | SEQ ID: 40 | PR26807 | PR26808 |
| BB10120 | D. virilis desaturase | SEQ ID: 53 | PR26809 | PR26810 |
| BB10121 | H. armigera fatty acyl-CoA | SEQ ID: 100 | PR26811 | PR26812 |
| | reductase | | | |

*(Holkenbrink, et al., 2018)
**(Holkenbrink, et al., 2020)

TABLE 3
Vectors.

| Plasmid ID | Resistance | Parent plasmid ID | Biobricks/primer |
|---|---|---|---|
| pBP9002 | Nourseothricin | pCfB3405* | BB8955 |
| pBP9004 | Hygromycin | pCfB3431** | BB8955 |
| pBP9556 | Hygromycin | pBP9004 | BB2093, BB9376 |
| pCfB6630* | Nourseothricin | | |
| pCfB6638* | Nourseothricin | | |
| pBP7912 | — | pCfB6684* | BB2719, BB2693 |
| pBP7980 | — | pCfB6371* | BB1688, BB1740 |
| pBP8033 | Hygromycin | pCfB3431** | BB1635, BB1636, |
| | | | PR18241, PR18242 |
| pBP8138 | — | pCfB6679* | BB8250 |
| pBP8575 | Nourseothricin | pCfB3405* | BB1635, BB1636, |
| | | | PR23190, PR23191 |
| pBP8674 | Nourseothricin | pCfB3405* | BB1635, BB1636, |
| | | | PR23176, PR23177 |
| pBP8782 | — | pBP8660 | BB2093, BB8816 |
| pBP9374 | — | pBP8662 | BB2093, BB8971 |
| pCfB3405** | Nourseothricin | | |
| pCfB6684* | Hygromycin | | |
| pCfB6371* | — | | |
| pCfB3431** | Hygromycin | | |
| pCfB6679* | — | | |
| pBP8660 | — | | BB1135, BB8665, |
| | | | BB1631, BB8666 |
| pBP8662 | — | | BB1135, BB8679, |
| | | | BB8680 |
| pCfB7088* | Nourseothricin | | |
| pBP9546 | Nourseothricin | pBP9002 | BB2093, BB9373 |
| pBP9550 | Nourseothricin | pBP9002 | BB2093, BB9377 |
| pBP9553 | Hygromycin | pBP9004 | BB2093, BB9373 |
| pBP9557 | Hygromycin | pBP9004 | BB2093, BB9377 |
| pBP9554 | Hygromycin | pBP9004 | BB2093, BB9374 |
| pBP9556 | Hygromycin | pBP9004 | BB2093, BB9376 |
| pBP9547 | Nourseothricin | pBP9002 | BB2093, BB9374 |
| pBP9549 | Nourseothricin | pBP9002 | BB2093, BB9376 |
| pBP8032 | Nourseothricin | pCfB3405 | BB1635, BB1636, |
| | | | PR18239, PR18240 |
| pBP8676 | Hygromycin | pCfB3431 | BB1635, BB1636, |
| | | | PR23190, PR23191 |
| pBP10607 | | pBP8621 | BB10104, BB10105, |
| | | | BB9713, BB9457 |
| pBP10608 | | pBP8621 | BB10104, BB10106, |
| | | | BB9713, BB9457 |
| pBP10532 | | pBP9703*** | BB9510, BB10118 |
| pBP10533 | | pBP9702*** | BB9511, BB10119 |
| pBP10534 | | pBP9702*** | BB9511, BB10120 |
| pBP10535 | | pBP9702*** | BB9511, BB10121 |

*(Holkenbrink, et al., 2018)
**(Holkenbrink, et al., 2020)
***(Jensen et al., 2014)

### Example 2—Construction of Yeast Strains

Yeast strains were constructed by transformation of DNA vectors as described in Holkenbrink et al., 2018 and Jensen et al., 2014. Integrative vectors were linearized with FastDigest NotI prior to transformation. When needed, helper vectors to promote the integration into specific genomic regions were co-transformed with the integrative plasmid or DNA repair fragments listed in Tables 2 and 3. Strains were selected on yeast peptone dextrose (YPD) agar with appropriate antibiotics selection. Correct genotype was confirmed by colony PCR and when needed by sequencing. Strains ST6629 is described in (Holkenbrink, et al., 2020). The resulting strains are listed in Table 4.

TABLE 4
Yeast strains

| StrainID | Overexpressed gene | Parent strain | DNA |
|---|---|---|---|
| ST6629* | — | — | — |
| ST7982 | — | ST6629 | pCfB7088 |
| | | | BB2313 |
| ST8377 | FAR1 Desat35 | ST7982 | |
| ST8378 | FAR1 Desat36 | ST7982 | |
| ST8373 | FAR1 Desat30 | ST7982 | |
| ST8407 | Desat38 | ST6629 | pBP8138 |
| | | | pCfB6638 |
| ST8524 | FAR1 | ST6629 | pBP7980 |
| | | | pCfB6630 |
| ST9992 | Desat30 | ST7982 | pBP7912 |
| | | | pBP8033 |
| ST10609 | FAR12 | ST6629 | pBP8782 |
| | | | pBP8674 |
| ST10610 | — | ST9992 | pBP9002 |
| ST10612 | Desat30 DmNcb5or | ST9992 | pBP9546 |
| ST10614 | Desat30 SlitNcb5or | ST9992 | pBP9550 |
| ST10615 | — | ST8407 | pBP9004 |
| ST10617 | Desat38 DmNcb5or | ST8407 | pBP9553 |
| ST10618 | Desat38 SlitNcb5or | ST8407 | pBP9557 |
| ST10623 | — | ST8524 | pBP9004 |
| ST10625 | FAR1 DmNcb5or | ST8524 | pBP9553 |
| ST10626 | FAR1 SlitNcb5or | ST8524 | pBP9557 |
| ST10628 | Desat61 | ST7982 | pBP9374 |
| | | | pBP8575 |
| ST10665 | — | ST10609 | pBP9004 |
| ST10667 | FAR12 DmNcb5or | ST10609 | pBP9553 |
| ST10668 | FAR12 SlitNcb5or | ST10609 | pBP9557 |
| ST10672 | — | ST10628 | pBP9004 |
| ST10674 | Desat61 DmNcb5or | ST10628 | pBP9553 |
| ST10675 | Desat61 SlitNcb5or | ST10628 | pBP9557 |
| ST10783 | Desat38 DgNcb5or | ST8407 | pBP9554 |
| ST10785 | Desat38 HsNcb5or | ST8407 | pBP9556 |
| ST10789 | Desat61 DgNcb5or | ST10628 | pBP9547 |
| ST10791 | Desat61 HsNcb5or | ST10628 | pBP9549 |
| ST8544 | FAR1 Desat38 | ST8407 | pBP7980 |
| | | | pBP8032 |
| ST10766 | FAR1 Desat38 | ST8544 | pBP9002 |
| ST10768 | FAR1 Desat38 DmNcb5or | ST8544 | pBP9546 |
| ST10772 | FAR1 Desat38 SlitNcb5or | ST8544 | pBP9550 |
| ST9253** | FAR1 Desat30 | — | — |
| ST10773 | FAR1 Desat30 | ST9253 | pBP9002 |
| ST10775 | FAR1 Desat30 DmNcb5or | ST9253 | pBP9546 |
| ST10776 | FAR1 Desat30 DgNcb5or | ST9253 | pBP9547 |
| ST10778 | FAR1 Desat30 HsNcb5or | ST9253 | pBP9549 |
| ST10779 | FAR1 Desat30 SlitNcb5or | ST9253 | pBP9550 |
| ST8520 | FAR1 | ST7982 | pBP7980 |
| | | | pBP6630 |
| ST10629 | FAR1 Desat61 | ST8520 | pBP9374 |
| | | | pBP8676 |
| ST10661 | FAR1 Desat61 | ST10629 | pBP9002 |
| ST10663 | FAR1 Desat61 DmNcb5or | ST10629 | pBP9546 |
| ST10780 | FAR1 Desat61 DgNcb5or | ST10629 | pBP9547 |
| ST10897 | CpNcb5or | ST9259 | |
| ST10469 | CpNcb5or | ST10435 | |
| ST12511 | FAR1 | Saccharomyces | pBP9701 |
| | | cerevisiae | pBP10535 |
| | | CEN.PK102-5B | pBP9703 |
| ST12514 | FAR1 CpNcb5or | Saccharomyces | pBP9701 |
| | | cerevisiae | pBP10532 |
| | | CEN.PK102-5B | pBP9703 |
| ST12510 | Desat61 | Saccharomyces | pBP9701 |
| | | cerevisiae | pBP10534 |
| | | CEN.PK102-5B | pBP9703 |
| ST12513 | Desat61 CpNcb5or | Saccharomyces | pBP9701 |
| | | cerevisiae | pBP10534 |
| | | CEN.PK102-5B | pBP9703 |
| ST10624*** | FAR1 CpNcb5or | | |
| ST10786*** | FAR1 DgNcb5or | | |
| ST10787*** | FAR1 LboNcb5or | | |
| ST10788*** | FAR1 HsNcb5or | | |
| ST10666*** | FAR12 CpNcb5or | | |
| ST10616*** | Desat38 CpNcb5or | | |
| ST10784*** | Desat38 LboNcb5or | | |
| ST10611*** | Desat30 CpNcb5or | | |
| ST10613*** | Desat30 LboNcb5or | | |
| ST10673*** | Desat61 CpNcb5or | | |
| ST10790*** | Desat61 LboNcb5or | | |
| ST12195*** | Desat70 | | |
| ST12178*** | Desat70 CpNcb5or | | |
| ST12003*** | CpNcb5or | | |
| ST12521*** | Desat70 MaCytb5 | ST12050 | |
| | MaCytB5Red2 | | |
| ST12050 | MaCytb5 MaCytB5Red2 | ST7982 | pBP10608 |
| ST12520*** | Desat70 MaCytb5 | ST12049 | |
| | MaCytB5Red1 | | |
| ST12049 | MaCytb5 MaCytB5Red1 | ST7982 | pBP10607 |
| ST12073*** | FAR1 CpNcb5or | | |
| ST12074*** | FAR4 CpNcb5or | | |
| ST12076*** | FAR6 CpNcb5or | | |
| ST12077*** | FAR8 CpNcb5or | | |
| ST12081*** | FAR42 CpNcb5or | | |
| ST12082*** | FAR11 CpNcb5or | | |
| ST12083*** | FAR25 CpNcb5or | | |
| ST12084*** | FAR13 CpNcb5or | | |
| ST12087*** | FAR16 CpNcb5or | | |
| ST12088*** | FAR17 CpNcb5or | | |
| ST12089*** | FAR18 CpNcb5or | | |
| ST12096*** | FAR28 CpNcb5or | | |
| ST12098*** | FAR30 CpNcb5or | | |
| ST12100*** | FAR43 CpNcb5or | | |
| ST12101*** | FAR33 CpNcb5or | | |
| ST12102*** | FAR34 CpNcb5or | | |
| ST12103*** | FAR35 CpNcb5or | | |
| ST12104*** | FAR44 CpNcb5or | | |
| ST12107*** | FAR45 CpNcb5or | | |
| ST12112*** | FAR41 CpNcb5or | | |
| ST12116*** | FAR46 CpNcb5or | | |
| ST12115*** | FAR47 CpNcb5or | | |
| ST12241*** | Desat69 CpNcb5or | | |
| ST12163*** | Desat16 CpNcb5or | | |
| ST12164*** | Desat19 CpNcb5or | | |
| ST12166*** | Desat21 CpNcb5or | | |
| ST12169*** | Desat44 CpNcb5or | | |
| ST12170*** | Desat45 CpNcb5or | | |
| ST12535*** | Desat37 CpNcb5or | | |
| ST12173*** | Desat63 CpNcb5or | | |
| ST12174*** | Desat65 CpNcb5or | | |
| ST12175*** | Desat71 CpNcb5or | | |
| ST12177*** | Desat72 CpNcb5or | | |
| ST12178*** | Desat70 CpNcb5or | | |
| ST12179*** | Desat73 CpNcb5or | | |
| ST12197*** | Desat24 CpNcb5or | | |
| ST12198*** | Desat56 CpNcb5or | | |
| ST12200*** | Desat59 CpNcb5or | | |
| ST12201*** | Desat60 CpNcb5or | | |
| ST12202*** | Desat61 CpNcb5or | | |
| ST12205*** | Desat43 CpNcb5or | | |
| ST12208*** | Desat27 CpNcb5or | | |
| ST12209*** | Desat75 CpNcb5or | | |
| ST12223*** | Desat30 CpNcb5or | | |
| ST12224*** | Desat35 CpNcb5or | | |
| ST12225*** | Desat36 CpNcb5or | | |
| ST12226*** | Desat52 CpNcb5or | | |
| ST12227*** | Desat76 CpNcb5or | | |
| ST12229*** | Desat77 CpNcb5or | | |
| ST12231*** | Desat78 CpNcb5or | | |
| ST12518*** | AseNcb5or Desat70 | | |
| ST12007*** | AseNcb5or | | |
| ST12519*** | BterNcb5or Desat70 | | |
| ST12008*** | BterNcb5or | | |
| ST12118*** | FAR1 | | |
| ST12395*** | AseNcb5or FAR1 | | |
| ST12397*** | BterNcb5or FAR1 | | |
| ST12118*** | FAR1 | | |
| ST12119*** | FAR4 | | |
| ST12121*** | FAR6 | | |
| ST12122*** | FAR8 | | |
| ST12126*** | FAR42 | | |
| ST12127*** | FAR11 | | |
| ST12128*** | FAR25 | | |
| ST12129*** | FAR13 | | |
| ST12132*** | FAR16 | | |
| ST12133*** | FAR17 | | |
| ST12134*** | FAR18 | | |
| ST12141*** | FAR28 | | |
| ST12143*** | FAR30 | | |
| ST12145*** | FAR43 | | |
| ST12146*** | FAR33 | | |
| ST12147*** | FAR34 | | |
| ST12148*** | FAR35 | | |
| ST12149*** | FAR44 | | |
| ST12152*** | FAR45 | | |
| ST12157*** | FAR41 | | |
| ST12161*** | FAR46 | | |
| ST12160*** | FAR47 | | |
| ST12242*** | Desat69 | | |
| ST12180*** | Desat16 | | |
| ST12181*** | Desat19 | | |
| ST12183*** | Desat21 | | |
| ST12186*** | Desat44 | | |
| ST12187*** | Desat45 | | |
| ST12534*** | Desat37 | | |
| ST12190*** | Desat63 | | |
| ST12191*** | Desat65 | | |
| ST12192*** | Desat71 | | |
| ST12194*** | Desat72 | | |
| ST12195*** | Desat70 | | |
| ST12196*** | Desat73 | | |
| ST12210*** | Desat24 | | |
| ST12211*** | Desat56 | | |
| ST12213*** | Desat59 | | |
| ST12214*** | Desat60 | | |
| ST12215*** | Desat61 | | |
| ST12218*** | Desat43 | | |
| ST12221*** | Desat27 | | |
| ST12222*** | Desat75 | | |
| ST12232*** | Desat30 | | |
| ST12233*** | Desat35 | | |
| ST12234*** | Desat36 | | |
| ST12235*** | Desat52 | | |
| ST12236*** | Desat76 | | |
| ST12238*** | Desat77 | | |
| ST12240*** | Desat78 | | |

*(Holkenbrink, et al., 2020)
**(Petkevicius, et al., 2021)
***For description of strain constructions see text

### Example 3—Cultivation of Strains and Analysis of Fatty Alcohols and Fatty Acid Methyl Esters (FAME)

Strains were inoculated from a YPD agar plate (10 g/L yeast extract, 10 g/L peptone, 20 g/L glucose, 15 g/L agar agar) to an initial OD600 of 0.1-0.2 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 24 h, the plates were centrifuged for 5 min at 3,000×g. The supernatant was discarded and the cells were resuspended in 1.25 mL production medium per well (50 g/L glycerol, 5 g/L yeast extract, 4 g/L KH₂PO₄, 1.5 g/L MgSO₄, 0.2 g/L NaCl, 0.265 g/L CaCl₂·2H₂O, 2 mL/L trace elements solution: 4.5 g/L CaCl₂·2H₂O, 4.5 g/L ZnSO₄O·7H₂O, 3 g/L FeSO₄·7H₂O, 1 g/L H₃BO₃, 1 g/L MnCl₂·4H₂O, 0.4 g/L N Na₂MoO₄·2H₂O, 0.3 g/L CoCl₂·6H₂O, 0.1 g/L CuSO₄·5H₂O, 0.1 g/L KI, 15 g/L EDTA). The media was supplemented with antibiotics if necessary. The plate was incubated for 26 hours at 28° C., shaken at 300 rpm.

For analysis of fatty alcohols, 1 mL of fermentation broth was harvested from each vial and biomass was separated by centrifugation for 5 min at 3,000×g. Biomass pellets were extracted with 990 μL of ethyl acetate:ethanol (84:15) and 10 μL of 19:Me (2 mg/mL) as internal standard. The samples were vortexed for 20 sec and incubated for 1 h at room temperature, followed by 5 min of vortexing. 300 μL of H2O was added to each sample. The samples were vortexed and centrifuged for 5 min at 21° C. and 3,000×g. The upper organic phase was analyzed via gas chromatography-mass spectrometry (GC-MS). GC-MS analyses were performed on an Agilent 7820A GC coupled to a mass selective detector Agilent 5977B. The GC was equipped with a DB Fatwax column (30 m×0.25 mm×0.25 μm), and helium was used as carrier gas. The MS was operated in electron impact mode (70 eV), scanning between m/z 30 and 400, and the injector was configured in split mode 20:1 at 220° C. Oven temperature was set to 80° C. for 1 min, then increased at a rate of 20° C./min to 210° C., followed by a hold at 210° C. for 7 min, and then increased at a rate of 20 C/min to 230° C. Compounds were identified by comparison of retention times and mass spectra of the reference compounds. Compounds were quantified by the ion 55.1 m/z. Data were analyzed by the Agilent Masshunter software. The concentrations of fatty alcohols were calculated based on standard calibration curves prepared with reference standards.

For analysis of FAMEs, 1 mL of fermentation broth was harvested from each vial and biomass was separated by centrifugation for 5 min at 3,000 xg. Biomass pellets were extracted with 1000 μL 1M HCl in Methanol (anhydrous). The samples were vortexed for 20 sec and placed in the 80° C. water bath for 2 h. The samples were vortexed every 30 min for 10 sec. After cooling down of the samples to room temperature, 1000 μL of 1M NaOH in Methanol (anhydrous), 500 μL of NaCl saturated H₂O, 990 μL of hexane and 10 μL of 19:Me (2 mg/mL) as internal standard were added. The samples were vortexed and centrifuged for 5 min at 21° C. and 3,000×g. The upper organic phase was analyzed via GC-MS as described above.

### Example 4—Putative Ncb5or-Coding Genes from Insects

Putative Ncb5or proteins from Lepidoptera can be seen in Table 5. Each protein contains a cytochrome b5, a cytochrome b5 reductase, and an SGD1-CHORD domain.

TABLE 5
Non-exhaustive list of Lepidoptera Ncb5ors.

| Description | Species | Accession |
|---|---|---|
| cytochrome b5 reductase 4 isoform X1 | Manduca sexta | XP_030040351.1 |
| PREDICTED: cytochrome b5 reductase | Amyelois transitella | XP_013199405.1 |
| 4 isoform X1 | | |
| cytochrome b5 reductase 4 isoform X1 | Spodoptera litura | XP_022822658.1 |
| hypothetical protein evm_009546 | Chilo suppressalis | RVE45796.1 |
| cytochrome b5 reductase 4 isoform X1 | Galleria mellonella | XP_026756471.1 |
| cytochrome b5 reductase 4 | Bombyx mori | XP_012550604.1 |
| cytochrome b5 reductase 4 isoform X1 | Bombyx mandarina | XP_028042050.1 |
| cytochrome b5 reductase 4 isoform X1 | Helicoverpa armigera | XP_021195006.1 |
| cytochrome b5 reductase 4 isoform X1 | Danaus plexippus | XP_032518662.1 |
| | plexippus | |
| cytochrome b5 reductase 4 isoform X1 | Bicyclus anynana | XP_023948592.1 |
| cytochrome b5 reductase 4 isoform X1 | Vanessa tameamea | XP_026495955.1 |
| PREDICTED: cytochrome b5 reductase | Papilio polytes | XP_013148264.1 |
| 4-like | | |
| PREDICTED: cytochrome b5 reductase | Papilio xuthus | XP_013179866.1 |
| 4-like isoform X1 | | |
| PREDICTED: cytochrome b5 reductase | Papilio xuthus | XP_013179867.1 |
| 4-like isoform X2 | | |
| PREDICTED: cytochrome b5 reductase | Papilio machaon | XP_014360030.1 |
| 4-like | | |
| cytochrome b5 reductase 4-like | Pieris rapae | XP_022127972.1 |
| isoform X1 | | |
| cytochrome b5 reductase 4 isoform X1 | Hyposmocoma kahamanoa | XP_026321155.1 |
| cytochrome b5 reductase 4 isoform X2 | Bicyclus anynana | XP_023948593.1 |
| cytochrome b5 reductase 4 isoform X2 | Spodoptera litura | XP_022822662.1 |
| PREDICTED: cytochrome b5 reductase | Amyelois transitella | XP_013199409.1 |
| 4 isoform X3 | | |
| PREDICTED: cytochrome b5 reductase | Papilio polytes | XP_013148114.1 |
| 4-like isoform X1 | | |
| cytochrome b5 reductase 4 isoform X3 | Manduca sexta | XP_030040353.1 |
| cytochrome b5 reductase 4 isoform X3 | Spodoptera litura | XP_022822663.1 |
| cytochrome b5 reductase 4 isoform X3 | Helicoverpa armigera | XP_021195008.1 |
| hypothetical protein | Helicoverpa armigera | PZC74467.1 |
| B5X24_HaOG207824 | | |
| cytochrome b5 reductase 4 isoform X2 | Helicoverpa armigera | XP_021195007.1 |
| PREDICTED: cytochrome b5 reductase | Amyelois transitella | XP_013199408.1 |
| 4 isoform X2 | | |
| cytochrome b5 reductase 4 isoform X2 | Hyposmocoma kahamanoa | XP_026321156.1 |
| cytochrome b5 reductase 4 isoform X3 | Galleria mellonella | XP_026756473.1 |
| cytochrome b5 reductase 4 isoform X2 | Bombyx mandarina | XP_028042054.1 |
| cytochrome b5 reductase 4 isoform X2 | Galleria mellonella | XP_026756472.1 |
| cytochrome b5 reductase 4 isoform X2 | Vanessa tameamea | XP_026495958.1 |
| cytochrome b5 reductase 4 isoform X3 | Vanessa tameamea | XP_026495959.1 |
| Cytochrome b5 reductase 4 | Papilio xuthus | KPI97000.1 |
| PREDICTED: cytochrome b5 reductase | Papilio xuthus | XP_013179870.1 |
| 4-like isoform X3 | | |
| cytochrome b5 reductase 4 isoform X3 | Danaus plexippus | XP_032518665.1 |
| | plexippus | |
| cytochrome b5 reductase 4 isoform X2 | Danaus plexippus | XP_032518664.1 |
| | plexippus | |
| cytochrome b5 reductase 4 isoform X4 | Danaus plexippus | XP_032518666.1 |
| | plexippus | |
| cytochrome b5 reductase 4-like | Pieris rapae | XP_022127974.1 |
| isoform X2 | | |
| cytochrome b5 reductase 4-like | Pieris rapae | XP_022127975.1 |
| isoform X3 | | |
| PREDICTED: cytochrome b5 reductase | Papilio machaon | XP_014360076.1 |
| 4-like | | |
| PREDICTED: cytochrome b5 reductase | Papilio xuthus | XP_013179773.1 |
| 4-like isoform X2 | | |
| PREDICTED: cytochrome b5 reductase | Papilio xuthus | XP_013179772.1 |
| 4-like isoform X1 | | |
| hypothetical protein KGM_216032 | Danaus plexippus | OWR44507.1 |
| | plexippus | |
| Cytochrome b5 reductase 4 | Papilio machaon | KPJ13840.1 |
| cytochrome b5 reductase 4-like | Pieris rapae | XP_022122274.1 |
| Cytochrome b5 reductase 4 | Papilio xuthus | KPI97001.1 |

### Example 5—Ncb5or Co-Expression Increases the Activity of Fatty Acyl-CoA Reductases

Two insect Ncb5or genes were co-expressed in Y. lipolytica in combination with a fatty acyl-CoA reductase from Helicoverpa armigera (FAR1) or a fatty acyl-CoA reductase from Agrotis segetum (FAR12), respectively. The strains were cultivated, and fatty alcohols analyzed as described in Example 3.

The presence of insect Ncb5or improved the activity of FAR1 reductase in Y. lipolytica by 15-25% (Table 6) and of FAR12 by 16% (Table 7).

TABLE 6
Effect of Ncb5or on the activity of
reductase FAR1 from H. armigera.

| Strain | Ncb5or | Total fatty alcohols (mg/L) | Improvement (%) |
|---|---|---|---|
| ST10623 | — | 361 ± 34 | Reference |
| ST10625 | DmNcb5or | 415 ± 38 | 15 |
| ST10626 | SlitNcb5or | 451 ± 10 | 25 |

TABLE 7
Effect of Ncb5or on the activity of
reductase FAR12 from A. segetum.

| Strain | Ncb5or | Total fatty alcohols (mg/L) | Improvement (%) |
|---|---|---|---|
| ST10665 | — | 80 ± 18 | Reference |
| ST10667 | DmNcb5or | 93 ± 32 | 16 |
| ST10668 | SlitNcb5or | 62 ± 7 | NA |

Example 6—Ncb5or co-expression increases the activity of fatty acyl-CoA desaturases Three insect and one human Ncb5or genes were co-expressed in Y. lipolytica in combination with a fatty acyl-CoA desaturase from Spodoptera litura (Desat38), or a fatty acyl-CoA desaturase from Lobesia botrana (Desat30), or a fatty acyl-CoA desaturase from Drosophila virilis (Desat61), respectively. The strains were cultivated and FAMEs analyzed as described in Example 3.

The presence of insect Ncb5or improved the activity of Desat38 desaturase in Y. lipolytica by 13 to 42% (Table 8), Desat30 by 9-36% (Table 9), and Desat61 by 3-34% (Table 10).

TABLE 8
Effect of Ncb5or on the activity of
desaturase Desat38 from S. litura.

| Strain | Ncb5or | Z11-16:Me purity (%) | Improvement (%) |
|---|---|---|---|
| ST10615 | — | 26 ± 0 | Reference |
| ST10617 | DmNcb5or | 36 ± 2 | 38 |
| ST10618 | SlitNcb5or | 36 ± 2 | 42 |
| ST10783 | DgNcb5or | 32 ± 3 | 24 |
| ST10785 | HsNcb5or | 29 ± 2 | 13 |

TABLE 9
Effect of Ncb5or on the activity of desaturase Desat30 from
L. botrana. Strains in this table carry a mutation in
Fas2p that increases tetradecanoyl-CoA biosynthesis.

| Strain | Ncb5or | Z11-14:Me (mg/L) | Improvement (%) |
|---|---|---|---|
| ST10610 | — | 11 ± 2 | Reference |
| ST10612 | DmNcb5or | 15 ± 0 | 36 |
| ST10614 | SlitNcb5or | 12 ± 1 | 9 |

TABLE 10
Effect of Ncb5or on the activity of desaturase Desat61 from
D. virilis. Strains in this table carry a mutation in
Fas2p that increases tetradecanoyl-CoA biosynthesis.

| Strain | Ncb5or | Z9-14:Me purity (%) | Improvement (%) |
|---|---|---|---|
| ST10672 | — | 3 ± 0 | Reference |
| ST10674 | DmNcb5or | 4 ± 1 | 9 |
| ST10675 | SlitNcb5or | 4 ± 0 | 12 |
| ST10789 | DgNcb5or | 3 ± 0 | 3 |
| ST10791 | HsNcb5or | 5 ± 0 | 34 |

### Example 7—Ncb5or Co-Expression Increases the Production of Z11-16:OH in Y. lipolytica

Two insect Ncb5or genes were co-expressed in Y. lipolytica previously engineered for production of Z11-16:OH. The strains were cultivated and fatty alcohols analyzed as described in Example 3.

The presence of insect Ncb5or improved the titer of Z11-16:OH by 69-82% (Table 11). Furthermore, both Ncb5or proteins, DmNcb5or and SlitNcb5or, increased the purity of Z11-16:OH (Table 11).

TABLE 11
Effect of Ncb5or on the production of Z11-16:OH.

| | | | Improvement | Total | Z11- |
|---|---|---|---|---|---|
| | | Z11- | of Z11- | fatty | 16:OH |
| | | 16:OH | 16:OH | alcohols | purity |
| Strains | Ncb5or | (mg/L) | (%) | (mg/L) | (%) |
| ST10766 | — | 87 ± 9 | Reference | 184 ± 15 | 47 ± 2 |
| ST10768 | DmNcb5or | 159 ± 2 | 82 | 241 ± 9 | 66 ± 2 |
| ST10772 | SlitNcb5or | 260 ± 8 | 69 | 378 ± 5 | 69 ± 2 |

### Example 8—Ncb5or Co-Expression Increases the Production of Z11-14:OH in Y. lipolytica

Three insect Ncb5or and one human Ncb5or genes were co-expressed in Y. lipolytica previously engineered for production of Z11-14:OH. The strains were cultivated and fatty alcohols analyzed as described in Example 3.

The presence of insect Ncb5or improved the titer of Z11-14:OH by 8-13% (Table 12). Furthermore, several Ncb5or proteins (DmNcb5or, DgNcb5or) increased the purity of Z11-14:OH (Table 12).

TABLE 12
Effect of Ncb5or on the production of Z11-14:OH.

| | | | Improve- | | |
|---|---|---|---|---|---|
| | | | ment | Total | Z11- |
| | | Z11- | of Z11- | fatty | 14:OH |
| | | 14:OH | 14:OH | alcohol | purity |
| Strains | Ncb5or | (mg/L) | (%) | (mg/L) | (%) |
| ST10773 | — | 161 ± 19 | Reference | 1053 ± 112 | 15 ± 1 |
| ST10775 | DmNcb5or | 174 ± 16 | 8 | 837 ± 77 | 21 ± 0 |
| ST10776 | DgNcb5or | 181 ± 15 | 13 | 1026 ± 110 | 18 ± 0 |
| ST10778 | HsNcb5or | 122 ± 78 | NA | 706 ± 415 | 20 ± 8 |
| ST10779 | SlitNcb5or | 154 ± 13 | NA | 711 ± 55 | 27 ± 2 |

### Example 9—Ncb5or Co-Expression Increases the Production of Z9-14:OH in Y. lipolytica

Two insect Ncb5or genes were co-expressed in Y. lipolytica previously engineered for production of Z9-14:OH. The strains were cultivated and fatty alcohols analyzed as described in Example 3.

The presence of insect Ncb5or improved the titer of Z9-14:OH by 25-46% (Table 13). Furthermore, the DmNcb5or proteins increased the purity of Z9-14:OH (Table 13).

TABLE 13
Effect of Ncb5or on the production of Z9-14:OH.

| | | | Improvement | Total | Z9- |
|---|---|---|---|---|---|
| | | Z9- | of Z9- | fatty | 14:OH |
| | | 14:OH | 14:OH | alcohol | purity |
| Strains | Ncb5or | (mg/L) | (%) | (mg/L) | (%) |
| ST10661 | — | 75 ± 13 | Reference | 251 ± 43 | 30 ± 1 |
| ST10663 | DmNcb5or | 93 ± 3 | 25 | 278 ± 10 | 34 ± 0 |
| ST10780 | DgNcb5or | 109 ± 23 | 46 | 347 ± 59 | 31 ± 1 |

### Example 10—Ncb5or Expression in Strain Engineered for High Production of Z11-16:OH and Z9-14:OH

The Ncb5or from Cydia pomonella, CpNcb5or, was expressed in Y. lipolytica strains ST9259 and ST10435 engineered to produce high titers of Z11-16:OH and Z9-14:OH, respectively, resulting in strain ST10897 and ST10469, respectively. The strains were cultivated and fatty alcohols analyzed as described in Example 3. Strain ST10897 and ST10469 produced 44% more of Z11-16:OH and 75% more of Z9-14:OH than the control strains ST9259 and ST10435, respectively.

### Example 11—Co-Expression of Multiple Ncb5or in One Production Strain

Two Ncb5or are expressed in strain ST8544 and/or in a Y. lipolytica strain producing high titers of Z11-16:OH or Z9-14:OH. The strains are cultivated and fatty alcohols analyzed as described in Example 3.

### Example 12—Ncb5or Expression in the Yeast S. cerevisiae

An insect Ncb5or was co-expressed in the yeast S. cerevisiae together with either the Desat61 desaturase, or the fatty acyl reductase FAR1. The strains were inoculated in 2.5 ml yeast synthetic drop-out medium (1.39 g/L yeast synthetic drop-out medium without histidine, leucine, tryptophan and uracil (Sigma Aldrich, Y2001), 6.7 g/L yeast nitrogen base without amino acids (Sigma Aldrich Y0626), 20 g/L glucose) to an OD600 of 0.2. The strains were cultivated in 24-well deep-well plates (EnzyScreen) in triplicates and incubated for 24 hours at 28 h with shaking at 250 rpm. After 24 hours the cells were pelleted by centrifugation, the supernatant discarded and replaced by 1.5 ml fresh yeast synthetic drop-out medium. The fatty alcohols/FAMEs were analyzed as described in Example 3. Strain ST12511 expressing fatty acyl reductase FAR1 produced 3.2±0.1 mg/L total fatty alcohols while strain ST12514 co-expressing the Ncb5or of Cydia pomonella with FAR1 produced 3.9±0.7 mg/L total fatty alcohols, corresponding to an 22% improvement. In strain ST12510, expressing ΔZ9-14 desaturase Desat61 from Drosophila virilis, Z9-14:Me accounts for 9.3±0.2% of the total fatty acids, while in strain ST12513 co-expressing the Ncb5or of Cydia pomonella and Desat61 Z9-14:Me accounted for 9.4±0.3% of the total fatty acids.

### Example 13—Co-Expression of Lepidoptera Desaturase and Ncb5or in Plants

Wild-type Nicotiana benthamiana plants are grown in a greenhouse or growth chamber. An insect Ncb5or and desaturase are cloned into a plant expression vector and electroporated into Agrobacterium tumefaciens. The transformed strain was cultivated in LB medium and the expression of virulence genes was induced by addition of acetosyringone to the medium. The culture was diluted in infiltration buffer and applied to the underside of N. benthamiana leaf and slight pressure applied to the leaf. Plants were grown for four more days. For lipid analysis, −100 mg of fresh leaf is used. The lipid extraction was as for yeast cells described in Example 3.

Instead of Nicotiana benthamiana plants and oleogenious plant could be chosen as a host. The plants are cultivated. The desired desaturated fatty acids accumulate in plant lipids. The fatty acids can be recovered from plant lipids by methods known in the art, e.g. after homogenisation of the plants and recovery of the lipids by methods known in the art. The recovered lipids are hydrolyzed into free fatty acids and esterified to fatty acid alkyl ester, followed by a reduction to either fatty alcohols or fatty aldehydes.

### Example 14—Desaturase Specificity

The activities and specificities of desaturases were tested in a S. cerevisiae strain with deletions of OLE1 and ELO1 genes, encoding for Δ9-fatty acid desaturase and medium-chain acyl elongase respectively. Three individual colonies of strains ST_Desat18, ST_Desat17, ST_Desat22, ST_Desat23, ST_ScOLE1 and ST_DmeD9 were inoculated into 1 mL selective media (SC-Ura-Leu) and incubated at 30° C. and 300 rpm for 48 h. The cultures were diluted to an OD600 of 0.4 in 5 mL selective medium supplemented with 2 mM CuSO₄ and the 0.5 mM methyl myristate (14:Me) (Larodan Fine Chemicals, Sweden). The methyl myristate stock solution was prepared to a concentration of 100 mM in 96% ethanol. The yeast cultures were incubated at 30° C. at 300 rpm for 48 hours.

1 mL of culture was sampled and 3.12 μg of nonadecylic acid methyl ester was added as internal standard. Total lipids were extracted using 3.75 mL of methanol/chloroform (2:1, v/v), in a glass vial. One mL of acetic acid (0.15 M) and 1.25 mL of water were added to the tube. Tubes were vortexed vigorously and centrifuged at 2,000×g for 2 min. The bottom chloroform phase, about 1 mL, containing the total lipids, was transferred to a new glass vial and the solvent was evaporated to dryness. Fatty acid methyl esters (FAMEs) were made from this total lipid extract by acid methanolysis. One mL of 2% sulfuric acid in methanol (v/v) was added to the tube, vortexed vigorously, and incubated at 90° C. for 1 h. After incubation, 1 mL of water was added and mixed well, and then 1 mL of hexane was used to extract the FAMEs.

The methyl ester samples were subjected to GC-MS analyses on a Hewlett Packard 6890 GC coupled to a mass selective detector HP 5973. The GC was equipped with an INNOWax column (30 m×0.25 mm×0.25 μm), and helium was used as the carrier gas (average velocity: 33 cm/s). The MS was operated in electron impact mode (70 eV), and the injector was configured in splitless mode at 220° C. The oven temperature was set to 80° C. for 1 min, then increased at a rate of 10° C./min up to 210° C., followed by a hold at 210° C. for 15 min, and then increased at a rate of 10° C./min up to 230° C. followed by a hold at 230° C. for 20 min. The monounsaturated fatty-acid products were identified by comparing their retention times and mass spectra with those of synthetic standards. Data were analyzed by the ChemStation software (Agilent Technologies, USA).

The measured concentrations of Z9-14:Me and Z9-16:Me (Table 14) show that strain ST_DmeD9, expressing desaturase from D. melanogaster, resulted in the highest concentration of Z9-14:Me (3.67 mg/L) and in the maximal ratio of Z9-14:Me and Z9-16:Me. This indicates that among the tested desaturases, D. melanogaster desaturase has the highest activity and specificity towards C14-CoA substrate.

TABLE 14
Activity and specificity of heterologous desaturases in yeast.

| | | | Vectors | | | |
|---|---|---|---|---|---|---|
| | Over- | Parent strain | introduced | | | Ratio of |
| | expressed | (key charac- | into parent | Z9-14:Me | Z9-16:Me | 14:1/16:1 |
| Strain name | desaturase | teristics) | strain | (mg/L) | (mg/L) | [specificity] |
| ST_Desat18 | Desat18 | Δole1Δelo1 | pYEX-CHT- | 0.25 | 0.56 | 0.45 |
| | | | Desat18 | | | |
| ST_Desat17 | Desat17 | Δole1Δelo1 | pYEX-CHT- | 0.03 | 0.13 | 0.20 |
| | | | Desat17 | | | |
| ST_Desat22 | Desat22 | Δole1Δelo1 | pYEX-CHT- | 0.00 | 0.00 | 0.00 |
| | | | Desat22 | | | |
| ST_Desat23 | Desat23 | Δole1Δelo1 | pYEX-CHT- | 0.00 | 0.00 | 0.00 |
| | | | Desat23 | | | |
| ST_ScOLE1 | ScOLE1 | Δole1Δelo1 | pYEX-CHT- | 0.39 | 3.01 | 0.13 |
| | | | OLE1 | | | |
| ST_DmeD9 | Desat24 | Δole1Δelo1 | pYEX-CHT- | 3.67 | 0.24 | 15.29 |
| | | | Desat24 | | | |

Strains ST8377, ST8378 and ST8373 were also tested for production of desaturated 014 compounds. Fatty alcohols were extracted from these strains after cultivation and analysed by GC-MS. Titers are shown in Table 15.

TABLE 15
Titers obtained in strains expressing FAR1 and a desaturase as indicated.
Titers are in mg/L. Columns indicate the titers of the desaturated
fatty alcohols E11-14:OH, Z11-14:OH, Z9-16:OH and Z11-16:OH.

| Strain | Desaturase | | Z9- | E11- | Z11- | | Z9- | Z11- |
|---|---|---|---|---|---|---|---|---|
| ID | introduced | 14:OH | 14:OH | 14:OH | 14:OH | 16:OH | 16:OH | 16:OH |
| ST8225 | none | 214.75 ± | 1.26 ± | 0.00 ± | 0.00 ± | 76.98 ± | 26.56 ± | 1.31 ± |
| (control) | | 36.86 | 0.21 | 0.00 | 0.00 | 14.71 | 2.12 | 0.88 |
| ST8377 | Desat35 | 169.57 ± | 2.31 ± | 0.90 ± | 2.09 ± | 57.13 ± | 20.62 ± | 2.51 ± |
| | | 8.58 | 0.18 | 0.10 | 0.03 | 1.63 | 0.22 | 0.10 |
| ST8378 | Desat36 | 193.04 ± | 3.29 ± | 0.30 ± | 0.00 ± | 59.42 ± | 24.45 ± | 2.68 ± |
| | | 12.42 | 0.09 | 0.13 | 0.00 | 1.10 | 1.24 | 0.19 |
| ST8373 | Desat30 | 296.40 ± | 2.29 ± | 1.46 ± | 21.53 ± | 107.77 ± | 37.91 ± | 3.09 ± |
| | | 17.20 | 0.07 | 0.22 | 2.16 | 8.03 | 1.99 | 0.62 |

As can be seen, the control strain expressing the FAR1 reductase but no desaturase was able to produce the 016 fatty alcohols Z9-16:OH and Z11-16:OH, but did not produce any detectable 014 fatty alcohols (E11-14:OH and Z11-14:OH). The three tested strains were also able to produce the 016 fatty alcohols, and all of them were in addition able to produce the 014 fatty alcohols Z11-14:OH, and to a lesser degree also E11-14:OH. The above data also show that none of the three tested desaturases, when introduced in the cell, results in significant changes in production of Z11-16:OH compared to the control strain without desaturase. The data thus show that these three desaturases also have a higher specificity towards C14 substrates than towards C16 substrates.

Example 15—Co-expression of fatty acyl-CoA reductases with Ncb5or Three insect Ncb5or and one human Ncb5or gene were co-expressed in Y. lipolytica in combination with a fatty acyl-CoA reductase from Helicoverpa armigera (FAR1) or one insect Ncb5or was co-expressed with a fatty acyl-CoA reductase from Agrotis segetum (FAR12). The strains were cultivated, and fatty alcohols analyzed as described in Example 3.

The presence of insect Ncb5or improved the activity of FAR1 reductase in Y. lipolytica by 3-10% (Table 16) and of FAR12 by 4% (Table 17).

TABLE 16
Effect of Ncb5or on the activity of
reductase FAR1 from H. armigera.

| | | Total fatty | |
|---|---|---|---|
| Strain | Ncb5or | alcohols (mg/L) | Improvement (%) |
| ST10623 | — | 819 ± 84 | Reference |
| ST10624 | CpNcb5or | 900 ± 23 | 10 |
| ST10786 | DgNcb5or | 890 ± 250 | 9 |
| ST10787 | LboNcb5or | 841 ± 21 | 3 |
| ST10788 | HsNcb5or | 687 ± 50 | −16 |

TABLE 17
Effect of Ncb5or on the activity of
reductase FAR12 from A. segetum.

| | | Total fatty | |
|---|---|---|---|
| Strain | Ncb5or | alcohols (mg/L) | Improvement (%) |
| ST10665 | — | 80 ± 18 | Reference |
| ST10666 | CpNcb5or | 83 ± 15 | 4 |

### Example 16—Co-Expression of Fatty Acyl-CoA Desaturases with Ncb5or

Two insect Ncb5or genes were co-expressed in Y. lipolytica in combination with a fatty acyl-CoA desaturase from Spodoptera litura (Desat38), or a fatty acyl-CoA desaturase from Lobesia botrana (Desat30), or a fatty acyl-CoA desaturase from Drosophila virilis (Desat61), respectively. The strains were cultivated and FAMEs analyzed as described in Example 3.

The presence of insect Ncb5or improved the activity of Desat38 desaturase in Y. lipolytica by 26 to 36% (Table 18), Desat30 by 9-27% (Table 19), and Desat61 by 11-21% (Table 20).

TABLE 18
Effect of Ncb5or on the activity of
desaturase Desat38 from S. litura.

| | | Z11-16:Me | |
|---|---|---|---|
| Strain | Ncb5or | purity (%) | Improvement (%) |
| ST10615 | — | 26 ± 0 | Reference |
| ST10616 | CpNcb5or | 36 | 38 |
| ST10784 | LboNcb5or | 33 | 26 |

TABLE 19
Effect of Ncb5or on the activity of desaturase Desat30 from
L. botrana. Strains in this table carry a mutation in
Fas2p that increases tetradecanoyl-CoA biosynthesis.

| Strain | Ncb5or | Z11-14:Me (mg/L) | Improvement (%) |
|---|---|---|---|
| ST10610 | — | 11 ± 2 | Reference |
| ST10611 | CpNcb5or | 14 ± 0 | 27 |
| ST10613 | LboNcb5or | 12 ± 3 | 9 |

TABLE 20
Effect of Ncb5or on the activity of desaturase Desat61 from
D. virilis. Strains in this table carry a mutation in
Fas2p that increases tetradecanoyl-CoA biosynthesis.

| | | Z9-14:Me | |
|---|---|---|---|
| Strain | Ncb5or | purity (%) | Improvement (%) |
| ST10672 | — | 3 ± 0 | Reference |
| ST10673 | CpNcb5or | 4 ± 0 | 21 |
| ST10790 | LboNcb5or | 4 ± 0 | 11 |

### Example 17—Cytochrome B5 and Cytochrome B5 Reductase from Mortierella alpina Co-Expression with Desaturase

A cytochrome B5 (MaCytB5) and cytochrome B5 reductase 1 or 2 (MaCytB5 Red1 or 2), respectively, of Mortierella alpina or Ncb5or from Cydia pomonella (CpNcb5or) were co-expressed in Y. lipolytica in combination with fatty acyl-CoA Δ11 desaturase from Cadra cautella (Desat70). The resulting strains are listed in Table 21.

Combining the expression of Desat70 with the C. pomonella Ncb5or improved the purity of Z11-16:Me by 46% compared to a strain expressing only Desat70, while when combined with the cytochrome B5 and cytochrome B5 reductase pairs from M. alpina the purity improved only 14 and 29%.

TABLE 21
Effect of Ncb5or from C. pomonella or cytochrome B5 and B5
reductase from M. alpina on the activity of fatty acyl-CoA
desaturase Desat70. Strains in this table carry a mutation
in Fas2p that increases tetradecanoyl-CoA biosynthesis.

| | | Z11-16:Me | |
|---|---|---|---|
| Strain | Genes expressed | purity (%) | Improvement (%) |
| ST12195 | Desat70 | 13.8 ± 1 | — |
| ST12178 | Desat70 | 20.2 ± 0.1 | 46 |
| | CpNcb5or | | |
| ST12521 | Desat70 | 15.7 ± 2 | 14 |
| | MaCytb5 | | |
| | MaCytB5Red2 | | |
| ST12520 | Desat70 | 17.8 ± 3 | 29 |
| | MaCytb5 | | |
| | MaCytB5Red1 | | |

### Example 18—Ncb5or from Cydia pomonella Co-Expression with Various Fatty Acyl Co-A Reductase

Fatty acyl-CoA reductases from different organisms were either expressed alone (control strains) or in combination with Ncb5or of Cydia pomonella (CpNcb5or). The strains were cultivated, and fatty alcohols analyzed as described in Example 3, except from that the cells were inoculated into YPG medium without adjustment of the optical density and the incubation in YPG medium was prolonged from 24 to 48 hours. The total fatty alcohol production titers of the strains are shown in Table 22. Total fatty alcohol production improvements between 0-50% in comparison to the control are marked with a “+”, 51-100% with a “++” and >100% with a “+++”.

The expression of Ncb5or from Cydia pomonella improved the activity all fatty acyl Co-A reductases in Y. lipolytica (Table 22).

TABLE 22
Effect of Ncb5or from Cydia pomonella on production of
fatty alcohols by indicated fatty acyl-CoA reductases

| Strain | Fatty acyl- | Control strain | Improvement in total |
|---|---|---|---|
| expressing | CoA | without | fatty alcohol production |
| CpNcb5or | reductase | CpNcb5or | over control |
| ST12073 | FAR2 | ST12118 | + |
| ST12074 | FAR4 | ST12119 | + |
| ST12076 | FAR6 | ST12121 | + |
| ST12077 | FAR8 | ST12122 | ++ |
| ST12081 | FAR42 | ST12126 | +++ |
| ST12082 | FAR11 | ST12127 | + |
| ST12083 | FAR25 | ST12128 | + |
| ST12084 | FAR13 | ST12129 | ++ |
| ST12087 | FAR16 | ST12132 | +++ |
| ST12088 | FAR17 | ST12133 | +++ |
| ST12089 | FAR18 | ST12134 | + |
| ST12096 | FAR28 | ST12141 | ++ |
| ST12098 | FAR30 | ST12143 | + |
| ST12100 | FAR43 | ST12145 | + |
| ST12101 | FAR33 | ST12146 | ++ |
| ST12102 | FAR34 | ST12147 | +++ |
| ST12103 | FAR35 | ST12148 | ++ |
| ST12104 | FAR44 | ST12149 | + |
| ST12107 | FAR45 | ST12152 | + |
| ST12112 | FAR41 | ST12157 | + |
| ST12116 | FAR46 | ST12161 | ++ |
| ST12115 | FAR47 | ST12160 | + |

### Example 19—Ncb5or from Cydia pomonella Co-Expression with Various Fatty Acyl Co-A Desaturases

Fatty acyl-CoA desaturases from different organisms were either expressed alone (control strains) or in combination with Ncb5or of Cydia pomonella (CpNcb5or). The strains were cultivated, and FAME analysis were performed as described in Example 3, except from that the cells were inoculated into YPG medium without adjustment of optimal density and the incubation time in YPG medium was prolonged from 24 to 48 hours. The purity of the target desaturated fatty acid methyl esters are shown in Table 23. Purity improvements between 0-50% in comparison to the control are marked with a “+”, 51-100% with a “++” and >100% with a “+++”.

All desaturases listed below showed a higher activity when combined with Ncb5or from Cydia pomonella.

TABLE 23
Effect of Ncb5or from Cydia pomonella on production of desaturated
fatty acid methyl esters by indicated fatty acyl-CoA desaturases

| Strain | Fatty acyl- | Target fatty | Control strain | |
|---|---|---|---|---|
| expressing | CoA | acid methyl | without | Improvement |
| CpNcb5or | desaturase | ester | CpNcb5or | in purity (%) |
| ST12241 | Desat69 | Z9-14:Me | ST12242 | + |
| ST12241 | Desat69 | Z9-16:Me | ST12242 | + |
| ST12241 | Desat69 | Z9-18:Me | ST12242 | + |
| ST12163 | Desat16 | Z11-16:Me | ST12180 | + |
| ST12164 | Desat19 | Z11-16:Me | ST12181 | + |
| ST12166 | Desat21 | Z11-16:Me | ST12183 | + |
| ST12169 | Desat44 | Z11-16:Me | ST12186 | + |
| ST12170 | Desat45 | Z11-16:Me | ST12187 | + |
| ST12535 | Desat37 | Z11-16:Me | ST12534 | + |
| ST12173 | Desat63 | Z11-16:Me | ST12190 | +++ |
| ST12174 | Desat65 | Z11-16:Me | ST12191 | + |
| ST12175 | Desat71 | Z11-16:Me and | ST12192 | ++ |
| | | Z11-18:Me | | |
| ST12177 | Desat72 | Z11-16:Me and | ST12194 | + |
| | | Z11-18:Me | | |
| ST12178 | Desat70 | Z11-16:Me | ST12195 | + |
| ST12179 | Desat73 | Z11-16:Me | ST12196 | +++ |
| ST12197 | Desat24 | Z9-14:Me | ST12210 | ++ |
| ST12198 | Desat56 | Z9-14:Me | ST12211 | ++ |
| ST12200 | Desat59 | Z9-14:Me | ST12213 | ++ |
| ST12201 | Desat60 | Z9-14:Me | ST12214 | ++ |
| ST12202 | Desat61 | Z9-14:Me | ST12215 | ++ |
| ST12205 | Desat43 | Z9-16:Me | ST12218 | + |
| ST12208 | Desat27 | Z9-14:Me and | ST12221 | + |
| | | Z9-16:Me | | |
| ST12209 | Desat75 | Z9-14:Me and | ST12222 | +++ |
| | | Z9-16:Me | | |
| ST12223 | Desat30 | E11-14:Me and | ST12232 | +++ |
| | | Z11-14:Me | | |
| ST12224 | Desat35 | E11-14:Me and | ST12233 | +++ |
| | | Z11-14:Me | | |
| ST12225 | Desat36 | E11-14:Me and | ST12234 | +++ |
| | | Z11-14:Me | | |
| ST12226 | Desat52 | E11-14:Me and | ST12235 | +++ |
| | | Z11-14:Me | | |
| ST12227 | Desat76 | E11-14:Me and | ST12236 | +++ |
| | | Z11-14:Me | | |
| ST12229 | Desat77 | E14-16:Me | ST12238 | + |
| ST12231 | Desat78 | Z11-18:Me | ST12240 | + |

### Example 20—Co-Expression of Ncb5or from Agrotis segetum or Bombus terrestris with Desaturase Desat70 or Fatty Acyl Co-A Reductase

Ncb5or genes from Agrotis segetum and Bombus terrestris were co-expressed in Y. lipolytica in combination with the Cadra cautella desaturase Desat70 or a fatty acyl-CoA reductase from Helicoverpa armigera (FAR1), respectively. The strains were cultivated, FAMEs and fatty alcohols analyzed as described in Example 3.

The presence of Ncb5or from Agrotis segetum and Bombus terrestris improved the activity of Desat70 in Y. lipolytica by 26.8 and 28.8%, respectively (Table 24). The activity of FAR1 reductase in Y. lipolytica improved by 25.8 and 0.65% when combined with Ncb5or from Agrotis segetum and Bombus terrestris, respectively (Table 25).

TABLE 24
Effect of Ncb5or from Agrotis segetum and Bombus terrestris
on activity of desaturase Desat70 from Cadra cautella.

| Strain | Genes expressed | Improvement purity (%) |
|---|---|---|
| ST12195 | Desat70 | Reference |
| ST12518 | AseNcb5or Desat70 | 26.8 |
| ST12519 | BterNcb5or Desat70 | 28.2 |

TABLE 25
Effect of Ncb5or from Agrotis segetum and Bombus terrestris
on activity of reductase FAR1 from H. armigera.

| | | Improvement in total |
|---|---|---|
| Strain | Genes expressed | fatty alcohol titer (%) |
| ST12118 | FAR1 | Reference |
| ST12395 | AseNcb5or FAR1 | 25.8 |
| ST12397 | BterNcb5or FAR1 | 0.6 |

### Sequence Overview

Please note that SEQ ID NOs: 40, 43-74, 94-100, 104-110, 115-118, 125, 140-153, 168-180 and 186-189 have been codon optimized for Yarrowia lipolytica. SEQ ID NOs: 39, 75, 101-103 and 181 have been codon optimized for Saccharomyces cerevisiae.

Desaturases

| SEQ | SEQ | | | |
|---|---|---|---|---|
| ID NO, | ID NO, | | | |
| Protein | DNA | Name | Native to | Desaturase |
| 1 | 39 | Desat19 | Agrotis segetum | Δ11 desaturase |
| 2 | 40 | Desat16 | Amyelois transitella | Δ11 desaturase |
| 3 | 41 | Desat17 | Amyelois transitella | Δ9 desaturase |
| 4 | 42 | Desat18 | Amyelois transitella | Δ9 desaturase |
| 5 | 43 | Desat25 | Chauliognathus lugubris | Δ9 desaturase |
| 6 | 44 | Desat47 | Chilo supprealis | Desaturase |
| 7 | 45 | Desat36 | Choristoneura parallela | Δ11 desaturase |
| 8 | 46 | Desat35 | Choristoneura rosaceana | Δ11 desaturase |
| 9 | 47 | Desat4 | Cydia pomonella | Desaturase |
| 10 | 48 | Desat2 | Cydia pomonella | Desaturase |
| 11 | 49 | Desat1 | Cydia pomonella | Desaturase |
| 12 | 50 | Desat40 | Dendrolimus punctatus | Δ9 desaturase |
| 13 | 51 | Desat59 | Drosophila grimshawi | Δ9 desaturase |
| 14 | 52 | Desat24 | Drosophila melanogaster | Δ9 desaturase |
| 15 | 53 | Desat61 | Drosophila virilis | Δ9 desaturase |
| 16 | 54 | Desat33 | Epiphyas postvittana | Desaturase |
| 17 | 55 | Desat31 | Grapholita molesta | Δ10 desaturase |
| 18 | 56 | Desat55 | Grapholita molesta | desaturase |
| 19 | 57 | Desat51 | Helicoverpa zea | Desaturase |
| 20 | 58 | Desat30 | Lobesia botrana | Desaturase |
| 21 | 59 | Desat43 | Lobesia botrana | Desaturase |
| 22 | 60 | Desat52 | Manducta sexta | Δ11 desaturase |
| 23 | 61 | Desat32 | Ostrinia nubilalis | Δ11 desaturase |
| 24 | 62 | Desat48 | Pectinophora gossypiella | Δ11 desaturase |
| 25 | 63 | Desat22 | Pelargonium hortorum | Δ9 desaturase |
| 26 | 64 | Desat45 | Plutella xylostella | Δ11 desaturase |
| 27 | 65 | Desat23 | Ricinus communis | Δ9 desaturase |
| 28 | 66 | Desat42 | Saccharomyces cerevisiae | Δ9 desaturase |
| 29 | 67 | Desat37 | Spodoptera exigua | Δ11 desaturase |
| 30 | 68 | Desat20 | Spodoptera littoralis | Δ11 desaturase |
| 31 | 69 | Desat20 | Spodoptera littoralis | Δ11 desaturase |
| 32 | 70 | Desat38 | Spodoptera litura | Δ11 desaturase |
| 33 | 71 | Desat26 | Spodoptera litura | Desaturase |
| 34 | 72 | Desat34 | Thaumetopoea pityocampa | ΔZ11-14 |
| | | | | desaturase |
| 35 | 73 | Desat28 | Tribolium castaneum | Desaturase |
| 36 | 74 | Desat29 | Tribolium castaneum | Desaturase |
| 37 | 75 | Desat21 | Trichoplusia ni | Δ11 desaturase |
| 38 | 76 | Desat69 | Yarrowia lipolytica | Δ9 desaturase |
| 126 | 140 | Desat72 | Antheraea pernyi | Δ11 desaturase |
| 127 | 141 | Desat76 | Argyrotaenia velutinana | Δ11 desaturase |
| 128 | 142 | Desat75 | Bombus lapidarius | Δ9 desaturase |
| 129 | 143 | Desat78 | Bombyx mori | Δ11 desaturase |
| 130 | 144 | Desat44 | Chilo supprealis | Δ11 desaturase |
| 131 | 145 | Desat60 | Drosophila ananassae | Δ9 desaturase |
| 132 | 146 | Desat63 | Diatraea saccharalis | Z11 desaturase |
| 133 | 147 | Desat56 | Drosophila yakuba | Δ9 desaturase |
| 134 | 148 | Desat70 | Cadra cautella | Δ11 desaturase |
| 135 | 149 | Desat71 | Lobesia botrana | Δ11 desaturase |
| 136 | 150 | Desat77 | Ostrinia furnacalis | ΔE14 |
| | | | | desaturase |
| 137 | 151 | Desat65 | Plodia interpunctella | Δ11 desaturase |
| 138 | 152 | Desat27 | Tribolium castaneum | Δ9 desaturase |
| 139 | 153 | Desat73 | Yponomeuta padella | Δ11 desaturase |

FARs

| SEQ ID NO, | SEQ ID NO, | | |
|---|---|---|---|
| Protein | DNA | Name | Native to |
| 77 | 94 | FAR12 | Agrotis segetum |
| 78 | 95 | FAR18 | Agrotis ipsilon |
| 79 | 96 | FAR11 | Bicyclus anynana |
| 80 | 97 | FAR14 | Bombus lapidarius |
| 81 | 98 | FAR13 | Chilo suppressalis |
| 82 | 99 | FAR23 | Cydia pomonella |
| 83 | 100 | FAR1 | Helicoverpa armigera |
| 84 | 101 | FAR6 | Helicoverpa assulta |
| 85 | 102 | FAR4 | Heliothis subflexa |
| 86 | 103 | FAR5 | Heliothis virescens |
| 87 | 104 | FAR27 | Plutella xylostella |
| 88 | 105 | FAR16 | Spodoptera exigua |
| 89 | 106 | FAR22 | Spodoptera frugiperda |
| 90 | 107 | FAR15 | Spodoptera littoralis |
| 91 | 108 | FAR19 | Spodoptera litura |
| 92 | 109 | FAR25 | Tyta alba |
| 93 | 110 | FAR38 | Trichoplusia ni |
| 154 | 168 | FAR33 | Amyelois transitella |
| 155 | 169 | FAR34 | Amyelois transitella |
| 156 | 170 | FAR35 | Amyelois transitella |
| 157 | 171 | FAR47 | Chrysodeixis includens |
| 158 | 172 | FAR46 | Cydia pomonella |
| 159 | 173 | FAR42 | Marinobacter algicola |
| 160 | 174 | FAR43 | Manducta sexta |
| 161 | 175 | FAR44 | Ostrinia furnacalis |
| 162 | 176 | FAR28 | Plodia interpunctella |
| 163 | 177 | FAR30 | Plodia interpunctella |
| 164 | 178 | FAR17 | Spodoptera exigua |
| 165 | 179 | FAR45 | Spodoptera exigua |
| 166 | 180 | FAR41 | Trichoplusia ni |
| 167 | 181 | FAR8 | Yponomeuta rorellus |

Ncb5ors

| SEQ ID NO, | SEQ ID NO, | | |
|---|---|---|---|
| Protein | DNA | Name | Native to |
| 111 | 115 | DgNcb5or | Drosophila grimshawi |
| 112 | 116 | DmNcb5or | Drosophila melanogaster |
| 113 | 117 | HsNcb5or | Homo sapiens |
| 114 | 118 | SlitNcb5or | Spodoptera litura |
| 124 | 125 | CpoNcb5or1 | Cydia pomonella |
| 182 | 186 | CpNcb5or | Cydia pomonella |
| 183 | 187 | AseNcb5or | Agrotis segetum |
| 184 | 188 | BterNcb5or | Bombus terrestris |
| 185 | 189 | LboNcb5or | Lobesia botrana |

Other

| SEQ ID NO, | | |
|---|---|---|
| Protein | Name | Native to |
| 119 | Fao1 | Yarrowia lipolytica |
| 120 | GPAT | Yarrowia lipolytica |
| 121 | Hdf1 | Yarrowia lipolytica |
| 122 | Hfd4 | Yarrowia lipolytica |
| 123 | Pex10 | Yarrowia lipolytica |

Cytochrome b5 and cytochrome b5 reductases

| SEQ ID NO, | | |
|---|---|---|
| Protein | Name | Native to |
| 190 | MaCytB5 | Mortierella alpina |
| 191 | MaCytB5Red1 | Mortierella alpina |
| 192 | MaCytB5Red2 | Mortierella alpina |

### REFERENCES

Alfaro et al. 2009. Optimization of pheromone dispenser density for managing the rice striped stem borer, Chilo suppressalis (Walker), by mating disruption. Crop Protection. 28:567-572.
Bari, 2003. Development of pheromone mating disruption strategies for the suppression of the artichoke plume moth in artichokes grown on the central coast of California. ISHS Acta Horticulturae 660: V International Congress on Artichoke. doi: 10.17660/ActaHortic.2004.660.80
Deng, B et al. (2010). Study of the individual cytochrome b5 and cytochrome b5 reductase domains of Ncb5or reveals a unique heme pocket and a possible role the CS domain. The Journal of Biological Chemistry, 30181-30191.
Eizaguirre et al. 2002. Effects of mating disruption against the Mediterranean corn borer, Sesamia nonagrioides, on the European corn borer Ostrinia nubilalis. Use of pheromones and other semiochemicals in integrated production IOBC wprs Bulletin.
Ferrell & Yao (1972). Reductive and oxidative synthesis of saturated and unsaturated fatty aldehydes, J Lipid Res. 13(1):23-6.
Holkenbrink et al. (2018). EasyCloneYALI: CRISPR/Cas9-Based Synthetic Toolbox for Engineering of the Yeast Yarrowia lipolytica. Biotechnol J.
Holkenbrink et al. (2020). Production of moth sex pheromones for pest control by yeast fermentation. Metab Eng., 312-321.
Jensen, N., Strucko, T., Kildegaard, K., David, F., Maury, J., Mortensen, U., Borodina, I. (2014). EasyClone: method for iterative chromosomal integration of multiple genes in Saccharomyces cerevisiae. FEMS Yeast Research, 238-48.
Kehat & Dunkelblum, 1993. Sex Pheromones: achievements in monitoring and mating disruption of cotton pests in Israel, Achieves of Insect Biochemistry and Physiology. 22:425-431.
Larade et al. (2008). Loss of Ncb5or Results in Impaired Fatty Acid Desaturation, lipoatrophy and diabetes. The Journal of Biological Chemistry, 29285-29291.
Li et al. (2009). An environmentally benign TEMPO-catalyzed efficient alcohol oxidation system with a recyclable hypervalent iodine(III) reagent and its facile preparation. Synthesis, 1163-1169a.
Meyer & Schreiber (1994). Acceleration of the Dess-Martin oxidation by water. J. Org. Chem., 59, 7549-7552.
Okada (2014). Sodium hypochlorite pentahydrate (NaOCI-5H2O) crystals as an extra-ordinary oxidant for primary and secondary alcohols. Synlett, 25, 596-598.
Petkevicius et al. (2021). Biotechnological production of the European corn borer sex pheromone in the yeast Yarrowia lipolytica. Biotechnology Journal.
Steves & Stahl (2013). Copper(I)/ABNO-catalyzed aerobic alcohol oxidation: alleviating steric and electronic constraints of Cu/TEMPO catalyst systems. J. Am. Chem. Soc., 135, 15742-15745.
Tamura et al. (2012). Novel [4-Hydroxy-TEMPO+NaCl]/SiO2 as a reusable catalyst for aerobic oxidation of alcohols to carbonyls. Synlett, 23, 1397-1407.
Tupec et al. (2017). Biotechnological potential of insect fatty acid-modifying enzymes. Zeitschrift fQr Naturforschung.
Yadav et al. (2004). Recyclable 2^{nd} generation ionic liquids as green solvents for the oxidation of alcohols with hypervalent iodine reagents. Tetrahedron, 60, 2131-2135.
Wu et al. 2012. Management of diamondback moth, Plutella xylostella (Lepidoptera: Plutellidae) by mating disruption. Insect Science 19 (6), 643-648.
Zambo et al. (2020). Investigation of the putative rate-limiting role of electron. FEBS Letters, 530-539.

### Items

1. A cell expressing:
i) a first enzyme or group of enzymes capable of converting a fatty acyl-CoA to a compound selected from a desaturated fatty alcohol, a saturated fatty alcohol, a desaturated fatty alcohol acetate, a desaturated fatty acyl-CoA and a desaturated fatty acid; and
ii) a heterologous NAD(P)H cytochrome b5 oxidoreductase (Ncb5or);
whereby the cell is capable of producing the compound with a higher titer compared to a cell expressing the first group of enzymes but no heterologous Ncb5or when cultivated in the same conditions.

2. A cell expressing
i) a first enzyme or group of enzymes capable of converting a fatty acyl-CoA to a compound selected from a desaturated fatty alcohol, a saturated fatty alcohol, a desaturated fatty alcohol acetate and a desaturated fatty acyl-CoA; and
ii) a heterologous NAD(P)H cytochrome b5 oxidoreductase (Ncb5or);
whereby the cell is capable of producing the compound with a higher titer compared to a cell expressing the first group of enzymes but no heterologous Ncb5or when cultivated in the same conditions.

3. The cell according to any one of items 1 or 2, wherein the first enzyme or group of enzymes comprises or consists of one or more desaturase capable of converting a fatty acyl-CoA to a desaturated fatty acyl-CoA, whereby the cell is capable of producing a desaturated fatty acyl-CoA with a higher titer compared to a cell expressing said one or more desaturase but no heterologous Ncb5or when cultivated in the same conditions.

4. The cell according to any one of items 1 or 2, wherein the first enzyme or group of enzymes comprises or consists of one or more fatty acyl reductase (FAR) capable of converting a fatty acyl-CoA to a fatty alcohol, whereby the cell is capable of producing a fatty alcohol with a higher titer compared to a cell expressing said one or more FAR but no heterologous Ncb5or when cultivated in the same conditions.

5. The cell according to any one of items 1 or 2, wherein the first enzyme or group of enzymes comprises or consists of one or more FAR and one or more desaturase capable of converting a fatty acyl-CoA to a desaturated fatty alcohol, whereby the cell is capable of producing a desaturated fatty alcohol with a higher titer compared to a cell expressing said one or more FAR and said one or more desaturase but no heterologous Ncb5or when cultivated in the same conditions.

6. The cell according to any one of the preceding items, further expressing an acetyltransferase capable of converting a desaturated or a saturated fatty alcohol to a desaturated or a saturated fatty alcohol acetate, respectively, whereby the cell is capable of producing a desaturated or a saturated fatty alcohol acetate with a higher titer compared to a cell expressing the first group of enzymes and the acetyltransferase but no heterologous Ncb5or when cultivated in the same conditions.

7. The cell according to any one of the preceding items, wherein the Ncb5or is native to a plant, an insect or a mammal, such as Homo sapiens.

8. The cell according to any one of the preceding items, wherein the Ncb5or is native to an insect, such as an insect of the genus Agrotis, Amyelois, Aphantopus, Arctia, Bicyclus, Bombyx, Bombus, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia, and Vanessa.

9. The cell according to any one of the preceding items, wherein the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni, and Vanessa tameamea.

10. The cell according to any one of the preceding items, wherein the Ncb5or is selected from the group of Ncb5ors set forth in SEQ ID NOs: 111 to 114, SEQ ID NO: 124 and SEQ ID NO: 182 to 185, or variants having at least 70% identity thereto, such as at least 75% identity, such as at least 80% identity, such as at least 85% identity, such as at least 90% identity, such as at least 95% identity thereto.

11. The cell according to any one of the preceding items, wherein the Ncb5or is selected from:
a. an Ncb5or from Drosophila melanogaster or a variant thereof having at least 80% identity thereto, preferably the Ncb5or from Drosophila melanogasteras set forth in SEQ ID NO: 112 (DmNcb5or) or a functional variant thereof having at least 80% identity thereto;
b. an Ncb5or from Spodoptera litura or a variant thereof having at least 80% identity thereto, preferably the Ncb5or from Spodoptera litura (SlitNcb5or) as set forth in SEQ ID NO: 114 or a functional variant thereof having at least 80% identity thereto;
c. an Ncb5or from Drosophila grimshawi or a variant thereof having at least 80% identity thereto, preferably the Ncb5or from Drosophila grimshawi as set forth in SEQ ID NO: 111 (DmNcb5or) or a functional variant thereof having at least 80% identity thereto;
d. an Ncb5or from Homo sapiens or a variant thereof having at least 80% identity thereto, preferably the Ncb5or from Homo sapiens as set forth in SEQ ID NO: 113 (HsNcb5or) or a functional variant thereof having at least 80% identity thereto;
e. an Ncb5or from Cydia pomonella or a variant thereof having at least 80% identity thereto, preferably the Ncb5or from Cydia pomonella as set forth in SEQ ID NO: 124 (CpoNcb5or1) or SEQ ID NO: 182 (CpoNcb5or), or a functional variant thereof having at least 80% identity thereto;
f. an Ncb5or from Agrotis segetum or a variant thereof having at least 80% identity thereto, preferably the Ncb5or from Agrotis segetum as set forth in SEQ ID NO: 183 (AseNcb5or) or a functional variant thereof having at least 80% identity thereto;
g. an Ncb5or from Bombus terrestris or a variant thereof having at least 80% identity thereto, preferably the Ncb5or from Bombus terrestris as set forth in SEQ ID NO: 184 (BterNcb5or) or a functional variant thereof having at least 80% identity thereto; and
h. an Ncb5or from Lobesia botrana or a variant thereof having at least 80% identity thereto, preferably the Ncb5or from Lobesia botrana as set forth in SEQ ID NO: 185 (LboNcb5or) or a functional variant thereof having at least 80% identity thereto.

12. The cell according to any one of the previous items, wherein the heterologous Ncb5or is one or more heterologous Ncb5ors, such as a plurality of different heterologous Ncb5ors.

13. The cell according to any one of the preceding items, wherein the desaturase is native to a plant, such as Ricinus communis or Pelargonium hortorum, or an insect, such as the Diptera, the Coleoptera, or the Lepidoptera order, such as of the genus Agrotis, Antheraea, Argyrotaenia, Amyelois, Bombus, Bombyx, Cadra, Chauliognathus, Chilo, Choristoneura, Cydia, Dendrophilus, Diatraea, Drosophila, Ephestia, Epiphyas, Grapholita, Helicoverpa, Lampronia, Lobesia, Manducta, Ostrinia, Pectinophora, Plodia, Plutella, Thalassiosira, Thaumetopoea, Tribolium, Trichoplusia, Spodoptera or Yponomeuta, such as Agrotis segetum, Antheraea pernyi, Argyrotaenia velutiana, Amyelois transitella, Bombus lapidarius, Bombyx mori, Cadra cautella, Chauliognathus lugubris, Chilo supprealis, Choristoneura parallela, Choristoneura rosaceana, Cydia pomonella, Dendrophilus punctatus, Diatraea saccharalis, Drosophila ananassae, Drosophila melanogaster, Drosophila virilis, Drosophila yakuba, Ephestia elutella, Ephestia kuehniella, Epiphyas postvittana, Grapholita molesta, Helicoverpa assulta, Helicoverpa zea, Lampronia capitella, Lobesia botrana, Manducta sexta, Ostrinia furnacalis, Ostrinia nubilalis, Pectinophora gossypiella, Plodia interpunctella, Plutella xylostella, Spodoptera exigua, Spodoptera littoralis, Spodoptera litura, Thalassiosira pseudonana, Thaumetopoea pityocampa, Tribolium castaneum, Trichoplusia ni or Yponomeuta padella.

14. The cell according to any one of the preceding items, wherein the desaturase is selected from the group consisting of a Δ3 desaturase, a Δ5 desaturase, a Δ6 desaturase, a Δ7 desaturase, a Δ8 desaturase, a Δ9 desaturase, a Δ10 desaturase, a Δ11 desaturase, a Δ12 desaturase, a Δ13 desaturase and a Δ14 desaturase, preferably wherein the desaturase is a Δ9 desaturase or a Δ11 desaturase.

15. The cell according to any one of the preceding items, wherein the desaturase is selected from the group of desaturases set forth in SEQ ID NOs: 1 to 38 and SEQ ID NOs: 126 to 139, or variants having at least 70% identity thereto, such as at least 75% identity, such as at least 80% identity, such as at least 85% identity, such as at least 90% identity, such as at least 95% identity.

16. The cell according to any one of the preceding items, wherein the desaturase is selected from:
a. a Spodoptera litura desaturase, such as Desat38 as set forth in SEQ ID NO: 32;
b. a Lobesia botrana desaturase, such as Desat30 as set forth in SEQ ID NO: 20;
c. a Drosophila virilis desaturase, such as Desat61 as set forth in SEQ ID NO: 15;
d. a Cadra cautella desaturase, such as Desat70 as set forth in SEQ ID NO: 134;
e. a Yarrowia lipolytica desaturase, such as Desat69 as set forth in SEQ ID NO: 38;
f. an Amyelois transitella desaturase, such as Desat16 as set forth in SEQ ID NO: 2;
g. an Agrotis segetum desaturase, such as Desat19 as set forth in SEQ ID NO:1;
h. a Trichoplusia ni desaturase, such as Desat21 as set forth in SEQ ID NO: 37;
i. a Chilo supprealis desaturase, such as Desat44 as set forth in SEQ ID NO: 130;
j. a Plutella xylostella desaturase, such as Desat45 as set forth in SEQ ID NO: 26;
k. a Spodoptera exigua desaturase, such as Desat37 as set forth in SEQ ID NO: 29;
l. a Diatraea saccharalis Z11 desaturase, such as Desat63 as set forth in SEQ ID NO: 132;
m. a Plodia interpunctella desaturase, such as Desat65 as set forth in SEQ ID NO: 137;
n. a Lobesia botrana desaturase, such as Desat71 as set forth in SEQ ID NO: 135;
o. a Antheraea pernyi desaturase, such as Desat72 as set forth in SEQ ID NO: 126;
p. a Yponomeuta padella desaturase, such as Desat73 as set forth in SEQ ID NO: 139;
q. a Drosophila melanogaster desaturase, such as Desat24 as set forth in SEQ ID NO: 14;
r. a Drosophila yakuba desaturase, such as Desat56 as set forth in SEQ ID NO: 133;
s. a Drosophila grimshawi desaturase, such as Desat59 as set forth in SEQ ID NO: 13;
t. a Drosophila ananassae desaturase, such as Desat60 as set forth in SEQ ID NO: 131;
u. a Drosophila virilis desaturase, such as Desat61 as set forth in SEQ ID NO: 15;
v. a Lobesia botrana desaturase, such as Desat43 as set forth in SEQ ID NO: 21;
w. a Tribolium castaneum desaturase, such as Desat27 as set forth in SEQ ID NO: 138;
x. a Bombus lapidarius desaturase, such as Desat75 as set forth in SEQ ID NO: 128;
y. a Lobesia botrana desaturase, such as Desat30 as set forth in SEQ ID NO: 20;
z. a Choristoneura rosaceana desaturase, such as Desat35 as set forth in SEQ ID NO: 8;
aa. a Choristoneura parallela desaturase, such as Desat36 as set forth in SEQ ID NO: 7;
bb. a Manducta sexta desaturase, such as Desat52 as set forth in SEQ ID NO: 22;
cc. a Argyrotaenia velutinana desaturase, such as Desat76 as set forth in SEQ ID NO: 127;
dd. a Ostrinia furnacalis desaturase, such as Desat77 as set forth in SEQ ID NO: 136;
ee. a Bombyx mori desaturase, such as Desat78 as set forth in SEQ ID NO: 129;
or a functional variant thereof having at least 80% sequence identity thereto.

17. The cell according to any one of the preceding items, wherein the FAR is native to an insect such as an insect of the Lepidoptera order, such as of the genus Agrotis, Amyelois, Bicyclus, Bombus, Chilo, Chrysodeixis, Cydia, Helicoverpa, Heliothis, Manducta, Ostrinia, Plodia, Plutella, Spodoptera, Trichoplusia, Tyta or Yponomeuta, or wherein the FAR is native to a bacteria, such as of the genus Marinobacter.

18. The cell according to any one of the preceding items, wherein the FAR is a fatty acyl reductase native to Agrotis segetum, Amyelois transitella, Bicyclus anynana, Bombus lapidaries, Chilo suppressalis, Chrysodeixis includes, Cydia pomonella, Helicoverpa armigera, Helicoverpa assulta, Heliothis virescens, Heliothis subflexa, Manducta sexta, Marinobacter algicola, Ostrinia furnacalis, Plodia interpunctella, Plutella xylostella, Spodoptera exigua, Spodoptera frugiperda, Spodoptera littoralis, Spodoptera litura, Trichoplusia ni, Tyta alba or Yponomeuta rorellus, or a functional variant thereof having at least 80% identity thereto.

19. The cell according to any one of the preceding items, wherein the FAR is selected from the group of FARs set forth in SEQ ID NOs: 77 to 93 and SEQ ID NOs: 154 to 167, or variants having at least 70% identity thereto, such as at least 75% identity, such as at least 80% identity, such as at least 85% identity, such as at least 90% identity, such as at least 95% identity
20. The cell according to any one of the preceding items, wherein the FAR is selected from:
   a. a Spodoptera littoralis FAR, such as the FAR15 set forth in SEQ ID NO: 90;
   b. a Spodoptera exigua FAR, such as the FAR as set forth in SEQ ID NO: 88 (FAR16);
   c. a Helicoverpa armigera FAR, such as the FAR as set forth in SEQ ID NO: 83 (FAR1);
   d. an Agrotis segetum FAR, such as the FAR as set forth in SEQ ID NO: 77 (FAR12);
   e. a Bicyclus anyana FAR, such as the FAR as set forth in SEQ ID NO: 79 (FAR11);
   f. a Cydia pomonella FAR, such as the FAR as set forth in SEQ ID NO: 82 (FAR23);
   g. a Heliothis subflexa FAR, such as the FAR as set forth in SEQ ID NO: 85 (FAR4)
   h. a Helicoverpa assulta FAR, such as the FAR as set forth in SEQ ID NO: 84 (FAR6);
   i. a Tyta alba FAR, such as the FAR as set forth in SEQ ID NO: 92 (FAR25);
   j. a Heliothis virescens FAR, such as the FAR as set forth in SEQ ID NO: 86 (FAR5);
   k. a Yponomeuta rorellus FAR, such as the FAR as set forth in SEQ ID NO: 167 (FAR8);
   l. a Marinobacter algicola FAR, such as the FAR as set forth in SEQ ID NO: (FAR159);
   m. a Chilo suppressalis FAR, such as the FAR as set forth in SEQ ID NO: 81 (FAR13);
   n. a Spodoptera exigua FAR, such as the FAR as set forth in SEQ ID NO: 164 (FAR17);
   o. a Agrotis ipsilon FAR, such as the FAR as set forth in SEQ ID NO: 78 (FAR18);
   p. a Plodia interpunctella FAR, such as the FAR as set forth in SEQ ID NO: 162 (FAR28);
   q. a Plodia interpunctella FAR, such as the FAR as set forth in SEQ ID NO: 163 (FAR30);
   r. a Manducta sexta FAR, such as the FAR as set forth in SEQ ID NO: 160 (FAR43);
   s. a Amyelois transitella FAR, such as the FAR as set forth in SEQ ID NO: 154 (FAR33);
   t. a Amyelois transitella FAR, such as the FAR as set forth in SEQ ID NO: 155 (FAR34);
   u. a Amyelois transitella FAR, such as the FAR as set forth in SEQ ID NO: 156 (FAR35);
   v. a Ostrinia furnacalis FAR, such as the FAR as set forth in SEQ ID NO: 161 (FAR44);
   w. a Spodoptera exigua FAR, such as the FAR as set forth in SEQ ID NO: 165 (FAR45);
   x. a Trichoplusia ni FAR, such as the FAR as set forth in SEQ ID NO: 166 (FAR41);
   y. a Cydia pomonella FAR, such as the FAR as set forth in SEQ ID NO: 158 (FAR46);
   z. a Chrysodeixis includens FAR, such as the FAR as set forth in SEQ ID NO: 157 (FAR47);
   or a functional variant thereof having at least 80% identity thereto.

21. The cell according to any one of the preceding items, wherein at least one of the genes encoding a desaturase, a FAR, or an Ncb5or is present in high copy number.

22. The cell according to any one of the preceding items, wherein at least one of the genes encoding a desaturase, a FAR, or an Ncb5or is under the control of an inducible promoter.

23. The cell according to any one of the preceding items, wherein at least one of the genes encoding a desaturase, a FAR, or an Ncb5or is codon-optimized for said cell.

24. The cell according to any one of the preceding items, wherein the genes encoding a desaturase, a FAR, or an Ncb5or are each independently comprised within the genome of the cell or within a vector comprised within the cell.

25. The cell according to any one of the preceding items, wherein the desaturated fatty alcohol, the saturated fatty alcohol, the desaturated fatty alcohol acetate, and/or the saturated fatty alcohol acetate have a carbon chain length of 8, 9, 10, 11, 12, 13, 14, 15, 16, 17, 18, 19, 20, 21 or 22, preferably the carbon chain has a length of 11, 12, 13, 14, 15, 16, 17 or 18.

26. The cell according to any one of the preceding items, wherein the desaturated fatty alcohol is desaturated in at least one position, such as at least two positions.

27. The cell according to any one of the preceding items, wherein the desaturated fatty alcohol is desaturated at position 3, 4, 5, 6, 7, 8, 9, 10, 11, 12, 13, 14, 15, 16, 17, 18, 19, 20 or21.

28. The cell according to any one of the preceding items, wherein the desaturated fatty alcohol is selected from the group of desaturated fatty alcohols consisting of (Z)-9-tetradecen-1-ol (Z9-14:OH), (Z)-9-hexadecen-1-ol (Z9-16:OH), (Z)-11-tetradecen-1-ol (Z11-14:OH), (Z)-11-hexadecen-1-ol (Z11-16:OH), and codlemone (E8,E10-dodecadien-1-ol).

29. The cell according to any one of the preceding items, wherein the desaturated fatty alcohol acetate is desaturated in at least one position, such as at least two positions.

30. The cell according to any one of the preceding items, wherein the desaturated fatty alcohol acetate is desaturated at position 3, 4, 5, 6, 7, 8, 9, 10, 11, 12, 13, 14, 15, 16, 17, 18, 19, 20 or21.

31. The cell according to any one of the preceding items, wherein the desaturated fatty alcohol acetate is E8,E10-dodecadienyl acetate.

32. The cell according to any one of the preceding items, wherein the cell expresses an Ncb5or selected from the group consisting of SEQ ID NOs: 111 to 114, SEQ ID NO: 124 and SEQ ID NOs: 182 to 185, and
a. a desaturase selected from the group consisting of SEQ ID NOs: 1 to 38 and SEQ ID NOs: 126 to 139, and a FAR selected from the group consisting of SEQ ID NOs: 77 to 93 and SEQ ID NOs 154 to 167;
b. a desaturase from Spodoptera litura (Desat38) as set forth in SEQ ID NO: 32, and a FAR from Helicoverpa armigera (FAR1) as set forth in SEQ ID NO: 83; or
c. a desaturase from Lobesia botrana (Desat30) as set forth in SEQ ID NO: 20, and a FAR from Helicoverpa armigera (FAR1) as set forth in SEQ ID NO: 83; or
d. a desaturase from Drosophila virilis (Desat61) as set forth in SEQ ID NO: 15, and a FAR from Helicoverpa armigera (FAR1) as set forth in SEQ ID NO: 83;
or functional variants thereof having at least 80% identity thereto.

33. The cell according to any one of the preceding items, wherein the cell expresses:
a. a Spodoptera litura desaturase such as Desat38 as set forth in SEQ ID NO: 32; a Helicoverpa armigera FAR such as FAR1 as set forth in SEQ ID NO: 83; and an Nc5bor selected from the group consisting of a Drosophila melanogaster Ncb5or, such as DmNcb5or as set forth in SEQ ID NO: 112, and a Spodoptera litura Ncb5or such as SlitNcb5or as set forth in SEQ ID NO: 114; or
b. a Lobesia botrana desaturase such as Desat30 as set forth in SEQ ID NO: 20; a Helicoverpa armigera FAR such as FAR1 as set forth in SEQ ID NO: 83; and an Nc5bor selected from the group consisting of a Drosophila melanogaster Ncb5or such as DmNcb5or as set forth in SEQ ID NO: 112, a Spodoptera litura Ncb5or such as SlitNcb5or as set forth in SEQ ID NO: 114, a Drosophila grimshawi Ncb5or such as DgNcb5or as set forth in SEQ ID NO: 111, and a Homo sapiens Ncb5or such as HsNcb5or as set forth in SEQ ID NO:113; or
c. a Drosophila virilis desaturase such as Desat61 as set forth in SEQ ID NO: 15; a Helicoverpa armigera FAR such as FAR1 as set forth in SEQ ID NO: 83; and an Nc5bor selected from the group consisting of a Drosophila melanogaster Ncb5or such as DmNcb5or as set forth in SEQ ID NO: 112, and a Drosophila grimshawi Ncb5or such as DgNcb5or as set forth in SEQ ID NO: 111.

34. The cell according to any one of the preceding items, wherein said cell is a yeast cell, preferably an oleaginous yeast cell.

35. The yeast cell according to item 34, wherein the genus of said yeast cell is selected from the group consisting of Saccharomyces, Pichia, Yarrowia, Kluyveromyces, Candida, Rhodotorula, Rhodosporidium, Cryptococcus, Trichosporon and Lipomyces.

36. The yeast cell according to any one of items 34 to 35, wherein the yeast is selected from the group consisting of Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica.

37. The cell according to any one of items 1 to 33, wherein said cell is a plant cell.

38. The plant cell according to item 37, wherein the genus of said plant is selected from the group consisting of Nicotiana and Camelina.

39. The plant cell according to any one items 37 to 38, wherein said plant is selected from the group consisting of Nicotiana tabacum, Nicotiana benthamiana, and Camelina sativa.

40. A method for increasing the activity of at least one enzyme selected from the group consisting of desaturases and fatty acyl CoA reductases (FARs), said method comprising the steps of:
a. providing a desaturase capable of introducing at least one double bond in a fatty acyl-CoA, thereby converting at least part of said fatty acyl-CoA to a desaturated fatty-acyl-CoA; and/or
b. providing a FAR capable of converting at least part of said desaturated fatty acyl-CoA to a desaturated fatty alcohol, thereby producing said desaturated fatty alcohol; and
c. contacting said desaturase and/or FAR with an Ncb5or, thereby increasing the activity of said desaturase and/or FAR compared to the activity of said desaturase and/or FAR in the absence of said Ncb5or, wherein the activity is measured under the same conditions;
wherein the increase in activity is measured by measuring the concentration of product formed by the desaturase and/or the FAR.

41. The method according to item 40, wherein the method is performed in vitro.

42. The method according to item 40, wherein the method is performed in vivo, preferably wherein the method is performed in:
a. a yeast cell according to any one of items 34 to 36; or
b. a plant cell according to any one of items 37 to 39.

43. A method for production of a compound selected from a desaturated fatty alcohol, a saturated fatty alcohol, a desaturated fatty alcohol acetate, a desaturated fatty acid and a desaturated fatty acyl-CoA in a cell, said method comprising the steps of:
a. providing a cell and incubating said cell in a medium; and
b. expressing in said cell a first enzyme or group of enzymes capable of converting a fatty acyl-CoA to said compound, thereby converting at least part of said fatty acyl-CoA to said compound; and
c. expressing in said cell an Ncb5or;
d. optionally, recovering said compound.

44. A method for increasing the titer of a compound selected from a desaturated fatty alcohol, a saturated fatty alcohol, a desaturated fatty alcohol acetate, a desaturated fatty acid and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the titer of said compound compared to the titer from a cell not expressing said Ncb5or in the same conditions;
c. optionally, recovering said compound.

45. The method according to any one of items 43 to 44, wherein the first enzyme or group of enzymes consists of one or more desaturase capable of converting a fatty acyl-CoA to a desaturated fatty acyl-CoA and wherein said compound is a desaturated fatty acyl-CoA or a desaturated fatty acid.

46. The method according to any one of items 43 to 44, wherein the first enzyme or group of enzymes consists of one or more FAR capable of converting a fatty acyl-CoA to a saturated fatty alcohol and wherein said compound is a saturated fatty alcohol.

47. The method according any one of items 43 to 44, wherein the first enzyme or group of enzymes consists of one or more FAR and one or more desaturase capable of converting a fatty acyl-CoA to a desaturated fatty alcohol and wherein said compound is a desaturated fatty alcohol.

48. The method according to any one of items 43 to 47, wherein the compound is a desaturated or a saturated fatty alcohol and wherein the method further comprises the step of converting the desaturated or the saturated fatty alcohol to a desaturated or a saturated fatty alcohol acetate, respectively.

49. The method according to item 48, wherein the conversion of the desaturated or the saturated fatty alcohol to a desaturated or a saturated fatty alcohol acetate is performed in vitro.

50. The method according to item 48, wherein the conversion of the desaturated or the saturated fatty alcohol to a desaturated or a saturated fatty alcohol acetate is performed in vivo by further expressing in the cell an acetyltransferase capable of converting the desaturated or the saturated fatty alcohol to a desaturated or a saturated fatty alcohol acetate, respectively.

51. The method according to any one of items 43 to 50, wherein said compound is a saturated fatty alcohol or a desaturated fatty alcohol, and wherein the method further comprises a step of converting the saturated fatty alcohol or the desaturated fatty alcohol to a saturated fatty aldehyde or to a desaturated fatty aldehyde, respectively.

52. The method according to item 51, wherein the conversion to an aldehyde is a chemical conversion or an enzymatic conversion.

53. The method according to any one of items 43 to 52, wherein the cell is a yeast cell as defined in any one of items 34 to 36.

54. The method according to any one of items 43 to 53, wherein the total titer of fatty alcohols and optionally the total titer of fatty alcohol acetates is increased, wherein the total titer of fatty alcohols is the sum of the titer of desaturated fatty alcohols and the titer of saturated fatty alcohols, and the total titer of fatty alcohol acetates is the sum of the titer of desaturated fatty alcohol acetates and the titer of saturated fatty alcohol acetates.

55. The method according to any one of items 43 to 54, wherein the titer of saturated fatty alcohols and optionally the titer of saturated fatty alcohol acetates is increased.

56. The method according to any one of items 43 to 55, wherein the titer of desaturated fatty alcohols and/or fatty alcohol acetates and/or fatty acids and/or saturated fatty alcohols and/or fatty alcohol acetates and/or the total titer of saturated fatty alcohols and/or fatty alcohol acetates is increased at least 3% compared to the titer from a cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

57. The method according to any one of items 40 to 56, wherein the desaturase is as defined in any one of items 13 to 16.

58. The method according to any one of items 40 to 57, wherein the FAR is as defined in any one of items 17 to 20.

59. The method according to any one of items 40 to 58, wherein the Ncb5or is as defined in any one of items 7 to 12.

60. The method according to any one of items 40 to 59, wherein the desaturated fatty alcohol, the saturated fatty alcohol, the desaturated fatty alcohol acetate and/or the saturated fatty alcohol acetate is as defined in item 25.

61. The method according to any one of items 40 to 60, wherein the desaturated fatty alcohol is as defined in any one of items 26 to 28.

62. The method according to any one of items 40 to 61, wherein the desaturated fatty alcohol acetate is as defined in any one of items 29 to 31.

63. The method according to any one of items 40, 42 to 44, and 54 to 62, wherein the cell is a plant cell is as defined in any one of items 37 to 39.

64. The method according to any one of items 40 to 63, further comprising the step of formulating the desaturated fatty alcohol, the desaturated fatty alcohol acetate, and/or the desaturated fatty aldehyde in a pheromone composition.

65. The method according to item 64, wherein the pheromone composition further comprises one or more additional compounds such as a liquid or solid carrier or substrate.

66. The method according to any one of items 40 to 65, wherein the method yields said desaturated fatty alcohol, saturated fatty alcohol, desaturated fatty alcohol acetate, saturated fatty alcohol acetate, desaturated fatty aldehyde, and/or saturated fatty aldehyde with a titer of at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

67. The method according to any one of items 40 to 66, further comprising converting the saturated fatty alcohol or the desaturated fatty alcohol to a saturated fatty aldehyde or to a desaturated fatty aldehyde, respectively, by expression of at least one alcohol dehydrogenase and/or at least one fatty alcohol oxidase in said yeast cell.

68. A system of nucleic acid constructs comprising nucleic acids encoding an Ncb5or and:
a. a desaturase capable of introducing at least one double bond in a fatty acyl-CoA; and/or
b. a fatty acyl CoA reductase (FAR) capable of converting at least part of a desaturated fatty acyl-CoA to a desaturated fatty alcohol.

69. The system according to item 68, wherein the Ncb5or is encoded by any one of the sequences set forth in SEQ ID NOs: 115 to 118, SEQ ID NO: 125 and SEQ ID NOs: 186 to 189, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

70. The system according to any one of items 68 to 69, wherein the desaturase is encoded by any one of the sequences set forth in SEQ ID NOs: 39 to 76 and SEQ ID NOs: 140 to 153, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

71. The system according to any one of items 68 to 70, wherein the FAR is encoded by any one of the sequences set forth in SEQ ID NOs: 94 to 110 and SEQ ID NOs: 168 to 181, or variants thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

72. A kit of parts comprising:
a. the cell according to any one of items 1 to 33; and/or
b. the nucleic acid system according to any one of items 68 to 71, wherein said construct is for modifying a cell; and
c. instructions for use; and
d. optionally the cell to be modified.

73. The kit of parts according to item 72, wherein the cell is a yeast cell according to any one of items 34 to 36.

74. The kit of parts according to item 72, wherein the cell is a plant cell according to any one of items 37 to 39.

75. Use of an Ncb5or in a method for increasing the activity of one or more enzymes.

76. The use according to item 75, wherein the one or more enzymes is one or more membrane-bound enzymes.

77. The use according to any one of items 75 to 76, wherein the one or more enzymes is selected from the group consisting of desaturases and reductases.

78. The use according any one of items 75 to 77, wherein the increase in activity of said of one or more enzymes is at least 1.2-fold for the desaturase and/or the FAR, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, such as at least 30-fold, such as at least 40-fold, such as at least 50-fold; wherein the increase in activity of said of one or more enzymes is compared to the activity of said one or more enzymes in the absence of said Ncb5or, wherein the activity is measured under the same conditions, wherein the increase is measured by measuring the concentration of product formed by the one or more enzymes.

79. The use according to any one of items 75 to 78, wherein the increase in activity of said of one or more enzymes is at least 1.2-fold for the desaturase and/or the FAR, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, such as at least 30-fold, such as at least 40-fold, such as at least 50-fold; wherein the increase in activity of said of one or more enzymes is compared to the activity of said one or more enzymes in the absence of said Ncb5or, wherein the activity is measured under the same conditions, wherein the increase is measured by measuring the concentration of product formed by the desaturase and/or the FAR.

80. The use according to any one of items 75 to 79, wherein the Ncb5or, the desaturase and/or the FAR are as defined in any one of the preceding claims.

81. The use according to any one of items 75 to 80, wherein the Ncb5or is selected from the group consisting of SEQ ID NOs: 111 to 114, SEQ ID NO: 124 and SEQ ID NO: 182 to 185.

82. The use according to any one of items 75 to 81, wherein the one or more enzymes is selected from the group consisting of SEQ ID NOs: 1 to 38, SEQ ID NOs: 77 to 93, SEQ ID NOs: 140 to 153 and SEQ ID NOs: 168 to 181.

83. The use according to any one of items 75 to 82, wherein the one or more enzymes is selected from:
a. a desaturase from Spodoptera litura (Desat38) as set forth in SEQ ID NO: 32, and a FAR from Helicoverpa armigera (FAR1) as set forth in SEQ ID NO: 83; or
b. a desaturase from Lobesia botrana (Desat30) as set forth in SEQ ID NO: 20, and a FAR from Helicoverpa armigera (FAR1) as set forth in SEQ ID NO: 83; or
c. a desaturase from Drosophila virilis (Desat61) as set forth in SEQ ID NO: 15, and a FAR from Helicoverpa armigera (FAR1) as set forth in SEQ ID NO: 83;
or functional variants thereof having at least 80% identity thereto.

84. The use according to any one of items 75 to 83, wherein the Nc5bor is selected from the group consisting of a Drosophila melanogaster Ncb5or, such as DmNcb5or as set forth in SEQ ID NO: 112; a Spodoptera litura Ncb5or such as SlitNcb5or as set forth in SEQ ID NO: 114; a Drosophila grimshawi Ncb5or such as DgNcb5or as set forth in SEQ ID NO: 111; a Homo sapiens Ncb5or such as HsNcb5or as set forth in SEQ ID NO: 113; a Cydia pomonella Ncb5or such as CpoNcb5or1 as set forth in SEQ ID NO: 124 or CpNcb5or as set forth in SEQ ID NO: 182; an Agrotis segetum Ncb5or such as AseNcb5or as set forth in SEQ ID NO: 183; a Bombus terrestris Ncb5or such as BterNcb5or as set forth in SEQ ID NO: 184; a Lobesia botrana Ncb5or such as LboNcb5or as set forth in SEQ ID NO: 185; or functional variants thereof having at least 80% identity thereto.

85. A desaturated fatty alcohol, a saturated fatty alcohol, a desaturated fatty alcohol acetate, a saturated fatty alcohol acetate, a desaturated fatty aldehyde and/or a saturated fatty aldehyde obtainable by the method according to any one of items 40 to 67.

86. Use of a desaturated fatty alcohol, a saturated fatty alcohol, a desaturated fatty alcohol acetate, a saturated fatty alcohol acetate, a desaturated fatty aldehyde and/or a saturated fatty aldehyde obtainable by the method according to any one of items 40 to 67.

87. A method of monitoring the presence of pest or disrupting the mating of pest, said method comprising the steps of:
a. producing a desaturated fatty alcohol and optionally a desaturated fatty alcohol acetate and/or a desaturated fatty aldehyde according to the method of any one of items 40 to 67; and
b. formulating said fatty alcohol and optionally said fatty alcohol acetate and/or said desaturated fatty aldehyde as a pheromone composition; and
c. employing said pheromone composition as an integrated pest management composition.

88. A fermentation broth containing the yeast cell according to any one of items 34 to 36.

89. A fermentation system or a catalytic system comprising the yeast cell according to any one of items 34 to 36.

90. A device, such as a pheromone dispenser, for diffusing a pheromone composition, said pheromone composition comprising the desaturated fatty alcohol and/or the desaturated fatty alcohol acetate and/or the desaturated fatty aldehyde according to any one of items 25 to 31 or 85.

91. A method for producing at least 1 mg/L of a desaturated fatty alcohol, a saturated fatty alcohol, a desaturated fatty alcohol acetate, a saturated fatty alcohol acetate, a desaturated fatty aldehyde and/or a saturated fatty aldehyde in a cell, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more, wherein the method is according to any one of items 40 to 67.

92. The method according to any one of items 43 to 67 or 91, wherein the medium comprises an extractant in an amount equal to or greater than its cloud concentration in an aqueous solution, wherein the extractant a non-ionic surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone and ethoxylated and propoxylated C₁₆-C₁₈ alcohol-based antifoaming agents and combinations thereof.

93. The method according to item 92, wherein:
the non-ionic surfactant is an ethoxylated and propoxylated C₁₆-C₁₈ alcohol-based antifoaming agent, such as C₁₆-C₁₈ alkyl alcohol ethoxylate propoxylate (CAS number 68002-96-0), and wherein the culture medium comprises at least 1% vol/vol of C₁₆-C₁₈ alkyl alcohol ethoxylate propoxylate,
the non-ionic surfactant is a polyethylene polypropylene glycol, for example Kollliphor® P407 (CAS number 9003-11-6), and wherein the culture medium comprises at least 10% vol/vol of polyethylene polypropylene glycol such as Kolliphor® P407,
the non-ionic surfactant is a mixture of polyether dispersions, such as antifoam 204, and wherein the culture medium comprises at least 1% vol/vol of a mixture of polyether dispersions such as antifoam 204; and/or
the non-ionic surfactant is a non-ionic surfactant comprising polyethylene glycol monostearate such as simethicone, and wherein the culture medium comprises at least 1% vol/vol of polyethylene glycol monostearate or simethicone.

94. The method according to any one of items 43 to 67 or 91 to 93, wherein the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.

95. The method according to any one of items 43 to 67 and 91 to 94, wherein the cell is a yeast cell and wherein:
a. the desaturated fatty alcohol is (Z)-11-hexadecen-1-ol; and
b. the desaturase is a Δ11-desaturase selected from the group consisting of the Amyelois transitella Δ11-desaturase (Desat16; SEQ ID NO: 2), the Spodoptera littoralis Δ11-desaturase (Desat20; SEQ ID NO: 31), the Agrotis segetum Δ11-desaturase (Desat19; SEQ ID NO: 1) and the Trichoplusia ni Δ11-desaturase (Desat21; SEQ ID NO: 37) or a variant thereof having at least 65% identity, such as at least 70% identity, such as at least 71% identity, such as at least 72%, such as at least 73%, such as at least 74%, such as at least 75%, such as at least 80%, such as at least 85%, such as at least 90%, such as at least 95%, such as 100% identity to Desat16 (SEQ ID NO: 2), Desat20 (SEQ ID NO: 31), Desat19 (SEQ ID NO: 1), or Desat21 (SEQ ID NO: 37); and
c. the FAR is selected from the group consisting of FAR1 (SEQ ID NO: 83), FAR4 (SEQ ID NO: 85), and FAR6 (SEQ ID NO: 84), or a variant thereof having at least 80% identity, such as at least 85%, such as at least 90%, such as at least 95%, such as 100% identity to FAR1 (SEQ ID NO: 83), FAR4 (SEQ ID NO: 85), or FAR6 (SEQ ID NO: 84);
wherein said Δ11-desaturase is capable of converting at least part of hexadecanoyl-CoA to (Z)11-hexadecenoyl-CoA, and said FAR is capable of converting at least part of (Z)11-hexadecenoyl-CoA to (Z)-11-hexadecanol, thereby obtaining (Z)-11-hexadecen-1-ol with a titre of at least 0.2 mg/L.

96. The yeast cell according to any one of items 34 to 36, wherein said yeast cell is an oleaginous yeast cell, wherein said oleaginous yeast cell has a mutation resulting in reduced activity of the fatty alcohol oxidase Fao1 (SEQ ID NO: 119) and a mutation resulting in reduced activity of at least one of the fatty aldehyde dehydrogenase Hfd1 (SEQ ID NO: 121), the fatty aldehyde dehydrogenase Hfd4 (SEQ ID NO: 122), the peroxisome biogenesis factor Pex10 (SEQ ID NO: 123), and the glycerol-3-phosphate acyltransferase GPAT (SEQ ID NO: 120), or having a mutation resulting in reduced activity of at least one protein having at least 60% identity to Fao1 (SEQ ID NO: 119) and a mutation resulting in reduced activity of at least one of Hfd1 (SEQ ID NO: 121), Hfd4 (SEQ ID NO: 122), Pex10 (SEQ ID NO: 123) and GPAT (SEQ ID NO: 120), such as at least 65% identity, such as at least 70% identity, such as at least 75% identity, such as at least 80% identity, such as at least 81% identity, such as at least 82% identity, such as at least 83% identity, such as at least 84% identity, such as at least 85% identity, such as at least 86% identity, such as at least 87% identity, such as at least 88% identity, such as at least 89% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity to Fao1 (SEQ ID NO: 119) and at least one of Hfd1 (SEQ ID NO: 121), Hfd4 (SEQ ID NO: 122), Pex10 (SEQ ID NO: 123) and GPAT (SEQ ID NO: 120), preferably wherein the yeast cell has at least a mutation resulting in reduced activity of Fao1 and at least one mutation resulting in reduced activity of Hfd1, Hfd4, Pex10 and GPAT.

97. The yeast cell according to any one of items 34 to 36, wherein said yeast cell expresses a desaturase which has a higher specificity towards tetradecanoyl-CoA than towards hexadecanoyl-CoA and/or wherein the FAR has a higher specificity towards desaturated tetradecanoyl-CoA than towards desaturated hexadecanoyl-CoA.

98. The yeast cell according to any one of items 34 to 36, wherein said yeast cell:
a. has one or more mutations resulting in reduced activity of one or more native acyl-CoA oxidases; and
b. expresses at least one first group of enzymes comprising at least one acyl-CoA oxidase capable of oxidising a fatty acyl-CoA, wherein the first group of enzymes is capable of shortening a fatty acyl-CoA of a first carbon chain length X to a shortened fatty acyl-CoA having a second carbon chain length X′, wherein X′≤X−2; and
c. expresses at least one heterologous desaturase capable of introducing at least one double bond in said fatty acyl-CoA and/or in said shortened fatty acyl-CoA; and
d. expresses at least one heterologous FAR, capable of converting at least part of said desaturated fatty acyl-CoA to a desaturated fatty alcohol; and
e. optionally expresses at least one acetyltransferase capable of converting at least part of said desaturated fatty alcohol to a desaturated fatty alcohol acetate, and/or at least one alcohol dehydrogenase and/or fatty alcohol oxidase capable of converting at least part of said desaturated fatty alcohol to a desaturated fatty aldehyde.

99. The yeast cell according to any one of items 34 to 36, wherein said yeast cell is capable of producing E8,E10-dodecadienyl coenzyme A and optionally E8,E10-dodecadien-1-ol, said yeast cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA), wherein:
a. the at least one desaturase is Desat4 (SEQ ID NO: 9), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to SEQ ID NO: 9; or
b. the at least one desaturase is at least two desaturases, wherein at least one of said two desaturases is Desat4 (SEQ ID NO: 9), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to SEQ ID NO: 9, and the other desaturase is a desaturase capable of introducing at least one double bond in a fatty acyl-CoA having a carbon chain length of 12, such as a Z9-12 desaturase.

100. A method for increasing the purity of a compound selected from a a desaturated fatty alcohol, a desaturated fatty acid and a desaturated fatty acyl-CoA produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the production of said compound compared to the production from a cell not expressing said Ncb5or in the same conditions;
wherein the purity of said compound is the ratio or percentage of said compound in relation to all compounds within the same compound group produced by the cell, such as the percentage of said desaturated fatty alcohol in relation to all desaturated fatty alcohols produced by the cell, such as the percentage of desaturated fatty acid in relation to all fatty acids produced by the cell, and/or such as the percentage of desaturated fatty acyl-CoA in relation to all fatty acyl-CoA produced by the cell.

101. The method according to item 100, wherein the purity of desaturated fatty alcohol, desaturated fatty acid and/or desaturated fatty acyl-CoA is increased at least 3% compared to the purity from a cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

102. The method according to any one of items 100 to 101, wherein the first enzyme or group of enzymes comprises
a. a desaturase as defined in any one of items 13 to 16; and/or
b. a FAR as defined in any one of items 17 to 20.

103. The method according to any one of items 100 to 102, wherein the Ncb5or is as defined in any one of items 7 to 12.

104. The method according to any one of items 100 to 103, wherein the cell is a yeast, wherein the genus of said yeast cell is selected from the group consisting of Saccharomyces, Pichia, Yarrowia, Kluyveromyces, Candida, Rhodotorula, Rhodosporidium, Cryptococcus, Trichosporon and Lipomyces, such as a yeast selected from the group consisting of Saccharomyces cerevisiae, Saccharomyces boulardi, Pichia pastoris, Kluyveromyces marxianus, Candida tropicalis, Cryptococcus albidus, Lipomyces lipofera, Lipomyces starkeyi, Rhodosporidium toruloides, Rhodotorula glutinis, Trichosporon pullulan and Yarrowia lipolytica.

105. The method according to any one of items 100 to 103, wherein the cell is a plant cell, wherein the genus of said plant is selected from the group consisting of Nicotiana and Camelina, such as a plant selected from the group consisting of Nicotiana tabacum, Nicotiana benthamiana, and Camelina sativa.

106. The method according to any one of items 100 to 105, wherein the compound:
a. has a carbon chain length of 8, 9, 10, 11, 12, 13, 14, 15, 16, 17, 18, 19, 20, 21 or 22, preferably the carbon chain has a length of 11, 12, 13, 14, 15, 16, 17or18;
b. is desaturated at position 3, 4, 5, 6, 7, 8, 9, 10, 11, 12, 13, 14, 15, 16, 17, 18, 19, 20 or 21;
c. is desaturated in at least one position, such as at least two positions; and/or
d. is selected from the group consisting of Z9-14:acid, Z9-16:acid, Z11-14:acid, Z11-16:acid, E8,E10-12:acid, of (Z)-9-tetradecen-1-ol (Z9-14:OH), (Z)-9-hexadecen-1-ol (Z9-16:OH), (Z)-11-tetradecen-1-ol (Z11-14:OH), (Z)-11-hexadecen-1-ol (Z11-16:OH), and codlemone (E8,E10-dodecadien-1-ol).

1. A cell expressing:
i) a first enzyme or group of enzymes capable of converting a fatty acyl-CoA to a desaturated fatty alcohol, a saturated fatty alcohol, a desaturated fatty alcohol acetate, a desaturated fatty acyl-CoA, or a combination thereof, and
ii) a heterologous NAD(P)H cytochrome b5 oxidoreductase (Ncb5or);
whereby the cell is capable of producing the compound with a higher titer and/or purity compared to a cell expressing the first group of enzymes but no heterologous Ncb5or when cultivated in the same conditions,
preferably wherein said cell is a yeast cell, such as an oleaginous yeast cell, or a plant cell.

2. The cell according to claim 1, wherein the first enzyme or group of enzymes is:
a) one or more desaturase capable of converting a fatty acyl-CoA to a desaturated fatty acyl-CoA, whereby the cell is capable of producing a desaturated fatty acyl-CoA with a higher titer and/or purity compared to a cell expressing said one or more desaturase but no heterologous Ncb5or when cultivated in the same conditions;
b) one or more fatty acyl reductase (FAR) capable of converting a fatty acyl-CoA to a fatty alcohol, whereby the cell is capable of producing a fatty alcohol with a higher titer and/or purity compared to a cell expressing said one or more FAR but no heterologous Ncb5or when cultivated in the same conditions; or
c) one or more FAR and one or more desaturase capable of converting a fatty acyl-CoA to a desaturated fatty alcohol, whereby the cell is capable of producing a desaturated fatty alcohol with a higher titer and/or purity compared to a cell expressing said one or more FAR and said one or more desaturase but no heterologous Ncb5or when cultivated in the same conditions.

3. The cell according to claim 1, wherein the Ncb5or is native to a plant, an insect or a mammal, such as Homo sapiens, preferably wherein the Ncb5or is native to an insect, such as an insect of the genus Agrotis, Amyelois, Aphantopus, Arctia, Bicyclus, Bombus, Bombyx, Chilo, Cydia, Danaus, Drosophila, Eumeta, Galleria, Helicoverpa, Heliothis, Hyposmocoma, Leptidea, Lobesia, Manduca, Operophtera, Ostrinia, Papilio, Papilio, Papilio, Pieris, Plutella, Spodoptera, Trichoplusia and Vanessa, such as wherein the Ncb5or is native to an insect selected from Agrotis segetum, Amyelois transitella, Aphantopus hyperantus, Arctia plantaginis, Bicyclus anynana, Bombus terrestris, Bombyx mandarina, Bombyx mori, Chilo suppressalis, Cydia pomonella, Danaus plexippus, Drosophila grimshawi, Drosophila melanogaster, Eumeta japonica, Galleria mellonella, Helicoverpa armigera, Heliothis virescens, Hyposmocoma kahamanoa, Leptidea sinapis, Lobesia botrana, Manduca sexta, Operophtera brumata, Ostrinia furnacalis, Papilio machaon, Papilio polytes, Papilio xuthus, Pieris rapae, Plutella xylostella, Spodoptera frugiperda, Spodoptera litura, Trichoplusia ni and Vanessa tameamea.

4. The cell according to claim 1, wherein the Ncb5or is an Ncb5or set forth in SEQ ID NOs: 111 to 114, SEQ ID NO: 124 or SEQ ID NOs: 182 to 185, or variants having at least 70% identity thereto, such as at least 75% identity, such as at least 80% identity, such as at least 85% identity, such as at least 90% identity, such as at least 95% identity thereto.

5. The cell according to claim 1, wherein the desaturase is native to a plant, such as Ricinus communis or Pelargonium hortorum, or an insect, such as of the Diptera, the Coleoptera, or the Lepidoptera order, such as of the genus Agrotis, Antheraea, Argyrotaenia, Amyelois, Bombus, Bombyx, Cadra, Chauliognathus, Chilo, Choristoneura, Cydia, Dendrophilus, Diatraea, Drosophila, Ephestia, Epiphyas, Grapholita, Helicoverpa, Lampronia, Lobesia, Manducta, Ostrinia, Pectinophora, Plodia, Plutella, Thalassiosira, Thaumetopoea, Tribolium, Trichoplusia, Spodoptera or Yponomeuta, such as Agrotis segetum, Antheraea pernyi, Argyrotaenia velutiana, Amyelois transitella, Bombus lapidarius, Bombyx mori, Cadra cautella, Chauliognathus lugubris, Chilo supprealis, Choristoneura parallela, Choristoneura rosaceana, Cydia pomonella, Dendrophilus punctatus, Diatraea saccharalis, Drosophila ananassae, Drosophila melanogaster, Drosophila virilis, Drosophila yakuba, Ephestia elutella, Ephestia kuehniella, Epiphyas postvittana, Grapholita molesta, Helicoverpa assulta, Helicoverpa zea, Lampronia capitella, Lobesia botrana, Manducta sexta, Ostrinia furnacalis, Ostrinia nubilalis, Pectinophora gossypiella, Plodia interpunctella, Plutella xylostella, Spodoptera exigua, Spodoptera littoralis, Spodoptera litura, Thalassiosira pseudonana, Thaumetopoea pityocampa, Tribolium castaneum, Trichoplusia ni or Yponomeuta padella.

6. The cell according to claim 1, wherein the desaturase is a Δ3 desaturase, a Δ5 desaturase, a Δ6 desaturase, a Δ7 desaturase, a Δ8 desaturase, a Δ9 desaturase, a Δ10 desaturase, a Δ11 desaturase, a Δ12 desaturase, a Δ13 desaturase and a Δ14 desaturase, preferably wherein the desaturase is a Δ9 desaturase or a Δ11 desaturase, and/or wherein the desaturase is a desaturase set forth in SEQ ID NOs: 1 to 38 or SEQ ID NOs: 126 to 139, or variants having at least 70% identity thereto, such as at least 75% identity, such as at least 80% identity, such as at least 85% identity, such as at least 90% identity, such as at least 95% identity.

7. The cell according to claim 1, wherein the FAR is native to an insect such as an insect of the Lepidoptera order, such as of the genus Agrotis, Amyelois, Bicyclus, Bombus, Chilo, Chrysodeixis, Cydia, Helicoverpa, Heliothis, Manducta, Ostrinia, Plodia, Plutella, Spodoptera, Trichoplusia, Tyta or Yponomeuta, or wherein the FAR is native to a bacteria, such as of the genus Marinobacter, preferably wherein the FAR is a fatty acyl reductase native to Agrotis segetum, Amyelois transitella, Bicyclus anynana, Bombus lapidaries, Chilo suppressalis, Chrysodeixis includes, Cydia pomonella, Helicoverpa armigera, Helicoverpa assulta, Heliothis virescens, Heliothis subflexa, Manducta sexta, Marinobacter algicola, Ostrinia furnacalis, Plodia interpunctella, Plutella xylostella, Spodoptera exigua, Spodoptera frugiperda, Spodoptera littoralis, Spodoptera litura, Trichoplusia ni, Tyta alba or Yponomeuta rorellus, or a functional variant thereof having at least 80% identity thereto.

8. The cell according to claim 1, wherein the FAR is a FAR set forth in SEQ ID NOs: 77 to 93 or SEQ ID NOs: 154 to 167, or variants having at least 70% identity thereto, such as at least 75% identity, such as at least 80% identity, such as at least 85% identity, such as at least 90% identity, such as at least 95% identity.

9. A method for increasing the activity of desaturases or fatty acyl CoA reductases (FARs), or a combination thereof, said method comprising the steps of:
a. providing a desaturase capable of introducing at least one double bond in a fatty acyl-CoA, thereby converting at least part of said fatty acyl-CoA to a desaturated fatty-acyl-CoA; and/or
b. providing a FAR capable of converting at least part of said desaturated fatty acyl-CoA to a desaturated fatty alcohol, thereby producing said desaturated fatty alcohol; and
c. contacting said desaturase and/or FAR with an Ncb5or, thereby increasing the activity of said desaturase and/or FAR compared to the activity of said desaturase and/or FAR in the absence of said Ncb5or, wherein the activity is measured under the same conditions;
wherein the increase in activity is measured by measuring the concentration and/or purity of product formed by the desaturase and/or the FAR,
preferably wherein said cell is a cell according to claim 1.

10. A method for production of a desaturated fatty alcohol, a saturated fatty alcohol, a desaturated fatty alcohol acetate, or a desaturated fatty acyl-CoA, or a combination thereof, in a cell, said method comprising the steps of:
a. providing a cell and incubating said cell in a medium; and
b. expressing in said cell a first enzyme or group of enzymes capable of converting a fatty acyl-CoA to said compound, thereby converting at least part of said fatty acyl-CoA to said compound; and
c. expressing in said cell an Ncb5or;
d. optionally, recovering said compound,
preferably wherein said cell is a cell according to claim 1.

11. A method for increasing the titer and/or purity of a desaturated fatty alcohol, a saturated fatty alcohol, a desaturated fatty alcohol acetate, or a desaturated fatty acyl-CoA, or a combination thereof, produced in a cell capable of synthesising one or more fatty acyl-CoAs and/or capable of importing fatty acyl-CoAs from its environment, said method comprising the steps of:
a. expressing in said cell a first enzyme or group of enzymes capable of converting a fatty acyl-CoA to said compound, thereby converting at least part of said fatty acyl-CoA to said compound; and
b. expressing in said cell an Ncb5or, thereby increasing the titer and/or purity of said compound compared to the titer and/or purity from a cell not expressing said Ncb5or in the same conditions;
c. optionally, recovering said compound,
preferably wherein said cell is a cell according to claim 1.

12. The method according to claim 11, wherein
the first enzyme or group of enzymes is:
a) one or more desaturase capable of converting a fatty acyl-CoA to a desaturated fatty acyl-CoA and wherein said compound is a desaturated fatty acyl-CoA;
b) one or more FAR capable of converting a fatty acyl-CoA to a saturated fatty alcohol and wherein said compound is a saturated fatty alcohol; or
c) one or more FAR and one or more desaturase capable of converting a fatty acyl-CoA to a desaturated fatty alcohol and wherein said compound is a desaturated fatty alcohol;
and/or wherein
the titer and/or purity of desaturated fatty alcohols and/or fatty alcohol acetates, the titer and/or purity of saturated fatty alcohols and/or fatty alcohol acetates and/or the total titer of saturated fatty alcohols and/or fatty alcohol acetates is increased at least 3% compared to the titer and/or purity from a cell not expressing said Ncb5or, such as at least 4%, such as at least 5%, such as at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 70%, such as at least 80%, such as at least 90%, such as at least 100%, such as at least 150%, such as at least 200%, such as at least 250%.

13. A system of nucleic acid constructs comprising nucleic acids encoding an Ncb5or and:
a. a desaturase capable of introducing at least one double bond in a fatty acyl-CoA; and/or
b. a fatty acyl-CoA reductase capable of converting at least part of a desaturated fatty acyl-CoA to a desaturated fatty alcohol.

14. A method according to claim 9, wherein the increase in activity of said of one or more enzymes is at least 1.2-fold for the desaturase and/or the FAR, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 3-fold, such as at least 4-fold, such as at least 5-fold, such as at least 6-fold, such as at least 7-fold, such as at least 8-fold, such as at least 9-fold, such as at least 10-fold, such as at least 15-fold, such as at least 20-fold, such as at least 30-fold, such as at least 40-fold, such as at least 50-fold; wherein the increase in activity of said one or more enzymes is compared to the activity of said one or more enzymes in the absence of said Ncb5or, wherein the activity is measured under the same conditions, wherein the increase is measured by measuring the concentration of product formed by the one or more enzymes.

15. A method of monitoring the presence of pest or disrupting the mating of pest, said method comprising the steps of:
a. producing a desaturated fatty alcohol and optionally a desaturated fatty alcohol acetate and/or a desaturated fatty aldehyde according to the method of claim 9; and
b. formulating said desaturated fatty alcohol and optionally said desaturated fatty alcohol acetate and/or said desaturated fatty aldehyde as a pheromone composition; and
employing said pheromone composition as an integrated pest management composition.

16. A method of monitoring the presence of pest or disrupting the mating of pest, said method comprising the steps of:
c. producing a desaturated fatty alcohol and optionally a desaturated fatty alcohol acetate and/or a desaturated fatty aldehyde according to the method of claim 12; and
d. formulating said desaturated fatty alcohol and optionally said desaturated fatty alcohol acetate and/or said desaturated fatty aldehyde as a pheromone composition; and
employing said pheromone composition as an integrated pest management composition.
